(19) United States
(12) Patent Application Publication
(10) Pub. No.: US 2009/0035279 A1
(43) Pub. Date: Feb. 5, 2009

(54) GENETIC VARIANTS ON CHR 15Q24 AS MARKERS FOR USE IN DIAGNOSIS, PROGNOSIS AND TREATMENT OF EXFOLIATION SYNDROME AND GLAUCOMA

(75) Inventors: Gudmar Thorleifsson, Reykjavik (IS); Kristinn P. Magnusson, Reykjavik (IS)

Correspondence Address: MARSHALL, GERSTEIN & BORUN LLP, 233 S. WACKER DRIVE, SUITE 6300, SEARS TOWER, CHICAGO, IL, 60606, US

(73) Assignee: deCODE genetics efh, Reykjavik (IS)

(21) Appl. No.: 12/139,374

(22) Filed: Jun. 13, 2008

(30) Foreign Application Priority Data
Jun. 13, 2007 (IS) 8651
Jul. 13, 2007 (IS) 8664
Aug. 8, 2007 (IS) 8668

(51) Int. Cl. A61K 35/76; C12Q 1/68; C12M 1/00; A61K 38/00; A61P 27/00; A01K 67/027; A61K 31/7088

(52) U.S. Cl. 424/93.2; 435/6; 435/287.2; 514/12; 800/3; 514/44

(57) ABSTRACT

The present invention relates to methods of diagnosing a susceptibility to an ocular disorder, including glaucoma and exfoliation syndrome. The invention provides methods of diagnosing an increased or decreased susceptibility to exfoliation syndrome and glaucoma, and methods for risk assessment, treatment and prognosis. The invention further relates to kits for use in the methods of the invention.

This application claims priority benefit of Icelandic Patent Application Nos. 8651, filed Jun. 13, 2007; 8664, filed Jul. 13, 2007, and 8668, filed Aug. 8, 2007. All priority applications are hereby incorporated by reference in their entirety.

### BACKGROUND OF THE INVENTION

Glaucoma is a disease affecting over 2.2 million in the United States and is expected to affect 3.4 million by the year 2020 (Friedman et al., Arch Opthalmol 122, 532 (2004)). Glaucoma is the second most prevalent cause of blindness with 60 million cases worldwide (Resnikoff et al., Bull World Health Organ 82, 844 (2004)). Glaucoma is characterized by progressive loss of vision, and is painless and asymptomatic until late in the disease. The pathophysiology of glaucoma is poorly understood. Therefore, an understanding of its pathogenesis and complications is needed to face the challenge of providing improved risk assessment and better treatment.

Glaucoma is a heterogeneous group of eye disorders, which share a progressive degeneration of retinal ganglion cells and their axons, resulting in the appearance of optic disc and a concomitant pattern of visual loss. In most populations open angle glaucoma (OAG), characterized by painless loss of vision, constitutes the majority of glaucoma cases and is defined as a progressive loss of optic disc neuroretinal rim tissue and consequent excavation of the optic disc with corresponding loss of visual field (Foster, R. et al., Br J Opthalmol 86, 238 (2002); Jonasson, F. et al., Eye 17, 747 (2003)). Open angle glaucoma may be divided into primary open angle glaucoma (POAG) and secondary glaucoma. POAG is without an identifiable cause of aqueous outflow resistance, whereas in secondary glaucoma the outflow resistance is of a known cause and in exfoliation glaucoma (XFG) it is considered to be due to the exfoliative material from which the exfoliation syndrome (XFS) derives its name. POAG is often associated with an elevated intraocular pressure (IOP) (Hollows & Graham, Br J Opthalmol 50, 570 (1966)). POAG is highly familial and age-related (Hewitt, et al, Clin Experiment Opthalmol 34, 472 (2006)), and its risk indicators, vertical optic disc, vertical optic cup, vertical optic cup-to-disc ration and IOP parameters are have high heritability (Klein & Lee, Invest Opthalmol Vis Sci 45, 59 (2004)).

A number of genetic loci for congenital and juvenal glaucoma have been reported (Hewitt et al., Clin Experiment Opthalmol 34, 472 (2006)). However, in adult glaucoma only the MYOC gene, encoding for myocillin, has been shown to confer considerable impact to the disease (Stone et al., Science 275, 668 (1997)). The MYOC has a mutation prevalence of 2-4% in POAG, with over 40 different mutations reported to date, and over 10% in juvenile open angle glaucoma (JOAG). Mutations in the MYOC gene cause dysfunctions in the trabecular meshwork, and genotype-phenotype correlations are strong.

The risk of developing glaucoma increases with several known risk factors. Thus, increased intraocular pressure with increasing age is a major contributor to increased risk of developing glaucoma (Sommer et al., Arch. Opthalmol. 109:1090-95 (1991); Mitchell et al., Opthalmol. 103:1661-69 (1996)). Other risk factors include visual field abnormalities that are observed in otherwise baseline visual field examinations (Kass et al, Arch. Opthalmol. 120:701-13 (2002); Gordon et al., Arch Opthalmol. 120: 714-20 (2002)), high myopia and family history of glaucoma (Wolfs et al., Arch Opthalmol 116:1640-45 (1998); Tielsch et al., Arch Opthalmol. 112: 69-73 (1994)), thin cornea (central corneal thickness of less than 556 μm) and a vertical or horizontal cup-to-disc ratio of greater than 0.4 (Kass et al, Arch. Opthalmol. 120:701-13 (2002); Gordon et al., Arch Opthalmol. 120: 714-20 (2002)). More recently, additional risk factors have been identified, including systemic hypertension, cardiovascular disease, migraine headache, and peripheral vasospasm.

Exfoliation syndrome (XFS), also called pseudoexfoliation syndrome (PEX), is a common age-related eye disorder characterized by elastosis of the extra cellular matrix (ECM) ocular structures that line the anterior segment of the eye. XFS is the largest risk factor for glaucoma (Schlötzer-Schrehardt & Naumann, Am J Opthalmol 141, 921 (2006)), and is also frequently associated with cataract. The disease is characterized by excessive production of extracellular fibrillar material which accumulates on aqueous-bathed surfaces of the anterior segment (Ritch & Schlötzer-Schrehardt, Surv. Opthalmology 45, 265 (2001)). In addition to its intraocular effect, XFS has been shown to be systemic, and appears to be associated with increased cardiovascular and cerebrovascular morbidity (Schlötzer-Schrehardt & Naumann, Am J Opthalmol 141, 921 (2006)). The prevalence of XFS increases with age and a number of articles have pointed to a geographical clustering of XFS although this condition is found worldwide (Ringvold, A., Acta Opthalmol Scand 77, 371 (1999)). XFS is the most common identifiable cause of secondary glaucoma in most populations and is characterized by rapid progression, high resistance to medical therapy, and a worse prognosis than in POAG (Schlotzer-Schrehardt, U. & Naumann, G. O., Am J Opthalmol 141, 921 (2006)).

Genetic risk is conferred by subtle differences in genes among individuals in a population. Genes differ between individuals most frequently due to single nucleotide polymorphisms (SNP), although other variations are also important. SNP are located on average every 500 base pairs in the human genome. Accordingly, a typical human gene containing 250,000 base pairs may contain 500 different SNPs. Only a minor number of SNPs are located in exons and alter the amino acid sequence of the protein encoded by the gene. Most SNPs may have little or no effect on gene function, while others may alter transcription, splicing, translation, or stability of the mRNA encoded by the gene. Additional genetic polymorphism in the human genome is caused by insertion, deletion, translocation, or inversion of either short or long stretches of DNA. Genetic polymorphisms conferring disease risk may therefore directly alter the amino acid sequence of proteins, may increase the amount of protein produced from the gene, or may decrease the amount of protein produced by the gene.

As genetic polymorphisms conferring risk of common diseases are uncovered, genetic testing for such risk factors is becoming important for clinical medicine. Examples are apolipoprotein E testing to identify genetic carriers of the apoE4 polymorphism in dementia patients for the differential diagnosis of Alzheimer's disease, and of Factor V Leiden testing for predisposition to deep venous thrombosis. More importantly, in the treatment of cancer, diagnosis of genetic variants in tumor cells is used for the selection of the most appropriate treatment regime for the individual patient. In breast cancer, genetic variation in estrogen receptor expression or heregulin type 2 (Her2) receptor tyrosine kinase expression determine if anti-estrogenic drugs (tamoxifen) or anti-Her2 antibody (Herceptin) will be incorporated into the treatment plan. In chronic myeloid leukemia (CML) diagnosis of the Philadelphia chromosome genetic translocation fusing the genes encoding the Bcr and Abl receptor tyrosine kinases indicates that Gleevec (STI571), a specific inhibitor of the Bcr-Abl kinase should be used for treatment of the cancer. For CML patients with such a genetic alteration, inhibition of the Bcr-Abl kinase leads to rapid elimination of the tumor cells and remission from leukemia.

Due to the world-wide impact of glaucoma and XFS as major contributors to vision loss at elevated age, there is a great need to understand the biochemical and genetic factors contributing to the diseases. There is also a great need to provide methods of diagnosing susceptibility to these diseases, for use in disease management and individual risk assessment. Further, improved therapeutic methods for preventing and/or ameliorating symptoms associated with these disorders are of great utility.

### SUMMARY OF THE INVENTION

The present invention relates to methods of diagnosing a susceptibility to an ocular disorder (eye disorder), in particular glaucoma and exfoliation syndrome (XFS). The invention includes methods of diagnosing an increased susceptibility as well as methods of diagnosing a decreased susceptibility to XFS or glaucoma, by evaluating certain markers that have been found to be associated with increased and decreased susceptibility of glaucoma and XFS.

The present inventors have discovered that certain variants associated with the LOXL1 gene on chromosome 15q24 are associated with exfoliation syndrome and glaucoma. The inventors have found that certain alleles at certain polymorphic sites are more frequently present in individuals diagnosed with exfoliation syndrome and/or glaucoma than in the general population. Such markers are therefore useful in the various methods of the invention, as will be described in more detail herein. Methods for identifying particular alleles of such markers are provided herein, for use in diagnostic applications.

In one aspect, the invention provides a method of determining a susceptibility to at least one ocular condition selected from exfoliation syndrome and glaucoma in a human individual, the method comprising:
obtaining nucleic acid sequence data about a human individual identifying at least one allele of at least one polymorphic marker associated with the human LOXL1 gene, wherein different alleles of the at least one polymorphic marker are associated with different susceptibilities to the at least one condition in humans, and
determining a susceptibility to at least one condition selected from exfoliation syndrome and glaucoma from the nucleic acid sequence data.

In some embodiments, the glaucoma phenotype is exfoliation glaucoma (XFG). In a general sense, genetic markers lead to alternate sequences at the nucleic acid level. If the nucleic acid marker changes the codon of a polypeptide encoded by the nucleic acid, then the marker will also result in alternate sequence at the amino acid level of the encoded polypeptide (polypeptide markers). Determination of the identity of particular alleles at polymorphic markers in a nucleic acid or particular alleles at polypeptide markers comprises whether particular alleles are present at a certain position in the sequence. Sequence data identifying a particular allele at a marker comprises sufficient sequence to detect the particular allele. For single nucleotide polymorphisms (SNPs) or amino acid polymorphisms described herein, sequence data can comprise sequence at a single position, i.e. the identity of a nucleotide or amino acid at a single position within a sequence.

In certain embodiments, it may be useful to determine the nucleic acid sequence for at least two polymorphic markers. In other embodiments, the nucleic acid sequence for at least three, at least four or at least five or more polymorphic markers is determined. Haplotype information can be derived from an analysis of two or more polymorphic markers. Thus, in certain embodiments, a further step is performed, whereby haplotype information is derived based on sequence data for at least two polymorphic markers.

The invention also provides a method of determining a susceptibility to at least one ocular condition selected from exfoliation syndrome and glaucoma in a human individual, the method comprising obtaining nucleic acid sequence data about a human individual identifying both alleles of at least two polymorphic markers associated with the human LOXL1 gene, determine the identity of at least one haplotype based on the sequence data, and determining a susceptibility to at least one condition selected from exfoliation syndrome and glaucoma from the haplotype data. In some embodiments, the glaucoma phenotype is exfoliation glaucoma.

In certain embodiments, determination of a susceptibility comprises comparing the nucleic acid sequence data to a database containing correlation data between polymorphic markers of the human LOXL1 gene and susceptibility to the at least one condition. In some embodiments, the database comprises at least one risk measure of susceptibility to the at least one ocular condition for the polymorphic markers of the LOXL1 gene. The sequence database can for example be provided as a look-up table that contains data that indicates the susceptibility of the ocular disorder (e.g., glaucoma or exfoliation syndrome) for any one, or a plurality of, particular polymorphisms. The database may also contain data that indicates the susceptibility for a particular haplotype that comprises at least two polymorphic markers.

Obtaining nucleic acid sequence data can in certain embodiments comprise obtaining a biological sample from the human individual and analyzing sequence of the at least one polymorphic marker in nucleic acid in the sample. Analyzing sequence can comprise determining the presence or absence of at least one allele of the at least one polymorphic marker. Determination of the presence of a particular susceptibility allele (e.g., an at-risk allele) is indicative of susceptibility to the ocular condition in the human individual. Determination of the absence of a particular suscepbility allele is indicative that the particular susceptibility is not present in the individual.

In some embodiments, obtaining nucleic acid sequence data comprises obtaining nucleic acid sequence information from a preexisting record. The preexisting record can for example be a computer file or database containing sequence data, such as genotype data, for the human individual, for at least one polymorphic marker.

Susceptibility determined by the diagnostic methods of the invention can be reported to a particular entity. In some embodiments, the at least one entity is selected from the group consisting of the individual, a guardian of the individual, a genetic service provider, a physician, a medical organization, and a medical insurer. In another aspect, the invention relates to a method of diagnosing a susceptibility to symptoms associated with exfoliation syndrome and/or glaucoma in a human individual, the method comprising determining the presence or absence of at least one allele of at least one polymorphic marker in a nucleic acid sample obtained from the individual, wherein the at least one polymorphic marker is associated with the LOXL1 gene, and wherein the presence of the at least one allele is indicative of a susceptibility to symptoms associated with exfoliation syndrome and/or glaucoma. The method may also include determination of the presence or absence of the at least one allele of at least one polymorphic marker in a genotype dataset from the individual.

In certain embodiments, the at least one polymorphic marker is associated with the LOXL1 gene by virtue of being in linkage disequilibrium with the LOXL1 gene. In other words, the at least one polymorphic marker is in linkage disequilibrium with at least one genetic element within the LOXL1 gene.

Another aspect relates to a method of determining a susceptibility to symptoms associated with exfoliation syndrome and/or glaucoma in a human individual, the method comprising determining whether at least one allele of at least one polymorphic marker is present in a nucleic acid sample obtained from the individual, or in a genotype dataset derived from the individual, wherein the at least one polymorphic marker is associated with the LOXL1 gene, and wherein the presence of the at least one allele is indicative of a susceptibility to symptoms associated with exfoliation syndrome and/or glaucoma. The presence of at least one allele of at least one marker that occurs more frequently in individuals diagnosed with exfoliation syndrome and/or glaucoma is indicative of increased susceptibility to exfoliation syndrome and/or glaucoma in the individual. In certain embodiments, the at least one allele is an at-risk allele, i.e. the allele confers increased risk of developing, exfoliation syndrome and/or glaucoma.

Determination of the presence or absence of an allele implies the determination of the presence or absence of a particular allele, or alternatively multiple alleles. Determination of the presence or absence of one particular allele of a biallelic marker (for which there are only two alleles possible) indirectly provides information about the presence or absence of the alternate allele. For example, for a C/T SNP polymorphism, determination of the absence of a C at the SNP in a particular genome implies that the genome contains two copies of the alternate allele (the T allele). Determination of the presence of one copy of the C allele likewise indicates the presence of one copy of the alternate T allele. For polymorphisms that have more than two possible alleles, such as microsatellites, the determination of the presence or absence of an allele does not provide by itself provide information about the presence or absence of other alleles of the marker. In certain embodiments, the identity of particular alleles is performed, i.e. the nucleotide sequence at the particular allelic site is determined. Such embodiments provide a direct indication of the presence or absence of particular alleles.

Another aspect of the invention relates to a method of diagnosing a susceptibility of symptoms associated with exfoliation syndrome and/or glaucoma in an individual, the method comprising determining the identity of at least one allele of at least one polymorphic marker in a nucleic acid sample obtained from the individual, wherein the at least one marker is selected from the group of markers located within the LOXL1 LD block, wherein the presence of the at least one allele is indicative of a susceptibility of symptoms associated with exfoliation syndrome and/or glaucoma.

Another aspect relates to a method of assessing an individual for probability of response to a therapeutic agent for preventing and/or ameliorating symptoms associated with exfoliation syndrome and/or glaucoma, comprising: determining the presence or absence of at least one allele of at least one polymorphic marker in a nucleic acid sample obtained from the individual, wherein the at least one polymorphic marker is selected from the group consisting of the polymorphic markers listed in Table 4, and markers in linkage disequilibrium therewith, wherein the presence of the at least one allele of the at least one marker is indicative of a probability of a positive response to a symptoms associated with exfoliation syndrome and/or glaucoma therapeutic agent. In one embodiment, the at least one polymorphic marker is selected from the group of markers associated with the LOXL1 gene.

In certain embodiments, the therapeutic agent is selected from the agents set forth in Agent Table I. The therapeutic agent can in certain embodiments be selected from prostaglandin analogs, prostamides, a2 adrenergic agonists, carbonic anhydrase inhibitors, β-blockers, cholinergic agonists, or other therapeutic agents used to prevent or ameliorate symptoms associated with glaucoma and/or exfoliation syndrome.

In a preferred embodiment, the therapeutic agent is selected from latanoprost, travoprost, unoprostone, bimatoprost, brimonidine, apraclonidine, dorzolamide, brinzolamide, acetazolamide, methozolamide, betaxolol, carteolol, levobunolol, metipranolol, timolol, pilocarpine, carbachol, echothiophate and epinephrine.

The invention also relates to methods of determining whether an individual administered a particular therapy or therapeutic method is at increased risk of developing exfoliation syndrome and/or glaucoma, or symptoms associated therewith, as a consequence of being adminstered the medication or the therapeutic method.

Thus, another aspect of the invention relates to a method of determining whether a human individual is at risk for developing elevated intraocular pressure, exfoliation syndrome and/or glaucoma as a complication of being treated with a glucocorticoid therapeutic agent, the method comprising determining the presence or absence of at least one allele of at least one polymorphic marker in a nucleic acid sample obtained from the individual, wherein the at least one polymorphic marker is associated with the LOXL1 gene, and wherein the presence of the at least one allele is indicative of an increased risk of developing elevated intraocular pressure and/or glaucoma as a complication of being treated with a glucocorticoid therapeutic agent.

In preferred embodiments, the at least one polymorphic marker is selected from markers rs2165241 allele T, rs1048661 allele G and rs3825942 allele G, and markers in linkage disequilibrium therewith.

In a related aspect, the invention pertains to a method of determining whether a human individual is at decreased risk of developing elevated intraocular pressure, exfoliation syndrome and/or glaucoma as a complication of being treated with a glucocorticoid therapeutic agent, the method comprising determining the presence of rs1048661 allele G and rs3825942 allele G, or markers in linkage disequilibrium therewith, in a nucleic acid sample from the individual, wherein the presence of rs1048661 allele G or rs3825942 allele G is indicative of decreased risk for developing elevated intraocular pressure, exfoliation syndrome and/or glaucoma as a complication of being treated with the glucocorticoid therapeutic agent.

In one embodiment of this aspect, the presence of both rs1048661 allele G and rs3825942 allele G, or markers in linkage disequilibrium therewith, in the nucleic acid sample from the individual, is indicative of decreased risk for developing elevated intraocular pressure, exfoliation syndrome and/or glaucoma as a complication of being treated with the glucocorticoid therapeutic agent. In another embodiment, the presence of two copies of both rs1048661 allele G and rs3825942 allele G, or markers in linkage disequilibrium therewith, in the nucleic acid sample from the individual, is indicative of decreased risk for developing elevated intraocular pressure, exfoliation syndrome and/or glaucoma as a complication of being treated with the glucocorticoid therapeutic agent.

In some embodiments, the glucocorticoid therapeutic agent is selected from the agents set from the agents set forth in Agent Table I. The glucocorticoid therapeutic agent is in preferred embodiments selected from betamethoasone, clobetasone butyrate, dexamethasone, fluorometholone, hydrocortisone acetate, prednisolone, rimexolone, loteprednol, and medrysone. Furthermore, the invention provides a method of predicting prognosis of an individual experiencing symptoms associated with, or an individual diagnosed with, exfoliation syndrome and/or glaucoma, the method comprising determining the presence or absence of at least one allele of at least one polymorphic marker in a nucleic acid sample obtained from the individual, wherein the at least one polymorphic marker is selected from the group consisting of the polymorphic markers listed in Table 4, and markers in linkage disequilibrium therewith, wherein the presence of the at least one allele is indicative of a worse prognosis of the exfoliation syndrome and/or glaucoma in the individual. Prognosis can alternatively be predicted in the individual by obtaining nucleic acid sequence data about a human individual identifying at least one allele of at least one polymorphic marker associated with the human LOXL1 gene, wherein different alleles of the at least one polymorphic marker are associated with different susceptibilities to the at least one condition in humans, and predicting prognosis of the individual from the nucleic acid sequence data. In some embodiments, at least two polymorphic markers associated with LOXL1 gene are assessed.

Yet another aspect of the invention relates to a method of monitoring treatment outcome of an individual undergoing treatment for symptoms associated with exfoliation syndrome and/or glaucoma, the method comprising determining the presence or absence of at least one allele of at least one polymorphic marker in a nucleic acid sample obtained from the individual, wherein the at least one polymorphic marker is selected from the group consisting of the polymorphic markers listed in Table 4, and markers in linkage disequilibrium therewith, wherein the presence of the at least one allele is indicative of the treatment outcome of the individual. One embodiment of this method relates to markers associated with the LOXL1 gene for the monitoring of progress of treatment. The invention also provides a method of predicting treatment outcome of an individual undergoing treatment for exfoliation syndrome and/or glaucoma, the method comprising obtaining nucleic acid sequence data about a human individual identifying at least one allele of at least one polymorphic marker associated with the human LOXL1 gene, wherein different alleles of the at least one polymorphic marker are associated with different susceptibilities to the at least one condition in humans, and predicting treatment outcome of the individual from the nucleic acid sequence data.

Thus the markers described herein may be useful for predicting the effectiveness of a particular treatment for symptoms associated with exfoliation syndrome and/or glaucoma, or in an individual who has been diagnosed with one or more of these disorders. Treatment outcome is known to be highly variable between individuals, and in many cases it is difficult to determine a priori whether a particular individual will benefit from a particular treatment. It is contemplated that the variants described herein as being associated with risk of ocular disorders such as glaucoma and exfoliation syndrome are useful for predicting the particular treatment outcome. Such prediction can be useful in planning treatment strategies, for example, by first determining the genetic makeup of the individual at particular genetic markers and base a treatment plan on the results of such analysis.

The invention also pertains to determination of the amino acid sequence of LOXL1 protein in the methods described herein. The inventors have found that amino acid changes at polymorphic positions 141 and 153 in the LOXL1 amino acid sequence as set forth in SEQ ID NO:85 are associated with glaucoma and exfoliation syndrome. Thus, the invention in another aspect relates to a method of diagnosing an increased susceptibility to symptoms associated with exfoliation syndrome and/or glaucoma in a human individual, the method comprising determining the amino acid at position 141 or 153 in SEQ ID NO: 85 in a protein sample obtained from the individual, wherein the presence of Arginine at position 141 and/or Glycine at position 153 is indicative of an increased susceptibility to symptoms associated with exfoliation syndrome and/or glaucoma. The amino acid substitutions at these positions are caused by single nucleotide polymorphisms in codon 141 and codon 153 of the LOXL1 gene. Thus, conversely, the presence of the alternate amino acid at these positions is protective for glaucoma or exfoliation syndrome, i.e. they are associated with decreased risk of developing glaucoma or exfoliation syndrome. Thus, in another aspect, the invention relates to a method of diagnosing a decreased susceptibility to symptoms associated with exfoliation syndrome and/or glaucoma in a human individual, the method comprising determining the amino acid at position 141 or 153 in SEQ ID NO: 85 in a protein sample obtained from the individual, wherein the presence of Leucine at position 141 and/or Aspartic acid at position 153 is indicative of a decreased susceptibility to symptoms associated with exfoliation syndrome and/or glaucoma.

The invention also provides a method of diagnosing a susceptibility to at least one ocular condition selected from exfoliation syndrome and glaucoma in a human individual, the method comprising:

obtaining LOXL1 amino acid sequence data about at least one encoded LOXL1 protein of a human individual,
identifying at least one polymorphic site associated with the LOXL1 amino acid sequence, wherein different amino acids of the at least one polymorphic site are associated with different susceptibilities to the at least one condition in humans, and
diagnosing susceptibility to at least one condition selected from exfoliation syndrome and glaucoma from the amino acid sequence data.

In certain embodiments, determination of the presence of an Arginine at position 141 and/or a Glycine at position 153 in the LOXL1 protein as set forth in SEQ ID NO:85 is indicative of an increased susceptibility to exfoliation syndrome and/or glaucoma. Determination of presence of an Arginine at position 141 and a Glycine at position 153 in the LOXL1 protein as set forth in SEQ ID NO:85 is in certain other embodiments indicative of an increased susceptibility to exfoliation syndrome and/or glaucoma. Alternatively, determination of the presence of an Leucine at position 141 and/or an Aspartic acid at position 153 in the LOXL1 protein as set forth in SEQ ID NO:85 is indicative of a decreased susceptibility to the at least one condition. In certain other embodiments, determination of the presence of an Leucine at position 141 and an Aspartic acid at position 153 in the LOXL1 protein as set forth in SEQ ID NO:85 is indicative of a decreased suscepbility to exfoliation syndrome and/or glaucoma.

The discovery of the inventors can be used to identify additional markers that convey risk of developing glaucoma or exfoliation syndrome, by the fact that such markers are in linkage disequilibrium with the markers presented herein to be associated with glaucoma and exfoliation syndrome. Thus, another aspect of the invention relates to a method of identification of a marker for use in assessing susceptibility to exfoliation syndrome and/or glaucoma, the method comprising
a. identifying at least one polymorphism associated with the LOXL1 gene; and
b. determining the genotype status of a sample of individuals diagnosed with, or having a susceptibility to, exfoliation syndrome and/or glaucoma, and a control sample;
   wherein a significant difference in frequency of at least one allele in at least one polymorphism in individuals diagnosed with, or having a susceptibility to, exfoliation syndrome, as compared with the frequency of the at least one allele in the control sample is indicative of the at least one polymorphism being useful for assessing susceptibility to exfoliation syndrome and/or glaucoma.

In one embodiment, an increase in frequency of the at least one allele in the at least one polymorphism in individuals diagnosed with, or having a susceptibility to, exfoliation syndrome, as compared with the frequency of the at least one allele in the control sample is indicative of the at least one polymorphism being useful for assessing increased susceptibility to exfoliation syndrome and/or glaucoma.

In another embodiment, a decrease in frequency of the at least one allele in the at least one polymorphism in individuals diagnosed with, or having a susceptibility to, exfoliation syndrome, as compared with the frequency of the at least one allele in the control sample is indicative of the at least one polymorphism being useful for assessing decreased susceptibility to, or protection against, exfoliation syndrome and/or glaucoma.

Methods of genotyping are also within the scope of the invention. In one such aspect of the invention, a method of genotyping a nucleic acid sample obtained from a human individual at risk for, or diagnosed with, exfoliation syndrome and/or glaucoma is provided, comprising determining the presence or absence of at least one allele of at least one polymorphic marker in the sample. The marker can be any of the markers presented herein to be associated with glaucoma or exfoliation syndrome, or markers in linkage disequilibrium therewith.

The methods of the invention that related to the polymorphic markers described herein in a general sense relate to markers associated with the LOXL1 gene. In some embodiments, the invention relates to markers within the LOXL1 LD block as defined herein and set forth in SEQ ID NO:84. In another embodiment, the markers are selected from markers associated with the LOXL1 gene. In this context, this encompasses markers within the LOXL1 gene, or markers outside the gene that are either in linkage disequilibrium with markers in the gene or represent polymorphisms that affect the function (e.g., expression) of the gene. For example, this can include markers within the promoter of LOXL1 that may alter the expression levels of LOXL1. In one embodiment, the markers are selected from the group of markers listed in Table 4. In another embodiment, the markers are selected from the group of markers listed in Table 6. In another embodiment, the markers are selected from the group of markers listed in Table 6a. In another embodiment, the markers are selected from the group of markers listed in Table 15. n another embodiment, the markers are selected from the group of markers listed in Table 16. In yet another embodiment, the markers are selected from rs4886725 (SEQ ID NO:86), rs12915956 (SEQ ID NO:87), rs896590 (SEQ ID NO:88), rs12438872 (SEQ ID NO:89), rs4261482 (SEQ ID NO:42), rs2165241 (SEQ ID NO:15), rs1992314 (SEQ ID NO:44), rs4886776 (SEQ ID NO:45), rs2028386 (SEQ ID NO:46), rs4337252 (SEQ ID NO:47), rs2028387 (SEQ ID NO:48), rs4077284 (SEQ ID NO:49), rs893816 (SEQ ID NO:50), rs4886782 (SEQ ID NO:51), rs893817 (SEQ ID NO:17), rs893818 (SEQ ID NO:52), rs893820 (SEQ ID NO:53), rs12440667 (SEQ ID NO:54), rs1530169 (SEQ ID NO:19), rs893821 (SEQ ID NO:90), rs750460 (SEQ ID NO:55), rs4243042 (SEQ ID NO:56), rs2304722 (SEQ ID NO:91), rs4886623 (SEQ ID NO:92), rs2167648 (SEQ ID NO:93), rs1584738 (SEQ ID NO:94), rs1901570 (SEQ ID NO:95), rs8026593 (SEQ ID NO:6), rs4886660 (SEQ ID NO:96), rs4886421 (SEQ ID NO:97), rs4886663 (SEQ ID NO:98), rs4886664 (SEQ ID NO:99), rs4145873 (SEQ ID NO:100), rs4145874 (SEQ ID NO:101), rs1452389 (SEQ ID NO:102), rs1078967 (SEQ ID NO:103), rs8041642 (SEQ ID NO:104), rs8041685 (SEQ ID NO:16), rs8042039 (SEQ ID NO:105), rs2304719 (SEQ ID NO:18), rs12437465 (SEQ ID NO:57), rs1048661 (SEQ ID NO:106), and rs3825942 (SEQ ID NO: 107). In preferred embodiments, the markers are rs1048661 (SEQ ID NO:106) or rs3825942 (SEQ ID NO:107), or markers in linkage disequilibrium therewith. In especially preferred embodiments, the markers are rs1048661 (SEQ ID NO: 106) or rs3825942 (SEQ ID NO: 107). In some preferred embodiments, the marker is rs1048661 (SEQ ID NO: 106). In another preferred embodiment, the markers is rs3825942 (SEQ ID NO: 107).

All of the above-mentioned markers are useful in the methods, uses, apparatus, media and kits described further herein, and can be used alone or in any particular combinations of two or more, three or more, four or more, or five or more, in particular embodiments. The use of any one ore a combination of these markers is thus possible in the variant aspects of the invention as described herein. The skilled person will also appreciate that markers that are in linkage disequilibrium with the cited markers are also useful in the methods of the invention, and are thus also within the scope of the invention presented herein.

In some embodiments of the methods of the invention, a further step of assessing frequency of at least one haplotype in the individual is performed, wherein the presence of the at least one haplotype is indicative of a susceptibility to ocular disorders such as exfoliation syndrome and/or glaucoma, or symptoms associated with exfoliation syndrome and/or glaucoma. Exemplary haplotypes are provided by markers rs1048661 and rs3825942. Thus, in one embodiment, the invention relates to assessing the haplotype characterized by the presence of allele G in marker rs1048661 (SEQ ID NO: 106) and allele G in marker rs3825942 (SEQ ID NO: 107), and/or the haplotype characterized by the presence of allele T in marker rs1048661 (SEQ ID NO: 106) and allele G in marker rs3825942 (SEQ ID NO: 107), wherein the presence of either of these haplotypes is indicative of increased susceptibility of developing symptoms associated with exfoliation syndrome or glaucoma. In another embodiment, the presence of the haplotype characterized by the presence of allele G in marker rs1048661 (SEQ ID NO: 106) and allele A in marker rs3825942 (SEQ ID NO: 107) is indicative of decreased susceptibility of developing symptoms associated with exfoliation syndrome or glaucoma. Other haplotypes comprising any two or more markers as disclosed and described herein are also contemplated and within the scope of the invention.

In certain embodiments of the invention, linkage disequilibrium is characterized by particular numerical values of the linkage disequilibrium measures r² and |D′|. In certain embodiments, linkage disequilibrium between genetic elements (e.g., markers) is defined as r²>0.1 (r² greater than 0.1). In some embodiments, linkage disequilibrium is defined as r²>0.2. Other embodiments can include other definitions of linkage disequilibrium, such as r²>0.25, r²>0.3, r²>0.35, r²>0.4, r²>0.45, r²>0.5, r²>0.55, r²>0.6, r²>0.65, r²>0.7, r²>0.75, r²>0.8, r²>0.85, r²>0.9, r²>0.95, r²>0.96, r²>0.97, r²>0.98, or r²>0.99. Linkage disequilibrium can in certain embodiments also be defined as |D′|>0.2, or as |D′|>0.3, |D′|>0.4, |D′|>0.5, |D′|>0.6, |D′|>0.7, |D′|>0.8, |D′|>0.9, |D′|>0.95, |D′|>0.98 or |D′|>0.99. In certain embodiments, linkage disequilibrium is defined as fulfilling two criteria of r² and |D′|, such as r²>0.2 and |D′|>0.8. Other combinations of values for r² and |D′| are also possible and within scope of the present invention, including but not limited to the values for these parameters set forth in the above.

Linkage disequilibrium is in one embodiment determined using a collection of samples from a single population, as described herein. One embodiment uses a collection of Caucasian samples, such as Icelandic samples, Caucasian samples from the CEPH collection as described by the HapMap project (http://www.hapmap.org). Other embodiments use sample collections from other populations, including, but not limited to African American population samples, African samples from the Yuroban population (YRI), or Asian samples from China (CHB) or Japan (JPT).

In certain embodiments of the invention, the presence of a particular allele or haplotype is indicative of increased susceptibility, for example increased susceptibility to symptoms associated with exfoliation syndrome and/or glaucoma, or increased susceptibility to exfoliation syndrome and/or glaucoma. In such embodiments, the increased susceptibility is characterized by a certain relative risk value. Thus, in particular embodiments of the invention, the increased susceptibility is characterized by a relative risk of at least 1.5, including a relative risk of at least 2.0, a relative risk of at least 2.5, a relative risk of at least 3.0, a relative risk of at least 3.5, and a relative risk of at least 4.0. Other embodiments are characterized by relative risk of at least 1.75, 2.25, 2.75, 3.25, 3.75, and so on. Other values for the relative risk are however also within the scope of the present invention. In one embodiment of the invention, the at least one allele indicative of increased susceptibility is selected from the group consisting of rs2165241 allele T, rs1048661 allele G and rs3825942 allele G.

In certain other embodiments of the invention, certain alleles or haplotypes are found in decreased frequency in patients than in the population. Thus, certain alleles or haplotypes are found in decreased frequency in individuals diagnosed with exfoliation syndrome or glaucoma than in the general population. Such markers are indicative of a protection against exfoliation syndrome or glaucoma, or a decreased susceptibility of developing these disorders. Exemplary alleles are those of rs1048661 allele T and/or rs3825942 allele A.

Decreased susceptibility is in particular embodiments characterized by a relative risk of less than 0.7, including a relative risk of less than 0.6, a relative risk of less than 0.5, a relative risk of less than 0.4, a relative risk of less than 0.35, a relative risk of less than 0.3, and a relative risk of less than 0.25. Other values of relative risk characterizing the decreased susceptibility or decreased risk are however also possible and within scope of the invention, including, but not limited to, less than 0.8, less than 0.75, less than 0.65, less than 0.55, less than 0.45, less than 0.20, etc.

In certain embodiments of the invention as described herein, the symptoms associated with exfoliation syndrome and/or glaucoma are further accompanied by, or characterized by, a clinical diagnosis of exfoliation syndrome and/or glaucoma. Such symptoms are in some embodiments characterized by the presence of at least one of: degeneration of retinal ganglion cells, elevated intraocular pressure, optic disc changes and loss of peripheral vision. In particular, optic disc changes are in certain embodiments characterized by at least one of: (a) increased cup-to-disc ratio (thin neuroretinal rim), (b) progressive optic disc cupping, (c) asymmetric optic disc cupping (more than 0.2 difference), (d) acquired pit of the optic nerve, (e) parapapillary retinal nerve fibre layer loss.

In some further embodiments of the invention, the human subject is characterized by having at least one risk factor for exfoliation syndrome and/or glaucoma, said risk factor being selected from: increased intraocular pressure, high age, African-American race, visual field abnormalities, myopia, family history of glaucoma, thin cornea, systemic hypertension, cardiovascular disease, migraine headache and peripheral vasospasm.

In the methods of the invention that related to the assessment of genetic markers, any sample containing genomic DNA can be utilized. This includes a blood sample, sample of amniotic fluid, sample of cerebrospinal fluid, or tissue sample from skin, muscle, buccal or conjunctival mucosa (buccal swab), placenta, gastrointestinal tract or other organs, or other suitable samples containing genomic DNA. In preferred embodiments, the genomic DNA is amplified by Polymerase Chain Reaction (PCR) prior to analysis.

The genotyping methods that can be utilized in the methods of the invention include genotyping methods based on allele-specific probe hybridization, allele-specific primer extension, allele-specific amplification, nucleic acid sequencing (e.g., DNA sequencing), 5′-exonuclease digestion, molecular beacon assay, oligonucleotide ligation assay, size analysis, and single-stranded conformation analysis.

In certain embodiments of the invention, a biomarker is also assessed to provide a combined risk assessment. Biomarkers can be assessed in suitable tissues and fluids, such as in a blood sample or in fluid from the eye (tear fluid). In one embodiment, the biomarker is LOXL1 protein. In other embodiments, non-genetic information, such as information selected from age, gender, ethnicity, socioeconomic status, previous disease diagnosis, medical history of subject, family history of exfoliation syndrome and/or glaucoma, biochemical measurements, and clinical measurements, is furthermore assessed to make risk assessment, diagnosis, or prognosis of the individual.

The methods of the invention can furthermore include measurements of LOXL1 expression levels in a suitable biological sample, such as a blood sample. In one embodiment, expression levels are assessed by mRNA analysis, for determining the amount of LOXL1 transcript in the biological sample. In a preferred embodiment, a decreased expression level of LOXL1 is indicative of increased susceptibility to exfoliation syndrome or glaucoma.

The invention also provides kits for use in the various methods of the invention. The kits comprise reagents necessary for determining the presence or absence of certain alleles found to be associated with exfoliation syndrome and glaucoma, as described herein. In one embodiment, the kit comprises reagents for selectively detecting at least one allele of at least one polymorphic marker in the genome of a human individual, wherein the polymorphic marker is selected from the group consisting of the polymorphic markers listed in Table 4, Table 6 and Table 6a and markers in linkage disequilibrium therewith, and wherein the presence of the at least one allele is indicative of a susceptibility to symptoms associated with exfoliation syndrome and/or glaucoma.

In one embodiment, the kit comprises reagents for detecting at least one marker selected from rs4886725 (SEQ ID NO:86), rs12915956 (SEQ ID NO:87), rs896590 (SEQ ID NO:88), rs12438872 (SEQ ID NO:89), rs4261482 (SEQ ID NO:42), rs2165241 (SEQ ID NO:15), rs1992314 (SEQ ID NO:44), rs4886776 (SEQ ID NO:45), rs2028386 (SEQ ID NO:46), rs4337252 (SEQ ID NO:47), rs2028387 (SEQ ID NO:48), rs4077284 (SEQ ID NO:49), rs893816 (SEQ ID NO:50), rs4886782 (SEQ ID NO:51), rs893817 (SEQ ID NO:17), rs893818 (SEQ ID NO:52), rs893820 (SEQ ID NO:53), rs12440667 (SEQ ID NO:54), rs1530169 (SEQ ID NO:19), rs893821 (SEQ ID NO:90), rs750460 (SEQ ID NO:55), rs4243042 (SEQ ID NO:56), rs2304722 (SEQ ID NO:91), rs4886623 (SEQ ID NO:92), rs2167648 (SEQ ID NO:93), rs1584738 (SEQ ID NO:94), rs1901570 (SEQ ID NO:95), rs8026593 (SEQ ID NO:6), rs4886660 (SEQ ID NO:96), rs4886421 (SEQ ID NO:97), rs4886663 (SEQ ID NO:98), rs4886664 (SEQ ID NO:99), rs4145873 (SEQ ID NO:100), rs4145874 (SEQ ID NO:101), rs1452389 (SEQ ID NO:102), rs1078967 (SEQ ID NO:103), rs8041642 (SEQ ID NO:104), rs8041685 (SEQ ID NO:16), rs8042039 (SEQ ID NO:105), rs2304719 (SEQ ID NO:18), rs12437465 (SEQ ID NO:57), rs1048661 (SEQ ID NO:106), and rs3825942 (SEQ ID NO:107), and markers in linkage disequilibrium therewith. In a preferred embodiment, the kit comprises reagents for detecting rs1048661 (SEQ ID NO: 106), and/or rs3825942 (SEQ ID NO: 107). In another preferred embodiment, the alleles detected by the kit comprise rs1048661 allele G and marker rs3825942 allele G.

One embodiment of the kit includes reagents that comprise at least one contiguous oligonucleotide that hybridizes to a fragment of the genome of the individual comprising at least one polymorphic marker, a buffer and a detectable label. In another embodiment, the reagents comprise at least one pair of oligonucleotides that hybridize to opposite strands of a genomic nucleic acid segment obtained from the subject, wherein each oligonucleotide primer pair is designed to selectively amplify a fragment of the genome of the individual that includes one polymorphic marker, and wherein the fragment is at least 30 base pairs in size. In a preferred embodiment, the at least one oligonucleotide is completely complementary to the genome of the individual. The oligonucleotide may be of any suitable size, such as from 15-100 nucleotides, 18-50 nucleotides or 20-30 nucleotides in length.

Kits for use in other methods of the invention, as described herein, are also contemplated and within scope of the invention. Such kits have in common the characteristics of providing reagents for selectively detecting particular alleles and/or haplotypes associated with exfoliation syndrome and/or glaucoma, as described in the herein.

The invention also provides in another embodiment the use of an oligonucleotide probe in the manufacture of a diagnostic reagent for diagnosing and/or assessing susceptibility to exfoliation syndrome or glaucoma, wherein the probe hybridizes to a segment of a nucleic acid whose nucleotide sequence is given by SEQ ID NO: 84 that comprises at least one polymorphic site, wherein the fragment is 15-500 nucleotides in length. The polymorphic site is preferably selected from rs4886725 (SEQ ID NO:86), rs12915956 (SEQ ID NO:87), rs896590 (SEQ ID NO:88), rs12438872 (SEQ ID NO:89), rs4261482 (SEQ ID NO:42), rs2165241 (SEQ ID NO:15), rs1992314 (SEQ ID NO:44), rs4886776 (SEQ ID NO:45), rs2028386 (SEQ ID NO:46), rs4337252 (SEQ ID NO:47), rs2028387 (SEQ ID NO:48), rs4077284 (SEQ ID NO:49), rs893816 (SEQ ID NO:50), rs4886782 (SEQ ID NO:51), rs893817 (SEQ ID NO:17), rs893818 (SEQ ID NO:52), rs893820 (SEQ ID NO:53), rs12440667 (SEQ ID NO:54), rs1530169 (SEQ ID NO:19), rs893821 (SEQ ID NO:90), rs750460 (SEQ ID NO:55), rs4243042 (SEQ ID NO:56), rs2304722 (SEQ ID NO:91), rs4886623 (SEQ ID NO:92), rs2167648 (SEQ ID NO:93), rs1584738 (SEQ ID NO:94), rs1901570 (SEQ ID NO:95), rs8026593 (SEQ ID NO:6), rs4886660 (SEQ ID NO:96), rs4886421 (SEQ ID NO:97), rs4886663 (SEQ ID NO:98), rs4886664 (SEQ ID NO:99), rs4145873 (SEQ ID NO:100), rs4145874 (SEQ ID NO:101), rs1452389 (SEQ ID NO:102), rs1078967 (SEQ ID NO:103), rs8041642 (SEQ ID NO:104), rs8041685 (SEQ ID NO:16), rs8042039 (SEQ ID NO:105), rs2304719 (SEQ ID NO:18), rs12437465 (SEQ ID NO:57), rs1048661 (SEQ ID NO:106), and rs3825942 (SEQ ID NO:107), and polymorphisms in linkage disequilibrium therewith.

Also provided by the present invention is a computer readable medium. The medium can contain information for determining a suscepbility to exfoliation and/or glaucoma. In a particular aspect, the medium comprises
data indicative of at least one polymorphic marker;
a routine stored on the computer readable medium and adapted to be executed by a processor to determine risk of developing exfoliation syndrome and/or glaucoma for the at least one polymorphic marker;
wherein the at least one polymorphic marker is associated with the LOXL1 gene.

In certain embodiments, the computer readable medium contains data indicative of at least two polymorphic markers. In certain other embodiments, the medium contains data indicative of three or more, four or more or five or more polymorphic markers. In certain other embodiments, the medium further comprises data indicative of at least one haplotype associated with the LOXL1 gene. Such a haplotype can comprise two or more polymorphic markers, including three or more markers, four or more markers and five or more markers. The medium may also contain protein sequence data, that is data indicative of at least one polymorphic protein marker (amino acid polymorphism) that is encoded by a polymorphic marker within the coding sequence of LOXL1. In preferred embodiments, so protein markers contain data on polymorphisms at position 141 and/or position 153 in the LOXL1 protein as set forth in SEQ ID NO:85.

Yet another aspect of the invention relates to an apparatus for determining a genetic indicator for exfoliation syndrome or glaucoma in a human individual, comprising: a processor; a computer readable memory having computer executable instructions, adapted to be executed on the processor to analyze marker and/or haplotype information for at least one human individual with respect to at least one polymorphic marker associated with the LOXL1 gene, and generate an output based on the marker or haplotype information, wherein the output comprises a risk measure of the at least one marker or haplotype as a genetic indicator of exfoliation syndrome or glaucoma for the human individual. In certain embodiments, the computer readable memory further comprises data indicative of the frequency of at least one allele of at least one polymorphic marker or at least one haplotype in a plurality of individuals diagnosed with, or presenting symptoms associated with, exfoliation syndrome or glaucoma, and data indicative of the frequency of at the least one allele of at least one polymorphic marker or at least one haplotype in a plurality of reference individuals, and wherein a risk measure is based on a comparison of the at least one marker and/or haplotype status for the human individual to the data indicative of the frequency of the at least one marker and/or haplotype information for the plurality of individuals diagnosed with exfoliation syndrome or glaucoma. In certain other embodiments, the computer readable memory further comprises data indicative of the risk of developing exfoliation syndrome and/or glaucoma associated with at least one allele of at least one polymorphic marker or at least one haplotype, and wherein a risk measure for the human individual is based on a comparison of the at least one marker and/or haplotype status for the human individual to the risk associated with the at least one allele of the at least one polymorphic marker or the at least one haplotype. In other embodiments, the computer readable memory further comprises data indicative of the frequency of at least one allele of at least one polymorphic marker or at least one haplotype in a plurality of individuals diagnosed with, or presenting symptoms associated with, exfoliation syndrome and/or glaucoma, and data indicative of the frequency of at the least one allele of at least one polymorphic marker or at least one haplotype in a plurality of reference individuals, and wherein risk of developing exfoliation syndrome and/or glaucoma is based on a comparison of the frequency of the at least one allele or haplotype in individuals diagnosed with, or presenting symptoms associated with, exfoliation syndrome and/or glaucoma, and reference individuals.

The present invention also provides pharmaceutical compositions and assays. In one such aspect, the invention provides a pharmaceutical composition for the treatment of symptoms associated with glaucoma or exfoliation syndrome in an individual in need thereof, comprising a polypeptide encoded by a human LOXL1 gene, or fragments thereof, and pharmacologically acceptable carriers and/or excipients. In certain embodiments, the polypeptide has the amino acid sequence as set forth in SEQ ID NO: 85. In one embodiment, the polypeptide is characterized by the presence of a Leucine at position 141 in SEQ ID NO: 85. In another embodiment, the polypeptide is characterized by the presence of an Aspartic Acid at position 153 in SEQ ID NO:85. In a further embodiment, the composition is characterized by the presence of a Leucine at position 141 in SEQ ID NO: 85 and an Aspartic Acid at position 153 in SEQ ID NO:85.

An assay for screening compounds for preventing or ameliorating symptoms associated with exfoliation syndrome and/or glaucoma is also provided, comprising
(i) administering a test compound to an animal having symptoms associated with exfoliation syndrome and/or glaucoma, or a cell population isolated therefrom;
(ii) determining the level of gene expression of LOXL1 in a sample from the animal or in the cell population isolated therefrom;
(iii) determining the level of gene expression of LOXL1 in a sample from at least one control animal that does not have symptoms associated with exfoliation syndrome or glaucoma, or in a cell population isolated therefrom, in the absence of the compound;
(iv) comparing the expression levels obtained in (ii) and (iii);
   wherein a test compound that provides expression levels that are similar in a treated animal and the at least one control animal are identified as candidates for drugs for preventing, or ameliorating symptoms associated with, exfoliation syndrome or glaucoma. The animal is preferably a human individual. In another preferred embodiment the method further comprises determining the genotype of the human individual for markers rs1048661 (SEQ ID NO: 106) or rs3825942 (SEQ ID NO: 107), or markers in linkage disequilibrium therewith, prior to administration of the test compound, wherein the genotype status of the human individual is used for determining whether the animal is suitable for screening the test compound. Preferably, the genotype of the human individual for markers rs1048661 (SEQ ID NO: 106) or rs3825942 (SEQ ID NO: 107) is determined, wherein the presence of allele G at marker rs1048661 and/or allele G at marker rs3825942 is a measure of the human individual being suitable for screening the test compound.

The invention further provides a method for treating a human individual for symptoms associated with exfoliation syndrome and/or glaucoma, comprising administering a composition that is identified by the screening assay.

Yet another aspect of the invention relates to a method for treating a human individual for symptoms associated with exfoliation syndrome and/or glaucoma, comprising expressing a LOXL1 gene in vivo in an amount sufficient to treat the disease. Preferably, the LOXL1 gene is characterized by the sequence set forth in SEQ ID NO:84, optionally containing at least one polymorphic site. In one embodiment, the sequence of the LOXL1 gene is characterized by the presence of a T at position 7142 in SEQ ID NO:84. In another embodiment, the sequence is characterized by the presence of an A at position 7178 in SEQ ID NO:84. In yet another embodiment, the sequence is characterized by the presence of a T a position 7142 and an A at position 7178. The method may in a preferred embodiment include the steps of (a) administering to the human individual a vector comprising a LOXL1 gene; and (b) allowing LOXL1 protein to be expressed in an amount sufficient to treat the symptoms associated with exfoliation syndrome or glaucoma. The vector is suitably selected from an adenoviral vector and a lentiviral vector, or the vector is a replication-defective viral vector. The vector may be administered by a method selected from topical administration, intravitreal administration, intraocular administration, parenteral administration, intranasal administration, intratracheal administration, intrabronchial administration and subcutaneous administration. Preferably, the vector is delivered by intraocular administration.

The invention also provides a method of treating symptoms associated with exfoliation syndrome or glaucoma, or a method of treating an ocular condition selected from exfoliation syndrome and glaucoma, comprising administering an agent that regulates the expression, activity of physical state of the LOXL1 gene or its encoding RNA or protein. The agent is in one embodiment selected from a small molecule compound, an oligonucleotide, a peptide and an antibody. In another embodiment, the agent is an antisense molecule or interfering RNA. In yet another embodiment, the agent is an expression modifier, such as an activator or a repressor. Thus, therapy by antisense technology or utilizing interfering RNA is within the scope of the invention.

It is contemplated that LOXL1 polypeptide may be used for treating glaucoma or exfoliation syndrome. Thus the invention in one aspect pertains to human LOXL1 polypeptide for the treatment of glaucoma or exfoliation syndrome. In one embodiment, the LOXL1 polypeptide has the sequence as set forth in SEQ ID NO:85. In another embodiment, the LOXL1 polypeptide has the sequence as set forth in SEQ ID NO:85 that contains at least one polymorphic site. In one embodiment, the polypeptide is characterized by the amino acid sequence as set forth in SEQ ID NO:85 having a Leucine in position 141. The polypeptide may also be characterized by the amino acid sequence as set forth in SEQ ID NO:85 having a Aspartic Acid in position 153. The polypeptide may furthermore be characterized by the amino acid sequence as set forth in SEQ ID NO:85 having a Leucine in position 141 and an Aspartic Acid in position 153. In certain embodiments, the polypeptide is a fragment of full-length LOXL1 polypeptide. Such fragments can in certain embodiments include 20 or more amino acids, including 25 or more, 30 or more, 35 or more, 40 or more, 50 or more or 100 or more amino acids. In certain embodiments, the fragment is biologically active, i.e. it retains at least a partial activity associated with LOXL1 in vivo. In certain embodiments, the fragments retains an activity that is substantially identical to human LOXL1 activity. In another aspect, the invention provides a method of preventing or ameliorating symptoms associated with glaucoma or exfoliation syndrome, the method comprising administering to an individual in need thereof a composition comprising LOXL1 polypeptide in a therapeutically effective amount. The polypeptide is in one embodiment characterized by the presence of a Leucine in position 141 in the sequence as set forth in SEQ ID NO:85. In another embodiment, the polypeptide is characterized by the presence of an Aspartic Acid in position 153 in the sequence as set forth in SEQ ID NO:85. In yet another embodiment, the polypeptide is characterized by the presence of a Leucine in position 141 and an Aspartic Acid in position 153 in the sequence as set forth in SEQ ID NO:85.

Administration of the polypeptide can be performed by a method selected from topical administration, intraocular administration, parenteral administration, intranasal administration, intratracheal administration, intrabronchial administration and subcutaneous administration. Preferably, the LOXL1 protein is administered by intraocular administration.

Markers associated with risk of ocular conditions can also be useful for selecting individuals for treatment with therapeutic agents for such conditions. Individual carrying particular LOXL1 variants are contemplated to be suitable for administration for therapy. Thus, one aspect of the invention provides a method of prophylaxis therapy for an ocular condition selected from glaucoma and exfoliation syndrome, comprising steps of selecting a human subject at risk for an ocular condition selected from glaucoma and exfoliation syndrome, and administering to the subject a therapeutically effective amount of a composition comprising a therapeutic agent for glaucoma, elevated intraocular pressure or exfoliation syndrome, wherein the selecting comprises determining a LOXL1 variant for the human subject, and selecting for prophylaxis therapy a human subject with a LOXL1 variant that correlates with an increased risk for the ocular condition. The LOXL1 variant can be a protein variant or a gene variant that correlates with risk of the ocular condition. In certain embodiments, the selecting comprises determining the presence or absence of a genotype or haplotype in the LOXL1 gene that correlates with increased risk of the ocular condition. In one embodiment, the genotype comprises at least one marker selected from rs1048661 and marker rs3825942, and markers in linkage disequilibrium therewith. In another embodiment, the selecting comprises selecting a human subject having a genotype that comprises rs1048661 allele G and/or rs3825942 allele G. In another embodiment, the haplotype is selected from the haplotype characterized by the presence of allele G in marker rs1048661 (SEQ ID NO:106) and allele G in marker rs3825942 (SEQ ID NO: 107); and the haplotype characterized by the presence of allele T in marker rs1048661 (SEQ ID NO: 106) and allele G in marker rs3825942 (SEQ ID NO: 107). In one preferred embodiment, the selecting comprises determining the presence of a LOXL1 protein in the human subject having an Arginine at position 141 and/or a Glycine at position 153 in the sequence as set forth in SEQ ID NO:85. In an especially preferred embodiment, the selecting comprises determining the presence of a LOXL1 protein in the human subject having an Arginine at position 141 and a Glycine at position 153 in the sequence as set forth in SEQ ID NO:85.

The invention also provides a therapeutic agent for an ocular condition selected from glaucoma, elevated intraocular pressure and exfoliation syndrome for treating a human subject with a LOXL1 variant that correlates with an increased risk for the ocular condition. Also provided is a therapeutic agent for treating a LOXL1-related ocular condition selected from exfoliation syndrome and glaucoma. A “LOXL1-related ocular condition” is, in this context, a condition selected from exfoliation syndrome and glaucoma in an individual who has at least one risk variant for the condition as described herein. Preferably, the risk variant is rs1048661 allele G or rs3825942 allele G. The risk variant can also comprise two markers shown herein to be associated with exfoliation syndrome and glaucoma. In certain embodiments, the risk variant is a haplotype selected from G-rs1048661 G-rs3825942 and T-1048661 G-rs3825942. Also provided is the use of a therapeutic agent for an ocular condition selected from glaucoma, elevated intraocular pressure and exfoliation syndrome for the manufacture of a medicament for treating the ocular condition in a human subject with a LOXL1 variant that correlates with an increased risk for the ocular condition. The invention also provides in another aspect use of a therapeutic agent for an ocular condition selected from glaucoma, elevated intraocular pressure and exfoliation syndrome for the manufacture of a medicament for treating a LOXL-1 related ocular condition. The ocular condition is in one embodiment selected from exfoliation syndrome and glaucoma. In certain embodiments, the human subject has at least one genotype or haplotype in the LOXL1 gene that correlates with increased risk of the ocular condition. The genotype can in some embodiments comprise at least one marker selected from rs1048661 and marker rs3825942, and markers in linkage disequilibrium therewith. In preferred embodiments, the human subject has a genotype that comprises rs1048661 allele G and/or rs3825942 allele G. In some embodiments, the haplotype is selected from the haplotype characterized by the presence of allele G in marker rs1048661 (SEQ ID NO: 106) and allele G in marker rs3825942 (SEQ ID NO: 107), and the haplotype characterized by the presence of allele T in marker rs1048661 (SEQ ID NO: 106) and allele G in marker rs3825942 (SEQ ID NO: 107). The protein variant is in certain embodiments, a LOXL1 variant, wherein the LOXL1 variant comprises a LOXL1 protein having an Arginine at position 141 and/or a Glycine at position 153 in the sequence as set forth in SEQ ID NO:85.

The therapeutic agent can be any suitable agent that is useful for treating the ocular condition, or an agent that is useful for preventing or ameliorating symptoms associated with the ocular condition. In one embodiment, the agent is selected from the agents set forth in Agent Table I. Any one or a combination of the agents set forth herein can be administered, including any prodrugs, salts or derivatives thereof. In certain embodiment, the individual presents with symptoms of the ocular disorder prior to treatment. It is however contemplated that presymptomatic treatment may be especially beneficial. Individuals carrying at least one at-risk variant for glaucoma and/or exfoliation syndrome are at especially high risk of developing these disorders. As a consequence, it is important to recognize the risk at an early stage to minimize the devastating consequence of these disorders. Ideally, presymptomatic treatment can prevent or at least substantially delay, the onset of the disease, or symptoms associated with the disease. Thus, early intervention can be very important for minimizing consequences of elevated intraocular pressure and other symptoms of exfoliation syndrome and glaucoma.

The invention is generally described herein in the context of glaucoma and exfoliation syndrome. The various aspects of the invention are however also contemplated in the context of subphenotypes of glaucoma, in particular exfoliation glaucoma. Thus, in certain embodiments, the ocular condition is selected from exfoliation glaucoma (XFG) or exfoliation syndrome (XFS).

It should be understood that all combinations of features described herein are contemplated, even if the combination of feature is not specifically found in the same sentence or paragraph herein. This includes in particular the use of all markers disclosed herein, alone or in combination, for analysis individually or in haplotypes, in all aspects of the invention as described herein. Where embodiments pertain to evaluation of a polymorphism in an individual, it should be understood that a related embodiment involves evaluation of both alleles of the marker in the individual, and the same applies for haplotype evaluation.

The original claims appended hereto are hereby incorporated by reference into the summary of the invention.

The foregoing summary is not intended to define every aspect of the invention, and additional aspects are described in other sections, such as the Drawing or Detailed Description. The entire document is intended to be related as a unified disclosure, and it should be understood that all combinations of features described herein are contemplated, even if the combination of features are not found together in the same sentence, or paragraph, or section of this document. Where protein therapy is described, embodiments involving polynucleotide therapy (using polynucleotides that encode the protein) are specifically contemplated, and the reverse also is true.

In addition to the foregoing, the invention includes, as an additional aspect, all embodiments of the invention narrower in scope in any way than the variations specifically mentioned above. With respect to aspects of the invention described as a genus, all individual species are individually considered separate aspects of the invention. With respect to evaluating polymorphisms, all combinations of the predictive polymorphisms described herein are contemplated, even though not all individual combinations are specifically recited. With respect to aspects of the invention that are described with reference to exemplary numerical values, it should be understood that such values are intended to describe ranges or sub-ranges that include the recited values. With respect to aspects described with numerical ranges, it should be understood that all subranges are contemplated.

Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention. Additional features and variations of the invention will be apparent to those skilled in the art from the entirety of this application, and all such features are intended as aspects of the invention.

### BRIEF DESCRIPTION OF THE DRAWINGS

The foregoing and other objects, features and advantages of the invention will be apparent from the following more particular description of preferred embodiments of the invention.

FIG. 1 shows the genomic structure of a region on chromosome 15 that includes the LOXL1 gene. The positions (in bp) on the chromosome are from NCBI Build 34. Shown is the exon-intron structure of the LOXL1 gene, and Oxford recombination hotspots, which define the boundaries of the LOXL1 LD block.

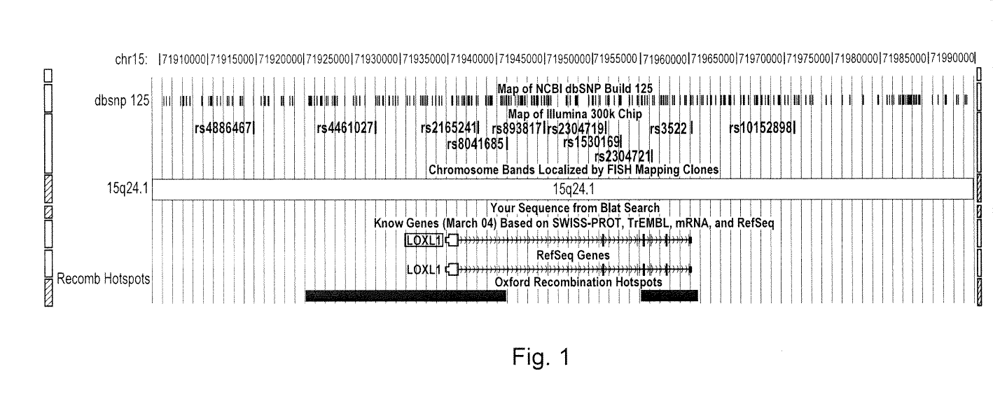

FIG. 2 shows a schematic view of the association of glaucoma to 15q24.1. a) The pair-wise correlation structure in a 400 kb interval (71.8-72.2 Mb, NCBI Build 34) on chromosome 15. The upper plot includes pair-wise D′ for 269 common SNPs (with MAF>5%) from the HapMap release 22 for the CEU population, while the lower plot includes pair-wise r² values for the same set of SNPs. b) Location of recombination hot-spots in this interval based on the HapMap dataset (Nature 437, 1299-1320 (2005)). c) Location of eight known genes in this region. d) Schematic view of the genome-wide association results in the interval from the genome-wide association study of 195 Icelandic glaucoma cases and 14474 controls. Plotted is −log P, where P is the adjusted P-value, against the chromosomal location of the markers. All four panels use the same horizontal Mb scale indicated at the bottom of panel d).

FIG. 3 shows the association of XFG to haplotypes formed by the two non-synonymous SNPs, rs1048661 and rs3825942. The figure illustrates pair-wise comparison of the risk of the three haplotypes, (G,G), (G,A) and (T,G), formed by the alleles of the two non-synonymous SNPs rs1048661 (R141L) and rs3825942 (G153D). Each arrow indicates the comparison of two haplotypes and the OR is that of the haplotype the arrow is pointing to relative to the other haplotype. The results are shown for a) Iceland and b) Sweden separately, and c) for the two populations combined. Confidence intervals (CI) for the OR's are included in parenthesis. Estimated haplotype frequencies in cases and controls are given in parenthesis below each haplotype. For the populations combined, the haplotype frequencies given are the simple averages of the frequencies in the two populations. Computing ORs from these averages would not give the same results as the ORs displayed, the latter calculated using a Mantel-Haenszel model, but the numbers are close. Note that the haplotype formed by the protective alleles, (T,A), is not observed in the Icelandic or the Swedish cases-control groups.

FIG. 4 shows the correlation between genotypes of rs1048661 (R141L) and expression of LOXL1 in adipose tissue. Expression of LOXL1 measured in adipose tissue from 659 individuals for the different genotypes of the non-synonymous at-risk SNP rs1048661 (R141L). The expression of LOXL1 is shown as 10̂(average MLR), where MLR is the mean log expression ratio and the average is over individuals with a particular genotype. Regressing the MLR values on the number of copies of the at-risk variant G an individual carries we find that the expression of LOXL1 is reduced by an estimated 7.7% with G allele carried (P=0.00000083). The effect of age and sex is taken into account by including an Age×Sex term among the explanatory variables in the regression. The vertical bars indicate the standard error of the mean (s.e.m.). The correlation remains if the expression is adjusted for weight of the individual by including body-mass index (BMI) as an explanatory variable (P=0.0000013). Similar results are obtained if the 275 males and the 384 females are analyzed separately (P=0.00029 and P=0.00060, respectively).

FIG. 5 shows the correlation between genotypes of rs1048661 (R141L) and expression of LOXL1 in adipose tissue measured with real-time PCR. Expression of LOXL1 measured in adipose tissue from 564 individuals for the different genotypes of the non-synonymous at-risk SNP rs1048661 (R141L) measured with real-time PCR. The expression of LOXL1 is shown relative to the expression of GUSP (as a housekeeping gene). Regressing the log-transformed expression values on the number of copies of the at-risk variant G an individual carries we find that the expression of LOXL1 is reduced by an estimated 9.1% with G allele carried (P=0.000037). The effect of age and sex is taken into account by including an Age×Sex term among the explanatory variables in the regression. The vertical bars indicate the standard error of the mean (s.e.m.).

FIG. 6 shows an exemplary computer environment on which the methods and apparatus as described and claimed herein can be implemented.

### DETAILED DESCRIPTION OF THE INVENTION

A description of preferred embodiments of the invention follows.

The present invention discloses polymorphic variants and haplotypes that have been found to be associated with ocular conditions, in particular exfoliation syndrome (XFS) and glaucoma. Particular alleles at polymorphic markers (e.g., the markers associated with the LOXL1 gene, as defined herein, and markers in linkage disequilibrium therewith) and haplotypes comprising such alleles have been found to be associated with XFS and glaucoma. Such markers and haplotypes are useful for diagnostic purposes, as described in further detail herein. Further applications of the present invention includes methods for assessing response to therapeutic agents for XFS and/or glaucoma utilizing the polymorphic markers of the invention, methods for predicting prognosis of an individual experiencing symptoms associated with, or an individual diagnosed with, XFS and/or glaucoma, methods of monitoring progress of a treatment of an individual undergoing treatment for symptoms associated with XFS and/or glaucoma, computer-implemented aspects, as well as kits for assessing susceptibility of an individual to XFS and/or glaucoma.

### Definitions

Unless otherwise indicated, nucleic acid sequences are written left to right in a 5′ to 3′ orientation. Numeric ranges recited within the specification are inclusive of the numbers defining the range and include each integer or any non-integer fraction within the defined range. Unless defined otherwise, all technical and scientific terms used herein have the same meaning as commonly understood by the ordinary person skilled in the art to which the invention pertains.

The following terms shall, in the present context, have the meaning as indicated:

A “polymorphic marker”, sometimes referred to as a “marker”, as described herein, refers to a genomic polymorphic site. Each polymorphic marker has at least two sequence variations characteristic of particular alleles at the polymorphic site. Thus, genetic association to a polymorphic marker implies that there is association to at least one specific allele of that particular polymorphic marker. The marker can comprise any allele of any variant type found in the genome, including single nucleotide polymorphisms (SNPs), mini- or microsatellites, translocations and copy number variations (insertions, deletions, duplications). Polymorphic markers can be of any measurable frequency in the population. For mapping of disease genes, polymorphic markers with population frequency higher than 5-10% are in general most useful. However, polymorphic markers may also have lower population frequencies, such as 1-5% frequency, or even lower frequency, in particular copy number variations (CNVs). The term shall, in the present context, be taken to include polymorphic markers with any population frequency.

An “allele” refers to the nucleotide sequence of a given locus (position) on a chromosome. A polymorphic marker allele thus refers to the composition (i.e., sequence) of the marker on a chromosome. Genomic DNA from an individual contains two alleles for any given polymorphic marker, representative of each copy of the marker on each chromosome. Sequence codes for nucleotides used herein are: A=1, C=2, G=3, T=4. For microsatellite alleles, the CEPH sample (Centre d'Etudes du Polymorphisme Human, genomics repository, CEPH sample 1347-02) is used as a reference, the shorter allele of each microsatellite in this sample is set as 0 and all other alleles in other samples are numbered in relation to this reference. Thus, e.g., allele 1 is 1 bp longer than the shorter allele in the CEPH sample, allele 2 is 2 bp longer than the shorter allele in the CEPH sample, allele 3 is 3 bp longer than the lower allele in the CEPH sample, etc., and allele −1 is 1 bp shorter than the shorter allele in the CEPH sample, allele −2 is 2 bp shorter than the shorter allele in the CEPH sample, etc.

Sequence conucleotide ambiguity as described herein is as proposed by IUPAC-IUB. These codes are compatible with the codes used by the EMBL, GenBank, and PIR databases.

| IUB code | Meaning |
|---|---|
| A | Adenosine |
| C | Cytidine |
| G | Guanine |
| T | Thymidine |
| R | G or A |
| Y | T or C |
| K | G or T |
| M | A or C |
| S | G or C |
| W | A or T |
| B | C G or T |
| D | A G or T |
| H | A C or T |
| V | A C or G |
| N | A C G or T (Any base) |

A nucleotide position at which more than one sequence is possible in a population (either a natural population or a synthetic population, e.g., a library of synthetic molecules) is referred to herein as a “polymorphic site”.

A “Single Nucleotide Polymorphism” or “SNP” is a DNA sequence variation occurring when a single nucleotide at a specific location in the genome differs between members of a species or between paired chromosomes in an individual. Most SNP polymorphisms have two alleles. Each individual is in this instance either homozygous for one allele of the polymorphism (i.e. both chromosomal copies of the individual have the same nucleotide at the SNP location), or the individual is heterozygous (i.e. the two sister chromosomes of the individual contain different nucleotides). The SNP nomenclature as reported herein refers to the official Reference SNP (rs) ID identification tag as assigned to each unique SNP by the National Center for Biotechnological Information (NCBI).

A “variant”, as described herein, refers to a segment of DNA that differs from the reference DNA. A “marker” or a “polymorphic marker”, as defined herein, is a variant. Alleles that differ from the reference are referred to as “variant” alleles.

A “fragment” of a nucleotide or a protein, as described herein, comprises all or a part of the nucleotide or the protein.

An “animal”, as described herein, refers to any domestic animal (e.g., cats, dogs, etc.), agricultural animal (e.g., cows, horses, sheep, chicken, etc.), or test species (e.g., rabbit, mouse, rat, etc.), and also includes humans.

A “microsatellite” is a polymorphic marker that has multiple small repeats of bases that are 2-8 nucleotides in length (such as CA repeats) at a particular site, in which the number of repeat lengths varies in the general population.

An “indel” is a common form of polymorphism comprising a small insertion or deletion that is typically only a few nucleotides long.

A “haplotype,” as described herein, refers to a segment of genomic DNA within one strand of DNA that is characterized by a specific combination of alleles arranged along the segment. For diploid organisms such as humans, a haplotype comprises one member of the pair of alleles for each polymorphic marker or locus. In a certain embodiment, the haplotype can comprise two or more alleles, three or more alleles, four or more alleles, or five or more alleles. Haplotypes are described herein in the context of the marker name and the allele of the marker in that haplotype, e.g., “3 rs1048661” refers to the 3 allele of marker rs1048661 being in the haplotype, and is equivalent to “rs1048661 allele 3”. Furthermore, allelic codes in haplotypes are as for individual markers, i.e. 1=A, 2=C, 3=G and 4=T.

The term “susceptibility”, as described herein, encompasses both increased susceptibility and decreased susceptibility. Thus, particular polymorphic markers and/or haplotypes of the invention may be characteristic of increased susceptibility (i.e., increased risk) of glaucoma, as characterized by a relative risk (RR) of greater than one. Alternatively, the markers and/or haplotypes of the invention are characteristic of decreased susceptibility (i.e., decreased risk) of glaucoma, as characterized by a relative risk of less than one.

The term “and/or” shall in the present context be understood to indicate that either or both of the items connected by it are involved. In other words, the term herein shall be taken to mean “one or the other or both”.

The term “look-up table”, as described herein, is a table that correlates one form of data to another form, or one or more forms of data to a predicted outcome to which the data is relevant, such as phenotype or trait. For example, a look-up table can comprise a correlation between allelic data for at least one polymorphic marker and a particular trait or phenotype, such as a particular disease diagnosis, that an individual who comprises the particular allelic data is likely to display, or is more likely to display than individuals who do not comprise the particular allelic data. Look-up tables can be multidimensional, i.e. they can contain information about multiple alleles for single markers simultaneously, or the can contain information about multiple markers, and they may also comprise other factors, such as particulars about diseases diagnoses, racial information, biomarkers, biochemical measurements, therapeutic methods or drugs, etc.

A “computer-readable medium”, is an information storage medium that can be accessed by a computer using a commercially available or custom-made interface. Exemplary compute-readable media include memory (e.g., RAM, ROM, flash memory, etc.), optical storage media (e.g., CD-ROM), magnetic storage media (e.g., computer hard drives, floppy disks, etc.), punch cards, or other commercially available media. Information may be transferred between a system of interest and a medium, between computers, or between computers and the computer-readable medium for storage or access of stored information. Such transmission can be electrical, or by other available methods, such as IR links, wireless connections, etc.

A “nucleic acid sample” is a sample obtained from an individual that contains nucleic acid (DNA or RNA). In certain embodiments, i.e. the detection of specific polymorphic markers and/or haplotypes, the nucleic acid sample comprises genomic DNA. Such a nucleic acid sample can be obtained from any source that contains genomic DNA, including as a blood sample, sample of amniotic fluid, sample of cerebrospinal fluid, or tissue sample from skin, muscle, buccal or conjunctival mucosa (buccal swab), placenta, gastrointestinal tract or other organs.

The term “polypeptide”, as described herein, refers to a polymer of amino acids, and not to a specific length; thus, peptides, oligopeptides and proteins are included within the definition of a polypeptide.

The term “XFS”, as described herein, refers to exfoliation syndrome. An alternative name of this disorder is pseudoexfoliation syndrome (PEX). As described herein, the terms XFS and PEX have the same meaning, as is well known to the skilled person in the medical field.

The term “glaucoma”, as described herein, refers to the heterogeneous group of disorders jointly called glaucoma in the medical field. The term is meant to include the various subtypes of glaucoma, including open angle glaucoma (OAG), primary open angle glaucoma (POAG), secondary glaucoma and exfoliation glaucoma (XFG).

The term “XFS and/or glaucoma therapeutic agent”, as described herein, refers to an agent that can be used to ameliorate or prevent symptoms associated with XFS and/or glaucoma, i.e. symptoms associated with XFS only, symptoms associated with glaucoma only, or symptoms associated with XFS and glaucoma.

The term “XFS and/or glaucoma-associated nucleic acid”, as described herein, refers to a nucleic acid that has been found to be associated to XFS and/or glaucoma. This includes, but is not limited to, the markers and haplotypes described herein and markers and haplotypes in strong linkage disequilibrium (LD) therewith. In one embodiment, a XFS and/or glaucoma-associated nucleic acid refers to an LD-block (e.g., LOXL1 LD block) found to be associated with XFS and/or glaucoma through at least one polymorphic marker located within the LD block or associated with the LD block.

The term “LOXL1 LD block”, or “LOXL1 linkage disequilibrium block”, also called “LD Block C15”, as described herein, refers to the Linkage Disequilibrium (LD) block on Chromosome 15 between positions 71,928,222 and 71,966,707 of NCBI (National Center for Biotechnology Information) Build 34, and between positions 71,999,458 and 72,037,943 in NCBI Build 36. The LOXL1 LD block is 38,486 bp in size in both sequence builds. The nucleotide sequence of the LOXL1 LD block, as defined in NCBI Build 34, is provided herein as SEQ ID NO: 84.

The term “LOXL1 gene”, or “LOXL1” as described herein, refers to the Lysyl Oxidase-Like 1 gene, which is homologous to the lysyl oxidase gene. The gene is located on chromosome 15q24.1, and spans approximately positions 71,934,605-71,960,295 (NCBI Build 34) or positions 72,005,841-72,031,531 (NCBI Build 36). The gene spans 25,690 bp of genomic sequence in both these Builds of the human genome assembly.

The term “high myopia”, as described herein, refers to myopia characterized by 6.0 dioptres (D) or greater. In general, myopia up to 3.0 dioptres is termed mild degree, 3.0 to 6.0D is moderate degree and high degree myopia, or high myopia, is 6.0D and over.

The term “thin cornea”, as described herein, refers to cornea characterized by a central cornea thickness of less than 556 μm.

Through association analysis of a population of individuals diagnosed with XFS and/or glaucoma, it has been discovered that certain alleles at certain polymorphic markers are associated with XFS and glaucoma. A genome-wide analysis for variants associated with glaucoma revealed association of glaucoma to a distinct region of chromosome 15, i.e. Chr15q24.1. Follow-up analysis revealed association to XFS and glaucoma. Particular markers were found to be associated with an increased risk of XFS and glaucoma in this region.

Thus, the SNP marker rs2165241 was found to be associated with XFS and glaucoma, as illustrated in Table 1. Several markers that are correlated with rs2165241 were also found to be strongly associated with XFS, as shown in Table 2. Those markers, and other markers in linkage disequilibrium with rs2165241, could therefore also be used as surrogate markers for detecting the association between rs2165241 and XFS/glaucoma. These markers thus identified a region, defined herein as the LOXL1 LD block, which harbors polymorphic variants (e.g., SNPs and microsatellites) that associated with XFS and glaucoma, and could therefore be used in the methods and kits of the present invention, as disclosed further herein. Follow-up sequencing of the LOXL1 gene, including all of its exons, and the 5′ and 3′ flanking regions, revealed several novel SNP markers. Additional analysis has revealed that the greatest contribution to the association signal in the LOXL1 gene is provided by markers rs1048661 and rs3825942, both of which are located in exon 1 of the gene (see Table 7). These two markers lead to amino acid substitutions at positions 141 (R141L) and 153 (G135D), respectively. These markers, and markers in linkage disequilibrium therewith (e.g., the markers listed in Table 15) are therefore particularly suitable for determining an increased or decreased risk of glaucoma or exfoliation syndrome. Preferred embodiments of the invention therefore relate to the use of rs1048661, rs3825942, and markers in linkage disequilibrium therewith, such as the markers provided in Table 15.

The rs2165241 marker on chromosome 15q24.1 associated to XFS and Glaucoma is located in the first intron of the LOXL1 gene, while the rs1048661 and rs3825942 markers are located in exon 1. The LOXL1 gene is a member of the family of lysyl oxidases that catalyse oxidative deamination of lysine residues of tropoelastin in the formation of the elastin polymer fibers. The five LOX family members encode the prototypic LOX protein and LOX-like proteins 1 through 4 (LOXL1, LOXL2, LOXL3 and LOXL4) their individual roles in elastogenesis being unclear (Kagan, H M & Li, W, J Cell Biochem 88:660-672 (2003)). LOXL1-deficient mice fail to maintain elastic fiber homeostasis that leads to pelvic floor disorders (Liu X, et al., Am J Pathol 168: 519-528 (2006)), and show elastic fiber defects in number of tissues (Liu X, et al., Nat Genet. 36: 178-182 (2004)). Publicly available gene expression databases (Gene Expression Omnibus (GEO) (Barrett T, et al. Nucleic Acids Res 35: D760-765 (2007)) have been used to verify expression of LOXL1 in both normal and glaucomatous ocular tissues like lamina cribrosa and trabecular meshwork (Hernandez M R, et al., Glia 38: 45-64 (2002)). In a model where human lamina cribrosa cells were used as a model for primary POAG, LOXL1 was upregulated upon stimulation with 10 ng/ml TGF-beta1 for 24 hours (Kirwan R P, et al., Glia 52: 309-324 (2005)).

Glaucomatous optic nerve head is characterized by a cup formation and excavation caused by extensive remodeling of the extracellular matrix and collapse of the underlying connective tissue lamina cribrosa. The lamina cribrosa is a perforated connective tissue plate that protects the retinal canglion cell axons in the optic nerve head. In a monkey model, the axoplasmic flow in retinal ganglion cell axons is arrested upon collapse of the lamina cribrosa (Pena J D, et al., Br J Opthalmol 83: 209-218 (1999)). Also, the compliance of lamina cribrosa reduces markedly with age (Hernandez M R, et al., Am J Opthalmol 107: 476-484 (1989); Albon J, et al., Br J Opthalmol 84: 311-317 (2000)). Quantitative studies of elastin in the lamina cribrosa show no difference between normal and glaucomatous eyes in amount, number or structure (Quigley E N, et al., Opthalmology 103: 1680-1685 (1996)). Further, elastosis of the lamina cribrosa has been shown to occur in patients with exfoliation syndrome and POAG, demonstrated by marked and site-specific elastosis, suggesting an abnormal regulation of elastin synthesis (Pena J D, et al., Exp Eye Res 67: 517-524 (1998)). Elevated IOP subjects the extracellular matrix of the lamina cribrosa to an immense strain that leads to damage and requires active remodeling. To repair and to replace damaged fibers requires the coordinated re-expression of all of the molecules that make up the matrix as well as the enzymes critical for cross-linking elastin. Inadequate expression of LOXL1 may be insufficient to crosslink and organize into a functional three-dimensional fiber. The hypothesized disease process in glaucoma is the inability of LC cells to repair damaged elastic fibers in lamina cribrosa, which leads to permanently neurodegeneration. Therapeutic value of regulating LOXL1 to counteract elastosis caused in glaucoma is immense and is feasible through intervention of the LC cells.

A number of genetic loci for congenital and juvenal glaucoma have been reported (Hewitt A W, et al., Clin Experiment Opthalmol 34: 472-484 (2006)). However, in adult glaucoma only one disease gene have shown considerable impact, MYOC (Stone E M, et al. Science 275: 668-670 (1997)) that codes for myocillin shows a mutation prevalence of 2-4% of POAG and over 10% in juvenile open angle glaucoma (JOAG). MYOC mutations cause dysfunctions in the trabecular meshwork, and genotype-phenotype correlations are strong.

The genetic contribution to glaucoma of the at-risk variants in LOXL, is much greater than observed in MYOC or any known genetic variant known in adult onset glaucoma, POAG or XFS. The common at-risk variants described herein give the opportunity to develop a cost-effective population based screening program to enable early therapeutic intervention. Because glaucoma is initially asymptomatic it is important to identify individuals with a genetic predisposition for developing the disease. Individuals carrying one or two copies of the at-risk variants in LOXL1 described herein run in excess of a 3-fold (one copy) and 10-fold (two copies) risk of developing XFS compared with non-carriers, respectively. Furthermore, carriers of allele G at marker rs1048661 and allele G at marker rs3825942 on the same chromosome, i.e. carriers of the G-rs1048661 G-rs3825942 haplotype, are at 27-fold increase in risk of exfoliation glaucoma (XFG) compared with carriers of the G-rs1048661 A-rs3825942 haplotype. Based on carrier status the variants disclosed herein, a cost effective decision can be made with respect to how to monitor surveillance, early therapeutic intervention and to prevent blindness.

### Potential for Utilization of the LOXL1 Gene in the Development of Improved Therapies for Glaucoma and XFS

Most cases of glaucoma are not discovered until vision has been permanently lost, because clinical signs of the disease are subtle. However, the loss of vision caused by glaucoma could be limited or prevented by currently available therapies. It is therefore imperative to characterize and utilize the risk factors associated with increased risk of the disease. The present invention provides markers that can be used to identify those individuals that are at increased risk of developing symptoms associated with glaucoma and other eye disorders, including XFS. The present invention thus provides methods and kits for detecting and identifying those individuals who are at increased risk of developing glaucoma. Thus, the present invention provides methods for providing a more efficient and cost-effective way of identifying individuals likely to develop the disease. Using measurements of intraocular pressure to screen populations for glaucoma is not an effective method (Weinreb & Khaw, Lancet 363: 1711-20 (2004)). The most widely method used, Goldmann tonometry, underestimates the true intraocular pressure of patients with thin corneas and overestimates the pressure in patients with thick ones. Furthermore, half of all patients with primary open-angle glaucoma have pressure below 22 mm Hg (Mitchell et al., Opthalmol 103:1661-69 (1996)). In addition, most individuals with raised pressure do not have, and may never develop, optic nerve damage. Therefore, current methods need to rely on assessment of the optic disc, retinal nerve fibre layer and visual function, in addition to pressure.

The additional application of genetic risk factors may thus facilitate the development of more cost-effective and reliable prevention programs that are aimed at detecting eye disorders such as glaucoma, XFS or cataract, at an early stage. Such programs may provide individuals who are at risk of developing the disease, or individuals with early symptoms of the disease, hope for a successful therapeutic intervention based, in part, on the result of the genetic testing. Thus, individuals positive for the genetic test may be selected for more rigorous or frequent examination, or, if they also present with early symptoms of the disease, may undergo more aggressive therapeutic intervention. Also, the variants of the invention may be used to screen for those individuals who are most likely to benefit from irridectomy, which is an invasive medical procedure.

A number of medications for glaucoma and lowering intraocular pressure have been developed. Prostaglandin analogs and prostamides, including latanoprost, travoprost, unoprostone and bimatoprost, reduce intraocular pressure by increasing the outflow of aqueous humour, and have in general become the first line of treatment. Some prostaglandins activate matrix metalloproteinases, which then remodel extracellular matrix and reduce outflow resistance, allowing the aqueous humour to flow out through this route. Other types of medications for reducing intraocular pressure include a2 adrenergic agonists, such as brimonidine and apraclonidine, carbonic anhydrase inhibitors, such as dorzolamide, brinzolamide, acetazolamide and methozolamide, β-blockers such as betaxolol, carteolol, levobunolol, metipranolol and timolol, and cholinergic agonists, for example pilocarpine and carbachol. Further information on glaucoma medications is provided in Agent Table I. Lowering intraocular pressure can, on average, reduce the number of ocular hypertensive patients progressing to glaucoma by one-half, and can also prevent progression in patients with pre-existing glaucoma. However, early intervention is of great importance.

AGENT TABLE I

| | | | US patent |
|---|---|---|---|
| Name | Brand name | Active compound | docs |
| Epinephrine | | (R)-4-(1-hydroxy- | |
| | | 2-(methylamino)ethyl)benzene-1,2-diol | |
| echothiophate | Phospholine | 2-diethoxyphosphorylsulfanylethyl-trimethyl- | |
| | | ammonium iodide | |
| timolol | betimol | (S)-1-(tert-butylamino)-3-(4-morpholino-1,2,5- | 5231095 |
| | | thiadiazol-3-yloxy)propan-2-ol | |
| latanoprost | Xalatan | isopropyl (Z)-7-[(1R,2R,3R,5S)-3,5-dihydroxy-2- | 5296504, |
| | | [(3R)3-hydroxy-5-phenylpentyl]-cyclopentyl] | 5422368, |
| | | hept-5-enoate | 6429226, |
| | | | 7163959 |
| travoprost | Travatan | propan-2-yl 7-[3,5-dihydroxy-2-[3-hydroxy-4-[3- | 5510383, |
| | | (trifluoromethyl)phenoxy]-but-1-enyl]- | 5631287, |
| | | cyclopentyl]hept-5-enoate | 5849792, |
| | | | 5889052, |
| | | | 6011062 |
| unoprostone | Rescula | (Z)-7-[(1R,2R,3R,5S)-3,5-dihydroxy-2-(3- | |
| | | oxodecyl)cyclopentyl]hept-5-enoic acid | |
| bimatoprost | Lumigan | 7-[3,5-dihydroxy-2-(3-hydroxy-5-phenyl-pent-1- | 5688819, |
| | | enyl)-cyclopentyl]-N-ethyl-hept-5-enamide | 6403649 |
| brimonidine | Alphagan | 5-Bromo-N-(4,5-dihydro-1H-imidazol-2-yl) | 5424078, |
| | | quinoxalin-6-amine | 6562873, |
| | | | 6627210, |
| | | | 6641834, |
| | | | 6673337 |
| apraclonidine | Iopidine | 2,6-dichloro-N-(4,5-dihydro-1H-imidazol-2-yl) | |
| | | benzene-1,4-diamine | |
| dorzolamide | Trusopt | 2-ethylamino-4-methyl-5, 5-dioxo-5$\|\hat{ }{6}, 7- | 4797413 |
| | | dithiabicyclo[4.3.0] nona-8,10-diene-8- | |
| | | sulfonamide | |
| brinzolamide | Azopt | (5R)-5-ethylamino-3-(3-methoxypropyl)-2,2- | 5240923, |
| | | dioxo-2λ6,9-dithia-3-azabicyclo[4.3.0]nona-7,10- | 5378703, |
| | | diene-8-sulfonamide | 5461081 |
| acetazolamide | Diamox | N-(5-(aminosulfonyl)-1,3,4-thiadiazol-2-yl)- | |
| | | acetamide | |
| methozolamide | Neptazane | N-(3-methyl-5-sulfamoyl-3H-1,3,4-thiadiazol-2- | |
| | | ylidene) ethanamide | |
| betaxolol | Betoptic, Betoptic S, | 1-[4-[2-(cyclopropylmethoxy)ethyl]phenoxy]-3- | 4911920 |
| | Lokren, Kerlone | (1-methylethylamino)propan-2-ol | |
| carteolol | | 5-[2-hydroxy-3-(tert-butylamino) propoxy]-3,4- | |
| | | dihydro-1H-quinolin-2-one | |
| levobunolol | | 5-(2-hydroxy-3-tert-butylamino-propoxy) | |
| | | tetralin-1-one | |
| metipranolol | OptiPranolol | [4-[2-hydroxy-3-(1-methylethylamino) propoxy]- | |
| | | 2,3,6-trimethyl-phenyl] ethanoate | |
| pilocarpine | Diocarpine, Isopto | (3S,4R)-3-ethyl-4-((1-methyl-1H-imidazol-5-yl) | |
| | Carpine, Miocarpine, | methyl)dihydrofuran-2(3H)-one | |
| | Ocusert, Piolopine | | |
| | HS, Salagen, Timpilo | | |
| carbachol | Carbastat, Barboptic, | 2-carbamoyloxyethyl-trimethyl-azanium | |
| | Isopto Carbachol, | | |
| | Miostat | | |
| BVT-28949 | | | |
| (Biovitrum) | | | |
| PF-03187207 | | | |
| (NicOx) | | | |
| tafluprost (AFP- | | 1-methylethyl (5Z)-7-[(1R,2R,3R,5S)-2-[(1E)- | |
| 168) | | 3,3-difluoro-4-phenoxy-1-butenyl]-3,5- | |
| | | dihydroxycyclopentyl]-5-heptenoate | |
| mifepristone | Mifeprex | 11β-[p-(Dimethylamino)phenyl]-17β-hydroxy-17- | |
| (RU-486) | | (1-propynyl)estra-4,9-dien-3-one | |
| betaxoxime | | betaxolone oxime | |
| (IVAX Corp) | | | |
| SPI-015 | | | |
| (Sucampo) | | | |

Recently, drug delivery to the eye by means of intraocular injections has been marketed. Examples of successful therapeutic applications include Lucentis (ranibizumab injection) and Macugen (pegaptanib sodium injection), both of which are prescribed for the treatment of wet age-related macular degeneration.

Laser treatment for glaucoma is also available, the most widely-used form for open-angle glaucoma being laser trabeculoplasty, whereby laser light is directed at the trabecular meshwork to reduce the resistance to aqueous humour outflow. Another procedure is laser diode cyclophotocoagulation, used in more advanced cases, and has a more temporary effect. Surgical methods have also been developed, including trabeculectomy, in which a small portion of the trabecular meshwork is excised.

Although the present invention is exemplified by the very strong association between XFS and glaucoma and markers in the LOXL1gene, it is contemplated that the markers of the invention also are of diagnostic and/or therapeutic value for related disorders, including cataract, for which XFS is a risk factor, or risk factors associated with these disorders.

### Assessment for Markers and Haplotypes

The genomic sequence within populations is not identical when individuals are compared. Rather, the genome exhibits sequence variability between individuals at many locations in the genome. Such variations in sequence are commonly referred to as polymorphisms, and there are many such sites within each genome For example, the human genome exhibits sequence variations which occur on average every 500 base pairs. The most common sequence variant consists of base variations at a single base position in the genome, and such sequence variants, or polymorphisms, are commonly called Single Nucleotide Polymorphisms (“SNPs”). These SNPs are believed to have occurred in a single mutational event, and therefore there are usually two possible alleles possible at each SNP site; the original allele and the mutated allele. Due to natural genetic drift and possibly also selective pressure, the original mutation has resulted in a polymorphism characterized by a particular frequency of its alleles in any given population. Many other types of sequence variants are found in the human genome, including mini- and microsatellites, and insertions, deletions, and inversions (also called copy number variations (CNVs)). A polymorphic microsatellite has multiple small repeats of bases (such as CA repeats, TG on the complimentary strand) at a particular site in which the number of repeat lengths varies in the general population. In general terms, each version of the sequence with respect to the polymorphic site represents a specific allele of the polymorphic site. These sequence variants can all be referred to as polymorphisms, occurring at specific polymorphic sites characteristic of the sequence variant in question. In general terms, polymorphisms can comprise any number of specific alleles. Thus in one embodiment of the invention, the polymorphism is characterized by the presence of two or more alleles in any given population. In another embodiment, the polymorphism is characterized by the presence of three or more alleles. In other embodiments, the polymorphism is characterized by four or more alleles, five or more alleles, six or more alleles, seven or more alleles, nine or more alleles, or ten or more alleles. All such polymorphisms can be utilized in the methods and kits of the present invention, and are thus within the scope of the invention.

Due to their abundance, SNPs account for a majority of sequence variation in the human genome. Over 6 million SNPs have been validated to date (http://www.ncbi.nlm.nih.gov/projects/SNP/snp_summary.cgi). However, CNVs are receiving increased attention. These large-scale polymorphisms (typically 1 kb or larger) account for polymorphic variation affecting a substantial proportion of the assembled human genome; known CNVs covery over 15% of the human genome sequence (Estivill, X Armengol; L., PloS Genetics 3: 1787-99 (2007). A http://projects.tcag.ca/variation/). Most of these polymorphisms are however very rare, and on average affect only a fraction of the genomic sequence of each individual. CNVs are known to affect gene expression, phenotypic variation and adaptation by disrupting gene dosage, and are also known to cause disease (microdeletion and microduplication disorders) and confer risk of common complex diseases, including HIV-1 infection and glomerulonephritis (Redon, R., et al. Nature 23:444-454 (2006)). It is thus possible that either previously described or unknown CNVs represent causative variants in linkage disequilibrium with the variants described herein to be associated with XFS and glaucoma. Methods for detecting CNVs include comparative genomic hybridization (CGH) and genotyping, including use of genotyping arrays, as described by Carter (Nature Genetics 39:S16-S21 (2007)). The Database of Genomic Variants (http://projects.tcag.ca/variation/) contains updated information about the location, type and size of described CNVs. The database currently contains data for over 15,000 CNVs.

In some instances, reference is made to different alleles at a polymorphic site without choosing a reference allele. Alternatively, a reference sequence can be referred to for a particular polymorphic site. The reference allele is sometimes referred to as the “wild-type” allele and it usually is chosen as either the first sequenced allele or as the allele from a “non-affected” individual (e.g., an individual that does not display a trait or disease phenotype).

Alleles for SNP markers as referred to herein refer to the bases A, C, G or T as they occur at the polymorphic site in the SNP assay employed. The allele codes for SNPs used herein are as follows: 1=A, 2=C, 3=G, 4=T. The person skilled in the art will however realize that by assaying or reading the opposite DNA strand, the complementary allele can in each case be measured. Thus, for a polymorphic site (polymorphic marker) characterized by an A/G polymorphism, the assay employed may be designed to specifically detect the presence of one or both of the two bases possible, i.e. A and G. Alternatively, by designing an assay that is designed to detect the opposite strand on the DNA template, the presence of the complementary bases T and C can be measured. Quantitatively (for example, in terms of relative risk), identical results would be obtained from measurement of either DNA strand (+ strand or − strand).

Typically, a reference sequence is referred to for a particular sequence. Alleles that differ from the reference are sometimes referred to as “variant” alleles. A variant sequence, as used herein, refers to a sequence that differs from the reference sequence but is otherwise substantially similar. Alleles at the polymorphic genetic markers described herein are variants. Variants can include changes that affect a polypeptide. Sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence. Such sequence changes can alter the polypeptide encoded by the nucleic acid. For example, if the change in the nucleic acid sequence causes a frame shift, the frame shift can result in a change in the encoded amino acids, and/or can result in the generation of a premature stop codon, causing generation of a truncated polypeptide. Alternatively, a polymorphism associated with a disease or trait can be a synonymous change in one or more nucleotides (i.e., a change that does not result in a change in the amino acid sequence). Such a polymorphism can, for example, alter splice sites, affect the stability or transport of mRNA, or otherwise affect the transcription or translation of an encoded polypeptide. It can also alter DNA to increase the possibility that structural changes, such as amplifications or deletions, occur at the somatic level. The polypeptide encoded by the reference nucleotide sequence is the “reference” polypeptide with a particular reference amino acid sequence, and polypeptides encoded by variant alleles are referred to as “variant” polypeptides with variant amino acid sequences.

A haplotype refers to a segment of DNA that is characterized by a specific combination of alleles arranged along the segment. For diploid organisms such as humans, a haplotype comprises one member of the pair of alleles for each polymorphic marker or locus. In a certain embodiment, the haplotype can comprise two or more alleles, three or more alleles, four or more alleles, or five or more alleles, each allele corresponding to a specific polymorphic marker along the segment. Haplotypes can comprise a combination of various polymorphic markers, e.g., SNPs and microsatellites, having particular alleles at the polymorphic sites. The haplotypes thus comprise a combination of alleles at various genetic markers.

Detecting specific polymorphic markers and/or haplotypes can be accomplished by methods known in the art for detecting sequences at polymorphic sites. For example, standard techniques for genotyping for the presence of SNPs and/or microsatellite markers can be used, such as fluorescence-based techniques (Chen, X. et al., Genome Res. 9(5): 492-98 (1999)), utilizing PCR, LCR, Nested PCR and other techniques for nucleic acid amplification. Specific commercial methodologies available for SNP genotyping include, but are not limited to, TaqMan genotyping assays and SNPlex platforms (Applied Biosystems), gel electrophoresis (Applied Biosystems), mass spectrometry (e.g., MassARRAY system from Sequenom), minisequencing methods, real-time PCR, Bio-Plex system (BioRad), CEQ and SNPstream systems (Beckman), array hybridization technology (e.g., Affymetrix GeneChip; Perlegen), BeadArray Technologies (e.g., Illumina GoldenGate and Infinium methodology), Due to their abundance, SNPs account for a majority of sequence variation in the human genome. Over 6 million SNPs have been validated to date (http://www.ncbi.nlm.nih.gov/projects/SNP/snp_summary.cgi). However, CNVs are receiving increased attention. These large-scale polymorphisms (typically 1 kb or larger) account for polymorphic variation affecting a substantial proportion of the assembled human genome; known CNVs covery over 15% of the human genome sequence (Estivill, X Armengol; L., PloS Genetics 3:1787-99 (2007). A http://projects.tcag.ca/variation/). Most of these polymorphisms are however very rare, and on average affect only a fraction of the genomic sequence of each individual. CNVs are known to affect gene expression, phenotypic variation and adaptation by disrupting gene dosage, and are also known to cause disease (microdeletion and microduplication disorders) and confer risk of common complex diseases, including HIV-1 infection and glomerulonephritis (Redon, R., et al. Nature 23:444-454 (2006)). It is thus possible that either previously described or unknown CNVs represent causative variants in linkage disequilibrium with the markers described herein to be associated with exfoliation syndrome and glaucoma. Methods for detecting CNVs include comparative genomic hybridization (CGH) and genotyping, including use of genotyping arrays, as described by Carter (Nature Genetics 39:S16-S21 (2007)). The Database of Genomic Variants (http://projects.tcag.ca/variation/) contains updated information about the location, type and size of described CNVs. The database currently contains data for over 15,000 CNVs.

In some instances, reference is made to different alleles at a polymorphic site without choosing a reference allele. Alternatively, a reference sequence can be referred to for a particular polymorphic site. The reference allele is sometimes referred to as the “wild-type” allele and it usually is chosen as either the first sequenced allele or as the allele from a “non-affected” individual (e.g., an individual that does not display a trait or disease phenotype).

Alleles for SNP markers as referred to herein refer to the bases A, C, G or T as they occur at the polymorphic site in the SNP assay employed. The allele codes for SNPs used herein are as follows: 1=A, 2=C, 3=G, 4=T. The person skilled in the art will however realise that by assaying or reading the opposite DNA strand, the complementary allele can in each case be measured. Thus, for a polymorphic site (polymorphic marker) characterized by an A/G polymorphism, the assay employed may be designed to specifically detect the presence of one or both of the two bases possible, i.e. A and G. Alternatively, by designing an assay that is designed to detect the complimentary strand on the DNA template, the presence of the complementary bases T and C can be measured. Quantitatively (for example, in terms of relative risk), identical results would be obtained from measurement of either DNA strand (+ strand or − strand).

Typically, a reference sequence is referred to for a particular sequence. Alleles that differ from the reference are sometimes referred to as “variant” alleles. A variant sequence, as used herein, refers to a sequence that differs from the reference sequence but is otherwise substantially similar. Alleles at the polymorphic genetic markers described herein are variants. Variants can include changes that affect a polypeptide. Sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence. Such sequence changes can alter the polypeptide encoded by the nucleic acid. For example, if the change in the nucleic acid sequence causes a frame shift, the frame shift can result in a change in the encoded amino acids, and/or can result in the generation of a premature stop codon, causing generation of a truncated polypeptide. Alternatively, a polymorphism associated with a disease or trait can be a synonymous change in one or more nucleotides (i.e., a change that does not result in a change in the amino acid sequence). Such a polymorphism can, for example, alter splice sites, affect the stability or transport of mRNA, or otherwise affect the transcription or translation of an encoded polypeptide. It can also alter DNA to increase the possibility that structural changes, such as amplifications or deletions, occur at the somatic level. The polypeptide encoded by the reference nucleotide sequence is the “reference” polypeptide with a particular reference amino acid sequence, and polypeptides encoded by variant alleles are referred to as “variant” polypeptides with variant amino acid sequences.

A haplotype refers to a segment of DNA that is characterized by a specific combination of alleles arranged along the segment. For diploid organisms such as humans, a haplotype comprises one member of the pair of alleles for each polymorphic marker or locus. In a certain embodiment, the haplotype can comprise two or more alleles, three or more alleles, four or more alleles, or five or more alleles, each allele corresponding to a specific polymorphic marker along the segment. Haplotypes can comprise a combination of various polymorphic markers, e.g., SNPs and microsatellites, having particular alleles at the polymorphic sites. The haplotypes thus comprise a combination of alleles at various genetic markers.

Detecting specific polymorphic markers and/or haplotypes can be accomplished by methods known in the art for detecting sequences at polymorphic sites. For example, standard techniques for genotyping for the presence of SNPs and/or microsatellite markers can be used, such as fluorescence-based techniques (e.g., Chen, X. et al., Genome Res. 9(5): 492-98 (1999); Kutyavin et al., Nucleic Acid Res. 34:e128 (2006)), utilizing PCR, LCR, Nested PCR and other techniques for nucleic acid amplification. Specific commercial methodologies available for SNP genotyping include, but are not limited to, TaqMan genotyping assays and SNPlex platforms (Applied Biosystems), gel electrophoresis (Applied Biosystems), mass spectrometry (e.g., MassARRAY system from Sequenom), minisequencing methods, real-time PCR, Bio-Plex system (BioRad), CEQ and SNPstream systems (Beckman), array hybridization technology (e.g., Affymetrix GeneChip; Perlegen), BeadArray Technologies (e.g., Illumina GoldenGate and Infinium assays), array tag technology (e.g., Parallele), and endonuclease-based fluorescence hybridization technology (Invader; Third Wave). Some of the available array platforms, including Affymetrix SNP Array 6.0 and Illumina CNV370-Duo and 1M BeadChips, include SNPs that tag certain CNVs. This allows detection of CNVs via surrogate SNPs included in these platforms. Thus, by use of these or other methods available to the person skilled in the art, one or more alleles at polymorphic markers, including microsatellites, SNPs or other types of polymorphic markers, can be identified.

In certain methods described herein, an individual who is at an increased susceptibility (i.e., increased risk) for any specific disease or trait under study, is an individual in whom at least one specific allele at one or more polymorphic marker or haplotype conferring increased susceptibility for the disease or trait is identified (i.e., at-risk marker alleles or haplotypes). In one aspect, the at-risk marker or haplotype is one that confers a significant increased risk (or susceptibility) of the disease or trait. In one embodiment, significance associated with a marker or haplotype is measured by a relative risk (RR). In another embodiment, significance associated with a marker or haplotype is measured by an odds ratio (OR). In a further embodiment, the significance is measured by a percentage. In one embodiment, a significant increased risk is measured as a risk (relative risk and/or odds ratio) of at least 1.2, including but not limited to: at least 1.2, at least 1.3, at least 1.4, at least 1.5, at least 1.6, at least 1.7, 1.8, at least 1.9, at least 2.0, at least 2.5, at least 3.0, at least 4.0, and at least 5.0. In a particular embodiment, a risk (relative risk and/or odds ratio) of at least 1.2 is significant. In another particular embodiment, a risk of at least 1.3 is significant. In yet another embodiment, a risk of at least 1.4 is significant. In a further embodiment, a relative risk of at least about 1.5 is significant. In another further embodiment, a significant increase in risk is at least about 1.7 is significant. However, other cutoffs are also contemplated, e.g. at least 1.15, 1.25, 1.35, and so on, and such cutoffs are also within scope of the present invention. In other embodiments, a significant increase in risk is at least about 20%, including but not limited to about 25%, 30%, 35%, 40%, 45%, 50%, 55%, 60%, 65%, 70%, 75%, 80%, 85%, 90%, 95%, 100%, 150%, 200%, 300%, and 500%. In one particular embodiment, a significant increase in risk is at least 20%. In other embodiments, a significant increase in risk is at least 30%, at least 40%, at least 50%, at least 60%, at least 70%, at least 80%, at least 90% and at least 100%. Other cutoffs or ranges as deemed suitable by the person skilled in the art to characterize the invention are however also contemplated, and those are also within scope of the present invention.

An at-risk polymorphic marker or haplotype of the present invention is one where at least one allele of at least one marker or haplotype is more frequently present in an individual at risk for the disease or trait (affected), or diagnosed with the disease or trait, compared to the frequency of its presence in a comparison group (control), such that the presence of the marker or haplotype is indicative of susceptibility to the disease or trait. The control group may in one embodiment be a population sample, i.e. a random sample from the general population. In another embodiment, the control group is represented by a group of individuals who are disease-free. Such disease-free control may in one embodiment be characterized by the absence of one or more specific disease-associated symptoms (e.g., symptoms associated with XFS and/or galucoma). In another embodiment, the disease-free control group is characterized by the absence of one or more disease-specific risk factors. Such risk factors are in one embodiment at least one environmental risk factor. Representative environmental factors are natural products, minerals or other chemicals which are known to affect, or contemplated to affect, the risk of developing the specific disease or trait. Other environmental risk factors are risk factors related to lifestyle, including but not limited to food and drink habits, geographical location of main habitat, and occupational risk factors. In another embodiment, the risk factors are at least one genetic risk factor.

As an example of a simple test for correlation would be a Fisher-exact test on a two by two table. Given a cohort of chromosomes, the two by two table is constructed out of the number of chromosomes that include both of the markers or haplotypes, one of the markers or haplotypes but not the other and neither of the markers or haplotypes.

In other embodiments of the invention, an individual who is at a decreased susceptibility (i.e., at a decreased risk) for a disease or trait is an individual in whom at least one specific allele at one or more polymorphic marker or haplotype conferring decreased susceptibility for the disease or trait is identified. The marker alleles and/or haplotypes conferring decreased risk are also said to be protective. In one aspect, the protective marker or haplotype is one that confers a significant decreased risk (or susceptibility) of the disease or trait. In one embodiment, significant decreased risk is measured as a relative risk of less than 0.9, including but not limited to less than 0.9, less than 0.8, less than 0.7, less than 0.6, less than 0.5, less than 0.4, less than 0.3, less than 0.2 and less than 0.1. In one particular embodiment, significant decreased risk is less than 0.7. In another embodiment, significant decreased risk is less than 0.5. In yet another embodiment, significant decreased risk is less than 0.3. In another embodiment, the decrease in risk (or susceptibility) is at least 20%, including but not limited to at least 25%, at least 30%, at least 35%, at least 40%, at least 45%, at least 50%, at least 55%, at least 60%, at least 65%, at least 70%, at least 75%, at least 80%, at least 85%, at least 90%, at least 95% and at least 98%. In one particular embodiment, a significant decrease in risk is at least about 30%. In another embodiment, a significant decrease in risk at least about 50%. In another embodiment, the decrease in risk is at least about 70%. Other cutoffs or ranges as deemed suitable by the person skilled in the art to characterize the invention are however also contemplated, and those are also within scope of the present invention.

The person skilled in the art will appreciate that for markers with two alleles present in the population being studied, and wherein one allele is found in increased frequency in a group of individuals with a trait or disease in the population, compared with controls, the other allele of the marker will be found in decreased frequency in the group of individuals with the trait or disease, compared with controls. In such a case, one allele of the marker (the one found in increased frequency in individuals with the trait or disease) will be the at-risk allele, while the other allele will be a protective allele.

A genetic variant associated with a disease or a trait (e.g. XFS and/or glaucoma, such as XFG) can be used alone to predict the risk of the disease for a given genotype. For a biallelic marker, such as a SNP, there are 3 possible genotypes: homozygote for the at risk variant, heterozygote, and non-carrier of the at risk variant. Risk associated with variants at multiple loci can be used to estimate overall risk. For multiple SNP variants, there are k possible genotypes k=3ⁿ×2^{p}; where n is the number autosomal loci and p the number of gonosomal (sex chromosomal) loci. Overall risk assessment calculations usually assume that the relative risks of different genetic variants multiply, i.e. the overall risk (e.g., RR or OR) associated with a particular genotype combination is the product of the risk values for the genotype at each locus. If the risk presented is the relative risk for a person, or a specific genotype for a person, compared to a reference population with matched gender and ethnicity, then the combined risk—is the product of the locus specific risk values—and which also corresponds to an overall risk estimate compared with the population. If the risk for a person is based on a comparison to non-carriers of the at risk allele, then the combined risk corresponds to an estimate that compares the person with a given combination of genotypes at all loci to a group of individuals who do not carry risk variants at any of those loci. The group of non-carriers of any at risk variant has the lowest estimated risk and has a combined risk, compared with itself (i.e., non-carriers) of 1.0, but has an overall risk, compare with the population, of less than 1.0. It should be noted that the group of non-carriers can potentially be very small, especially for large number of loci, and in that case, its relevance is correspondingly small.

The multiplicative model is a parsimonious model that usually fits the data of complex traits reasonably well. Deviations from multiplicity have been rarely described in the context of common variants for common diseases, and if reported are usually only suggestive since very large sample sizes are usually required to be able to demonstrate statistical interactions between loci.

By way of an example, let us consider a total of eight variants that have been described to associate with prostate cancer (Gudmundsson, J., et al., Nat Genet. 39:631-7 (2007), Gudmundsson, J., et al., Nat Genet. 39:977-83 (2007); Yeager, M., et al, Nat Genet. 39:645-49 (2007), Amundadottir, L., et al., Nat Genet. 38:652-8 (2006); Haiman, C. A., et al., Nat Genet. 39:638-44 (2007)). Seven of these loci are on autosomes, and the remaining locus is on chromosome X. The total number of theoretical genotypic combinations is then 3⁷×2¹=4374. Some of those genotypic classes are very rare, but are still possible, and should be considered for overall risk assessment. It is likely that the multiplicative model applied in the case of multiple genetic variant will also be valid in conjugation with non-genetic risk variants assuming that the genetic variant does not clearly correlate with the “environmental” factor. In other words, genetic and non-genetic at-risk variants can be assessed under the multiplicative model to estimate combined risk, assuming that the non-genetic and genetic risk factors do not interact.

Using the same quantitative approach, the combined or overall risk associated with a plurality of variants associated with XFS and glaucoma may be assessed.

### Linkage Disequilibrium

The natural phenomenon of recombination, which occurs on average once for each chromosomal pair during each meiotic event, represents one way in which nature provides variations in sequence (and biological function by consequence). It has been discovered that recombination does not occur randombly in the genome; rather, there are large variations in the frequency of recombination rates, resulting in small regions of high recombination frequency (also called recombination hotspots) and larger regions of low recombination frequency, which are commonly referred to as Linkage Disequilibrium (LD) blocks (Myers, S. et al., Biochem Soc Trans 34:526-530 (2006); Jeffreys, A. J., et al., Nature Genet. 29:217-222 (2001); May, C. A., et al., Nature Genet. 31:272-275 (2002)).

Linkage Disequilibrium (LD) refers to a non-random assortment of two genetic elements. For example, if a particular genetic element (e.g., an allele of a polymorphic marker, or a haplotype) occurs in a population at a frequency of 0.50 (50%) and another element occurs at a frequency of 0.50 (50%), then the predicted occurrence of a person's having both elements is 0.25 (25%), assuming a random distribution of the elements. However, if it is discovered that the two elements occur together at a frequency higher than 0.25, then the elements are said to be in linkage disequilibrium, since they tend to be inherited together at a higher rate than what their independent frequencies of occurrence (e.g., allele or haplotype frequencies) would predict. Roughly speaking, LD is generally correlated with the frequency of recombination events between the two elements. Allele or haplotype frequencies can be determined in a population by genotyping individuals in a population and determining the frequency of the occurrence of each allele or haplotype in the population. For populations of diploids, e.g., human populations, individuals will typically have two alleles for each genetic element (e.g., a marker, haplotype or gene).

Many different measures have been proposed for assessing the strength of linkage disequilibrium (LD; reviewed in Devlin, B. & Risch, N., Genomics 29:311-22 (1995)). Most capture the strength of association between pairs of biallelic sites. Two important pairwise measures of LD are r² (sometimes denoted □Δ²) and |D′| (Lewontin, R., Genetics 49:49-67 (1964); Hill, W. G. & Robertson, A. Theor. Appl. Genet. 22:226-231 (1968)). Both measures range from 0 (no disequilibrium) to 1 (‘complete’ disequilibrium), but their interpretation is slightly different. |D′| is defined in such a way that it is equal to 1 if just two or three of the possible haplotypes are present, and it is <1 if all four possible haplotypes are present. Therefore, a value of |D′| that is <1 indicates that historical recombination may have occurred between two sites (recurrent mutation can also cause |D′| to be <1, but for single nucleotide polymorphisms (SNPs) this is usually regarded as being less likely than recombination). The measure r² represents the statistical correlation between two sites, and takes the value of 1 if only two haplotypes are present.

The r² measure is arguably the most relevant measure for association mapping, because there is a simple inverse relationship between r² and the sample size required to detect association between susceptibility loci and SNPs. These measures are defined for pairs of sites, but for some applications a determination of how strong LD is across an entire region that contains many polymorphic sites might be desirable (e.g., testing whether the strength of LD differs significantly among loci or across populations, or whether there is more or less LD in a region than predicted under a particular model). Measuring LD across a region is not straightforward, but one approach is to use the measure r, which was developed in population genetics. Roughly speaking, r measures how much recombination would be required under a particular population model to generate the LD that is seen in the data. This type of method can potentially also provide a statistically rigorous approach to the problem of determining whether LD data provide evidence for the presence of recombination hotspots. For the methods described herein, a significant r² value can be at least 0.1 such as at least 0.1, 0.15, 0.2, 0.25, 0.3, 0.35, 0.4, 0.45, 0.5, 0.55, 0.6, 0.65, 0.7, 0.75, 0.8, 0.85, 0.9, 0.91, 0.92, 0.93, 0.94, 0.95, 0.96, 0.97, 0.98, 0.99 or 1.0. In one preferred embodiment, the significant r² value can be at least 0.2. Alternatively, linkage disequilibrium as described herein, refers to linkage disequilibrium characterized by values of |D′| of at least 0.2, such as 0.3, 0.4, 0.5, 0.6, 0.7, 0.8, 0.85, 0.9, 0.95, 0.96, 0.97, 0.98, 0.99. Thus, linkage disequilibrium represents a correlation between alleles of distinct markers. It is measured by correlation coefficient or |D′| (r² up to 1.0 and |D′| up to 1.0). Linkage disequilibrium can be determined in a single human population, as defined herein, or it can be determined in a collection of samples comprising individuals from more than one human population. In one embodiment of the invention, LD is determined in a sample from one or more of the HapMap populations. These include samples from the Yoruba people of Ibadan, Nigeria (YRI), samples from individuals from the Tokyo area in Japan (JPT), samples from individuals Beijing, China (CHB), and samples from U.S. residents with northern and western European ancestry (CEU), as described (The International HapMap Consortium, Nature 426:789-796 (2003); see also http://www.hapmap.org). In one such embodiment, LD is determined in the Caucasian CEU population of the HapMap samples. In another embodiment, LD is determined in the African YRI population. In yet another embodiment, LD is determined in samples from the Icelandic population.

If all polymorphisms in the genome were independent at the population level (i.e. no LD between polymorphisms), then every single one of them would need to be investigated in association studies, to assess all different polymorphic states. However, due to linkage disequilibrium between polymorphisms, tightly linked polymorphisms are strongly correlated, which reduces the number of polymorphisms that need to be investigated in an association study to observe a significant association. Another consequence of LD is that many polymorphisms may give an association signal due to the fact that these polymorphisms are strongly correlated.

Genomic LD maps have been generated across the genome, and such LD maps have been proposed to serve as framework for mapping disease-genes (Risch, N. & Merkiangas, K, Science 273:1516-1517 (1996); Maniatis, N., et al., Proc Natl Acad Sci USA 99:2228-2233 (2002); Reich, D E et al, Nature 411:199-204 (2001)).

It is now established that many portions of the human genome can be broken into series of discrete haplotype blocks containing a few common haplotypes; for these blocks, linkage disequilibrium data provides little evidence indicating recombination (see, e.g., Wall., J. D. and Pritchard, J. K., Nature Reviews Genetics 4:587-597 (2003); Daly, M. et al., Nature Genet. 29:229-232 (2001); Gabriel, S. B. et al., Science 296:2225-2229 (2002); Patil, N. et al., Science 294:1719-1723 (2001); Dawson, E. et al., Nature 418:544-548 (2002); Phillips, M. S. et al., Nature Genet. 33:382-387 (2003)).

There are two main methods for defining these haplotype blocks: blocks can be defined as regions of DNA that have limited haplotype diversity (see, e.g., Daly, M. et al., Nature Genet. 29:229-232 (2001); Patil, N. et al., Science 294:1719-1723 (2001); Dawson, E. et al., Nature 418:544-548 (2002); Zhang, K. et al., Proc. Nat. Acad. Sci. USA 99:7335-7339 (2002)), or as regions between transition zones having extensive historical recombination, identified using linkage disequilibrium (see, e.g., Gabriel, S. B. et al., Science 296:2225-2229 (2002); Phillips, M. S. et al., Nature Genet. 33:382-387 (2003); Wang, N. et al., Am. J. Hum. Genet. 71:1227-1234 (2002); Stumpf, M. P., and Goldstein, D. B., Curr. Biol. 13:1-8 (2003)). More recently, a fine-scale map of recombination rates and corresponding hotspots across the human genome has been generated (Myers, S., et al., Science 310:321-32324 (2005); Myers, S. et al., Biochem Soc Trans 34:526530 (2006)). The map reveals the enormous variation in recombination across the genome, with recombination rates as high as 10-60 cM/Mb in hotspots, while closer to 0 in intervening regions, which thus represent regions of limited haplotype diversity and high LD. The map can therefore be used to define haplotype blocks/LD blocks as regions flanked by recombination hotspots. As used herein, the terms “haplotype block” or “LD block” includes blocks defined by any of the above described characteristics, or other alternative methods used by the person skilled in the art to define such regions.

Haplotype blocks (LD blocks) can be used to map associations between phenotype and haplotype status, using single markers or haplotypes comprising a plurality of markers. The main haplotypes can be identified in each haplotype block, and then a set of “tagging” SNPs or markers (the smallest set of SNPs or markers needed to distinguish among the haplotypes) can then be identified. These tagging SNPs or markers can then be used in assessment of samples from groups of individuals, in order to identify association between phenotype and haplotype. If desired, neighboring haplotype blocks can be assessed concurrently, as there may also exist linkage disequilibrium among the haplotype blocks.

It has thus become apparent that for any given observed association to a polymorphic marker in the genome, it is likely that additional markers in the genome also show association. This is a natural consequence of the uneven distribution of LD across the genome, as observed by the large variation in recombination rates. The markers used to detect association thus in a sense represent “tags” for a genomic region (i.e., a haplotype block or LD block) that is associating with a given disease or trait, and as such are useful for use in the methods and kits of the present invention. One or more causative (functional) variants or mutations may reside within the region found to be associating to the disease or trait. The functional variant may be another SNP, a tandem repeat polymorphism (such as a minisatellite or a microsatellite), a transposable element, or a copy number variation, such as an inversion, deletion or insertion. Such variants in LD with other variants used to detect an association to a disease or trait (e.g., the variants described herein to be associated with risk of XFG and glaucoma) may confer a higher relative risk (RR) or odds ratio (OR) than observed for the tagging markers used to detect the association. The present invention thus refers to the markers used for detecting association to the disease, as described herein, as well as markers in linkage disequilibrium with the markers. Thus, in certain embodiments of the invention, markers that are in LD with the markers and/or haplotypes of the invention, as described herein, may be used as surrogate markers. The surrogate markers have in one embodiment relative risk (RR) and/or odds ratio (OR) values smaller than for the markers or haplotypes initially found to be associating with the disease, as described herein. In other embodiments, the surrogate markers have RR or OR values greater than those initially determined for the markers initially found to be associating with the disease, as described herein. An example of such an embodiment would be a rare, or relatively rare (<10% allelic population frequency) variant in LD with a more common variant (>10% population frequency) initially found to be associating with the disease, such as the variants described herein. Identifying and using such markers for detecting the association discovered by the inventors as described herein can be performed by routine methods well known to the person skilled in the art, and are therefore within the scope of the present invention.

### Determination of Haplotype Frequency

The frequencies of haplotypes in patient and control groups can be estimated using an expectation-maximization algorithm (Dempster A. et al, J. R. Stat. Soc. B, 39:1-38 (1977)). An implementation of this algorithm that can handle missing genotypes and uncertainty with the phase can be used. Under the null hypothesis, the patients and the controls are assumed to have identical frequencies. Using a likelihood approach, an alternative hypothesis is tested, where a candidate at-risk-haplotype, which can include the markers described herein, is allowed to have a higher frequency in patients than controls, while the ratios of the frequencies of other haplotypes are assumed to be the same in both groups. Likelihoods are maximized separately under both hypotheses and a corresponding 1-df likelihood ratio statistic is used to evaluate the statistical significance.

To look for at-risk and protective markers and haplotypes within a susceptibility region, for example within an LD block, association of all possible combinations of genotyped markers within the region is studied. The combined patient and control groups can be randomly divided into two sets, equal in size to the original group of patients and controls. The marker and haplotype analysis is then repeated and the most significant p-value registered is determined. This randomization scheme can be repeated, for example, over 100 times to construct an empirical distribution of p-values. In a preferred embodiment, a p-value of <0.05 is indicative of an significant marker and/or haplotype association.

### Haplotype Analysis

One general approach to haplotype analysis involves using likelihood-based inference applied to NEsted MOdels (Gretarsdottir S., et al., Nat. Genet. 35:131-38 (2003)). The method is implemented in the program NEMO, which allows for many polymorphic markers, SNPs and microsatellites. The method and software are specifically designed for case-control studies where the purpose is to identify haplotype groups that confer different risks. It is also a tool for studying LD structures. In NEMO, maximum likelihood estimates, likelihood ratios and p-values are calculated directly, with the aid of the EM algorithm, for the observed data treating it as a missing-data problem.

Even though likelihood ratio tests based on likelihoods computed directly for the observed data, which have captured the information loss due to uncertainty in phase and missing genotypes, can be relied on to give valid p-values, it would still be of interest to know how much information had been lost due to the information being incomplete. The information measure for haplotype analysis is described in Nicolae and Kong (Technical Report 537, Department of Statistics, University of Statistics, University of Chicago; Biometrics, 60(2):368-75 (2004)) as a natural extension of information measures defined for linkage analysis, and is implemented in NEMO.

For single marker association to a disease, the Fisher exact test can be used to calculate two-sided p-values for each individual allele. Usually, all p-values are presented unadjusted for multiple comparisons unless specifically indicated. The presented frequencies (for microsatellites, SNPs and haplotypes) are allelic frequencies as opposed to carrier frequencies. To minimize any bias due the relatedness of the patients who were recruited as families to the study, first and second-degree relatives can be eliminated from the patient list. Furthermore, the test can be repeated for association correcting for any remaining relatedness among the patients, by extending a variance adjustment procedure previously described (Risch, N. & Teng, J. (Genome Res., 8: 1273-1288 (1998)), for sibships so that it can be applied to general familial relationships, and present both adjusted and unadjusted p-values for comparison. The method of genomic controls (Devlin, B. & Roeder, K. Biometrics 55:997 (1999)) can also be used to adjust for the relatedness of the individuals and possible stratification. The differences are in general very small as expected. To assess the significance of single-marker association corrected for multiple testing we can carry out a randomization test using the same genotype data. Cohorts of patients and controls can be randomized and the association analysis redone multiple times (e.g., up to 500,000 times) and the p-value is the fraction of replications that produced a p-value for some marker allele that is lower than or equal to the p-value we observed using the original patient and control cohorts.

For both single-marker and haplotype analyses, relative risk (RR) and the population attributable risk (PAR) can be calculated assuming a multiplicative model (haplotype relative risk model) (Terwilliger, J. D. & Ott, J., Hum. Hered. 42:337-46 (1992) and Falk, C. T. & Rubinstein, P, Ann. Hum. Genet. 51 (Pt 3):227-33 (1987)), i.e., that the risks of the two alleles/haplotypes a person carries multiply. For example, if RR is the risk of A relative to a, then the risk of a person homozygote AA will be RR times that of a heterozygote Aa and RR² times that of a homozygote aa. The multiplicative model has a nice property that simplifies analysis and computations—haplotypes are independent, i.e., in Hardy-Weinberg equilibrium, within the affected population as well as within the control population. As a consequence, haplotype counts of the affecteds and controls each have multinomial distributions, but with different haplotype frequencies under the alternative hypothesis. Specifically, for two haplotypes, hᵢ and hⱼ, risk(hᵢ)/risk(hⱼ)=(fᵢ/pᵢ)/(fⱼ/pⱼ), where f and p denote, respectively, frequencies in the affected population and in the control population. While there is some power loss if the true model is not multiplicative, the loss tends to be mild except for extreme cases. Most importantly, p-values are always valid since they are computed with respect to null hypothesis.

An association signal detected in one association study may be replicated in a second cohort, ideally from a different population (e.g., different region of same country, or a different country) of the same or different ethnicity. The advantage of replication studies is that the number of tests performed in the replication study, and hence the less stringent the statistical measure that is applied. For example, for a genome-wide search for susceptibility variants for a particular disease or trait using 300,000 SNPs, a correction for the 300,000 tests performed (one for each SNP) can be performed. Since many SNPs on the arrays typically used are correlated (i.e., in LD), they are not independent. Thus, the correction is conservative. Nevertheless, applying this correction factor requires an observed P-value of less than 0.05/300,000=1.7×10⁻⁷ for the signal to be considered significant applying this conservative test on results from a single study cohort. Obviously, signals found in a genome-wide association study with P-values less than this conservative threshold are a measure of a true genetic effect, and replication in additional cohorts is not necessarily from a statistical point of view. However, since the correction factor depends on the number of statistical tests performed, if one signal (one SNP) from an initial study is replicated in a second case-control cohort, the appropriate statistical test for significance is that for a single statistical test, i.e., P-value less than 0.05. Replication studies in one or even several additional case-control cohorts have the added advantage of providing assessment of the association signal in additional populations, thus simultaneously confirming the initial finding and providing an assessment of the overall significance of the genetic variant(s) being tested in human populations in general.

The results from several case-control cohorts can also be combined to provide an overall assessment of the underlying effect. The methodology commonly used to combine results from multiple genetic association studies is the Mantel-Haenszel model (Mantel and Haenszel, J Natl Cancer Inst 22:719-48 (1959)). The model is designed to deal with the situation where association results from different populations, with each possibly having a different population frequency of the genetic variant, are combined. The model combines the results assuming that the effect of the variant on the risk of the disease, a measured by the OR or RR, is the same in all populations, while the frequency of the variant may differ between the populations. Combining the results from several populations has the added advantage that the overall power to detect a real underlying association signal is increased, due to the increased statistical power provided by the combined cohorts. Furthermore, any deficiencies in individual studies, for example due to unequal matching of cases and controls or population stratification will tend to balance out when results from multiple cohorts are combined, again providing a better estimate of the true underlying genetic effect.

### Risk Assessment and Diagnostics

Within any given population, there is an absolute risk of developing a disease or trait, defined as the chance of a person developing the specific disease or trait over a specified time-period. For example, a woman's lifetime absolute risk of breast cancer is one in nine. That is to say, one woman in every nine will develop breast cancer at some point in their lives. Risk is typically measured by looking at very large numbers of people, rather than at a particular individual. Risk is often presented in terms of Absolute Risk (AR) and Relative Risk (RR). Relative Risk is used to compare risks associating with two variants or the risks of two different groups of people. For example, it can be used to compare a group of people with a certain genotype with another group having a different genotype. For a disease, a relative risk of 2 means that one group has twice the chance of developing a disease as the other group. The Risk presented is usually the relative risk for a person, or a specific genotype of a person, compared to the population with matched gender and ethnicity. Risks of two individuals of the same gender and ethnicity could be compared in a simple manner. For example, if, compared to the population, the first individual has relative risk 1.5 and the second has relative risk 0.5, then the risk of the first individual compared to the second individual is 1.5/0.5=3.

As described herein, certain polymorphic markers and haplotypes comprising such markers are found to be useful for risk assessment of XFS andor glaucoma. Risk assessment can involve the use of the markers for determining a susceptibility to XFS and/or glaucoma. Particular alleles of polymorphic markers are found more frequently in individuals with XFS and glaucoma, in particular exfoliation glaucoma (XFG) than in individuals without diagnosis of XFS and/or glaucoma. Therefore, these marker alleles have predictive value for detecting XFS and/or glaucoma, or a susceptibility to XFS and/or glaucoma, in an individual. Tagging markers within haplotype blocks or LD blocks (e.g., the LOXL1 LD block) comprising at-risk markers, such as the markers of the present invention, can be used as surrogates for other markers and/or haplotypes within the haplotype block or LD block. Markers with values of r² equal to 1 are perfect surrogates for the at-risk variants, i.e. genotypes for one marker perfectly predicts genotypes for the other. Markers with smaller values of r² than 1 can also be surrogates for the at-risk variant, or alternatively represent variants with relative risk values as high or possibly even higher than the at-risk variant. The at-risk variant identified may not be the functional variant itself, but is in this instance in linkage disequilibrium with the true functional variant. The present invention encompasses the assessment of such surrogate markers for the markers as disclosed herein. Such markers are annotated, mapped and listed in public databases, as well known to the skilled person, or can alternatively be readily identified by sequencing the region or a part of the region identified by the markers of the present invention in a group of individuals, and identify polymorphisms in the resulting group of sequences. As a consequence, the person skilled in the art can readily and without undue experimentation genotype surrogate markers in linkage disequilibrium with the markers and/or haplotypes as described herein. The tagging or surrogate markers in LD with the at-risk variants detected, also have predictive value for detecting association to XFS and/or glaucoma, or a susceptibility to XFS and/or glaucoma, in an individual. These tagging or surrogate markers that are in LD with the markers of the present invention can also include other markers that distinguish among haplotypes, as these similarly have predictive value for detecting susceptibility to XFS and/or glaucoma.

The present invention can in certain embodiments be practiced by assessing a sample comprising genomic DNA from an individual for the presence of variants described herein to be associated with XFS and glaucoma. Such assessment includes steps of detecting the presence or absence of at least one allele of at least one polymorphic marker, using methods well known to the skilled person and further described herein, and based on the outcome of such assessment, determine whether the individual from whom the sample is derived is at increased or decreased risk (increased or decreased susceptibility) of developing XFS and/or glaucoma, in particular XFG. Alternatively, the invention can be practiced utilizing a dataset comprising information about the genotype status of at least one polymorphic marker described herein to be associated with XFS and glaucoma (or markers in linkage disequilibrium with at least one marker shown herein to be associated with these disorders). In other words, a dataset containing information about such genetic status, for example in the form of genotype counts at a certain polymorphic marker, or a plurality of markers (e.g., an indication of the presence or absence of certain at-risk alleles), or actual genotypes for one or more markers, can be queried for the presence or absence of certain at-risk alleles at certain polymorphic markers shown by the present inventors to be associated with risk of XFS and glaucoma (e.g., XFG). A positive result for a variant (e.g., marker allele) associated with XFG and/or glaucoma, as shown herein, is indicative of the individual from which the dataset is derived is at increased susceptibility (increased risk) of developing XFG and/or glaucoma, in particular exfoliation glaucoma.

In certain embodiments of the invention, a polymorphic marker is correlated to XFS and/or glaucoma by referencing genotype data for the polymorphic marker to a look-up table that comprises correlations between at least one allele of the polymorphism and XFS and/or glaucoma. In some embodiments, the table comprises a correlation for one polymorhpism. In other embodiments, the table comprises a correlation for a plurality of polymorhpisms. In both scenarios, by referencing to a look-up table that gives an indication of a correlation between a marker and XFS and/or glaucoma, a risk for XFS and/or glaucoma, or a susceptibility to XFS and/or glaucoma, can be identified in the individual from whom the sample is derived. In some embodiments, the correlation is reported as a statistical measure. The statistical measure may be reported as a risk measure, such as a relative risk (RR), an absolute risk (AR) or an odds ratio (OR).

The markers of the invention, e.g., the markers presented in Tables 4 and 6a, may be useful for risk assessment and diagnostic purposes for XFS and glaucoma, either alone or in combination. Thus, even in cases where the increase in risk by individual markers is relatively modest, i.e. on the order of 10-30%, the association may have significant implications. Thus, relatively common variants may have significant contribution to the overall risk (Population Attributable Risk is high), or combination of markers can be used to define groups of individual who, based on the combined risk of the markers, is at significant combined risk of developing the disease.

Thus, in one embodiment of the invention, a plurality of variants (markers and/or haplotypes) is used for overall risk assessment. These variants are in one embodiment selected from the variants as disclosed herein. Other embodiments include the use of the variants of the present invention in combination with other variants known to be useful for diagnosing a susceptibility to XFS and/or glaucoma. In such embodiments, the genotype status of a plurality of markers and/or haplotypes is determined in an individual, and the status of the individual compared with the population frequency of the associated variants, or the frequency of the variants in clinically healthy subjects, such as age-matched and sex-matched subjects. Methods known in the art, such as multivariate analyses or joint risk analyses, may subsequently be used to determine the overall risk conferred based on the genotype status at the multiple loci. Assessment of risk based on such analysis may subsequently be used in the methods and kits of the invention, as described herein.

As described in the above, the haplotype block structure of the human genome has the effect that a large number of variants (markers and/or haplotypes) in linkage disequilibrium with the variant originally associated with a disease or trait may be used as surrogate markers for assessing association to the disease or trait. The number of such surrogate markers will depend on factors such as the historical recombination rate in the region, the mutational frequency in the region (i.e., the number of polymorphic sites or markers in the region), and the extent of LD (size of the LD block) in the region. These markers are usually located within the physical boundaries of the LD block or haplotype block in question as defined using the methods described herein, or by other methods known to the person skilled in the art. However, sometimes marker and haplotype association is found to extend beyond the physical boundaries of the haplotype block as defined. Such markers and/or haplotypes may in those cases be also used as surrogate markers and/or haplotypes for the markers and/or haplotypes physically residing within the haplotype block as defined. As a consequence, markers in LD (typically characterized by r² greater than 0.1, such as r² greater than 0.2, including r² greater than 0.3, also including r² greater than 0.4) with the markers and haplotypes of the present invention are also within the scope of the invention, even if they are physically located beyond the boundaries of the haplotype block as defined. This includes markers that are described herein (e.g., Tables 4 and 6a), but may also include other markers that are in strong LD (characterized by r² of at least 0.2 and/or |D′|>0.8) with one or more of the markers listed in Tables 4 and 6a.

Preferred embodiments of the invention relate to markers rs1048661 and rs3825942. The surrogate marker tables below (Table 15 and Table 16) lists markers that are in linkage disequilibrium, characterized by r² of at least 0.2 to markers rs1048661 and rs3825942, illustrating that LD to specific markers may extend over relatively large distances. The tables also illustrates how markers in LD with the markers used to detect an association can suitably also be used to detect a comparable association. Thus, preferred embodiments also relate to the markers listed in Table 15 and Table 16.

TABLE 15
Markers in LD, determined as r² > 0.2 in the HapMap CEU samples,
to markers rs1048661 and rs3825942.

| SNP | Position Build 34 | | SEQ ID NO: |
|---|---|---|---|
| | | r² to rs1048661 | |
| rs4886725 | 71886823 | 0.220975 | 86 |
| rs12915956 | 71891620 | 0.218888 | 87 |
| rs896590 | 71893370 | 0.237179 | 88 |
| rs12438872 | 71893581 | 0.237179 | 89 |
| rs4261482 | 71916249 | 0.437932 | 42 |
| rs2165241 | 71938019 | 0.449749 | 15 |
| rs1992314 | 71939584 | 0.898282 | 44 |
| rs4886776 | 71940813 | 0.897254 | 45 |
| rs2028386 | 71942525 | 0.358672 | 46 |
| rs4337252 | 71942582 | 0.433252 | 47 |
| rs2028387 | 71942854 | 0.897325 | 48 |
| rs4077284 | 71944208 | 0.77534 | 49 |
| rs893816 | 71944281 | 0.898282 | 50 |
| rs4886782 | 71944627 | 0.238052 | 51 |
| rs893817 | 71944882 | 0.77534 | 17 |
| rs893818 | 71945012 | 0.898282 | 52 |
| rs893820 | 71945420 | 0.896954 | 53 |
| rs12440667 | 71947256 | 0.365256 | 54 |
| rs1530169 | 71952937 | 0.375365 | 19 |
| rs893821 | 71957052 | 0.363904 | 90 |
| rs750460 | 71957323 | 0.324985 | 55 |
| rs4243042 | 71957441 | 0.324985 | 56 |
| rs2304722 | 71957566 | 0.327507 | 91 |
| | | r² to rs3825942 | |
| rs4886623 | 71808745 | 0.785579 | 92 |
| rs2167648 | 71832650 | 1 | 93 |
| rs1584738 | 71832895 | 0.845844 | 94 |
| rs1901570 | 71834642 | 0.835538 | 95 |
| rs8026593 | 71836202 | 0.837098 | 6 |
| rs4886660 | 71838009 | 0.837098 | 96 |
| rs4886421 | 71838041 | 0.837098 | 97 |
| rs4886663 | 71838661 | 0.837098 | 98 |
| rs4886664 | 71838733 | 0.835538 | 99 |
| rs4145873 | 71839090 | 0.837098 | 100 |
| rs4145874 | 71839381 | 0.837098 | 101 |
| rs1452389 | 71854639 | 0.600083 | 102 |
| rs1078967 | 71938804 | 1 | 103 |
| rs8041642 | 71940923 | 1 | 104 |
| rs8041685 | 71941010 | 1 | 16 |
| rs8042039 | 71941335 | 1 | 105 |
| rs2304719 | 71951317 | 1 | 18 |
| rs12437465 | 71959063 | 1 | 57 |

TABLE 16
Markers in LD with markers rs1048661 and rs3825942 in the HapMap populations
(CEU = Caucasian samples; YRI = African samples; CHB = Chinese samples;
JPT = Japanese samples; see also http://www.hapmap.org).
The correlated markers are shown in the first column, their position in NCBI Build 36 in the second column,
and their D′ and r² values to marker rs1048661 and rs3825942 in the four HapMap populations in
subsequent columns, followed by reference to the SEQ ID of the flanking sequences for these markers.

| | | rs1048661 | | | | | | | | rs3825942 | | | | | | | | |
|---|---|---|---|---|---|---|---|---|---|---|---|---|---|---|---|---|---|---|
| | | CEU | | YRI | | CHB | | JPT | | CEU | | YRI | | CHB | | JPT | | SEQ ID |
| SNP | PosB36 | D′ | R2 | D′ | R2 | D′ | R2 | D′ | R2 | D′ | R2 | D′ | R2 | D′ | R2 | D′ | R2 | NO: |
| rs3825942 | 72006635 | 1.00 | 0.17 | 1.00 | 0.12 | 1.00 | 0.16 | 1.00 | 0.12 | — | — | — | — | — | — | — | — | 107 |
| rs1048661 | 72006599 | — | — | — | — | — | — | — | — | 1.00 | 0.17 | 1.00 | 0.12 | 1.00 | 0.16 | 1.00 | 0.12 | 106 |
| rs8035197 | 71017803 | 0.09 | 0.00 | 1.00 | 0.00 | 1.00 | 0.04 | 1.00 | 0.01 | 0.52 | 0.06 | 1.00 | 0.01 | 1.00 | 0.20 | 1.00 | 0.08 | 122 |
| rs8035210 | 71017819 | 0.10 | 0.00 | 0.09 | 0.01 | 1.00 | 0.04 | 1.00 | 0.01 | 0.59 | 0.09 | 0.34 | 0.02 | 1.00 | 0.20 | 1.00 | 0.08 | 123 |
| rs8034178 | 71017902 | 0.10 | 0.00 | 0.42 | 0.04 | 1.00 | 0.04 | 1.00 | 0.02 | 0.60 | 0.09 | 0.08 | 0.00 | 1.00 | 0.28 | 1.00 | 0.16 | 124 |
| rs8034355 | 71018064 | 0.10 | 0.00 | 0.42 | 0.04 | 1.00 | 0.04 | 1.00 | 0.02 | 0.60 | 0.09 | 0.08 | 0.00 | 1.00 | 0.28 | 1.00 | 0.16 | 125 |
| rs4776620 | 71018775 | 0.10 | 0.00 | 1.00 | 0.00 | 1.00 | 0.04 | 1.00 | 0.01 | 0.60 | 0.09 | 1.00 | 0.01 | 1.00 | 0.20 | 1.00 | 0.08 | 126 |
| rs6495030 | 71021089 | 0.10 | 0.00 | 1.00 | 0.00 | 1.00 | 0.04 | 1.00 | 0.01 | 0.58 | 0.09 | 1.00 | 0.01 | 1.00 | 0.20 | 1.00 | 0.08 | 127 |
| rs2680346 | 71314823 | 0.65 | 0.03 | 0.53 | 0.02 | 1.00 | 0.09 | 0.68 | 0.05 | 1.00 | 0.01 | 0.08 | 0.00 | 0.60 | 0.21 | 0.04 | 0.00 | 128 |
| rs7170655 | 71503449 | 1.00 | 0.03 | 0.04 | 0.00 | 0.15 | 0.01 | 1.00 | 0.06 | 1.00 | 0.21 | 0.07 | 0.00 | 1.00 | 0.03 | 0.05 | 0.00 | 129 |
| rs16958177 | 71807690 | 1.00 | 0.01 | — | — | 0.03 | 0.00 | 0.48 | 0.22 | 1.00 | 0.00 | — | — | 0.00 | 0.00 | 1.00 | 0.10 | 130 |
| rs16958188 | 71809182 | 0.10 | 0.00 | 1.00 | 0.01 | 0.02 | 0.00 | 0.49 | 0.21 | 1.00 | 0.01 | 0.05 | 0.00 | 0.03 | 0.00 | 1.00 | 0.10 | 131 |
| rs1548099 | 71812981 | 0.10 | 0.00 | 0.11 | 0.01 | 0.02 | 0.00 | 0.52 | 0.22 | 1.00 | 0.01 | 0.35 | 0.03 | 0.03 | 0.00 | 1.00 | 0.09 | 132 |
| rs7402796 | 71821849 | 1.00 | 0.01 | 0.14 | 0.00 | 0.00 | 0.00 | 0.49 | 0.21 | 1.00 | 0.00 | 0.53 | 0.06 | 0.07 | 0.00 | 1.00 | 0.10 | 133 |
| rs896598 | 71823682 | 1.00 | 0.01 | 0.16 | 0.00 | 0.00 | 0.00 | 0.49 | 0.21 | 1.00 | 0.00 | 0.53 | 0.06 | 0.07 | 0.00 | 1.00 | 0.10 | 134 |
| rs4887140 | 71833716 | 0.09 | 0.00 | 0.60 | 0.01 | 0.16 | 0.00 | 0.50 | 0.20 | 0.43 | 0.00 | 0.56 | 0.05 | 0.37 | 0.01 | 1.00 | 0.09 | 135 |
| rs4886623 | 71879981 | 0.26 | 0.02 | 0.15 | 0.01 | 0.36 | 0.12 | 0.68 | 0.17 | 0.79 | 0.32 | 0.02 | 0.00 | 0.18 | 0.00 | 0.15 | 0.01 | 92 |
| rs7174255 | 71891648 | 0.27 | 0.01 | 0.15 | 0.00 | 0.62 | 0.04 | 1.00 | 0.27 | 0.32 | 0.08 | 0.25 | 0.03 | 0.23 | 0.03 | 0.12 | 0.01 | 136 |
| rs4886647 | 71897154 | 0.72 | 0.08 | — | — | 0.28 | 0.06 | 0.66 | 0.29 | 1.00 | 0.03 | — | — | 0.28 | 0.01 | 0.00 | 0.00 | 137 |
| rs4886415 | 71897620 | 0.61 | 0.10 | 0.40 | 0.01 | 0.65 | 0.06 | 1.00 | 0.27 | 0.42 | 0.01 | 0.30 | 0.06 | 0.03 | 0.00 | 1.00 | 0.04 | 138 |
| rs4886416 | 71898049 | 0.72 | 0.08 | — | — | 0.28 | 0.06 | 0.66 | 0.29 | 1.00 | 0.03 | — | — | 0.28 | 0.01 | 0.00 | 0.00 | 139 |
| rs2198294 | 71899427 | 0.73 | 0.09 | — | — | 0.28 | 0.06 | 0.65 | 0.28 | 1.00 | 0.03 | — | — | 0.28 | 0.01 | 0.00 | 0.00 | 140 |
| rs2061803 | 71901822 | 0.34 | 0.09 | 0.12 | 0.01 | 0.62 | 0.05 | 1.00 | 0.27 | 0.21 | 0.01 | 0.13 | 0.00 | 0.00 | 0.00 | 0.83 | 0.03 | 141 |
| rs999742 | 71902570 | 0.38 | 0.08 | 0.20 | 0.02 | 0.28 | 0.06 | 0.66 | 0.29 | 0.19 | 0.02 | 0.19 | 0.00 | 0.28 | 0.01 | 0.00 | 0.00 | 5 |
| rs2167648 | 71903886 | 1.00 | 0.05 | 0.32 | 0.01 | — | — | 1.00 | 0.01 | 1.00 | 0.29 | 0.20 | 0.02 | — | — | 1.00 | 0.00 | 142 |
| rs1584738 | 71904131 | 0.66 | 0.03 | 0.46 | 0.03 | — | — | — | — | 0.85 | 0.26 | 0.19 | 0.02 | — | — | — | — | 143 |
| rs1901570 | 71905878 | 0.68 | 0.03 | 0.32 | 0.01 | — | — | — | — | 0.84 | 0.23 | 0.20 | 0.02 | — | — | — | — | 144 |
| rs8026593 | 71907438 | 0.67 | 0.03 | 0.12 | 0.00 | — | — | — | — | 0.84 | 0.23 | 0.58 | 0.02 | — | — | — | — | 6 |
| rs4886660 | 71909245 | 0.67 | 0.03 | 0.30 | 0.01 | — | — | — | — | 0.84 | 0.23 | 0.18 | 0.01 | — | — | — | — | 96 |
| rs4886421 | 71909277 | 0.67 | 0.03 | 0.56 | 0.03 | — | — | — | — | 0.84 | 0.23 | 0.21 | 0.02 | — | — | — | — | 97 |
| rs4886663 | 71909897 | 0.67 | 0.03 | 0.30 | 0.01 | — | — | — | — | 0.84 | 0.23 | 0.18 | 0.01 | — | — | — | — | 98 |
| rs4886664 | 71909969 | 0.67 | 0.03 | 0.41 | 0.02 | — | — | — | — | 0.84 | 0.23 | 0.18 | 0.01 | — | — | — | — | 99 |
| rs4145873 | 71910326 | 0.67 | 0.03 | 0.30 | 0.01 | — | — | — | — | 0.84 | 0.23 | 0.18 | 0.01 | — | — | — | — | 100 |
| rs4145874 | 71910617 | 0.67 | 0.03 | 0.30 | 0.01 | — | — | — | — | 0.84 | 0.23 | 0.18 | 0.01 | — | — | — | — | 101 |
| rs1452389 | 71925875 | 0.44 | 0.05 | 0.20 | 0.01 | 0.51 | 0.15 | 0.51 | 0.07 | 0.60 | 0.21 | 0.00 | 0.00 | 0.31 | 0.03 | 0.11 | 0.00 | 102 |
| rs4577028 | 71928035 | 0.64 | 0.16 | 0.17 | 0.02 | 0.58 | 0.19 | 0.72 | 0.23 | 0.43 | 0.01 | 0.07 | 0.00 | 0.33 | 0.04 | 0.00 | 0.00 | 145 |
| rs1349642 | 71930673 | 0.13 | 0.01 | 0.24 | 0.02 | 1.00 | 0.04 | 0.72 | 0.06 | 0.03 | 0.00 | 0.05 | 0.00 | 1.00 | 0.28 | 0.04 | 0.00 | 146 |
| rs921541 | 71947156 | 0.66 | 0.17 | 0.54 | 0.06 | 1.00 | 0.06 | 0.79 | 0.11 | 0.49 | 0.02 | 0.60 | 0.21 | 1.00 | 0.28 | 0.67 | 0.01 | 147 |
| rs744616 | 71950238 | 0.20 | 0.01 | — | — | 1.00 | 0.06 | 0.18 | 0.00 | 0.28 | 0.08 | — | — | 1.00 | 0.28 | 0.00 | 0.00 | 148 |
| rs4886725 | 71958059 | 0.52 | 0.22 | 0.06 | 0.00 | 0.67 | 0.27 | 0.70 | 0.20 | 0.65 | 0.06 | 0.11 | 0.00 | 0.10 | 0.00 | 0.04 | 0.00 | 86 |
| rs2084851 | 71958421 | 0.48 | 0.15 | 0.07 | 0.00 | 0.61 | 0.18 | 0.77 | 0.20 | 0.04 | 0.00 | 0.11 | 0.00 | 1.00 | 0.04 | 0.03 | 0.00 | 149 |
| rs7170291 | 71960691 | 0.22 | 0.01 | 1.00 | 0.07 | 0.62 | 0.21 | 0.27 | 0.02 | 0.26 | 0.06 | 0.54 | 0.17 | 0.07 | 0.00 | 0.14 | 0.01 | 150 |
| rs7496005 | 71960721 | 0.42 | 0.17 | 0.04 | 0.00 | 0.60 | 0.23 | 0.71 | 0.21 | 0.73 | 0.10 | 0.11 | 0.00 | 0.07 | 0.00 | 0.03 | 0.00 | 151 |
| rs12915956 | 71962856 | 0.51 | 0.21 | 0.26 | 0.05 | 0.57 | 0.19 | 0.70 | 0.20 | 0.71 | 0.07 | 0.38 | 0.01 | 0.49 | 0.07 | 0.04 | 0.00 | 87 |
| rs896590 | 71964606 | 0.52 | 0.23 | 0.44 | 0.06 | 0.60 | 0.23 | 0.71 | 0.21 | 0.72 | 0.08 | 0.64 | 0.16 | 0.48 | 0.07 | 0.03 | 0.00 | 88 |
| rs12438872 | 71964817 | 0.52 | 0.23 | 0.19 | 0.02 | 0.60 | 0.23 | 0.71 | 0.21 | 0.72 | 0.08 | 0.32 | 0.01 | 0.48 | 0.07 | 0.03 | 0.00 | 89 |
| rs13638 | 71968591 | 0.20 | 0.01 | — | — | 0.35 | 0.01 | 0.18 | 0.00 | 0.27 | 0.07 | — | — | 0.81 | 0.37 | 0.00 | 0.00 | 12 |
| rs12440507 | 71972300 | — | — | 0.20 | 0.02 | 0.63 | 0.23 | 0.78 | 0.23 | — | — | 0.49 | 0.02 | 0.41 | 0.05 | 0.02 | 0.00 | 152 |
| rs11855607 | 71972433 | — | — | 1.00 | 0.05 | 0.73 | 0.20 | 0.80 | 0.11 | — | — | 0.58 | 0.14 | 0.62 | 0.01 | 0.07 | 0.00 | 153 |
| rs4886467 | 71985866 | 0.57 | 0.18 | 0.53 | 0.07 | — | — | 1.00 | 0.07 | 0.33 | 0.02 | 0.79 | 0.31 | — | — | 1.00 | 0.01 | 13 |
| rs4261482 | 71987485 | 0.85 | 0.43 | 0.07 | 0.00 | 0.60 | 0.23 | 0.81 | 0.28 | 1.00 | 0.10 | 0.19 | 0.00 | 0.48 | 0.07 | 0.03 | 0.00 | 42 |
| rs4558370 | 71991963 | 0.29 | 0.01 | 0.71 | 0.03 | 0.35 | 0.01 | 0.18 | 0.00 | 0.35 | 0.12 | 0.32 | 0.04 | 0.81 | 0.37 | 0.00 | 0.00 | 154 |
| rs4464030 | 71992240 | 0.39 | 0.02 | 1.00 | 0.00 | 0.35 | 0.01 | 0.18 | 0.00 | 0.36 | 0.12 | 1.00 | 0.01 | 0.81 | 0.37 | 0.00 | 0.00 | 155 |
| rs4283181 | 71992508 | 0.38 | 0.02 | 0.28 | 0.01 | 0.35 | 0.01 | 0.18 | 0.00 | 0.35 | 0.12 | 0.13 | 0.01 | 0.81 | 0.37 | 0.00 | 0.00 | 156 |
| rs11634339 | 71992860 | 0.42 | 0.07 | 0.36 | 0.01 | 0.59 | 0.16 | 0.88 | 0.23 | 0.43 | 0.02 | 0.23 | 0.04 | 1.00 | 0.04 | 0.07 | 0.00 | 157 |
| rs4461027 | 71998601 | 0.58 | 0.14 | 0.17 | 0.02 | 0.68 | 0.29 | 1.00 | 0.42 | 0.44 | 0.02 | 0.71 | 0.07 | 1.00 | 0.05 | 0.03 | 0.00 | 14 |
| rs2165241 | 72009255 | 0.95 | 0.45 | 1.00 | 0.04 | 1.00 | 0.09 | 1.00 | 0.18 | 1.00 | 0.14 | 1.00 | 0.17 | 1.00 | 0.01 | 0.67 | 0.01 | 15 |
| rs1078967 | 72010040 | 1.00 | 0.17 | 1.00 | 0.12 | 1.00 | 0.17 | 1.00 | 0.13 | 1.00 | 1.00 | 1.00 | 0.97 | 1.00 | 1.00 | 1.00 | 1.00 | 103 |
| rs1992314 | 72010820 | 0.96 | 0.90 | 1.00 | 0.83 | 1.00 | 0.95 | 1.00 | 0.95 | 1.00 | 0.16 | 0.87 | 0.11 | 1.00 | 0.20 | 1.00 | 0.12 | 44 |
| rs1440101 | 72011684 | — | — | 1.00 | 0.05 | 1.00 | 0.48 | 0.85 | 0.22 | — | — | 1.00 | 0.20 | 1.00 | 0.04 | 1.00 | 0.05 | 158 |
| rs2289414 | 72011853 | — | — | — | — | 1.00 | 0.04 | 1.00 | 0.02 | — | — | — | — | 1.00 | 0.37 | 1.00 | 0.16 | 159 |
| rs4886776 | 72012049 | 0.96 | 0.90 | 1.00 | 0.93 | 1.00 | 0.90 | 1.00 | 1.00 | 1.00 | 0.16 | 0.84 | 0.10 | 1.00 | 0.21 | 1.00 | 0.15 | 45 |
| rs8041642 | 72012159 | 1.00 | 0.17 | 1.00 | 0.09 | 1.00 | 0.17 | 1.00 | 0.13 | 1.00 | 1.00 | 1.00 | 0.74 | 1.00 | 1.00 | 1.00 | 0.81 | 104 |
| rs8041685 | 72012246 | 1.00 | 0.17 | 1.00 | 0.07 | 1.00 | 0.14 | 1.00 | 0.06 | 1.00 | 1.00 | 1.00 | 0.53 | 1.00 | 0.89 | 1.00 | 0.51 | 16 |
| rs8042039 | 72012571 | 1.00 | 0.17 | 1.00 | 0.07 | 1.00 | 0.16 | 1.00 | 0.12 | 1.00 | 1.00 | 1.00 | 0.53 | 1.00 | 1.00 | 1.00 | 1.00 | 105 |
| rs2028386 | 72013761 | 0.88 | 0.36 | 1.00 | 0.12 | 1.00 | 0.62 | 1.00 | 0.54 | 1.00 | 0.12 | 1.00 | 0.47 | 1.00 | 0.05 | 1.00 | 0.11 | 46 |
| rs4337252 | 72013818 | 0.90 | 0.43 | 1.00 | 0.13 | 1.00 | 0.59 | 1.00 | 0.57 | 1.00 | 0.15 | 1.00 | 0.51 | 1.00 | 0.05 | 1.00 | 0.10 | 47 |
| rs2028387 | 72014090 | 0.96 | 0.90 | 1.00 | 0.93 | 1.00 | 0.95 | 1.00 | 1.00 | 1.00 | 0.15 | 0.85 | 0.09 | 1.00 | 0.22 | 1.00 | 0.14 | 48 |
| rs12594472 | 72014193 | — | — | 1.00 | 0.05 | 1.00 | 0.44 | 1.00 | 0.27 | — | — | 1.00 | 0.20 | 1.00 | 0.03 | 1.00 | 0.05 | 160 |
| rs4077284 | 72015444 | 0.93 | 0.77 | 1.00 | 0.66 | 1.00 | 0.95 | 1.00 | 1.00 | 1.00 | 0.18 | 0.89 | 0.15 | 1.00 | 0.19 | 1.00 | 0.11 | 49 |
| rs893816 | 72015517 | 0.96 | 0.90 | 1.00 | 0.94 | 1.00 | 0.95 | 1.00 | 1.00 | 1.00 | 0.16 | 0.86 | 0.10 | 1.00 | 0.19 | 1.00 | 0.12 | 50 |
| rs4886782 | 72015863 | 0.92 | 0.24 | 0.88 | 0.01 | 1.00 | 0.09 | 1.00 | 0.13 | 1.00 | 0.07 | 1.00 | 0.03 | 1.00 | 0.01 | 0.70 | 0.01 | 51 |
| rs893817 | 72016118 | 0.93 | 0.77 | 1.00 | 0.41 | 1.00 | 0.95 | 1.00 | 1.00 | 1.00 | 0.18 | 0.31 | 0.03 | 1.00 | 0.19 | 1.00 | 0.12 | 17 |
| rs893818 | 72016248 | 0.96 | 0.90 | 1.00 | 0.83 | 1.00 | 0.95 | 1.00 | 1.00 | 1.00 | 0.16 | 0.87 | 0.11 | 1.00 | 0.19 | 1.00 | 0.12 | 52 |
| rs893820 | 72016656 | 0.96 | 0.90 | 1.00 | 0.20 | 1.00 | 0.27 | 1.00 | 0.36 | 1.00 | 0.15 | 0.60 | 0.22 | 1.00 | 0.55 | 1.00 | 0.34 | 53 |
| rs16958494 | 72018402 | — | — | 1.00 | 0.00 | 0.88 | 0.37 | 1.00 | 0.29 | — | — | 1.00 | 0.01 | 1.00 | 0.03 | 0.56 | 0.01 | 161 |
| rs12440667 | 72018492 | 0.93 | 0.36 | 1.00 | 0.03 | 1.00 | 0.07 | 0.79 | 0.09 | 0.82 | 0.08 | 1.00 | 0.10 | 1.00 | 0.01 | 0.21 | 0.00 | 54 |
| rs12441130 | 72021955 | — | — | — | — | 0.84 | 0.47 | 1.00 | 0.73 | — | — | — | — | 0.62 | 0.02 | 0.13 | 0.00 | 162 |
| rs2304719 | 72022553 | 0.01 | 0.00 | 1.00 | 0.09 | 0.24 | 0.02 | 0.05 | 0.00 | 1.00 | 0.51 | 1.00 | 0.70 | 1.00 | 0.26 | 1.00 | 0.52 | 18 |
| rs1530169 | 72024173 | 0.74 | 0.37 | 1.00 | 0.16 | 0.17 | 0.01 | 0.40 | 0.09 | 1.00 | 0.11 | 0.60 | 0.29 | 1.00 | 0.20 | 1.00 | 0.22 | 19 |
| rs2304721 | 72027407 | 1.00 | 0.07 | 1.00 | 0.00 | 1.00 | 0.46 | 1.00 | 0.27 | 1.00 | 0.01 | 1.00 | 0.01 | 1.00 | 0.04 | 1.00 | 0.05 | 20 |
| rs893821 | 72028288 | 0.88 | 0.36 | — | — | 1.00 | 0.40 | 1.00 | 0.57 | 1.00 | 0.07 | — | — | 1.00 | 0.09 | 1.00 | 0.06 | 90 |
| rs750460 | 72028559 | 0.88 | 0.32 | 1.00 | 0.03 | 1.00 | 0.11 | 1.00 | 0.18 | 1.00 | 0.12 | 1.00 | 0.12 | 1.00 | 0.01 | 0.67 | 0.01 | 55 |
| rs4243042 | 72028677 | 0.88 | 0.32 | 1.00 | 0.03 | 1.00 | 0.09 | 1.00 | 0.18 | 1.00 | 0.12 | 1.00 | 0.12 | 1.00 | 0.01 | 0.67 | 0.01 | 56 |
| rs2304722 | 72028802 | 0.93 | 0.32 | 0.82 | 0.65 | 1.00 | 0.38 | 0.90 | 0.44 | 1.00 | 0.06 | 0.47 | 0.02 | 1.00 | 0.08 | 1.00 | 0.06 | 91 |
| rs12437465 | 72030299 | 0.40 | 0.12 | 0.47 | 0.11 | 0.51 | 0.16 | 0.59 | 0.32 | 1.00 | 0.20 | 0.31 | 0.05 | 1.00 | 0.13 | 1.00 | 0.17 | 57 |
| rs12441138 | 72030950 | 1.00 | 0.02 | 1.00 | 0.02 | 1.00 | 0.26 | 1.00 | 0.15 | 1.00 | 0.01 | 0.26 | 0.01 | 1.00 | 0.02 | 0.24 | 0.00 | 163 |
| rs12904518 | 72034997 | 0.43 | 0.03 | 0.65 | 0.02 | 0.83 | 0.41 | 0.80 | 0.25 | 0.17 | 0.02 | 0.07 | 0.00 | 0.39 | 0.05 | 0.54 | 0.08 | 164 |
| rs12102015 | 72035413 | 0.56 | 0.04 | 1.00 | 0.06 | 0.67 | 0.22 | 0.78 | 0.22 | 0.26 | 0.05 | 0.42 | 0.09 | 0.38 | 0.06 | 0.48 | 0.07 | 165 |
| rs12101466 | 72035441 | 0.55 | 0.04 | 0.60 | 0.03 | 0.69 | 0.32 | 0.66 | 0.19 | 0.20 | 0.03 | 0.30 | 0.06 | 0.40 | 0.04 | 0.53 | 0.08 | 166 |
| rs8042532 | 72042283 | 1.00 | 0.01 | — | — | 1.00 | 0.22 | 0.86 | 0.17 | 1.00 | 0.04 | — | — | 0.51 | 0.00 | 0.27 | 0.04 | 167 |
| rs4886809 | 72045502 | 0.25 | 0.05 | 0.33 | 0.00 | 0.85 | 0.22 | 0.42 | 0.09 | 0.04 | 0.00 | 0.07 | 0.00 | 0.43 | 0.01 | 0.33 | 0.01 | 168 |
| rs2507 | 72062729 | 0.60 | 0.05 | 0.26 | 0.01 | 0.65 | 0.24 | 0.65 | 0.17 | 0.27 | 0.06 | 0.16 | 0.02 | 0.40 | 0.06 | 0.56 | 0.09 | 23 |
| rs2289413 | 72063281 | — | — | 0.04 | 0.00 | 1.00 | 0.20 | 0.85 | 0.15 | — | — | 0.16 | 0.00 | 1.00 | 0.02 | 0.29 | 0.05 | 169 |
| rs4993000 | 72069396 | 0.58 | 0.09 | 0.18 | 0.00 | 0.64 | 0.23 | 0.65 | 0.17 | 0.50 | 0.17 | 0.11 | 0.01 | 0.41 | 0.06 | 0.56 | 0.09 | 170 |
| rs2301273 | 72073935 | — | — | 0.30 | 0.01 | 1.00 | 0.20 | 0.85 | 0.16 | — | — | 1.00 | 0.01 | 1.00 | 0.02 | 0.28 | 0.04 | 171 |
| rs11072464 | 72092235 | 0.05 | 0.00 | 0.66 | 0.05 | 0.21 | 0.03 | 0.19 | 0.03 | 0.40 | 0.03 | 0.45 | 0.20 | 0.68 | 0.06 | 0.27 | 0.01 | 172 |
| rs3825941 | 72104415 | 0.45 | 0.12 | 1.00 | 0.00 | 0.87 | 0.27 | 0.39 | 0.10 | 0.76 | 0.05 | 1.00 | 0.01 | 0.50 | 0.02 | 0.26 | 0.01 | 173 |
| rs743582 | 72115259 | 1.00 | 0.03 | 1.00 | 0.01 | 1.00 | 0.22 | 0.89 | 0.24 | 0.70 | 0.10 | 0.14 | 0.00 | 0.09 | 0.01 | 0.59 | 0.13 | 174 |
| rs10851869 | 72118136 | 0.16 | 0.01 | 0.30 | 0.01 | 0.64 | 0.23 | 0.65 | 0.17 | 0.40 | 0.05 | 0.28 | 0.06 | 0.42 | 0.07 | 0.56 | 0.09 | 175 |
| rs1550435 | 72118438 | 0.16 | 0.01 | 0.32 | 0.02 | 0.65 | 0.25 | 0.65 | 0.17 | 0.40 | 0.05 | 0.34 | 0.08 | 0.41 | 0.06 | 0.56 | 0.09 | 176 |
| rs3784556 | 72119241 | 0.55 | 0.02 | 0.12 | 0.01 | 1.00 | 0.22 | 0.89 | 0.24 | 0.06 | 0.00 | 0.27 | 0.01 | 0.09 | 0.01 | 0.59 | 0.13 | 29 |
| rs4886574 | 72248773 | 0.10 | 0.00 | 0.03 | 0.00 | 0.69 | 0.29 | 0.07 | 0.00 | 0.77 | 0.03 | 0.05 | 0.00 | 0.09 | 0.00 | 0.68 | 0.08 | 38 |
| rs4887066 | 72249682 | 0.10 | 0.00 | 0.03 | 0.00 | 0.69 | 0.29 | 0.07 | 0.00 | 1.00 | 0.01 | 0.16 | 0.01 | 0.09 | 0.00 | 0.68 | 0.08 | 177 |
| rs6495089 | 72249854 | 0.12 | 0.01 | 0.05 | 0.00 | 0.67 | 0.24 | 0.08 | 0.00 | 0.77 | 0.04 | 0.06 | 0.00 | 0.02 | 0.00 | 0.38 | 0.03 | 178 |
| rs1385650 | 72253519 | 0.06 | 0.00 | — | — | 0.67 | 0.24 | 0.08 | 0.00 | 0.72 | 0.02 | — | — | 0.23 | 0.01 | 0.38 | 0.03 | 179 |
| rs10910 | 72259252 | 0.08 | 0.00 | 0.14 | 0.01 | 0.66 | 0.25 | 0.06 | 0.00 | 0.73 | 0.03 | 0.11 | 0.00 | 0.18 | 0.00 | 0.34 | 0.02 | 180 |
| rs2959010 | 72400150 | 0.14 | 0.00 | — | — | 1.00 | 0.06 | 0.68 | 0.07 | 0.02 | 0.00 | — | — | 1.00 | 0.01 | 0.63 | 0.35 | 181 |
| rs2277603 | 72409586 | 0.11 | 0.00 | — | — | 0.62 | 0.20 | 0.44 | 0.03 | 1.00 | 0.02 | — | — | 0.01 | 0.00 | 0.08 | 0.01 | 182 |
| rs16969393 | 72570423 | 0.18 | 0.01 | — | — | 0.63 | 0.20 | 0.28 | 0.06 | 0.13 | 0.01 | — | — | 0.46 | 0.02 | 0.37 | 0.02 | 183 |
| rs12439113 | 72643200 | 0.04 | 0.00 | 0.17 | 0.01 | 0.60 | 0.20 | 0.20 | 0.03 | 0.48 | 0.01 | 0.20 | 0.03 | 0.47 | 0.02 | 0.30 | 0.02 | 184 |
| rs6495122 | 72912698 | 0.12 | 0.01 | 0.24 | 0.00 | 0.34 | 0.03 | 0.26 | 0.01 | 0.18 | 0.00 | 0.05 | 0.00 | 0.51 | 0.23 | 0.61 | 0.21 | 185 |
| rs2290573 | 72916647 | 0.18 | 0.03 | — | — | 0.05 | 0.00 | 0.39 | 0.03 | 0.23 | 0.01 | — | — | 0.32 | 0.09 | 0.62 | 0.25 | 186 |
| rs12487 | 72923747 | 0.10 | 0.01 | — | — | 0.36 | 0.02 | 0.21 | 0.00 | 0.15 | 0.00 | — | — | 0.55 | 0.23 | 0.21 | 0.02 | 187 |
| rs11630918 | 72942949 | 0.05 | 0.00 | 1.00 | 0.00 | 0.42 | 0.05 | 0.19 | 0.01 | 0.08 | 0.00 | 0.14 | 0.00 | 0.35 | 0.10 | 0.70 | 0.24 | 188 |
| rs7497393 | 72962510 | 0.05 | 0.00 | — | — | 0.34 | 0.03 | 0.32 | 0.02 | 0.10 | 0.00 | — | — | 0.38 | 0.13 | 0.60 | 0.20 | 189 |
| rs1127796 | 72980057 | 0.06 | 0.00 | 1.00 | 0.00 | 0.21 | 0.01 | 0.32 | 0.02 | 0.11 | 0.00 | 1.00 | 0.01 | 0.31 | 0.07 | 0.73 | 0.28 | 190 |
| rs11856413 | 72986945 | 0.06 | 0.00 | — | — | 0.41 | 0.03 | 0.56 | 0.06 | 0.11 | 0.00 | — | — | 0.46 | 0.19 | 0.63 | 0.28 | 191 |
| rs11072512 | 72991079 | 0.06 | 0.00 | 1.00 | 0.00 | 0.21 | 0.01 | 0.32 | 0.02 | 0.11 | 0.00 | 1.00 | 0.01 | 0.31 | 0.07 | 0.73 | 0.28 | 192 |
| rs1133323 | 72999278 | 0.07 | 0.00 | — | — | 0.41 | 0.03 | 0.73 | 0.08 | 0.11 | 0.00 | — | — | 0.46 | 0.19 | 0.65 | 0.35 | 193 |
| rs1133322 | 72999410 | 0.06 | 0.00 | 1.00 | 0.00 | 0.21 | 0.01 | 0.32 | 0.02 | 0.13 | 0.00 | 1.00 | 0.01 | 0.31 | 0.07 | 0.73 | 0.28 | 194 |
| rs11072513 | 73008021 | 0.06 | 0.00 | — | — | 0.41 | 0.03 | 0.39 | 0.03 | 0.13 | 0.00 | — | — | 0.46 | 0.19 | 0.62 | 0.25 | 195 |
| rs11072514 | 73008918 | 0.06 | 0.00 | — | — | 0.41 | 0.03 | 0.56 | 0.06 | 0.13 | 0.00 | — | — | 0.46 | 0.19 | 0.63 | 0.28 | 196 |
| rs2415251 | 73029208 | 0.04 | 0.00 | — | — | 0.43 | 0.02 | 0.73 | 0.08 | 0.06 | 0.00 | — | — | 0.50 | 0.12 | 0.53 | 0.23 | 197 |

Another preferred embodiment relates to haplotypes comprising markers rs1048661 and rs3825942, or markers in linkage disequilibrium therewith. Thus, individuals carrying the G-rs1048661 G-rs3825942 haplotype or the T-rs1048661 G-rs3825942 haplotypes are significantly increased risk of developing exfoliation glaucoma (XFG) compared with individuals who are carriers of the G-rs1048661 A-rs3825942 haplotype.

For the SNP markers described herein, the opposite allele to the allele found to be in excess in patients (at-risk allele) is found in decreased frequency in individuals diagnosed with, or having symptoms associated with, XFS and/or glaucoma. For example, the T allele of marker rs1048661 and the A allele of marker rs3825942 are found in decreased frequency in XFS and glaucoma patients. These marker alleles are thus protective for XFS and/or glaucoma, i.e. they confer a decreased risk or susceptibility of individuals carrying these markers and/or haplotypes developing XFS and/or glaucoma.

Certain haplotypes useful for detecting association to XFS and glaucoma can in certain cases comprise a combination of various genetic markers, e.g., SNPs and microsatellites. Detecting such haplotypes can be accomplished by methods known in the art and/or described herein for detecting sequences at polymorphic sites. Furthermore, correlation between certain haplotypes or sets of markers and disease phenotype can be verified using standard techniques. A representative example of a simple test for correlation would be a Fisher-exact test on a two by two table.

In specific embodiments, a marker allele or haplotype found to be associated with increased risk of XFS and/or glaucoma, (e.g., markers as listed in Tables 4 and 6a) is one in which the marker allele or haplotype is more frequently present in an individual at risk for XFS and/or glaucoma (affected), compared to the frequency of its presence in a healthy individual (control), wherein the presence of the marker allele or haplotype is indicative of XFS and/or glaucoma or a susceptibility to XFS and/or glaucoma or symptoms associated with XFS and/or glaucoma. In other embodiments, at-risk markers in linkage disequilibrium with one or more markers found to be associated with XFS and/or glaucoma (e.g., markers as listed in Tables 4 and 6a) are tagging markers that are more frequently present in an individual at risk for XFS and/or glaucoma (affected), compared to the frequency of their presence in a healthy individual (control), wherein the presence of the tagging markers is indicative of increased susceptibility to XFS and/or glaucoma. In a further embodiment, at-risk markers (i.e. conferring increased susceptibility) in linkage disequilibrium with one or more markers found to be associated with XFS and/or glaucoma (e.g., markers as listed in Tables 4 and 6a), are markers comprising one or more allele that is more frequently present in an individual at risk for XFS and/or glaucoma, compared to the frequency of their presence in a healthy individual (control), wherein the presence of the markers is indicative of increased susceptibility to XFS and/or glaucoma.

### Study Population

In a general sense, the methods and kits of the invention can be utilized from samples containing genomic DNA from any source, i.e. any individual. In preferred embodiments, the individual is a human individual. The individual can be an adult, child, or fetus. The present invention also provides for assessing markers and/or haplotypes in individuals who are members of a target population. Such a target population is in one embodiment a population or group of individuals at risk of developing XFS and/or glaucoma, based on other genetic factors, biomarkers, biophysical parameters, history of XFS and/or glaucoma or related diseases, previous diagnosis of XFS and/or glaucoma, family history of XFS and/or glaucoma or general health and/or lifestyle parameters.

The invention provides for embodiments that include individuals from specific age subgroups, such as those over the age of 40, over age of 45, or over age of 50, 55, 60, 65, 70, 75, 80, or 85. Risk of XFS and glaucoma increases with age. Thus, in preferred embodiments, the invention relates to individuals over age 50, such as individuals over age 55, individuals over age 60, individuals over age 65 or individuals over age 70. It will however be appreciated by the skilled person that even individuals at lower age, such as age 35 or above or age 40 and above may benefit from the diagnostic applications of the invention. This is especially true for individuals with one or more additional risk factors for developing XFS and/or glaucoma. Such individuals may thus in particular benefit from monitoring beginning at relatively early age, so as to minimize the risk of loss of vision due to the development of XFS, glaucoma and/or cataract. Other embodiments of the invention pertain to other age groups, such as individuals aged less than 85, such as less than age 80, less than age 75, or less than age 70, 65, 60, 55, 50, 45, 40, 35, or age 30. Other embodiments relate to individuals with age at onset of XFS, cataract and/or glaucoma in any of the age ranges described in the above. It is also contemplated that a range of ages may be relevant in certain embodiments, such as age at onset at more than age 45 but less than age 60. Other age ranges are however also contemplated, including all age ranges bracketed by the age values listed in the above. The invention furthermore relates to individuals of either sex, males or females.

The Icelandic population is a Caucasian population of Northern European ancestry. A large number of studies reporting results of genetic linkage and association in the Icelandic population have been published in the last few years. Many of those studies show replication of variants, originally identified in the Icelandic population as being associating with a particular disease, in other populations (Styrkarsdottir, U., et al. N Engl J Med Apr. 29, 2008 (Epub ahead of print); Thorgeirsson, T., et al. Nature 452:638-42 (2008); Gudmundsson, J. et al. Nat Genet. 40:281-3 (2008); Stacey, S, N., et al., Nat Genet. 39:865-69 (2007); Helgadottir, A., et al., Science 316:1491-93 (2007); Steinthorsdottir, V., et al., Nat Genet. 39:770-75 (2007); Gudmundsson, J., et al., Nat. Genet. 39:631-37 (2007); Frayling, T M, Nature Reviews Genet. 8:657-662 (2007); Amundadottir, L. T., et al., Nat. Genet. 38:652-58 (2006); Grant, S. F., et al., Nat. Genet. 38:320-23 (2006)). Thus, genetic findings in the Icelandic population have in general been replicated in other populations, including populations from Africa and Asia. Moreover, the association of the variants described herein to be associated with increased risk of XFS and glaucoma has been replicated in several populations, including Caucasians from Europe and North America (Mossböck, G., et al. Mol Vis 14:857-61 (2001); Aragon-Martin, J. A., et al Mol Vis 14:533-41 (2008); Pasutto, F., et al. Invest Opthalmol Vis Sci 49:1459-63 (2008); Challa, P., et al. Mol Vis 14:146-9 (2008); Yang, X., et al. Cell Cycle 7:521-4 (2008); Fingert, J. H., et al. 144:974-975 (2007)); in Cacucasian Australians (Hewitt, A. W., et al. Hum Mol Genet. 17:710-6 (2008); in Indians (Ramprasa, V. L., et al. Mol Vis 14:318-22 (2008), in an ethnically mixed US cohort (Fan, B. J. et al. BMC Med Genet. 9:5 (2008); and in Japanese (Ozaki, M., et al. Invest Opthalmol Vis Sci Apr. 30, 2008 (Epub ahead of print); Hayashi, H., et al. Am J Opthalmol 145:582-585 (2008)).

The markers of the present invention found to be associated with exfoliation syndrome and glaucoma are therefore believed to show similar association in all human populations. Particular embodiments comprising individual human populations are thus also contemplated and within the scope of the invention. Such embodiments relate to human subjects that are from one or more human population including, but not limited to, Caucasian populations, European populations, American populations, Eurasian populations, Asian populations, Central/South Asian populations, East Asian populations, Middle Eastern populations, African populations, Hispanic populations, and Oceanian populations. European populations include, but are not limited to, Swedish, Norwegian, Finnish, Russian, Danish, Icelandic, Irish, Kelt, English, Scottish, Dutch, Belgian, French, German, Spanish, Portuguese, Italian, Polish, Bulgarian, Slavic, Serbian, Bosnian, Chech, Greek and Turkish populations. The invention furthermore in other embodiments can be practiced in specific human populations that include Bantu, Mandenk, Yoruba, San, Mbuti Pygmy, Orcadian, Adygel, Russian, Sardinian, Tuscan, Mozabite, Bedouin, Druze, Palestinian, Balochi, Brahui, Makrani, Sindhi, Pathan, Burusho, Hazara, Uygur, Kalash, Han, Dai, Daur, Hezhen, Lahu, Miao, Oroqen, She, Tujia, Tu, Xibo, Yi, Mongolan, Naxi, Cambodian, Japanese, Yakut, Melanesian, Papuan, Karitianan, Surui, Colombian, Maya and Pima.

In certain embodiments, the invention relates to populations that include black African ancestry such as populations comprising persons of African descent or lineage. Black African ancestry may be determined by self reporting as African-Americans, Afro-Americans, Black Americans, being a member of the black race or being a member of the negro race. For example, African Americans or Black Americans are those persons living in North America and having origins in any of the black racial groups of Africa. In another example, self-reported persons of black African ancestry may have at least one parent of black African ancestry or at least one grandparent of black African ancestry.

The racial contribution in individual subjects may also be determined by genetic analysis. Genetic analysis of ancestry may be carried out using unlinked microsatellite markers such as those set out in Smith et al. (Am J Hum Genet. 74, 1001-13 (2004)).

In certain embodiments, the invention relates to markers and/or haplotypes identified in specific populations, as described in the above. The person skilled in the art will appreciate that measures of linkage disequilibrium (LD) may give different results when applied to different populations. This is due to different population history of different human populations as well as differential selective pressures that may have led to differences in LD in specific genomic regions. It is also well known to the person skilled in the art that certain markers, e.g. SNP markers, have different population frequency in different populations, or are polymorphic in one population but not in another. The person skilled in the art will however apply the methods available and as taught herein to practice the present invention in any given human population. This may include assessment of polymorphic markers in the LD region of the present invention, so as to identify those markers that give strongest association within the specific population. Thus, the at-risk variants of the present invention may reside on different haplotype background and in different frequencies in various human populations. However, utilizing methods taught herein and the markers of the present invention, the invention can be practiced in any given human population.

### Utility of Genetic Testing

The person skilled in the art will appreciate and understand that the variants described herein in general do not, by themselves, provide an absolute identification of individuals who will develop a particular disease. The variants described herein do however indicate increased and/or decreased likelihood that individuals carrying the at-risk or protective variants of the invention will develop symptoms associated with XFS and/or glaucoma. This information is however extremely valuable in itself, as outlined in more detail in the below, as it can be used to, for example, initiate preventive measures at an early stage, perform regular physical and/or mental exams to monitor the progress and/or appearance of symptoms, or to schedule exams at a regular interval to identify the condition in question, so as to be able to apply treatment at an early stage.

The knowledge about a genetic variant that confers a risk of developing XFS and/or glaucoma offers the opportunity to apply a genetic test to distinguish between individuals with increased risk of developing XFS and/or glaucoma (i.e. carriers of the at-risk variant) and those with decreased risk of developing XFS and/or glaucoma (i.e. carriers of the protective variant). The core values of genetic testing, for individuals belonging to both of the above mentioned groups, are the possibilities of being able to diagnose XFS and/or glaucoma, or a predisposition to XFS and/or glaucoma, at an early stage and provide information to the clinician about prognosis/aggressiveness of XFS and/or glaucoma in order to be able to apply the most appropriate treatment.

Individuals with a family history of XFS and/or glaucoma and carriers of at-risk variants may benefit from genetic testing since the knowledge of the presence of a genetic risk factor, or evidence for increased risk of being a carrier of one or more risk factors, may provide increased incentive for implementing a healthier lifestyle, by avoiding or minimizing known environmental risk factors for XFS and/or glaucoma. Genetic testing of XFS and/or glaucoma patients may furthermore give valuable information about the primary cause of XFS and/or glaucoma and can aid the clinician in selecting the best treatment options and medication for each individual. Further, individuals who are carriers of the at-risk variants of the invention are likely to benefit from regular monitoring from the clinician, so as to minimize the risk of developing symptoms with, or being diagnosed with, XFS, glaucoma and/or cataract.

The present invention furthermore relates to risk assessment for XFS and/or glaucoma, including diagnosing whether an individual is at risk for developing XFS and/or glaucoma. The polymorphic markers of the present invention can be used alone or in combination, as well as in combination with other factors, including other genetic risk factors or biomarkers, for risk assessment of an individual for XFS and/or glaucoma. Many factors known to affect the predisposition of an individual towards developing risk of developing XFS and/or glaucoma are known to the person skilled in the art and can be utilized in such assessment. These include, but are not limited to, increased intraocular pressure, age, visual field abnormalities that are observed in otherwise baseline visual field examinations, high myopia, family history of glaucoma and/or XFS, thin cornea (central corneal thickness of less than 556 μm), a vertical or horizontal cup-to-disc ratio of greater than 0.4, systemic hypertension, cardiovascular disease, migraine headache, and peripheral vasospasm.

Yet another utility lies on the use of genetic markers to determine whether to apply particular treatment modalities. Thus, based on the carrier status of particular markers and haplotypes described herein to be associated with risk of glaucoma and/or exfoliation syndrome, a particular treatment is administred. This can for example be done by first determining whether an individual is carrying at least one particular risk allele of one or more markers, or by determining the carrier status of the individual with respect to at least one particular haplotype. Based on the result of the genetic analysis, the particular treatment modality is administered. The treatment modality can for example be a therapeutic agent for glaucoma or exfoliation syndrome, as described herein, or a therepeutic agent for lowering intraocular pressure.

Methods known in the art can be used for such assessment, including multivariate analyses or logistic regression.

### METHODS

Methods for risk assessment and risk management of XFS and glaucoma are described herein and are encompassed by the invention. The invention also encompasses therapeutic aspects, methods of assessing an individual for probability of response to a therapeutic agent for XFS and/or glaucoma, as well as methods for predicting the effectiveness of a therapeutic agent for XFS and/or glaucoma. Kits for assaying a sample from a subject to detect susceptibility to XFS and/or glaucoma are also encompassed by the invention.

### Diagnostic and Screening Methods

In certain embodiments, the present invention pertains to methods of determining a susceptibility to, or diagnosing, or aiding in the diagnosis of, XFS and/or glaucoma, in particular exfoliation glaucoma, by detecting particular alleles at genetic markers that appear more frequently in subjects diagnosed with XFS and/or glaucoma or subjects who are susceptible to symptoms associated with XFS and/or glaucoma. In a particular embodiment, the invention is a method of diagnosing a susceptibility to XFS and/or glaucoma, by detecting or determining whether at least one allele of at least one polymorphic marker (e.g., the markers described herein) is present in a nucleic acid sample from the individual or in a genotype dataset derived from the individual. The present invention describes methods whereby detection of particular alleles of particular markers or haplotypes is indicative of a susceptibility to XFS and glaucoma. Such prognostic or predictive assays can also be used to determine prophylactic treatment of a subject prior to the onset of symptoms of XFS and/or glaucoma.

The present invention pertains in some embodiments to methods of clinical applications of diagnosis, e.g., diagnosis performed by a medical professional. In other embodiments, the invention pertains to methods of diagnosis or determination of a susceptibility performed by a layman. The layman can be the customer of a genotyping service. The layman may also be a genotype service provider, who performs genotype analysis on a DNA sample from an individual, in order to provide service related to genetic risk factors for particular traits or diseases, based on the genotype status of the individual (i.e., the customer). Recent technological advances in genotyping technologies, including high-throughput genotyping of SNP markers, such as Molecular Inversion Probe array technology (e.g., Affymetrix GeneChip), and BeadArray Technologies (e.g., Illumina GoldenGate and Infinium assays) have made it possible for individuals to have their own genome assessed for up to one million SNPs simultaneously, at relatively little cost. The resulting genotype information, which can be made available to the individual, can be compared to information about disease or trait risk associated with various SNPs, including information from public literature and scientific publications. The diagnostic application of disease-associated alleles as described herein, can thus for example be performed by the individual, through analysis of his/her genotype data, by a health professional based on results of a clinical test, or by a third party, including the genotype service provider. The third party may also be service provider who interprets genotype information derived from the customer, or which is representative of the individual (i.e. derived from the individual through genotyping of particular polymorphic markers) to provide service related to specific genetic risk factors, including the genetic markers described herein. In other words, the diagnosis or determination of a susceptibility of genetic risk can be made by health professionals, genetic counselors, third parties providing genotyping service, third parties providing risk assessment service or by the layman (e.g., the individual), based on information about the genotype status of an individual and knowledge about the risk conferred by particular genetic risk factors (e.g., particular SNPs). In the present context, the term “diagnosing”, “diagnose a susceptibility” and “determine a susceptibility” is meant to refer to the determination of disease susceptibility by any particular person or any particular procedure, including those described above.

In certain embodiments, a sample containing genomic DNA from an individual is collected. Such sample can for example be a buccal swab, a saliva sample, a blood sample, or other suitable sample type containing genomic DNA, as described further herein. The genomic DNA is subsequently isolated from the sample and then analyzed using any suitable genotyping technique available to the skilled person, such as by high-throughput array technologies or other methods described herein. Results from such genotyping are stored in a convenient data storage unit, such as a data carrier, including computer databases, data storage disks, or by other convenient data storage means. In certain embodiments, the computer database is an object database, a relational database or a post-relational database. The genotype data is subsequently analyzed for the presence of certain variants known to be susceptibility variants for a particular human conditions, such as the genetic variants described herein. Genotype data can be retrieved from the data storage unit using any convenient data query method. Calculating risk conferred by a particular genotype for the individual can be based on comparing the genotype of the individual to previously determined risk (expressed as a relative risk (RR) or and odds ratio (OR), for example) for the genotype, for example for an heterozygous carrier of an at-risk variant for the particular disease or trait (e.g. XFS and/or glaucoma, e.g. exfoliation glaucoma). In certain embodiments, haplotypes comprising two or more polymorphic markers are analyzed for risk assessment. The calculated risk for the individual can be the relative risk for a person, or for a specific genotype of a person, compared to the average population with matched gender and ethnicity. The average population risk can be expressed as a weighted average of the risks of different genotypes, using results from a reference population, and the appropriate calculations to calculate the risk of a genotype group relative to the population can then be performed. Alternatively, the risk for an individual is based on a comparison of particular genotypes, for example heterozygous carriers of an at-risk allele of a marker compared with non-carriers of the at-risk allele. Using the population average may in certain embodiments be more convenient, since it provides a measure which is easy to interpret for the user, i.e. a measure that gives the risk for the individual, based on his/her genotype, compared with the average in the population. The calculated risk estimated can be made available to the customer via a website, preferably a secure website.

In certain embodiments, a service provider will include in the provided service all of the steps of isolating genomic DNA from a sample provided by the customer, performing genotyping of the isolated DNA, calculating genetic risk based on the genotype data, and report the risk to the customer. In some other embodiments, the service provider will include in the service the interpretation of genotype data for the individual, i.e., risk estimates for particular genetic variants based on the genotype data for the individual. In some other embodiments, the service provider may include service that includes genotyping service and interpretation of the genotype data, starting from a sample of isolated DNA from the individual (the customer).

Overall risk for multiple risk variants can be performed using standard methodology. For example, assuming a multiplicative model, i.e. assuming that the risk of individual risk variants multiply to establish the overall effect, allows for a straight-forward calculation of the overall risk for multiple markers.

In addition, in certain other embodiments, the present invention pertains to methods of diagnosing, or aiding in the diagnosis of, a decreased susceptibility to XFS and/or glaucoma, by detecting particular genetic marker alleles or haplotypes that appear less frequently in cardiovascular disease, including XFS and/or glaucoma patients than in individual not diagnosed with XFS and/or glaucoma, or in the general population. For example, while the at-risk alleles of the markers disclosed herein (e.g. G-rs1048661 and G-rs3825942 in the LOXL1 gene, and R141 and G153 at the amino acid level of LOXL1) confer high risk of developing XFS and exfoliation glaucoma, the alternate allele for these markers (T-rs1048661 and A-rs3825942, L141 and D153) are protective, since individuals carrying at least one of those alleles are at decreased risk of developing XFS and/or exfoliation glaucoma, compared with the population average or carriers of at-risk alleles.

As described and exemplified herein, particular marker alleles or haplotypes (e.g. the markers and haplotypes as listed in Tables 4 and 6a and markers in linkage disequilibrium therewith, e.g. rs1048661 and rs3825942, and rs1048661-rs3825942 haplotypes) are associated with risk of XFS and/or glaucoma. In one embodiment, the marker allele or haplotype is one that confers a significant risk or susceptibility to XFS and/or glaucoma. In another embodiment, the invention relates to a method of diagnosing a susceptibility to XFS and/or glaucoma in a human individual, the method comprising determining the presence or absence of at least one allele of at least one polymorphic marker in a nucleic acid sample obtained from the individual, wherein the at least one polymorphic marker is selected from the group consisting of the polymorphic markers listed in Table 4 and 6a, and markers in linkage disequilibrium (e.g., defined as r²>0.2) therewith. In another embodiment, the invention pertains to methods of diagnosing a susceptibility to XFS and/or glaucoma in a human individual, by screening for at least one marker allele or haplotype as listed in Table 15 or 16 or markers in linkage disequilibrium therewith.

In another embodiment, the invention pertains to methods of diagnosing a susceptibility to XFS and/or glaucoma in a human individual, by screening for at least one marker selected from rs4886725 (SEQ ID NO:86), rs12915956 (SEQ ID NO:87), rs896590 (SEQ ID NO:88), rs12438872 (SEQ ID NO:89), rs4261482 (SEQ ID NO:42), rs2165241 (SEQ ID NO:15), rs1992314 (SEQ ID NO:44), rs4886776 (SEQ ID NO:45), rs2028386 (SEQ ID NO:46), rs4337252 (SEQ ID NO:47), rs2028387 (SEQ ID NO:48), rs4077284 (SEQ ID NO:49), rs893816 (SEQ ID NO:50), rs4886782 (SEQ ID NO:51), rs893817 (SEQ ID NO:17), rs893818 (SEQ ID NO:52), rs893820 (SEQ ID NO:53), rs12440667 (SEQ ID NO:54), rs1530169 (SEQ ID NO:19), rs893821 (SEQ ID NO:90), rs750460 (SEQ ID NO:55), rs4243042 (SEQ ID NO:56), rs2304722 (SEQ ID NO:91), rs4886623 (SEQ ID NO:92), rs2167648 (SEQ ID NO:93), rs1584738 (SEQ ID NO:94), rs1901570 (SEQ ID NO:95), rs8026593 (SEQ ID NO:6), rs4886660 (SEQ ID NO:96), rs4886421 (SEQ ID NO:97), rs4886663 (SEQ ID NO:98), rs4886664 (SEQ ID NO:99), rs4145873 (SEQ ID NO:100), rs4145874 (SEQ ID NO:101), rs1452389 (SEQ ID NO:102), rs1078967 (SEQ ID NO:103), rs8041642 (SEQ ID NO:104), rs8041685 (SEQ ID NO:16), rs8042039 (SEQ ID NO:105), rs2304719 (SEQ ID NO:18), rs12437465 (SEQ ID NO:57), rs1048661 (SEQ ID NO:106), and rs3825942 (SEQ ID NO:107).

In a preferred embodiment, the invention pertains to methods of diagnosing a susceptibility to XFS and/or glaucoma in a human individual, by screening for at least one marker allele or haplotype for marker rs1048661 and/or marker rs3825942. In another embodiment, the marker allele or haplotype is more frequently present in a subject having, or who is susceptible to, XFS and/or glaucoma (affected), as compared to the frequency of its presence in a healthy subject (control, such as population controls). In certain embodiments, the significance of association of the at least one marker allele or haplotype is characterized by a p value<0.05. In other embodiments, the significance of association is characterized by smaller p-values, such as <0.01, <0.001, <0.0001, <0.00001, <0.000001, <0.0000001, <0.00000001 or <0.000000001.

In these embodiments, the presence of the at least one marker allele or haplotype is indicative of a susceptibility to XFS and/or glaucoma or a risk or XFS and/or glaucoma. These diagnostic methods involve detecting the presence or absence of at least one marker allele or haplotype that is associated with XFS and/or glaucoma. The haplotypes described herein include combinations of alleles at various genetic markers (e.g., SNPs, microsatellites). The detection of the particular genetic marker alleles that make up the particular haplotypes can be performed by a variety of methods described herein and/or known in the art. For example, genetic markers can be detected at the nucleic acid level (e.g., by direct nucleotide sequencing or by other means known to the skilled in the art) or at the amino acid level if the genetic marker affects the coding sequence of a protein encoded by a XFS and/or glaucoma-associated nucleic acid (e.g., a LOXL1-encoding nucleic acid), by protein sequencing or by immunoassays using antibodies that recognize such a protein. The marker alleles or haplotypes of the present invention correspond to fragments of a genomic DNA sequence associated with XFS and/or glaucoma. Such fragments encompass the DNA sequence of the polymorphic marker or haplotype in question, but may also include DNA segments in strong LD (linkage disequilibrium) with the marker or haplotype. In one embodiment, such segments comprises segments in LD with the marker or haplotype as determined by a value of r² greater than 0.2 and/or |D′|>0.8).

In one embodiment, diagnosis of a susceptibility to XFS and/or glaucoma can be accomplished using hybridization methods (see Current Protocols in Molecular Biology, Ausubel, F. et al., eds., John Wiley & Sons, including all supplements). A biological sample from a test subject or individual (a “test sample”) of genomic DNA, RNA, or cDNA is obtained from a subject suspected of having, being susceptible to, experiencing symptoms associated with, or predisposed for XFS and/or glaucoma (the “test subject”). The subject can be an adult, child, or fetus. The test sample can be from any source that contains genomic DNA, such as a blood sample, sample of amniotic fluid, sample of cerebrospinal fluid, or tissue sample from skin, muscle, buccal or conjunctival mucosa (buccal swab), placenta, gastrointestinal tract or other organs. A test sample of DNA from fetal cells or tissue can be obtained by appropriate methods, such as by amniocentesis or chorionic villus sampling. The DNA, RNA, or cDNA sample is then examined. The presence of a specific marker allele can be indicated by sequence-specific hybridization of a nucleic acid probe specific for the particular allele. The presence of more than specific marker allele or a specific haplotype can be indicated by using several sequence-specific nucleic acid probes, each being specific for a particular allele. In one embodiment, a haplotype can be indicated by a single nucleic acid probe that is specific for the specific haplotype (i.e., hybridizes specifically to a DNA strand comprising the specific marker alleles characteristic of the haplotype). A sequence-specific probe can be directed to hybridize to genomic DNA, RNA, or cDNA. A “nucleic acid probe”, as used herein, can be a DNA probe or an RNA probe that hybridizes to a complementary sequence. One of skill in the art would know how to design such a probe so that sequence specific hybridization will occur only if a particular allele is present in a genomic sequence from a test sample. The invention can also be reduced to practice using any convenient genotyping method, including commercially available technologies or by other methods for genotyping particular polymorphic markers available to the skilled person.

To diagnose a susceptibility to XFS and/or glaucoma and or symptoms associated with XFS and/or glaucoma, a hybridization sample can be formed by contacting the test sample containing an XFS and/or glaucoma-associated nucleic acid, such as a genomic DNA sample, with at least one nucleic acid probe. A non-limiting example of a probe for detecting mRNA or genomic DNA is a labeled nucleic acid probe that is capable of hybridizing to mRNA or genomic DNA sequences described herein. The nucleic acid probe can be, for example, a full-length nucleic acid molecule, or a portion thereof, such as an oligonucleotide of at least 10, 15, 30, 50, 100, 250 or 500 nucleotides in length that is sufficient to specifically hybridize under stringent conditions to appropriate mRNA or genomic DNA. For example, the nucleic acid probe can comprise all or a portion of the nucleotide sequence of the LOXL1 gene and/or the LOXL1 LD block (SEQ ID NO:84), as described herein, optionally comprising at least one allele of a marker described herein, or at least one haplotype described herein, or the probe can be the complementary sequence of such a sequence. In a particular embodiment, the nucleic acid probe is a portion of the nucleotide sequence of the LOXL1 gene and/or the LOXL1 LD block, as described herein (SEQ ID NO:84), optionally comprising at least one allele of a marker described herein, or at least one allele of one polymorphic marker or haplotype comprising at least one polymorphic marker described herein, or the probe can be the complementary sequence of such a sequence. Other suitable probes for use in the diagnostic assays of the invention are described herein. Hybridization can be performed by methods well known to the person skilled in the art (see, e.g., Current Protocols in Molecular Biology, Ausubel, F. et al., eds., John Wiley & Sons, including all supplements). In one embodiment, hybridization refers to specific hybridization, i.e., hybridization with no mismatches (exact hybridization). In one embodiment, the hybridization conditions for specific hybridization are high stringency.

Specific hybridization, if present, is detected using standard methods. If specific hybridization occurs between the nucleic acid probe and the nucleic acid in the test sample, then the sample contains the allele that is complementary to the nucleotide that is present in the nucleic acid probe. The process can be repeated for any markers of the present invention, or markers that make up a haplotype of the present invention, or multiple probes can be used concurrently to detect more than one marker alleles at a time. It is also possible to design a single probe containing more than one marker alleles of a particular haplotype (e.g., a probe containing alleles complementary to 2, 3, 4, 5 or all of the markers that make up a particular haplotype). Detection of the particular markers of the haplotype in the sample is indicative that the source of the sample has the particular haplotype (e.g., a haplotype) and therefore is susceptible to XFS and/or glaucoma. Preferable haplotypes to be detected by this method include (1) rs1048661 allele G and rs3825942 allele G; (2) rs1048661 allele T and rs3825942 allele G; and (3) rs1048661 allele G and rs3825942 allele A.

In one preferred embodiment, a method utilizing a detection oligonucleotide probe comprising a fluorescent moiety or group at its 3′ terminus and a quencher at its 5′ terminus, and an enhancer oligonucleotide, is employed, as described by Kutyavin et al. (Nucleic Acid Res. 34:e128 (2006)). The fluorescent moiety can be Gig Harbor Green or Yakima Yellow, or other suitable fluorescent moieties. The detection probe is designed to hybridize to a short nucleotide sequence that includes the SNP polymorphism to be detected. Preferably, the SNP is anywhere from the terminal residue to −6 residues from the 3′ end of the detection probe. The enhancer is a short oligonucleotide probe which hybridizes to the DNA template 3′ relative to the detection probe. The probes are designed such that a single nucleotide gap exists between the detection probe and the enhancer nucleotide probe when both are bound to the template. The gap creates a synthetic abasic site that is recognized by an endonuclease, such as Endonuclease IV. The enzyme cleaves the dye off the fully complementary detection probe, but cannot cleave a detection probe containing a mismatch. Thus, by measuring the fluorescence of the released fluorescent moiety, assessment of the presence of a particular allele defined by nucleotide sequence of the detection probe can be performed.

The detection probe can be of any suitable size, although preferably the probe is relatively short. In one embodiment, the probe is from 5-100 nucleotides in length. In another embodiment, the probe is from 10-50 nucleotides in length, and in another embodiment, the probe is from 12-30 nucleotides in length. Other lengths of the probe are possible and within scope of the skill of the average person skilled in the art.

In a preferred embodiment, the DNA template containing the SNP polymorphism is amplified by Polymerase Chain Reaction (PCR) prior to detection. In such an embodiment, the amplified DNA serves as the template for the detection probe and the enhancer probe.

Certain embodiments of the detection probe, the enhancer probe, and/or the primers used for amplification of the template by PCR include the use of modified bases, including modified A and modified G. The use of modified bases can be useful for adjusting the melting temperature of the nucleotide molecule (probe and/or primer) to the template DNA, for example for increasing the melting temperature in regions containing a low percentage of G or C bases, in which modified A with the capability of forming three hydrogen bonds to its complementary T can be used, or for decreasing the melting temperature in regions containing a high percentage of G or C bases, for example by using modified G bases that form only two hydrogen bonds to their complementary C base in a double stranded DNA molecule. In a preferred embodiment, modified bases are used in the design of the detection nucleotide probe. Any modified base known to the skilled person can be selected in these methods, and the selection of suitable bases is well within the scope of the skilled person based on the teachings herein and known bases available from commercial sources as known to the skilled person.

In another hybridization method, Northern analysis (see Current Protocols in Molecular Biology, Ausubel, F. et al., eds., John Wiley & Sons, supra) is used to identify the presence of a polymorphism associated with XFS and/or glaucoma. For Northern analysis, a test sample of RNA is obtained from the subject by appropriate means. As described herein, specific hybridization of a nucleic acid probe to RNA from the subject is indicative of a particular allele complementary to the probe. For representative examples of use of nucleic acid probes, see, for example, U.S. Pat. Nos. 5,288,611 and 4,851,330.

Additionally, or alternatively, a peptide nucleic acid (PNA) probe can be used in addition to, or instead of, a nucleic acid probe in the hybridization methods described herein. A PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen, P., et al., Bioconjug. Chem. 5:3-7 (1994)). The PNA probe can be designed to specifically hybridize to a molecule in a sample suspected of containing one or more of the marker alleles or haplotypes that are associated with XFS and/or glaucoma. Hybridization of the PNA probe is thus diagnostic for XFS and/or glaucoma.

In one embodiment of the invention, a test sample containing genomic DNA obtained from the subject is collected and the polymerase chain reaction (PCR) is used to amplify a fragment comprising one or more markers or haplotypes of the present invention. As described herein, identification of a particular marker allele or haplotype associated with XFS and/or glaucoma, can be accomplished using a variety of methods (e.g., sequence analysis, analysis by restriction digestion, specific hybridization, single stranded conformation polymorphism assays (SSCP), electrophoretic analysis, etc.). In another embodiment, diagnosis is accomplished by expression analysis, for example using quantitative PCR (kinetic thermal cycling). This technique can, for example, utilize commercially available technologies, such as TaqMan (Applied Biosystems, Foster City, Calif.). The technique can assess the presence of an alteration in the expression or composition of a polypeptide or splicing variant(s) that is encoded by a nucleic acid associated with XFS and/or glaucoma. Further, the expression of the variant(s) can be quantified as physically or functionally different.

In another method of the invention, analysis by restriction digestion can be used to detect a particular allele if the allele results in the creation or elimination of a restriction site relative to a reference sequence. Restriction fragment length polymorphism (RFLP) analysis can be conducted, e.g., as described in Current Protocols in Molecular Biology, supra. The digestion pattern of the relevant DNA fragment indicates the presence or absence of the particular allele in the sample.

Sequence analysis can also be used to detect specific alleles or haplotypes associated with XFS and/or glaucoma (e.g. the polymorphic markers of Tables 4 and 6a, and markers in linkage disequilibrium therewith). Therefore, in one embodiment, determination of the presence or absence of a particular marker alleles or haplotypes comprises sequence analysis of a test sample of DNA or RNA obtained from a subject or individual. PCR or other appropriate methods can be used to amplify a portion of a nucleic acid associated with XFS and/or glaucoma, and the presence of a specific allele can then be detected directly by sequencing the polymorphic site (or multiple polymorphic sites in a haplotype) of the genomic DNA in the sample.

Allele-specific oligonucleotides can also be used to detect the presence of a particular allele in a nucleic acid associated with XFS and/or glaucoma (e.g. the polymorphic markers of Tables 4 and 6a, and markers in linkage disequilibrium therewith), through the use of dot-blot hybridization of amplified oligonucleotides with allele-specific oligonucleotide (ASO) probes (see, for example, Saiki, R. et al., Nature, 324:163-166 (1986)). An “allele-specific oligonucleotide” (also referred to herein as an “allele-specific oligonucleotide probe”) is an oligonucleotide of approximately 10-50 base pairs or approximately 15-30 base pairs, that specifically hybridizes to a nucleic acid associated with XFS and/or glaucoma, and which contains a specific allele at a polymorphic site (e.g., a marker or haplotype as described herein). An allele-specific oligonucleotide probe that is specific for one or more particular a nucleic acid associated with XFS and/or glaucoma can be prepared using standard methods (see, e.g., Current Protocols in Molecular Biology, supra). PCR can be used to amplify the desired region. The DNA containing the amplified region can be dot-blotted using standard methods (see, e.g., Current Protocols in Molecular Biology, supra), and the blot can be contacted with the oligonucleotide probe. The presence of specific hybridization of the probe to the amplified region can then be detected. Specific hybridization of an allele-specific oligonucleotide probe to DNA from the subject is indicative of a specific allele at a polymorphic site associated with cardiovascular disease, including coronary artery disease and Myocardial Infarction (see, e.g., Gibbs, R. et al., Nucleic Acids Res., 17:2437-2448 (1989) and WO 93/22456).

With the addition of such analogs as locked nucleic acids (LNAs), the size of primers and probes can be reduced to as few as 8 bases. LNAs are a novel class of bicyclic DNA analogs in which the 2′ and 4′ positions in the furanose ring are joined via an O-methylene (oxy-LNA), S-methylene (thio-LNA), or amino methylene (amino-LNA) moiety. Common to all of these LNA variants is an affinity toward complementary nucleic acids, which is by far the highest reported for a DNA analog. For example, particular all oxy-LNA nonamers have been shown to have melting temperatures (Tₘ) of 64° C. and 74° C. when in complex with complementary DNA or RNA, respectively, as opposed to 28° C. for both DNA and RNA for the corresponding DNA nonamer. Substantial increases in Tₘ are also obtained when LNA monomers are used in combination with standard DNA or RNA monomers. For primers and probes, depending on where the LNA monomers are included (e.g., the 3′ end, the 5′ end, or in the middle), the Tₘ could be increased considerably.

In another embodiment, arrays of oligonucleotide probes that are complementary to target nucleic acid sequence segments from a subject, can be used to identify polymorphisms in a nucleic acid associated with XFS and/or glaucoma (e.g. the polymorphic markers of Tables 4 and 6a and markers in linkage disequilibrium therewith). For example, an oligonucleotide array can be used. Oligonucleotide arrays typically comprise a plurality of different oligonucleotide probes that are coupled to a surface of a substrate in different known locations. These arrays can generally be produced using mechanical synthesis methods or light directed synthesis methods that incorporate a combination of photolithographic methods and solid phase oligonucleotide synthesis methods, or by other methods known to the person skilled in the art (see, e.g., Bier, F. F., et al. Adv Biochem Eng Biotechnol 109:433-53 (2008); Hoheisel, J. D., Nat Rev Genet 7:200-10 (2006); Fan, J. B., et al. Methods Enzymol 410:57-73 (2006); Raqoussis, J. & Elvidge, G., Expert Rev Mol Diagn 6:145-52 (2006); Mockler, T. C., et al Genomics 85:1-15 (2005), and references cited therein, the entire teachings of each of which are incorporated by reference herein). Many additional descriptions of the preparation and use of oligonucleotide arrays for detection of polymorphisms can be found, for example, in U.S. Pat. No. 6,858,394, U.S. Pat. No. 6,429,027, U.S. Pat. No. 5,445,934, U.S. Pat. No. 5,700,637, U.S. Pat. No. 5,744,305, U.S. Pat. No. 5,945,334, U.S. Pat. No. 6,054,270, U.S. Pat. No. 6,300,063, U.S. Pat. No. 6,733,977, U.S. Pat. No. 7,364,858, EP 619 321, and EP 373 203, the entire teachings of which are incorporated by reference herein.

Other methods of nucleic acid analysis that are available to those skilled in the art can be used to detect a particular allele at a polymorphic site associated with XFS and/or glaucoma (e.g. the polymorphic markers of Tables 4 and 6a and markers in linkage disequilibrium therewith). Representative methods include, for example, direct manual sequencing (Church and Gilbert, Proc. Nat. Acad. Sci. USA, 81: 1991-1995 (1988); Sanger, F., et al., Proc. Natl. Acad. Sci. USA, 74:5463-5467 (1977); Beavis, et al, U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield, V., et al., Proc. Natl. Acad. Sci. USA, 86:232-236 (1989)), mobility shift analysis (Orita, M., et al., Proc. Natl. Acad. Sci. USA, 86:2766-2770 (1989)), restriction enzyme analysis (Flavell, R., et al., Cell, 15:25-41 (1978); Geever, R., et al., Proc. Nat. Acad. Sci. USA, 78:5081-5085 (1981)); heteroduplex analysis; chemical mismatch cleavage (CMC) (Cotton, R., et al., Proc. Natl. Acad. Sci. USA, 85:4397-4401 (1985)); RNase protection assays (Myers, R., et al., Science, 230:1242-1246 (1985); use of polypeptides that recognize nucleotide mismatches, such as E. coli mutS protein; and allele-specific PCR.

In another embodiment of the invention, diagnosis of XFS and/or glaucoma can be made by examining expression and/or composition of a polypeptide encoded by a nucleic acid associated with XFS and/or glaucoma (e.g., a LOXL1 polypeptide encoded by the LOXL1 gene on Chr15q24 in those instances where the genetic marker(s) or haplotype(s) of the present invention result in a change in the composition or expression of the polypeptide (e.g., LOXL1 expression). Thus, diagnosis of a susceptibility to XFS and/or glaucoma can be made by examining expression and/or composition of LOXL1, or another polypeptide encoded by a nucleic acid associated with XFS and/or glaucoma, in those instances where the genetic marker or haplotype of the present invention results in a change in the composition or expression of the polypeptide. The haplotypes and markers of the present invention that show association to XFS and/or glaucoma may play a role through their effect on the LOXL1 gene, or one or more of the nearby genes. Possible mechanisms affecting these genes include, e.g., effects on transcription, effects on RNA splicing, alterations in relative amounts of alternative splice forms of mRNA, effects on RNA stability, effects on transport from the nucleus to cytoplasm, and effects on the efficiency and accuracy of translation. The present inventors have for example found that marker rs1048661 (SEQ ID NO: 106) correlates with LOXL1 expression. The presence of allele G at this polymorphic site correlates with decreased expression of LOXL1 in adipose tissue. Thus, in one embodiment, diagnosis of an increased susceptibility to glaucoma can be performed by determining the expression levels of LOXL1, wherein decreased expression levels are indicative of an increased susceptibility of glaucoma and/or XFS. Determining expression levels of LOXL1 can also be employed in other methods of the invention, including screening assays.

In another embodiment, the variants (markers or haplotypes) of the invention showing association to XFS and/or glaucoma affect the expression of a gene nearby to LOXL1. It is well known that regulatory element affecting gene expression may be located tenths or even hundreds of kilobases away from the promoter region of a gene. By assaying for the presence or absence of at least one allele of at least one polymorphic marker of the present invention, it is thus possible to assess the expression level of such nearby genes. It is thus contemplated that the detection of the markers or haplotypes of the present invention can be used for assessing expression for one or more of such nearby genes.

A variety of methods can be used for detecting protein expression levels, including enzyme linked immunosorbent assays (ELISA), Western blots, immunoprecipitations and immunofluorescence. A test sample from a subject is assessed for the presence of an alteration in the expression and/or an alteration in composition of the polypeptide encoded by a nucleic acid associated with XFS and/or glaucoma. An alteration in expression of a polypeptide encoded by a nucleic acid associated with XFS and/or glaucoma (e.g., LOXL1) can be, for example, an alteration in the quantitative polypeptide expression (i.e. the amount of polypeptide produced). An alteration in the composition of a polypeptide encoded by a nucleic acid associated with XFS and/or glaucoma (e.g., LOXL1) is an alteration in the qualitative polypeptide expression (e.g., expression of a mutant polypeptide or of a different splicing variant). In one embodiment, diagnosis of a susceptibility to XFS and/or glaucoma is made by detecting a particular splicing variant encoded by the LOXL1 gene, or a particular pattern of splicing variants encoded by the LOXL1 gene.

Both such alterations (quantitative and qualitative) can also be present. An “alteration” in the polypeptide expression or composition, as used herein, refers to an alteration in expression or composition in a test sample, as compared to the expression or composition of the polypeptide in a control sample. A control sample is a sample that corresponds to the test sample (e.g., is from the same type of cells), and is from a subject who is not affected by, and/or who does not have a susceptibility to, XFS and/or glaucoma. In one embodiment, the control sample is from a subject that does not possess a marker allele or haplotype as described herein. Similarly, the presence of one or more different splicing variants in the test sample, or the presence of significantly different amounts of different splicing variants in the test sample, as compared with the control sample, can be indicative of a susceptibility to XFS and/or glaucoma. An alteration in the expression or composition of the polypeptide in the test sample, as compared with the control sample, can be indicative of a specific allele in the instance where the allele alters a splice site relative to the reference in the control sample. Various means of examining expression or composition of a polypeptide encoded by a nucleic acid are known to the person skilled in the art and can be used, including spectroscopy, colorimetry, electrophoresis, isoelectric focusing, and immunoassays (e.g., David et al., U.S. Pat. No. 4,376,110) such as immunoblotting (see, e.g., Current Protocols in Molecular Biology, particularly chapter 10, supra).

For example, in one embodiment, an antibody (e.g., an antibody with a detectable label) that is capable of binding to a polypeptide encoded by a nucleic acid associated with XFS and/or glaucoma, e.g. the LOXL1 protein or a fragment of the LOXL1 protein, can be used. Antibodies can be polyclonal or monoclonal. An intact antibody, or a fragment thereof (e.g., Fv, Fab, Fab′, F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a labeled secondary antibody (e.g., a fluorescently-labeled secondary antibody) and end-labeling of a DNA probe with biotin such that it can be detected with fluorescently-labeled streptavidin.

In one embodiment of this method, the level or amount of polypeptide encoded by a nucleic acid associated with XFS and/or glaucoma in a test sample, e.g., the LOXL1 protein or a fragment or splice variant thereof, is compared with the level or amount of the polypeptide in a control sample. A level or amount of the polypeptide in the test sample that is higher or lower than the level or amount of the polypeptide in the control sample, such that the difference is statistically significant, is indicative of an alteration in the expression of the polypeptide encoded by the nucleic acid, and is diagnostic for a particular allele or haplotype responsible for causing the difference in expression. Alternatively, the composition of the polypeptide in a test sample is compared with the composition of the polypeptide in a control sample. In another embodiment, both the level or amount and the composition of the polypeptide can be assessed in the test sample and in the control sample.

In another embodiment, the diagnosis of a susceptibility to XFS and/or glaucoma is made by detecting at least one marker or haplotypes of the present invention (e.g., associated alleles of the markers listed in Tables 4 and 6a, and markers in linkage disequilibrium therewith), in combination with an additional protein-based, RNA-based or DNA-based assay. The methods of the invention can also be used in combination with an analysis of a subject's family history and risk factors (e.g., environmental risk factors, lifestyle risk factors).

### Kits

Kits useful in the methods of the invention comprise components useful in any of the methods described herein, including for example, primers for nucleic acid hybridization, hybridization probes, restriction enzymes (e.g., for RFLP analysis), allele-specific oligonucleotides, antibodies that bind to an altered polypeptide encoded by a nucleic acid of the invention as described herein (e.g., a genomic segment comprising at least one polymorphic marker and/or haplotype of the present invention) or to a non-altered (native) polypeptide encoded by a nucleic acid of the invention as described herein, means for amplification of a nucleic acid associated with XFS and/or glaucoma, means for analyzing the nucleic acid sequence of a nucleic acid associated with XFS and/or glaucoma, means for analyzing the amino acid sequence of a polypeptide encoded by a nucleic acid associated with XFS and/or glaucoma (e.g., the LOXL1 protein encoded by the LOXL1 gene), etc. The kits can for example include necessary buffers, nucleic acid primers for amplifying nucleic acids of the invention (e.g., a nucleic acid segment comprising one or more of the polymorphic markers as described herein), and reagents for allele-specific detection of the fragments amplified using such primers and necessary enzymes (e.g., DNA polymerase). Additionally, kits can provide reagents for assays to be used in combination with the methods of the present invention, e.g., reagents for use with other XFS and/or glaucoma diagnostic assays.

In one embodiment, the invention is a kit for assaying a sample from a subject to detect the presence of XFS and/or glaucoma, symptoms associated with XFS and/or glaucoma, or a susceptibility to XFS and/or glaucoma in a subject, wherein the kit comprises reagents necessary for selectively detecting at least one allele of at least one polymorphism of the present invention in the genome of the individual. In a particular embodiment, the reagents comprise at least one contiguous oligonucleotide that hybridizes to a fragment of the genome of the individual comprising at least one polymorphism of the present invention. In another embodiment, the reagents comprise at least one pair of oligonucleotides that hybridize to opposite strands of a genomic segment obtained from a subject, wherein each oligonucleotide primer pair is designed to selectively amplify a fragment of the genome of the individual that includes at least one polymorphism, wherein the polymorphism is selected from the group consisting of the polymorphisms as listed in Tables 4 and 6a, and polymorphic markers in linkage disequilibrium therewith. In yet another embodiment the fragment is at least 20 base pairs in size. Such oligonucleotides or nucleic acids (e.g., oligonucleotide primers) can be designed using portions of the nucleic acid sequence flanking polymorphisms (e.g., SNPs or microsatellites) that are indicative of XFS and/or glaucoma. In another embodiment, the kit comprises one or more labeled nucleic acids capable of allele-specific detection of one or more specific polymorphic markers or haplotypes associated with XFS and/or glaucoma, and reagents for detection of the label. Suitable labels include, e.g., a radioisotope, a fluorescent label, an enzyme label, an enzyme co-factor label, a magnetic label, a spin label, an epitope label.

In particular embodiments, the polymorphic marker or haplotype to be detected by the reagents of the kit comprises one or more markers, two or more markers, three or more markers, four or more markers or five or more markers selected from the group consisting of the markers in Table 4, 6a and 16. In another embodiment, the marker or haplotype to be detected comprises the markers listed in Table 4. In another embodiment, the marker or haplotype to be detected comprises the markers listed in Table 6a. In another embodiment, the marker or haplotype to be detected comprises at least one marker from the group of markers in strong linkage disequilibrium, as defined by values of r² greater than 0.2, to at least one of the group of markers listed in Table 15. In another embodiment, the marker or haplotype to be detected comprises at least one marker selected from the markers set forth in Table 16. In another embodiment, the marker or haplotype to be detected comprises markers rs1048661 and rs3825942.

In one preferred embodiment, the kit for detecting the markers of the invention comprises a detection oligonucleotide probe, that hybridizes to a segment of template DNA containing a SNP polymorphisms to be detected, an enhancer oligonucleotide probe and an endonuclease. As explained in the above, the detection oligonucleotide probe comprises a fluorescent moiety or group at its 3′ terminus and a quencher at its 5′ terminus, and an enhancer oligonucleotide, is employed, as described by Kutyavin et al. (Nucleic Acid Res. 34:e128 (2006)). The fluorescent moiety can be Gig Harbor Green or Yakima Yellow, or other suitable fluorescent moieties. The detection probe is designed to hybridize to a short nucleotide sequence that includes the SNP polymorphism to be detected. Preferably, the SNP is anywhere from the terminal residue to −6 residues from the 3′ end of the detection probe. The enhancer is a short oligonucleotide probe which hybridizes to the DNA template 3′ relative to the detection probe. The probes are designed such that a single nucleotide gap exists between the detection probe and the enhancer nucleotide probe when both are bound to the template. The gap creates a synthetic abasic site that is recognized by an endonuclease, such as Endonuclease IV. The enzyme cleaves the dye off the fully complementary detection probe, but cannot cleave a detection probe containing a mismatch. Thus, by measuring the fluorescence of the released fluorescent moiety, assessment of the presence of a particular allele defined by nucleotide sequence of the detection probe can be performed.

The detection probe can be of any suitable size, although preferably the probe is relatively short. In one embodiment, the probe is from 5-100 nucleotides in length. In another embodiment, the probe is from 10-50 nucleotides in length, and in another embodiment, the probe is from 12-30 nucleotides in length. Other lengths of the probe are possible and within scope of the skill of the average person skilled in the art.

In a preferred embodiment, the DNA template containing the SNP polymorphism is amplified by Polymerase Chain Reaction (PCR) prior to detection, and primers for such amplification are included in the reagent kit. In such an embodiment, the amplified DNA serves as the template for the detection probe and the enhancer probe.

Certain embodiments of the detection probe, the enhancer probe, and/or the primers used for amplification of the template by PCR include the use of modified bases, including modified A and modified G. The use of modified bases can be useful for adjusting the melting temperature of the nucleotide molecule (probe and/or primer) to the template DNA, for example for increasing the melting temperature in regions containing a low percentage of G or C bases, in which modified A with the capability of forming three hydrogen bonds to its complementary T can be used, or for decreasing the melting temperature in regions containing a high percentage of G or C bases, for example by using modified G bases that form only two hydrogen bonds to their complementary C base in a double stranded DNA molecule. In a preferred embodiment, modified bases are used in the design of the detection nucleotide probe. Any modified base known to the skilled person can be selected in these methods, and the selection of suitable bases is well within the scope of the skilled person based on the teachings herein and known bases available from commercial sources as known to the skilled person.

In one of such embodiments, the presence of the marker or haplotype is indicative of a susceptibility (increased susceptibility or decreased susceptibility) to XFS and/or glaucoma. In another embodiment, the presence of the marker or haplotype is indicative of response to a XFS and/or glaucoma therapeutic agent. In another embodiment, the presence of the marker or haplotype is indicative of XFS and/or glaucoma prognosis. In yet another embodiment, the presence of the marker or haplotype is indicative of progress of XFS and/or glaucoma treatment. Such treatment may include intervention by surgery, medication or by other means (e.g., lifestyle changes).

In a further aspect of the present invention, a pharmaceutical pack (kit) is provided, the pack comprising a therapeutic agent and a set of instructions for administration of the therapeutic agent to humans diagnostically tested for one or more variants of the present invention, as disclosed herein. The therapeutic agent can be a small molecule drug, an antibody, a peptide, an antisense or RNAi molecule, or other therapeutic molecules. In one embodiment, an individual identified as a carrier of at least one variant of the present invention is instructed to take a prescribed dose of the therapeutic agent. In one such embodiment, an individual identified as a homozygous carrier of at least one variant of the present invention is instructed to take a prescribed dose of the therapeutic agent. In another embodiment, an individual identified as a non-carrier of at least one variant of the present invention is instructed to take a prescribed dose of the therapeutic agent.

In certain embodiments, the kit further comprises a set of instructions for using the reagents comprising the kit.

### Therapeutic Agents

Variants of the present invention (e.g., the markers and/or haplotypes of the invention, e.g., the markers listed in Tables 4 and 6a) can be used to identify novel therapeutic targets for XFS and/or glaucoma. For example, genes containing, or in linkage disequilibrium with, variants (markers and/or haplotypes) associated with XFS and/or glaucoma (e.g., the LOXL1 gene), or their products, as well as genes or their products that are directly or indirectly regulated by or interact with these variant genes or their products, can be targeted for the development of therapeutic agents to treat XFS and/or glaucoma, or prevent or delay onset of symptoms associated with XFS and/or glaucoma. Therapeutic agents may comprise one or more of, for example, small non-protein and non-nucleic acid molecules, proteins, peptides, protein fragments, nucleic acids (DNA, RNA), PNA (peptide nucleic acids), or their derivatives or mimetics which can modulate the function and/or levels of the target genes or their gene products. In a preferred embodiment, the LOXL1 gene is targeted for development of therapeutic agents for preventing or ameliorating symptoms associated with glaucoma or XFS.

It is contemplated that therapy based on delivery of LOXL1 protein, either directly by protein delivery, or indirectly by delivery of vectors capable of inducing the production of LOXL1 in vivo is advantageous. The inventors have found that two non-synonymous (coding) polymorphisms in LOXL1 are associated with glaucoma (in particular exfoliation glaucoma) and XFS. It is possible that the proteins that have the amino acids of the at-risk variants (Arginine at position 141 in SEQ ID NO:85 and Glycine at position 153 in SEQ ID NO:85) of the invention are functionally impaired. Delivery of protein to a person in need thereof, either a person diagnosed with glaucoma or XFS, or a person at risk for developing glaucoma or XFS, may ameliorate or prevent the symptoms associated with the disease.

LOXL1 is a member of a family of enzymes which catalyze the oxidative deamination of lysine (and hydroxylysine) residues in extracellular collagens and elastin. The function of these enzymes is to produce reactive aldehydes, which spontanelusly react with other aldehydes or unmodified lysines to produce a variety of inter- and intramolecular cross-links found in collagen and elastin fibrils.

There are 5 known members of the LOX family of enzymes (Csiszar, K. Prog Nucleic Acid Res 70:1-32 (2001)). The catalytic domain is highly conserved and contains the residues required for catalytic function. The enzyme(s) utilize a unique post-translationally derived cofactor, which is formed via a cross-link between strictly conserved Tyr and Lys residues within the catalytic domain. This cofactor, termed Lysine-Tyrosine-Quinone (LTQ) is highly electrophilic and reacts directly with the amine substrates. The enzyme(s) also bind a catalytic Cu ion, which is involved in the oxidative chemistry and formation of hydrogen peroxide.

The main difference between the sequence of the LOX members is within the N-terminal domains of the proteins(s). All the proteins carry an approximately 20 amino acid signal sequence at the N-terminus, which directs the proteins to the extracellular matrix (ECM). The LOX and LOXL1 proteins also carry a long pro-sequence, which recent studies have shown to be important for correct deposition of the protein to the ECM (Thomassin, L. et al. J Biol Chem 280:42848-55 (2005)). The main difference between the substrate/functional roles of LOX and LOXL1 is that whereas LOX is mainly responsible for collagen crosslinking, LOXL1 mainly associates with sites of elastogenesis and interacts with fibulin-5 (Thomassin, L. et al. J Biol Chem 280:42848-55 (2005); Liu, X. et al. Nat Genet. 36:178-82 (2004)). In general, the existence of multiple LOX members suggests a non-redundant function of these proteins, which presumably is manifested in different tissue expression, distribution etc.

As mentioned above, the pro-sequence of LOX/LOXL1 is required for proper deposition of the protein in the ECM. In addition, the forms of the enzyme(s) containing the pro-sequence are velieved by be catalytically quiescent. This post-translational regulation is reminiscent of the activation of extracellular proteases, such as the serine proteases.

The main extracellular protease responsible for cleavage of the pro-sequence in LOX and LOXL1 is bone morphogenetic protein-1 (BMP-1), also referred to as procollagen-C-proteinase (PCP) (Csiszar, K. Prog Nucleic Acid Res 70:1-32 (2001); Trackman, P. C. J Cell Biochem 96:927-37 (2005)). Interestingly, this zinc containing protease also cleaves procollagen and this processing and subsequent aggregation of mature collagen into microfibrils is required for oxidation by LOX (Trackman, P. C. J Cell Biochem 96:927-37 (2005)). Thus, the same protease that processess procollagen to the collagen substrate of LOX is also the converter of proLOX to the functional, mature enzyme, representing a highly integrated mechanism for the formation of crosslinked collagen.

Alignment of several known substrates for BMP-1 is shown below. Although a rigorously defined

| | |
|---|---|
| Chordin | KLGDPMQA--DGPRGCRF |
| Laminin | DTGDCYSG--DENPDIEC |
| Procollagen α1(I) | DGGRYYRA--DDANVVRD |
| Procollagen α2(I) | YDGDFYRA--DQPRSAPS |
| Procollagen (II) | ...QYMRA--DQA..... |
| Procollagen (III) | GGFAPYYG--DEPMDFKI |
| Procollagen (VII) | ...PSYAA--DTAG.... |
| LOX | SHVDRMVG--DDPYNPYK |
| LOXL-1 (D153) | SVSQQRHG--DSASSVSA |
| Consensus | A/G--D |

consensus sequence for BMP-1 cleavage is not established, the enzyme shows preference for A or G at the P-site and D at the P′-site. BMP-1 mediated cleavage of human LOX occurs at the G168-D169 bond (Panchenko, M. V. et al J Biol Chem 271:7113-19 (1996); Cronshaw, A. D. et al. Biochem J 306:279-84 (1995)). This yields a 32 kDa mature protein from the 50 kDa proenzyme. The corresponding cleavage site in LOXL1 has not been clearly identified, although various products have been described, including products around 66, 56, 51, 42, 37 and 33 kDa (Csiszar, K. Prog Nucleic Acid Res 70:1-32 (2001); Borel, A. Et al J Biol Chem 276:48944-49 (2001)). Difference in glycosylation of LOXL1 (and other members) makes assignments based on molecular weight very difficult and N-terminal sequencing of isolated products has not yielded a unified cleavage site, although G-D pairs at sites 135 and 304 have been mentioned as plausible candidates.

Interestingly, the G153D variant (G-rs1048661 A-rs3825942 haplotype) reveals what may be a cryptic protelytic cleavage site for BMP-1. It is postulated that individuals carrying the G-rs1048661 A-rs3825942 haplotype are protected against XFG and XFS due to a more efficient and proper proteolytic processing of ProLOXL1, compared to the other haplotypes. Efficient processing of the enzyme could lead to an increase in overall total activity and or deposition of the enzyme in the ECM, which could be beneficial in abrogating the harmful accumulation of abnormal fibrillar material observed in XFG. Thus, therapeutic delivery of the G153D variant, either directly or through a nucleic acid delivery system which is capable of generating the variant in situ, is contemplated as a benefitial treatment option. Therefore, in a preferred embodiment, the delivered protein contains an arginine at position 141 in SEQ ID NO:85 and an aspartic acid at position 153 in SEQ ID NO:85.

The inventors have furthermore found that LOXL1 protein characterized by the presence of Arginine at position 141 in SEQ ID NO:85 correlates with decreased expression of LOXL1. This observation indicates that alternate alleles at this position, in addition to changing the encoded amino acid from Leucine (the rare variant) to Arginine (the common, or “wild type”, variant), also may affect expression of LOXL1. This observation also suggests that delivery of LOXL1 protein or induction of the production of LOXL1 protein in vivo is beneficial as a therapeutic means for glaucoma and XFS.

The nucleic acids and/or variants of the invention, or nucleic acids comprising their complementary sequence, may also be used as antisense constructs to control gene expression in cells, tissues or organs. The methodology associated with antisense techniques is well known to the skilled artisan, and is described and reviewed in AntisenseDrug Technology: Principles, Strategies, and Applications, Crooke, ed., Marcel Dekker Inc., New York (2001). In general, antisense nucleic acid molecules are designed to be complementary to a region of mRNA expressed by a gene, so that the antisense molecule hybridizes to the mRNA, thus blocking translation of the mRNA into protein. Several classes of antisense oligonucleotide are known to those skilled in the art, including cleavers and blockers. The former bind to target RNA sites, activate intracellular nucleases (e.g., RnaseH or Rnase L), that cleave the target RNA. Blockers bind to target RNA, inhibit protein translation by steric hindrance of the ribosomes. Examples of blockers include nucleic acids, morpholino compounds, locked nucleic acids and methylphosphonates (Thompson, Drug Discovery Today, 7:912-917 (2002)). Antisense oligonucleotides are useful directly as therapeutic agents, and are also useful for determining and validating gene function, for example by gene knock-out or gene knock-down experiments. Antisense technology is further described in Lavery et al., Curr. Opin. Drug Discov. Devel. 6:561-569 (2003), Stephens et al., Curr. Opin. Mol. Ther. 5:118-122 (2003), Kurreck, Eur. J. Biochem. 270:1628-44 (2003), Dias et al., Mol. Cancer. Ter. 1:347-55 (2002), Chen, Methods Mol. Med. 75:621-636 (2003), Wang et al., Curr. Cancer Drug Targets 1:177-96 (2001), and Bennett, Antisense Nucleic Acid Drug. Dev. 12:215-24 (2002).

The variants described herein can be used for the selection and design of antisense reagents that are specific for particular variants. Using information about the variants described herein, antisense oligonucleotides or other antisense molecules that specifically target mRNA molecules that contain one or more variants of the invention can be designed. In this manner, expression of mRNA molecules that contain one or more variant of the present invention (markers and/or haplotypes) can be inhibited or blocked. In one embodiment, the antisense molecules are designed to specifically bind a particular allelic form (i.e., one or several variants (alleles and/or haplotypes)) of the target nucleic acid, thereby inhibiting translation of a product originating from this specific allele or haplotype, but which do not bind other or alternate variants at the specific polymorphic sites of the target nucleic acid molecule.

As antisense molecules can be used to inactivate mRNA so as to inhibit gene expression, and thus protein expression, the molecules can be used to treat a disease or disorder, such as XFS and/or glaucoma. The methodology can involve cleavage by means of ribozymes containing nucleotide sequences complementary to one or more regions in the mRNA that attenuate the ability of the mRNA to be translated. Such mRNA regions include, for example, protein-coding regions, in particular protein-coding regions corresponding to catalytic activity, substrate and/or ligand binding sites, or other functional domains of a protein.

The phenomenon of RNA interference (RNAi) has been actively studied for the last decade, since its original discovery in C. elegans (Fire et al., Nature 391:806-11 (1998)), and in recent years its potential use in treatment of human disease has been actively pursued (reviewed in Kim & Rossi, Nature Rev. Genet. 8:173-204 (2007)). RNA interference (RNAi), also called gene silencing, is based on using double-stranded RNA molecules (dsRNA) to turn off specific genes. In the cell, cytoplasmic double-stranded RNA molecules (dsRNA) are processed by cellular complexes into small interfering RNA (siRNA). The siRNA guide the targeting of a protein-RNA complex to specific sites on a target mRNA, leading to cleavage of the mRNA (Thompson, Drug Discovery Today, 7:912-917 (2002)). The siRNA molecules are typically about 20, 21, 22 or 23 nucleotides in length. Thus, one aspect of the invention relates to isolated nucleic acid molecules, and the use of those molecules for RNA interference, i.e. as small interfering RNA molecules (siRNA). In one embodiment, the isolated nucleic acid molecules are 18-26 nucleotides in length, preferably 19-25 nucleotides in length, more preferably 20-24 nucleotides in length, and more preferably 21, 22 or 23 nucleotides in length.

Another pathway for RNAi-mediated gene silencing originates in endogenously encoded primary microRNA (pri-miRNA) transcripts, which are processed in the cell to generate precursor miRNA (pre-miRNA). These miRNA molecules are exported from the nucleus to the cytoplasm, where they undergo processing to generate mature miRNA molecules (miRNA), which direct translational inhibition by recognizing target sites in the 3′ untranslated regions of mRNAs, and subsequent mRNA degradation by processing P-bodies (reviewed in Kim & Rossi, Nature Rev. Genet. 8:173-204 (2007)).

Clinical applications of RNAi include the incorporation of synthetic siRNA duplexes, which preferably are approximately 20-23 nucleotides in size, and preferably have 3′ overlaps of 2 nucleotides. Knockdown of gene expression is established by sequence-specific design for the target mRNA. Several commercial sites for optimal design and synthesis of such molecules are known to those skilled in the art.

Other applications provide longer siRNA molecules (typically 25-30 nucleotides in length, preferably about 27 nucleotides), as well as small hairpin RNAs (shRNAs; typically about 29 nucleotides in length). The latter are naturally expressed, as described in Amarzguioui et al. (FEBS Lett. 579:5974-81 (2005)). Chemically synthetic siRNAs and shRNAs are substrates for in vivo processing, and in some cases provide more potent gene-silencing than shorter designs (Kim et al., Nature Biotechnol. 23:222-226 (2005); Siolas et al., Nature Biotechnol. 23:227-231 (2005)). In general siRNAs provide for transient silencing of gene expression, because their intracellular concentration is diluted by subsequent cell divisions. By contrast, expressed shRNAs mediate long-term, stable knockdown of target transcripts, for as long as transcription of the shRNA takes place (Marques et al., Nature Biotechnol. 23:559-565 (2006); Brummelkamp et al., Science 296: 550-553 (2002)).

Since RNAi molecules, including siRNA, miRNA and shRNA, act in a sequence-dependent manner, the variants of the present invention (e.g., markers and/or haplotypes associated with LOXL1 gene (SEQ ID NO: the markers and haplotypes set forth in Tables 4, 6a and 15) can be used to design RNAi reagents that recognize specific nucleic acid molecules comprising specific alleles and/or haplotypes (e.g., the alleles and/or haplotypes of the present invention), while not recognizing nucleic acid molecules comprising other alleles or haplotypes. These RNAi reagents can thus recognize and destroy the target nucleic acid molecules. As with antisense reagents, RNAi reagents can be useful as therapeutic agents (i.e., for turning off disease-associated genes or disease-associated gene variants), but may also be useful for characterizing and validating gene function (e.g., by gene knock-out or gene knock-down experiments).

Delivery of RNAi may be performed by a range of methodologies known to those skilled in the art. Methods utilizing non-viral delivery include cholesterol, stable nucleic acid-lipid particle (SNALP), heavy-chain antibody fragment (Fab), aptamers and nanoparticles. Viral delivery methods include use of lentivirus, adenovirus and adeno-associated virus. The siRNA molecules are in some embodiments chemically modified to increase their stability. This can include modifications at the 2′ position of the ribose, including 2′-O-methylpurines and 2′-fluoropyrimidines, which provide resistance to Rnase activity. Other chemical modifications are possible and known to those skilled in the art.

The following references provide a further summary of RNAi, and possibilities for targeting specific genes using RNAi: Kim & Rossi, Nat. Rev. Genet. 8:173-184 (2007), Chen & Rajewsky, Nat. Rev. Genet. 8: 93-103 (2007), Reynolds, et al., Nat. Biotechnol. 22:326-330 (2004), Chi et al., Proc. Natl. Acad. Sci. USA 100:6343-6346 (2003), Vickers et al., J. Biol. Chem. 278:7108-7118 (2003), Agami, Curr. Opin. Chem. Biol. 6:829-834 (2002), Lavery, et al., Curr. Opin. Drug Discov. Devel. 6:561-569 (2003), Shi, Trends Genet. 19:9-12 (2003), Shuey et al., Drug Discov. Today 7:1040-46 (2002), McManus et al., Nat. Rev. Genet. 3:737-747 (2002), Xia et al., Nat. Biotechnol. 20:1006-10 (2002), Plasterk et al., curr. Opin. Genet. Dev. 10:562-7 (2000), Bosher et al., Nat. Cell Biol. 2:E31-6 (2000), and Hunter, Curr. Biol. 9:R440-442 (1999).

Inhibitory agents, including antisense, small molecule drugs and RNAi, can also be used to perturb cellular expression control so as to increase the expression of LOXL1. Thus, inhibitory agents can be used to target inhibitory transcriptional regulators of LOXL1. Alleviation or reduction of the activity of such regulators will then lead to an increase in the transcription of LOXL1. Thus, the present invention also relates to the use of inhibitory agents that target transcriptional regulators of LOXL1. It is also contemplated that gene products located upstream in a cellular pathway leading to, or affecting, LOXL1, can be targets for such inhibitory agents. Such inhibitory agents may lead to the inhibition of LOXL1 expression, or they may lead to an increase in LOXL1 expression, depending on the normal biological function of the gene product in question.

The eye is a relatively isolated tissue compartment, making it ideal for protein therapy or gene therapy, including utilization of RNAi molecules. Local delivery to the eye by intraocular injection limits exposure to the rest of the body, and reduces the amount of therapeutic agent needed. For example, the amount of siRNA used in ocular delivery is small compared to systemic application. This allows local silencing of a gene, with little chance of affecting the gene in tissues outside of the eye. Several applications of ocular administration of siRNA have been reported, as reviewed by Campochiaro (Gene Therapy 13: 559-62 (2006)).

A genetic defect leading to increased predisposition or risk for development of a disease, including XFS and/or glaucoma, or a defect causing the disease, may be corrected permanently by administering to a subject carrying the defect a nucleic acid fragment that incorporates a repair sequence that supplies the normal/wild-type nucleotide(s) at the site of the genetic defect. Such site-specific repair sequence may encompass an RNA/DNA oligonucleotide that operates to promote endogenous repair of a subject's genomic DNA. The administration of the repair sequence may be performed by an appropriate vehicle, such as a complex with polyethelenimine, encapsulated in anionic liposomes, a viral vector such as an adenovirus vector, or other pharmaceutical compositions suitable for promoting intracellular uptake of the administered nucleic acid. The genetic defect may then be overcome, since the chimeric oligonucleotides induce the incorporation of the normal sequence into the genome of the subject, leading to expression of the normal/wild-type gene product. The replacement is propagated, thus rendering a permanent repair and alleviation of the symptoms associated with the disease or condition.

The present invention provides methods for identifying compounds or agents that can be used to treat XFS and/or glaucoma. Thus, the variants of the invention are useful as targets for the identification and/or development of therapeutic agents. Such methods may include assaying the ability of an agent or compound to modulate the activity and/or expression of a nucleic acid that includes at least one of the variants (markers and/or haplotypes) of the present invention, or the encoded product of the nucleic acid. This in turn can be used to identify agents or compounds that inhibit or alter the undesired activity or expression of the encoded nucleic acid product. Assays for performing such experiments can be performed in cell-based systems or in cell-free systems, as known to the skilled person. Cell-based systems include cells naturally expressing the nucleic acid molecules of interest, or recombinant cells that have been genetically modified so as to express a certain desired nucleic acid molecule.

Variant gene expression in a patient can be assessed by expression of a variant-containing nucleic acid sequence (for example, a gene containing at least one variant of the present invention, which can be transcribed into RNA containing the at least one variant, and in turn translated into protein), or by altered expression of a normal/wild-type nucleic acid sequence due to variants affecting the level or pattern of expression of the normal transcripts, for example variants in the regulatory or control region of the gene. Assays for gene expression include direct nucleic acid assays (mRNA), assays for expressed protein levels, or assays of collateral compounds involved in a pathway, for example a signal pathway. Furthermore, the expression of genes that are up- or down-regulated in response to the signal pathway can also be assayed. One embodiment includes operably linking a reporter gene, such as luciferase, to the regulatory region of the gene(s) of interest.

Modulators of gene expression can in one embodiment be identified when a cell is contacted with a candidate compound or agent, and the expression of mRNA is determined. The expression level of mRNA in the presence of the candidate compound or agent is compared to the expression level in the absence of the compound or agent. Based on this comparison, candidate compounds or agents for treating XFS and/or glaucoma can be identified as those modulating the gene expression of the variant gene. When expression of mRNA or the encoded protein is statistically significantly greater in the presence of the candidate compound or agent than in its absence, then the candidate compound or agent is identified as a stimulator or up-regulator of expression of the nucleic acid. When nucleic acid expression or protein level is statistically significantly less in the presence of the candidate compound or agent than in its absence, then the candidate compound is identified as an inhibitor or down-regulator of the nucleic acid expression. The invention further provides methods of treatment using a compound identified through drug (compound and/or agent) screening as a gene modulator (i.e. stimulator and/or inhibitor of gene expression).

In a further aspect of the present invention, a pharmaceutical pack (kit) is provided, the pack comprising a therapeutic agent and a set of instructions for administration of the therapeutic agent to humans diagnostically tested for one or more variants of the present invention, as disclosed herein. The therapeutic agent can be a small molecule drug, an antibody, a peptide, an antisense or RNAi molecule, or other therapeutic molecules. In one embodiment, an individual identified as a carrier of at least one variant of the present invention is instructed to take a prescribed dose of the therapeutic agent. In one such embodiment, an individual identified as a homozygous carrier of at least one variant of the present invention is instructed to take a prescribed dose of the therapeutic agent. In another embodiment, an individual identified as a non-carrier of at least one variant of the present invention is instructed to take a prescribed dose of the therapeutic agent.

### Methods of Assessing Probability of Response to Therapeutic Agents, Methods of Monitoring Progress of Treatment and Methods of Treatment

As is known in the art, individuals can have differential responses to a particular therapy (e.g., a therapeutic agent or therapeutic method). Pharmacogenomics addresses the issue of how genetic variations (e.g., the variants (markers and/or haplotypes) of the present invention) affect drug response, due to altered drug disposition and/or abnormal or altered action of the drug. Thus, the basis of the differential response may be genetically determined in part. Clinical outcomes due to genetic variations affecting drug response may result in toxicity of the drug in certain individuals (e.g., carriers or non-carriers of the genetic variants of the present invention), or therapeutic failure of the drug. Therefore, the variants of the present invention may determine the manner in which a therapeutic agent and/or method acts on the body, or the way in which the body metabolizes the therapeutic agent.

Accordingly, in one embodiment, the presence of a particular allele at a polymorphic site or haplotype is indicative of a different response, e.g. a different response rate, to a particular treatment modality. This means that a patient diagnosed with XFS and/or glaucoma, and carrying a certain allele at a polymorphic site or haplotype of the present invention (e.g., the at-risk markers of the invention) would respond better to, or worse to, a specific therapeutic, drug and/or other therapy used to treat the disease. Therefore, the presence or absence of the marker allele or haplotype could aid in deciding what treatment should be used for a the patient. For example, for a newly diagnosed patient, the presence of a marker or haplotype of the present invention may be assessed (e.g., through testing DNA derived from a blood sample, as described herein). If the patient is positive for a marker allele or haplotype at (that is, at least one specific allele of the marker, or haplotype, is present), then the physician recommends one particular therapy, while if the patient is negative for the at least one allele of a marker, or a haplotype, then a different course of therapy may be recommended (which may include recommending that no immediate therapy, other than serial monitoring for progression of the disease, be performed). Thus, the patient's carrier status could be used to help determine whether a particular treatment modality should be administered. The value lies within the possibilities of being able to diagnose the disease at an early stage, to select the most appropriate treatment, and provide information to the clinician about prognosis/aggressiveness of the disease in order to be able to apply the most appropriate treatment. This type of selection of individuals who would especially benefit from a particular treatment modality is contemplated to be applicable to a wide range of therapeutic agents for ocular conditions such as glaucoma and exfoliation syndrome, including the therapeutic agents listed in Agent Table I herein.

Another aspect of the invention relates to methods of select individuals suitable for a particular treatment modality, based on the their likelihood of developing particular complications or side effects of the particular treatment. It is well known that most therapeutic agents can lead to certain unwanted complications or side effects. Likewise, certain therapeutic procedures or operations may have complications associated with them. Complications or side effects of these particular treatments or associated with specific therapeutic agents can, just as diseases do, have a genetic component. It is therefore contemplated that selection of the appropriate treatment or therapeutic agent can in part be performed by determining the genotype of an individual, and using the genotype status of the individual to decide on a suitable therapeutic procedure or on a suitable therapeutic agent to treat the particular disease. It is therefore contemplated that the polymorphic markers of the present invention can be used in this manner. In particular, the polymorphic markers of the present invention can be used to determine whether administration of a particular therapeutic agent or treatment modality or method is suitable for the individual, based on estimating the likelihood that the individual will develop symptoms associated with exfoliation syndrome and/or glaucoma as a consequence of being administered the particular therapeutic agent or treatment modality or method. Indescriminate use of a such therapeutic agents or treatment modalities may lead to unnecessary and needless blindness in individuals due to the adverse complications.

In one embodiment of this aspect, the genetic markers of the invention are used to select individuals suitable for receiving intravitreal steroid injections. Intravitreal corticosteroids are commonly used to treat inflammation in the eye caused by various edematous and neovascular intraocular conditions, including macular edema secondary to diabetes, pseudophakia, central retinal vein occlusion, and uveitis, as well as radiation-induced edema, macular edema associated with retinitis pigmentosa, systoid macular edema secondary to birdshot retinochoroidopathy, and also exudative age-related macular degeneration, proliferative diabetic retionapthy, neovascular glaucoma, proliferative vitreaoretinopathy, chronic uveitis, acquired parafoveral teleangiectasia, choroidal neovascularization in ocular histoplasmosis syndrome, sympathetic ophthalmia, prephthisical ocular hypotony, and serous retinal detachment in Vogt-Koyanagi-Harada syndrome (Reichle, M. Optometry 76: 450-460 (2005)). Topical and systemic corticosteroids are known to be associated with increased intraocular pressure (IOP) in 30-40% of the general population and in about 60% of first-degree relatives of people with primary open angle glaucoma (POAG) (Reichle, M. Optometry 76: 450-460 (2005); Krishnadas, R. & Ramakrishnan, R., Community Eye Health 14: 40-42 (2001); Wordinger, R. J. & Clark, A. F., Progr Retinal Eye Research 18:629-667 (1999)). Increased IOP is known to be associated with exfoliation syndrome and glaucoma, and thus increased IOP due to steroid administration may lead to increased predisposition to exfoliation syndrome. In particular, it is noteworthy that exfoliation material characteristic of exfoliation syndrome is known to accumulate in the trabecular meshwork, and that the amount of the material correlates inversely with axon count in the eye, indicating a direct causative relationship between the buildup of the exfoliation material in the meshwork and the development of disease symptoms (Ritch, et al., Progr Retinal Eye Research 22: 253-275 (2003)).

AGENT TABLE II

| | | | US patent |
|---|---|---|---|
| Name | Brand name | Active compound | docs |
| timolol | betimol | (S)-1-(tert-butylamino)-3-(4-morpholino-1,2,5- | 5231095 |
| | | thiadiazol-3-yloxy)propan-2-ol | |
| dexamethazone | Ciprodex | (8S,9R,10S,11S,13S,14S,16R,17R)-9-Fluoro-11,17- | 4844902, |
| | | dihydroxy-17-(2-hydroxyacetyl)-10,13,16-trimethyl- | 6284804, |
| | | 6,7,8,11,12,14,15,16- | 6359016 |
| | | octahydrocyclopenta[a]phenanthren-3-one | |
| rimexolone | Vexol | (8S,9S,10R,11S,13S,14S,16R,17S)-11-Hydroxy- | 4686214 |
| | | 10,13,16,17-tetramethyl-17-propanoyl- | |
| | | 7,8,9,11,12,14,15,16-octahydro-6H- | |
| | | cyclopenta[a]phenanthren-3-one | |
| loteprednol | Alrex; Lotemax; Zylet | chloromethyl 17-ethoxycarbonyloxy-11-hydroxy- | 4996335, |
| | | 10,13-dimethyl-3-oxo-7,8,9,11,12,14,15,16- | 5540930, |
| | | octahydro-6H-cyclopenta[a] phenanthrene-17- | 5747061 |
| | | carboxylate | |
| latanolprost | Xalatan | isopropyl (Z)-7-[(1R,2R,3R,5S)-3,5-dihydroxy-2- | 5296504, |
| | | [(3R)3-hydroxy-5-phenylpentyl]-cyclopentyl] hept-5- | 5422368, |
| | | enoate | 6429226, |
| | | | 7163959 |
| travoprost | travatan | propan-2-yl 7-[3,5-dihydroxy-2-[3-hydroxy-4-[3- | 5510383, |
| | | (trifluoromethyl)phenoxy]-but-1-enyl]- | 5889052, |
| | | cyclopentyl]hept-5-enoate | 6503497, |
| | | | 6849253 |
| bimatoprost | Lumigan | 7-[3,5-dihydroxy-2-(3-hydroxy-5-phenyl-pent-1- | 5688819, |
| | | enyl)-cyclopentyl]-N-ethyl-hept-5-enamide | 6403649 |
| brimonidine | Alphagan; Alphagan-P | 5-Bromo-N-(4,5-dihydro-1H-imidazol-2-yl) quinoxalin- | 5424078, |
| | | 6-amine | 6562873, |
| | | | 6627210, |
| | | | 6641834, |
| | | | 6673337 |
| dorzolamide | Trucopt; Cosopt | 2-ethylamino-4-methyl-5, 5-dioxo-5$\|\hat{ }{6}, 7- | 4797413 |
| | | dithiabicyclo[4.3.0] nona-8,10-diene-8-sulfonamide | |
| brinzolamide | Azopt | (5R)-5-ethylamino-3-(3-methoxypropyl)-2,2-dioxo- | 5240923, |
| | | 2λ6,9-dithia-3-azabicyclo[4.3.0]nona-7,10-diene-8- | 5378703, |
| | | sulfonamide | 5461081 |
| betaxolol | Betoptic; Betoptic S; | 1-[4-[2-(cyclopropylmethoxy)ethyl]phenoxy]-3-(1- | 4911920 |
| | Lokren; Kerlone | methylethylamino)propan-2-ol | |
| Pegaptanib | Macugen | RNA, ((2′-deoxy-2′-fluoro)C-Gm-Gm-A-A-(2′-deoxy-2′- | 5932462, |
| | | fluoro)U-(2′-deoxy-2′-fluoro)C-Am-Gm-(2′-deoxy-2- | 5932462, |
| | | fluoro)U-Gm-Am-Am-(2′-deoxy-2′-fluoro)U-Gm-(2′- | 6011020, |
| | | deoxy-2′-fluoro)C-(2′-deoxy-2′-fluoro)U-(2′-deoxy-2′- | 6051698, |
| | | fluoro)U-Am-(2′-deoxy-2′-fluoro)U-Am-(2′-deoxy-2′- | 6051698, |
| | | fluoro)C-Am-(2′-deoxy-2′-fluoro)U-(2′-deoxy-2′- | 6113906, |
| | | fluoro)C-(2′-deoxy-2′-fluoro)C-Gm-(3′ ® 3′)-dT), 5′- | 6147204, |
| | | ester with a,a′-[4,12-dioxo-6-[[[5- | 6426335 |
| | | (phosphoonoxy)pentyl]amino]carbonyl]-3,13-dioxa- | |
| | | 5,11-diaza-1,15-pentadecanediyl]bis[w- | |
| | | methoxypoly(oxy-1,2-ethanediyl)], sodium salt | |

Exemplary corticosteroids are provided by the glucorticoids set forth in Agent Table II.

Identification of those individuals at highest risk of developing complications due to steroid administration prior to selection of appropriate therapy could significantly decrease unnecessary blindness caused by corticosteroids. As the polymorphic markers of the present invention can be used to determine whether an individual is at increased (or decreased) risk of developing glaucoma and pseudoexfoliation syndrome and symptoms associated therewith, including elevated intraocular pressure, it is contemplated that the markers may also be used to determine whether an individual is at an increased risk of developing elevated IOC and/or glaucoma as a consequence of being administered naturally occurring or synthetic corticosteroids. Thus, in one embodiment, the invention relates to a method of determining whether a human individual is at risk for developing elevated intraocular pressure and/or glaucoma as a complication of being treated with a glucocorticoid therapeutic agent, the method comprising determining the presence or absence of at least one allele of at least one polymorphic marker in a nucleic acid sample obtained from the individual, wherein the at least one polymorphic marker is associated with the LOXL1 gene, and wherein the presence of the at least one allele is indicative of an increased risk of developing elevated intraocular pressure and/or glaucoma as a complication of being treated with a glucocorticoid therapeutic agent. Those individuals who are carriers of the at-risk variants of the invention (e.g., markers rs2165241 allele T, rs1048661 allele G or rs3825942 allele G, or the G-rs1048661 G-rs3825942 haplotype, and markers in linkage disequilibrium therewith) are at increased risk of developing these complications, and are less suitable for receiving corticosteroid therapy. Individuals who are carriers of more than one at-risk marker of the invention (e.g., markers rs2165241 allele T, rs1048661 allele G or rs3825942 allele G, or the G-rs1048661 G-rs3825942 haplotype, and/or markers in linkage disequilibrium therewith) are contemplated to be especially vulnerable to such complications. Furthermore, individuals who are homozygous for at least one at-risk variant (marker or haplotype) of the invention (i.e., both chromosomes have the at-risk variant) are contemplated to be especially vulnerable. Such individuals are therefore contemplated to be especially vulnerable to developing elevated intraocular pressure and/or glaucoma as a complication of being treated with a glucocorticoid therapeutic agent.

Corticosteroid therapeutic agents that are commonly used for delivery to the eye include, but are not limited to, betamethoasone, clobetasone butyrate, dexamethasone, fluorometholone, hydrocortisone acetate, prednisolone, rimexolone, loteprednol, and medrysone.

Individuals who carry protective variants for the development of symptoms associated with exfoliation syndrome and/or glaucoma, e.g., the protective allele of the polymorphic markers of the invention may be suitable for administration of corticosteroid therapy, due to a decreased risk of developing elevated intraocular pressure and/or glaucoma as a complication of being treated with a glucocorticoid therapeutic agent. The protective variants include marker rs1048661 (SEQ ID NO: 106) allele T, marker rs3825942 (SEQ ID NO: 107) allele A, the G-rs1048661 A-rs3825942 haplotype, and markers or haplotypes in linkage disequilibrium therewith. Individuals who carry both marker rs1048661 (SEQ ID NO: 106) allele T and marker rs3825942 (SEQ ID NO: 107) allele A, or individuals who are carriers of the G-rs1048661 A-rs3825942 haplotype, are even more suitable. Particularly suitable individuals are those that carry two copies of marker rs1048661 (SEQ ID NO: 106) allele T and/or marker rs3825942 (SEQ ID NO: 107) allele A, or the G-rs1048661 A-rs3825942 or T-rs1048661 G-rs3825942 haplotype, i.e. they are homozygous for both markers or the haplotype.

Thus, individuals suitable for receiving particular therapy, i.e. corticosteroid therapy, are in certain embodiments those individuals who do not carry the at-risk variants of the invention, or those who are heterozygous carriers, including those individuals who are homozygous for the protective variants of the SNP markers or haplotypes of the invention.

Determination of the genotype status of the protective variants of the invention (e.g., marker rs1048661 (SEQ ID NO: 106) allele T and marker rs3825942 (SEQ ID NO: 107) allele A, or the G-rs1048661 A-rs3825942 haplotype) can be performed by assessing the genotype of the markers rs1048661 and marker rs3825942, or by assessing the genotype of markers in linkage disequilibrium therewith, so as to capture the genetic effect of the protective variants.

The present invention also relates to methods of monitoring progress or effectiveness of a treatment for XFS and/or glaucoma. This can be done based on the genotype and/or haplotype status of the markers and haplotypes of the present invention, i.e., by assessing the absence or presence of at least one allele of at least one polymorphic marker as disclosed herein, or by monitoring expression of genes that are associated with the variants (markers and haplotypes) of the present invention. The risk gene mRNA or the encoded polypeptide can be measured in a tissue sample (e.g., a peripheral blood sample, or a biopsy sample). Expression levels and/or mRNA levels can thus be determined before and during treatment to monitor its effectiveness. Alternatively, or concomitantly, the genotype and/or haplotype status of at least one risk variant for XFS and/or glaucoma as presented herein is determined before and during treatment to monitor its effectiveness.

Alternatively, biological networks or metabolic pathways related to the markers and haplotypes of the present invention can be monitored by determining mRNA and/or polypeptide levels. This can be done for example, by monitoring expression levels or polypeptides for several genes belonging to the network and/or pathway, in samples taken before and/or during treatment. Alternatively, metabolites belonging to the biological network or metabolic pathway can be determined before and during treatment. Effectiveness of the treatment is determined by comparing observed changes in expression levels/metabolite levels during treatment to corresponding data from healthy subjects.

In a further aspect, the markers of the present invention can be used to increase power and effectiveness of clinical trials. Thus, individuals who are carriers of at least one at-risk variant of the present invention, i.e. individuals who are carriers of at least one allele of at least one polymorphic marker conferring increased risk of developing XFS and/or glaucoma may be more likely to respond to a particular treatment modality. In one embodiment, individuals who carry at-risk variants for gene(s) in a pathway and/or metabolic network for which a particular treatment (e.g., small molecule drug) is targeting, are more likely to be responders to the treatment. In another embodiment, individuals who carry at-risk variants for a gene, which expression and/or function is altered by the at-risk variant, are more likely to be responders to a treatment modality targeting that gene, its expression or its gene product. This application can improve the safety of clinical trials, but can also enhance the chance that a clinical trial will demonstrate statistically significant efficacy, which may be limited to a certain sub-group of the population. Thus, one possible outcome of such a trial is that carriers of certain genetic variants, e.g., the markers and haplotypes of the present invention, are statistically significantly likely to show positive response to the therapeutic agent, i.e. experience alleviation of symptoms associated with XFS and/or glaucoma when taking the therapeutic agent or drug as prescribed.

In a further aspect, the markers and haplotypes of the present invention can be used for targeting the selection of pharmaceutical agents for specific individuals. Personalized selection of treatment modalities, lifestyle changes or combination of the two, can be realized by the utilization of the at-risk variants of the present invention. Thus, the knowledge of an individual's status for particular markers of the present invention, can be useful for selection of treatment options that target genes or gene products affected by the at-risk variants of the invention. Certain combinations of variants may be suitable for one selection of treatment options, while other gene variant combinations may target other treatment options. Such combination of variant may include one variant, two variants, three variants, or four or more variants, as needed to determine with clinically reliable accuracy the selection of treatment module.

In addition to the diagnostic and therapeutic uses of the variants of the present invention, the variants (markers and haplotypes) can also be useful markers for human identification, and as such be useful in forensics, paternity testing and in biometrics. The specific use of SNPs for forensic purposes is reviewed by Gill (Int. J. Legal Med. 114:204-10 (2001)). Genetic variations in genomic DNA between individuals can be used as genetic markers to identify individuals and to associated a biological sample with an individual. Genetic markers, including SNPs and microsatellites, can be useful to distinguish individuals. The more markers that are analyzed, the lower the probability that the allelic combination of the markers in any given individual is the same as in an unrelated individual (assuming that the markers are unrelated, i.e. that the markers are in perfect linkage equilibrium). Thus, the variants used for these purposes are preferably unrelated, i.e. they are inherited independently. Thus, preferred markers can be selected from available markers, such as the markers of the present invention, and the selected markers may comprise markers from different regions in the human genome, including markers on different chromosomes.

In certain applications, the SNPs useful for forensic testing are from degenerate codon positions (i.e., the third position in certain codons such that the variation of the SNP does not affect the amino acid encoded by the codon). In other applications, such for applications for predicting phenotypic characteristics including race, ancestry or physical characteristics, it may be more useful and desirable to utilize SNPs that affect the amino acid sequence of the encoded protein. In other such embodiments, the variant (SNP or other polymorphic marker) affects the expression level of a nearby gene, thus leading to altered protein expression.

### Nucleic Acids and Polypeptides

The nucleic acids and polypeptides described herein can be used in methods an kits of the present invention, as described in the above.

An “isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA library). For example, an isolated nucleic acid of the invention can be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material can be purified to essential homogeneity, for example as determined by polyacrylamide gel electrophoresis (PAGE) or column chromatography (e.g., HPLC). An isolated nucleic acid molecule of the invention can comprise at least about 50%, at least about 80% or at least about 90% (on a molar basis) of all macromolecular species present. With regard to genomic DNA, the term “isolated” also can refer to nucleic acid molecules that are separated from the chromosome with which the genomic DNA is naturally associated. For example, the isolated nucleic acid molecule can contain less than about 250 kb, 200 kb, 150 kb, 100 kb, 75 kb, 50 kb, 25 kb, 10 kb, 5 kb, 4 kb, 3 kb, 2 kb, 1 kb, 0.5 kb or 0.1 kb of the nucleotides that flank the nucleic acid molecule in the genomic DNA of the cell from which the nucleic acid molecule is derived.

The nucleic acid molecule can be fused to other coding or regulatory sequences and still be considered isolated. Thus, recombinant DNA contained in a vector is included in the definition of “isolated” as used herein. Also, isolated nucleic acid molecules include recombinant DNA molecules in heterologous host cells or heterologous organisms, as well as partially or substantially purified DNA molecules in solution. “Isolated” nucleic acid molecules also encompass in vivo and in vitro RNA transcripts of the DNA molecules of the present invention. An isolated nucleic acid molecule or nucleotide sequence can include a nucleic acid molecule or nucleotide sequence that is synthesized chemically or by recombinant means. Such isolated nucleotide sequences are useful, for example, in the manufacture of the encoded polypeptide, as probes for isolating homologous sequences (e.g., from other mammalian species), for gene mapping (e.g., by in situ hybridization with chromosomes), or for detecting expression of the gene in tissue (e.g., human tissue), such as by Northern blot analysis or other hybridization techniques.

The invention also pertains to nucleic acid molecules that hybridize under high stringency hybridization conditions, such as for selective hybridization, to a nucleotide sequence described herein (e.g., nucleic acid molecules that specifically hybridize to a nucleotide sequence containing a polymorphic site associated with a marker or haplotype described herein). Such nucleic acid molecules can be detected and/or isolated by allele- or sequence-specific hybridization (e.g., under high stringency conditions). Stringency conditions and methods for nucleic acid hybridizations are well known to the skilled person (see, e.g., Current Protocols in Molecular Biology, Ausubel, F. et al, John Wiley & Sons, (1998), and Kraus, M. and Aaronson, S., Methods Enzymol., 200:546-556 (1991), the entire teachings of which are incorporated by reference herein.

The percent identity of two nucleotide or amino acid sequences can be determined by aligning the sequences for optimal comparison purposes (e.g., gaps can be introduced in the sequence of a first sequence). The nucleotides or amino acids at corresponding positions are then compared, and the percent identity between the two sequences is a function of the number of identical positions shared by the sequences (i.e., % identity=# of identical positions/total # of positions×100). In certain embodiments, the length of a sequence aligned for comparison purposes is at least 30%, at least 40%, at least 50%, at least 60%, at least 70%, at least 80%, at least 90%, or at least 95%, of the length of the reference sequence. The actual comparison of the two sequences can be accomplished by well-known methods, for example, using a mathematical algorithm. A non-limiting example of such a mathematical algorithm is described in Karlin, S. and Altschul, S., Proc. Natl. Acad. Sci. USA, 90:5873-5877 (1993). Such an algorithm is incorporated into the NBLAST and XBLAST programs (version 2.0), as described in Altschul, S. et al., Nucleic Acids Res., 25:3389-3402 (1997). When utilizing BLAST and Gapped BLAST programs, the default parameters of the respective programs (e.g., NBLAST) can be used. See the website on the world wide web at ncbi.nlm.nih.gov. In one embodiment, parameters for sequence comparison can be set at score=100, wordlength=12, or can be varied (e.g., W=5 or W=20).

Other examples include the algorithm of Myers and Miller, CABIOS (1989), ADVANCE and ADAM as described in Torellis, A. and Robotti, C., Comput. Appl. Biosci. 10:3-5 (1994); and FASTA described in Pearson, W. and Lipman, D., Proc. Natl. Acad. Sci. USA, 85:2444-48 (1988).

In another embodiment, the percent identity between two amino acid sequences can be accomplished using the GAP program in the GCG software package (Accelrys, Cambridge, UK).

The present invention also provides isolated nucleic acid molecules that contain a fragment or portion that hybridizes under highly stringent conditions to a nucleic acid that comprises, or consists of, the nucleotide sequence of the LOXL1 LD block (SEQ ID NO:84), the nucleotide sequence of the LOXL1 gene, or a nucleotide sequence comprising, or consisting of, the complement of the nucleotide sequence of the LOXL1 LD block, or the LOXL1 gene, wherein the nucleotide sequence comprises at least one polymorphic allele contained in the markers and haplotypes described herein. The invention furthermore provides isolated nucleic acid molecules that contain a fragment or portion that hybridizes under highly stringent conditions to a nucleic acid that comprises, or consists of, the nucleotide sequence of the LOXL1 gene (SEQ ID NO:84), or a nucleotide sequence comprising, or consisting of, the complement of the nucleotide sequence of the LOXL1, wherein the nucleotide sequence comprises at least one polymorphic allele contained in the markers and haplotypes described herein. The nucleic acid fragments of the invention are at least about 15, at least about 18, 20, 23 or 25 nucleotides, and can be 30, 40, 50, 100, 200, 500, 1000, 10,000 or more nucleotides in length.

The nucleic acid fragments of the invention are used as probes or primers in assays such as those described herein. “Probes” or “primers” are oligonucleotides that hybridize in a base-specific manner to a complementary strand of a nucleic acid molecule. In addition to DNA and RNA, such probes and primers include polypeptide nucleic acids (PNA), as described in Nielsen, P. et al., Science 254:1497-1500 (1991). A probe or primer comprises a region of nucleotide sequence that hybridizes to at least about 15, typically about 20-25, and in certain embodiments about 40, 50 or 75, consecutive nucleotides of a nucleic acid molecule. In one embodiment, the probe or primer comprises at least one allele of at least one polymorphic marker or at least one haplotype described herein, or the complement thereof. In particular embodiments, a probe or primer can comprise 100 or fewer nucleotides; for example, in certain embodiments from 6 to 50 nucleotides, or, for example, from 12 to 30 nucleotides. In other embodiments, the probe or primer is at least 70% identical, at least 80% identical, at least 85% identical, at least 90% identical, or at least 95% identical, to the contiguous nucleotide sequence or to the complement of the contiguous nucleotide sequence. In another embodiment, the probe or primer is capable of selectively hybridizing to the contiguous nucleotide sequence or to the complement of the contiguous nucleotide sequence. Often, the probe or primer further comprises a label, e.g., a radioisotope, a fluorescent label, an enzyme label, an enzyme co-factor label, a magnetic label, a spin label, an epitope label.

The nucleic acid molecules of the invention, such as those described above, can be identified and isolated using standard molecular biology techniques well known to the skilled person. The amplified DNA can be labeled (e.g., radiolabeled) and used as a probe for screening a cDNA library derived from human cells. The cDNA can be derived from mRNA and contained in a suitable vector. Corresponding clones can be isolated, DNA can obtained following in vivo excision, and the cloned insert can be sequenced in either or both orientations by art-recognized methods to identify the correct reading frame encoding a polypeptide of the appropriate molecular weight. Using these or similar methods, the polypeptide and the DNA encoding the polypeptide can be isolated, sequenced and further characterized.

In general, the isolated nucleic acid sequences of the invention can be used as molecular weight markers on Southern gels, and as chromosome markers that are labeled to map related gene positions. The nucleic acid sequences can also be used to compare with endogenous DNA sequences in patients to identify XFS and/or glaucoma, and as probes, such as to hybridize and discover related DNA sequences or to subtract out known sequences from a sample (e.g., subtractive hybridization). The nucleic acid sequences can further be used to derive primers for genetic fingerprinting, to raise anti-polypeptide antibodies using immunization techniques, and/or as an antigen to raise anti-DNA antibodies or elicit immune responses.

The present invention also pertains to isolated polypeptides encoded by the LOXL1 gene and fragments and variants thereof, as well as polypeptides encoded by nucleotide sequences described herein. A polypeptide is said to be “isolated” or “purified” when it is substantially free of cellular material when it is isolated from recombinant and non-recombinant cells, or free of chemical precursors or other chemicals when it is chemically synthesized. A polypeptide, however, can be joined to another polypeptide with which it is not normally associated in a cell (e.g., in a “fusion protein”) and still be “isolated” or “purified.”

The polypeptides of the invention can be purified to homogeneity. It is understood, however, that preparations in which the polypeptide is not purified to homogeneity may also be useful. The critical feature is that the preparation allows for the desired function of the polypeptide, even in the presence of considerable amounts of other components. Thus, the invention encompasses various degrees of purity. In one embodiment, the preparations of the polypeptide have less than about 30% (by dry weight) other proteins (i.e., contaminating protein), less than about 20% other proteins, less than about 10% other proteins, or less than about 5% other proteins.

When a polypeptide is recombinantly produced, it can also be substantially free of culture medium, i.e., culture medium represents less than about 20%, less than about 10%, or less than about 5% of the volume of the polypeptide preparation. Polypeptides that are substantially free of chemical precursors or other chemicals include preparations of the polypeptide in which the polypeptide is separated from chemical precursors or other chemicals that are involved in its synthesis. In certain embodiments, preparations of the polypeptide have less than about 30% (by dry weight) chemical precursors or other chemicals, less than about 20% chemical precursors or other chemicals, less than about 10% chemical precursors or other chemicals, or less than about 5% chemical precursors or other chemicals.

In one embodiment, a polypeptide of the invention comprises an amino acid sequence encoded by a nucleic acid molecule comprising a nucleotide sequence of SEQ ID NO: 84 which may optionally comprise particular variant alleles for at least one polymorphism at positions 7142 and/or 7178 in SEQ ID NO:84, and complements and fragments thereof. However, the polypeptides of the invention also encompass fragments and other sequence variants. Variants include a substantially homologous polypeptide encoded by the same genetic locus in an organism, i.e., an allelic variant, as well as other splicing variants. Preferred variants include amino acid substitutions at positions 141 and/or 153 in SEQ ID NO:85 (e.g., R141L and/or G153D). Preferred variants include R141 D153 variants, L141 G153 variants and R141 G153 variants. Variants also include polypeptides substantially homologous or identical to human LOXL1 but derived from another organism, i.e., orthologs. Variants also include polypeptides that are substantially homologous or identical to these polypeptides that are produced by chemical synthesis. Variants also include polypeptides that are substantially homologous or identical to these polypeptides that are produced by recombinant methods.

As used herein, two polypeptides (or a region of the polypeptides) are substantially homologous or identical when the amino acid sequences are at least about 45-55%, typically at least about 70-75%, more typically at least about 80-85%, and most typically greater than about 90% or more homologous or identical. A substantially homologous amino acid sequence, according to the present invention, will be encoded by a nucleic acid molecule hybridizing to SEQ ID NO: 84, which may optionally comprise at least one polymorphism shown in Table 4 and/or 6a, or portion thereof, under stringent conditions as more particularly described above.

To determine the percent homology or identity of two amino acid sequences, or of two nucleic acid sequences, the sequences are aligned for optimal comparison purposes (e.g., gaps can be introduced in the sequence of one polypeptide or nucleic acid molecule for optimal alignment with the other polypeptide or nucleic acid molecule). The amino acid residues or nucleotides at corresponding amino acid positions or nucleotide positions are then compared. When a position in one sequence is occupied by the same amino acid residue or nucleotide as the corresponding position in the other sequence, then the molecules are homologous at that position. As used herein, amino acid or nucleic acid “homology” is equivalent to amino acid or nucleic acid “identity”. The percent homology between the two sequences is a function of the number of identical positions shared by the sequences (i.e., percent homology equals the number of identical positions/total number of positions times 100).

The invention also encompasses polypeptides having a lower degree of identity but having sufficient similarity so as to perform one or more of the same functions performed by a polypeptide encoded by a nucleic acid molecule of the invention. Similarity is determined by conserved amino acid substitution. Such substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe and Tyr. Guidance concerning which amino acid changes are likely to be phenotypically silent are found in Bowie et al., Science 247:1306-1310 (1990).

A variant polypeptide can differ in amino acid sequence by one or more substitutions, deletions, insertions, inversions, fusions, and truncations or a combination of any of these. Further, variant polypeptides can be fully functional or can lack function in one or more activities. Fully functional variants typically contain only conservative variation or variation in non-critical residues or in non-critical regions. Functional variants can also contain substitution of similar amino acids that result in no change or an insignificant change in function. Alternatively, such substitutions may positively or negatively affect function to some degree. Non-functional variants typically contain one or more non-conservative amino acid substitutions, deletions, insertions, inversions, or truncation or a substitution, insertion, inversion, or deletion in a critical residue or critical region.

Amino acids that are essential for function can be identified by methods known in the art, such as site-directed mutagenesis or alanine-scanning mutagenesis (Cunningham et al., Science, 244:1081-1085 (1989)). The latter procedure introduces single alanine mutations at every residue in the molecule. The resulting mutant molecules are then tested for biological activity in vitro, or in vitro proliferative activity. Sites that are critical for polypeptide activity can also be determined by structural analysis such as crystallization, nuclear magnetic resonance or photoaffinity labeling (Smith et al, J. Mol. Biol., 224:899-904 (1992); de Vos et al., Science, 255:306-312 (1992)).

The invention also includes polypeptide fragments of LOXL1, as set forth in SEQ ID NO: 85, and which are encoded by the LOXL1 gene (SEQ ID NO:84), and which may optionally comprise at least one polymorphism shown in Tables 4, 6a and 16, or a portion thereof and the complements thereof. As used herein, a fragment comprises at least 6 contiguous amino acids. Useful fragments include those that retain one or more of the biological activities of LOXL1 as well as fragments that can be used as an immunogen to generate polypeptide-specific antibodies.

Biologically active fragments (peptides which are, for example, 6, 9, 12, 15, 16, 20, 30, 35, 36, 37, 38, 39, 40, 50, 100 or more amino acids in length) can comprise a domain, segment, or motif that has been identified by analysis of the polypeptide sequence using well-known methods, e.g., signal peptides, extracellular domains, one or more transmembrane segments or loops, ligand binding regions, zinc finger domains, DNA binding domains, acylation sites, glycosylation sites, or phosphorylation sites.

Fragments can be discrete (not fused to other amino acids or polypeptides) or can be within a larger polypeptide. Further, several fragments can be comprised within a single larger polypeptide. In one embodiment a fragment designed for expression in a host can have heterologous pre- and pro-polypeptide regions fused to the amino terminus of the polypeptide fragment and an additional region fused to the carboxyl terminus of the fragment.

The invention thus provides chimeric or fusion polypeptides. These comprise a polypeptide of the invention operatively linked to a heterologous protein or polypeptide having an amino acid sequence not substantially homologous to the polypeptide. An operatively linked polypeptide indicates that the polypeptide and the heterologous protein are fused in-frame. The heterologous protein can be fused to the N-terminus or C-terminus of the polypeptide. In one embodiment the fusion polypeptide does not affect function of the polypeptide per se. For example, the fusion polypeptide can be a GST-fusion polypeptide in which the polypeptide sequences are fused to the C-terminus of the GST sequences. Other types of fusion polypeptides include, but are not limited to, enzymatic fusion polypeptides, for example β-galactosidase fusions, yeast two-hybrid GAL fusions, poly-His fusions and Ig fusions. Such fusion polypeptides, particularly poly-His fusions, can facilitate the purification of recombinant polypeptide. In certain host cells (e.g., mammalian host cells), expression and/or secretion of a polypeptide can be increased by using a heterologous signal sequence. Therefore, in another embodiment, the fusion polypeptide contains a heterologous signal sequence at its N-terminus.

Fusion proteins comprising various portions of immunoglobulin constant regions are disclosed in EP-A-O 464 533. The Fc is useful in therapy and diagnosis and thus results, for example, in improved pharmacokinetic properties (EP-A 0232 262). In drug discovery, for example, human proteins have been fused with Fc portions for the purpose of high-throughput screening assays to identify antagonists. Bennett et al. Journal of Molecular Recognition, 8:52-58 (1995) and Johanson et al., The Journal of Biological Chemistry, 270, 16:9459-9471 (1995). Thus, this invention also encompasses soluble fusion polypeptides containing a polypeptide of the invention and various portions of the constant regions of heavy or light chains of immunoglobulins of various subclass (IgG, IgM, IgA, IgE).

A chimeric or fusion polypeptide can be produced by standard recombinant DNA techniques. For example, DNA fragments coding for the different polypeptide sequences are ligated together in-frame in accordance with conventional techniques. In another embodiment, the fusion gene can be synthesized by conventional techniques including automated DNA synthesizers. Alternatively, PCR amplification of nucleic acid fragments can be carried out using anchor primers which give rise to complementary overhangs between two consecutive nucleic acid fragments which can subsequently be annealed and re-amplified to generate a chimeric nucleic acid sequence (see Ausubel et al., Current Protocols in Molecular Biology, 1992). Moreover, many expression vectors are commercially available that already encode a fusion moiety (e.g., a GST protein). A nucleic acid molecule encoding a polypeptide of the invention can be cloned into such an expression vector such that the fusion moiety is linked in-frame to the polypeptide.

The isolated polypeptide can be purified from cells that naturally express it, purified from cells that have been altered to express it (recombinant), or synthesized using known protein synthesis methods. In one embodiment, the polypeptide is produced by recombinant DNA techniques. For example, a nucleic acid molecule encoding the polypeptide is cloned into an expression vector, the expression vector introduced into a host cell and the polypeptide expressed in the host cell. The polypeptide can then be isolated from the cells by an appropriate purification scheme using standard protein purification techniques.

In general, polypeptides of the present invention can be used as a molecular weight marker on SDS-PAGE gels or on molecular sieve gel filtration columns using art-recognized methods. The polypeptides of the present invention can be used to raise antibodies or to elicit an immune response. The polypeptides can also be used as a reagent, e.g., a labeled reagent, in assays to quantitatively determine levels of the polypeptide or a molecule to which it binds (e.g., a receptor or a ligand) in biological fluids. The polypeptides can also be used as markers for cells or tissues in which the corresponding polypeptide is preferentially expressed, either constitutively, during tissue differentiation, or in a diseased state. The polypeptides can be used to isolate a corresponding binding agent, e.g., receptor or ligand, such as, for example, in an interaction trap assay, and to screen for peptide or small molecule antagonists or agonists of the binding interaction.

Preparation of protein for use as a therapeutic agent or in other methods of the invention is well known to the skilled person. LOXL1 protein or recombinant protein can be isolated from suitable hosts using methods known in the art, or produced by direct chemical synthesis. Human LOXL1 protein can be isolated from any human cell that expresses the protein, including cells that have been transfected with expression constructs for expressing LOXL1. Expression vectors can be utilized by known methods, and include necessary elements for the transcription and translation of the inserted coding sequence. Construction of expression vectors is within the scope of expertize by the skilled person, including in vitro recombinant DNA techniques, synthetic techniques and in vivo genetic recombination.

A variety of expression vectors or vector/host systems can be utilized to express sequences encoding LOXL1. These include, but are not limited to, microorganisms, such as bacteria transformed with bacteriophage, plasmid, or cosmid DNA expression vectors; yeast transformed with yeast expression vectors, insect cell systems infected with virus expression vectors (e.g., baculovirus), plant cell systems transformed with virus expression vectors (e.g., cauliflower mosaic virus, CaMV; tobacco mosaic virus, TMV) or with bacterial expression vectors, or with animal cell systems.

Suitable control elements or regulatory sequences are those non-translated regions of the vector—enhancers, promoters, 5′ and 4′ untranslated regions—which interact with host cellular proteins to carry out transcription and translation. Such elements vary in their strength and specificity. Thus, depending on the vector system and host utilized, any number of suitable transcription and translation elements, including constitutive and inducible promoters, can be used. For example, hybrid lacZ promoter of the BLUESCRIPT plasmid may be used in bacterial systems, baculovirus promoter can be used in insect cells, and promoters and enhancers derived from plant cell genomes (e.g., heat shock, rubisco and storage protein genes) or from plant viruses (e.g., viral promoters or leader sequences) can be cloned into the vector. In mammalian systems, promoters from mammalian genes or from mammalian viruses are preferable.

Host cells can be chosen for their ability to modulate the expression of the inserted sequences or to process the expressed protein (e.g., LOXL1) in the desired fashion. Such modification of the polypeptide includes glycosylation, but may also include acetylation, carboxylation, phosphorylation, lipidation, and acylation. Post-translational processing which cleaves a “pro” or “prepro” form of the polypeptide can also be used to facilitate correct insertion, folding and/or function. Different host cells have specific cellular machinery and characteristic mechanisms for post-translational activities (e.g., CHO, HeLa, MDCHK, HEK293, WI38), and are available from commercial sources. These can be chosen as deemed suitable by the skilled person to ensure the correct modification and processing of the foreign protein in the host cell system of choice.

Stable expression, which is preferable for long-term high-yield production of recombinant protein can be achieved by known methods. For example, cell lines which stably express LOXL1 can be transformed using expression vectors which can contain viral origins or replication and/or endogenous expression elements and a selectable marker gene on the same, or on a separate, vector. Tissue culture techniques can be used to proliferate stably transformed cells, as appropriate for each cell type. Various selection systems can be employed, such as herpes simplex virus thymidine kinase and adenine phosphoribosyltransferase genes. Also, antimetabolite, antibiotic or herbicide resistance can be used for selection. Visible markers that can be employed include anthocyanins, beta-glucuronidase and its substrate GUS, and luciferase and its substrate luciferin, and can be used for identifying transformants and to quantify amount of transient or stable protein expression.

Expressed protein can be isolated and purified using known techniques, including high performance liquid chromatography (HPLC), ion exchange chromatography, size exclusion chromatography, affinity chromatography, gel electrophoresis or other suitable methods available to the skilled person (see, e.g., Current Protocols in Protein Science, John Wiley & Sons (2007); ISBN: 978-0-471-11184-9).

Purified protein can be administered directly or in a pharmaceutical composition that may optionally include pharmaceutically acceptable carriers and/or excipients.

### Antibodies

Polyclonal antibodies and/or monoclonal antibodies that specifically bind one form of LOXL1, but not to the other form of the protein, are also provided. Antibodies are also provided which bind a portion of either the variant or the reference gene product that contains the polymorphic site or sites. The term “antibody” as used herein refers to immunoglobulin molecules and immunologically active portions of immunoglobulin molecules, i.e., molecules that contain antigen-binding sites that specifically bind an antigen. A molecule that specifically binds to a polypeptide of the invention is a molecule that binds to that polypeptide or a fragment thereof, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind to a polypeptide of the invention, e.g., LOXL1. The term “monoclonal antibody” or “monoclonal antibody composition”, as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared as described above by immunizing a suitable subject with a desired immunogen, e.g., polypeptide of the invention or a fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein, Nature 256:495-497 (1975), the human B cell hybridoma technique (Kozbor et al., Immunol. Today 4: 72 (1983)), the EBV-hybridoma technique (Cole et al., Monoclonal Antibodies and Cancer Therapy, Alan R. Liss,1985, Inc., pp. 77-96) or trioma techniques. The technology for producing hybridomas is well known (see generally Current Protocols in Immunology (1994) Coligan et al., (eds.) John Wiley & Sons, Inc., New York, N.Y.). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (see, e.g., Current Protocols in Immunology, supra; Galfre et al., Nature 266:55052 (1977); R. H. Kenneth, in Monoclonal Antibodies: A New Dimension In Biological Analyses, Plenum Publishing Corp., New York, N.Y. (1980); and Lerner, Yale J. Biol. Med. 54:387-402 (1981)). Moreover, the ordinarily skilled worker will appreciate that there are many variations of such methods that also would be useful.

Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., Bio/Technology 9: 1370-1372 (1991); Hay et al., Hum. Antibod. Hybridomas 3:81-85 (1992); Huse et al., Science 246: 1275-1281 (1989); and Griffiths et al., EMBO J. 12:725-734 (1993).

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In general, antibodies of the invention (e.g., a monoclonal antibody) can be used to isolate a polypeptide of the invention by standard techniques, such as affinity chromatography or immunoprecipitation. A polypeptide-specific antibody can facilitate the purification of natural polypeptide from cells and of recombinantly produced polypeptide expressed in host cells. Moreover, an antibody specific for a polypeptide of the invention can be used to detect the polypeptide (e.g., in a cellular lysate, cell supernatant, or tissue sample) in order to evaluate the abundance and pattern of expression of the polypeptide. Antibodies can be used diagnostically to monitor protein levels in tissue as part of a clinical testing procedure, e.g., to, for example, determine the efficacy of a given treatment regimen. The antibody can be coupled to a detectable substance to facilitate its detection. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibodies may also be useful in pharmacogenomic analysis. In such embodiments, antibodies against variant proteins encoded by nucleic acids according to the invention, such as variant proteins that are encoded by nucleic acids that contain at least one polymorphic marker of the invention, can be used to identify individuals that require modified treatment modalities.

Antibodies can furthermore be useful for assessing expression of variant proteins in disease states, such as in active stages of a disease, or in an individual with a predisposition to a disease related to the function of the protein (e.g., LOXL1), in particular XFS and/or glaucoma. Antibodies specific for a variant protein of the present invention (e.g., a variant of LOXL1) that is encoded by a nucleic acid that comprises at least one polymorphic marker or haplotype as described herein can be used to screen for the presence of the variant protein, for example to screen for a predisposition to XFS and/or glaucoma as indicated by the presence of the variant protein.

Antibodies can be used in other methods. Thus, antibodies are useful as diagnostic tools for evaluating proteins, such as variant proteins of the invention, in conjunction with analysis by electrophoretic mobility, isoelectric point, tryptic or other protease digest, or for use in other physical assays known to those skilled in the art. Antibodies may also be used in tissue typing. In one such embodiment, a specific variant protein has been correlated with expression in a specific tissue type, and antibodies specific for the variant protein can then be used to identify the specific tissue type.

Subcellular localization of proteins, including variant proteins, can also be determined using antibodies, and can be applied to assess aberrant subcellular localization of the protein in cells in various tissues. Such use can be applied in genetic testing, but also in monitoring a particular treatment modality. In the case where treatment is aimed at correcting the expression level or presence of the variant protein or aberrant tissue distribution or developmental expression of the variant protein, antibodies specific for the variant protein or fragments thereof can be used to monitor therapeutic efficacy.

Antibodies are further useful for inhibiting variant protein function, for example by blocking the binding of a variant protein (e.g., LOXL1 variants, as described herein, e.g. the R141 G153 variant) to a binding molecule or partner. Such uses can also be applied in a therapeutic context in which treatment involves inhibiting a variant protein's function. An antibody can be for example be used to block or competitively inhibit binding, thereby modulating (i.e., agonizing or antagonizing) the activity of the protein. Antibodies can be prepared against specific protein fragments containing sites required for specific function or against an intact protein that is associated with a cell or cell membrane. For administration in vivo, an antibody may be linked with an additional therapeutic payload, such as radionuclide, an enzyme, an immunogenic epitope, or a cytotoxic agent, including bacterial toxins (diphteria or plant toxins, such as ricin). The in vivo half-life of an antibody or a fragment thereof may be increased by pegylation through conjugation to polyethylene glycol.

The present invention further relates to kits for using antibodies in the methods described herein. This includes, but is not limited to, kits for detecting the presence of a variant protein in a test sample. One preferred embodiment comprises antibodies such as a labelled or labellable antibody and a compound or agent for detecting variant proteins in a biological sample, means for determining the amount or the presence and/or absence of variant protein in the sample, and means for comparing the amount of variant protein in the sample with a standard, as well as instructions for use of the kit.

### Pharmaceutical Compositions

The present invention also pertains to pharmaceutical compositions comprising agents described herein, particularly nucleotides encoding LOXL1; LOXL1 polypeptides described herein (e.g., SEQ ID NO:85 and variants thereof); antibodies; and/or an agent that alters (e.g., enhances or inhibits) LOXL1 gene expression or LOXL1 polypeptide activity as described herein. For instance, a LOXL1 polypeptide, LOXL1 protein, an agent that alters LOXL1 gene expression, or a LOXL1 binding agent or binding partner, fragment, fusion protein or product thereof, or a nucleotide or nucleic acid construct (vector) comprising a nucleotide of the present invention, or an agent that alters LOXL1 polypeptide activity, can be formulated with a physiologically acceptable carrier or excipient to prepare a pharmaceutical composition. The carrier and composition can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active agents.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrolidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. Other suitable methods of introduction can also include gene therapy (as described below), rechargeable or biodegradable devices, particle acceleration devises (“gene guns”) and slow release polymeric devices. The pharmaceutical compositions of this invention can also be administered as part of a combinatorial therapy with other agents.

The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

Agents described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The agents are administered in a therapeutically effective amount. The amount of agents which will be therapeutically effective in the treatment of a particular disorder or condition will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the symptoms of XFS and/or glaucoma, and should be decided according to the judgment of a practitioner and each patient's circumstances. Effective doses may be extrapolated from dose-response curves derived from in vitro or animal model test systems.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use of sale for human administration. The pack or kit can be labeled with information regarding mode of administration, sequence of drug administration (e.g., separately, sequentially or concurrently), or the like. The pack or kit may also include means for reminding the patient to take the therapy. The pack or kit can be a single unit dosage of the combination therapy or it can be a plurality of unit dosages. In particular, the agents can be separated, mixed together in any combination, present in a single vial or tablet. Agents assembled in a blister pack or other dispensing means is preferred. For the purpose of this invention, unit dosage is intended to mean a dosage that is dependent on the individual pharmacodynamics of each agent and administered in FDA approved dosages in standard time courses.

### Methods of Therapy

The present invention encompasses methods of treatment (prophylactic and/or therapeutic) XFS and/or glaucoma or a susceptibility to XFS and/or glaucoma, in particular exfoliation glaucoma, such as individuals in the target populations described herein, using a LOXL1 therapeutic agent. A “LOXL1 therapeutic agent” is an agent that alters (e.g., enhances or inhibits) LOXL1 polypeptide (enzymatic activity) and/or LOXL1 gene expression, as described herein (e.g., a LOXL1 agonist or antagonist). The LOXL1 therapeutic agents can alter LOXL1 polypeptide activity or nucleic acid expression by a variety of means, such as, for example, by providing additional LOXL1 polypeptide or by upregulating the transcription or translation of the LOXL1 gene; by altering posttranslational processing of the LOXL1 polypeptide; by altering transcription of LOXL1 splicing variants; or by interfering with LOXL1 polypeptide activity (e.g., by binding to a LOXL1 polypeptide), or by downregulating the transcription or translation of the LOXL1 gene.

In particular, the invention relates to methods of treatment for XFS and/or glaucoma or susceptibility to XFS and/or glaucoma (for example, for individuals in an at-risk population such as those described herein).

Representative LOXL1 therapeutic agents include the following:

nucleic acids or fragments or derivatives thereof described herein, particularly nucleotides encoding the LOXL1 polypeptides described herein and vectors comprising such nucleic acids (e.g., a gene, cDNA, and/or mRNA, double-stranded interfering RNA, a nucleic acid encoding a LOXL1 polypeptide or active fragment or derivative thereof, or an oligonucleotide; for example, SEQ ID NO: 84 or a fragment thereof which may optionally comprise at least one polymorphism shown in Table 4 or 6a, or fragments or derivatives thereof), antisense nucleic acids or small double-stranded interfering RNA;
polypeptides described herein (e.g., SEQ ID NO: 85, and/or other splicing variants encoded by LOXL1, or fragments or derivatives thereof), optionally comprising at least one amino acid substitution as listed in Table 6a, such as R141L and/or G135D;
other polypeptides (e.g., LOXL1-interacting proteins); LOXL1 binding agents; peptidomimetics; fusion proteins or prodrugs thereof; antibodies (e.g., an antibody to a mutant LOXL1 polypeptide, or an antibody to a non-mutant LOXL1 polypeptide, or an antibody to a particular splicing variant encoded by LOXL1, as described above); ribozymes; other small molecules;
and other agents that alter (e.g., inhibit or antagonize) LOXL1 gene expression or polypeptide activity, or that regulate transcription of LOXL1 splicing variants (e.g., agents that affect which splicing variants are expressed, or that affect the amount of each splicing variant that is expressed).

More than one LOXL1 therapeutic agent can be used concurrently, if desired.

The LOXL1 therapeutic agent that is a nucleic acid is used in the treatment of XFS and/or glaucoma. The term, “treatment” as used herein, refers not only to ameliorating symptoms associated with the disease, but also preventing or delaying the onset of the disease, and also lessening the severity or frequency of symptoms of the disease, preventing or delaying the occurrence of a second episode of the disease or condition; and/or also lessening the severity or frequency of symptoms of the disease or condition. The therapy is designed to alter (e.g., inhibit or enhance), replace or supplement activity of a LOXL1 polypeptide in an individual. For example, a LOXL1 therapeutic agent can be administered in order to upregulate or increase the expression or availability of the LOXL1 gene or of specific splicing variants of LOXL1, or, conversely, to downregulate or decrease the expression or availability of the LOXL1 gene or specific splicing variants of LOXL1. Upregulation or increasing expression or availability of a native LOXL1 gene or of a particular splicing variant could interfere with or compensate for the expression or activity of a defective gene or another splicing variant; downregulation or decreasing expression or availability of a native LOXL1 gene or of a particular splicing variant could minimize the expression or activity of a defective gene or the particular splicing variant and thereby minimize the impact of the defective gene or the particular splicing variant.

The LOXL1 therapeutic agent(s) are administered in a therapeutically effective amount (i.e., an amount that is sufficient to treat the disease, such as by ameliorating symptoms associated with the disease, preventing or delaying the onset of the disease, and/or also lessening the severity or frequency of symptoms of the disease). The amount which will be therapeutically effective in the treatment of a particular individual's disorder or condition will depend on the symptoms and severity of the disease, and can be determined by standard clinical techniques. In addition, in vitro or in vivo assays may optionally be employed to help identify optimal dosage ranges. The precise dose to be employed in the formulation will also depend on the route of administration, and the seriousness of the disease or disorder, and should be decided according to the judgment of a practitioner and each patient's circumstances. Effective doses may be extrapolated from dose-response curves derived from in vitro or animal model test systems.

In one embodiment, a nucleic acid of the invention (e.g., a nucleic acid encoding a LOXL1 polypeptide, such as SEQ ID NO: 85 which may optionally comprise at least one polymorphism shown in Table 4 or 6a) can be used, either alone or in a pharmaceutical composition as described above. For example, LOXL1 or a cDNA encoding the LOXL1 polypeptide, either by itself or included within a vector, can be introduced into cells (either in vitro or in vivo) such that the cells produce native LOXL1 polypeptide. If necessary, cells that have been transformed with the gene or cDNA or a vector comprising the gene or cDNA can be introduced (or re-introduced) into an individual affected with the disease. Thus, cells which, in nature, lack native LOXL1 expression and activity, or have mutant LOXL1 expression and activity, or have expression of a disease-associated LOXL1 splicing variant, can be engineered to express LOXL1 polypeptide or an active fragment of the LOXL1 polypeptide (or a different variant of LOXL1 polypeptide). In another embodiment, nucleic acid encoding the LOXL1 polypeptide, or an active fragment or derivative thereof, can be introduced into an expression vector, such as a viral vector, and the vector can be introduced into appropriate cells in an animal. Other gene transfer systems, including viral and nonviral transfer systems, can be used. Alternatively, nonviral gene transfer methods, such as calcium phosphate coprecipitation, mechanical techniques (e.g., microinjection, e.g., intraocular injection); membrane fusion-mediated transfer via liposomes; or direct DNA uptake, can also be used.

Alternatively, in another embodiment of the invention, a nucleic acid of the invention; a nucleic acid complementary to a nucleic acid of the invention; or a portion of such a nucleic acid (e.g., an oligonucleotide as described below), can be used in “antisense” therapy, in which a nucleic acid (e.g., an oligonucleotide) which specifically hybridizes to the mRNA and/or genomic DNA of LOXL1 is administered or generated in situ. The antisense nucleic acid that specifically hybridizes to the mRNA and/or DNA inhibits expression of the LOXL1 polypeptide, e.g., by inhibiting translation and/or transcription. Binding of the antisense nucleic acid can be by conventional base pair complementarity, or, for example, in the case of binding to DNA duplexes, through specific interaction in the major groove of the double helix.

An antisense construct of the present invention can be delivered, for example, as an expression plasmid as described above. When the plasmid is transcribed in the cell, it produces RNA that is complementary to a portion of the mRNA and/or DNA that encodes LOXL1 polypeptide. Alternatively, the antisense construct can be an oligonucleotide probe that is generated ex vivo and introduced into cells; it then inhibits expression by hybridizing with the mRNA and/or genomic DNA of LOXL1. In one embodiment, the oligonucleotide probes are modified oligonucleotides that are resistant to endogenous nucleases, e.g., exonucleases and/or endonucleases, thereby rendering them stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy are also described, for example, by Van der Krol et al. ((1988) Biotechniques 6:958-976); and Stein et al. ((1988) Cancer Res 48:2659-2668). With respect to antisense DNA, oligodeoxyribonucleotides derived from the translation initiation site, e.g., between the −10 and +10 regions of LOXL1 sequence, are preferred.

To perform antisense therapy, oligonucleotides (mRNA, cDNA or DNA) are designed that are complementary to mRNA encoding LOXL1. The antisense oligonucleotides bind to LOXL1 mRNA transcripts and prevent translation. Absolute complementarity, although preferred, is not required. A sequence “complementary” to a portion of an RNA, as referred to herein, indicates that a sequence has sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid, as described in detail above. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures. The oligonucleotides used in antisense therapy can be DNA, RNA, or chimeric mixtures or derivatives or modified versions thereof, single-stranded or double-stranded. The oligonucleotides can be modified at the base moiety, sugar moiety, or phosphate backbone, for example, to improve stability of the molecule, hybridization, etc. The oligonucleotides can include other appended groups such as peptides (e.g., for targeting host cell receptors in vivo), or agents facilitating transport across the cell membrane (see, e.g., Letsinger et al. (1989) Proc. Nat. Acad. Sci. USA 86:6553-6556; Lemaitre et al., (1987), Proc. Natl. Acad. Sci. USA 84:648-652; PCT International Publication No. WO88/09810) or the blood-brain barrier (see, e.g., PCT International Publication No. WO89/10134), or hybridization-triggered cleavage agents (see, e.g., Krol et al. (1988) BioTechniques 6:958-976) or intercalating agents. (See, e.g., Zon, (1988), Pharm. Res. 5:539-549). To this end, the oligonucleotide may be conjugated to another molecule (e.g., a peptide, hybridization triggered cross-linking agent, transport agent, hybridization-triggered cleavage agent).

The antisense molecules are delivered to cells that express LOXL1 in vivo. A number of methods can be used for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically. Alternatively, in another embodiment, a recombinant DNA construct is utilized in which the antisense oligonucleotide is placed under the control of a strong promoter (e.g., pol III or pol II). The use of such a construct to transfect target cells in the patient results in the transcription of sufficient amounts of single stranded RNAs that will form complementary base pairs with the endogenous LOXL1 transcripts and thereby prevent translation of the LOXL1 mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art and described above. For example, a plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systemically).

Methods of modulating LOXL1 expression by administering an RNA inhibitor of the activity of the target protein are also possible by RNA interference, as described in more detail herein. Administration of siRNA to induce gene silencing is thus also within scope of the methods of modulating LOXL1 expression.

Endogenous LOXL1 expression can be also reduced by inactivating or “knocking out” LOXL1 or its promoter using targeted homologous recombination (e.g., see Smithies et al. (1985) Nature 317:230-234; Thomas & Capecchi (1987) Cell 51:503-512; Thompson et al. (1989) Cell 5:313-321). For example, a mutant, non-functional LOXL1 (or a completely unrelated DNA sequence) flanked by DNA homologous to the endogenous LOXL1 (either the coding regions or regulatory regions of LOXL1) can be used, with or without a selectable marker and/or a negative selectable marker, to transfect cells that express LOXL1 in vivo. Insertion of the DNA construct, via targeted homologous recombination, results in inactivation of LOXL1. The recombinant DNA constructs can be directly administered or targeted to the required site in vivo using appropriate vectors, as described above. Alternatively, expression of non-mutant LOXL1 can be increased using a similar method: targeted homologous recombination can be used to insert a DNA construct comprising a non-mutant, functional LOXL1 (e.g., a gene having SEQ ID NO: 84 which may optionally comprise at least one polymorphism shown in Table 4 and 6a), or a portion thereof, in place of a mutant LOXL1 in the cell, as described above. In another embodiment, targeted homologous recombination can be used to insert a DNA construct comprising a nucleic acid that encodes a LOXL1 polypeptide variant that differs from that present in the cell.

Alternatively, endogenous LOXL1 expression can be reduced by targeting deoxyribonucleotide sequences complementary to the regulatory region of LOXL1 (i.e., the LOXL1 promoter and/or enhancers) to form triple helical structures that prevent transcription of LOXL1 in target cells in the body. (See generally, Helene, C. (1991) Anticancer Drug Des., 6(6):569-84; Helene, C., et al. (1992) Ann, N.Y. Acad. Sci., 660:27-36; and Maher, L. J. (1992) Bioassays 14(12):807-15). Likewise, the antisense constructs described herein, by antagonizing the normal biological activity of one of the LOXL1 proteins, can be used in the manipulation of tissue, e.g., tissue differentiation, both in vivo and for ex vivo tissue cultures. Furthermore, the anti-sense techniques (e.g., microinjection of antisense molecules, or transfection with plasmids whose transcripts are anti-sense with regard to a LOXL1 mRNA or gene sequence) can be used to investigate role of LOXL1 in developmental events, as well as the normal cellular function of LOXL1 in adult tissue. Such techniques can be utilized in cell culture, but can also be used in the creation of transgenic animals.

In yet another embodiment of the invention, other LOXL1 therapeutic agents as described herein can also be used in the treatment or prevention of XFS and/or glaucoma. The therapeutic agents can be delivered in a composition, as described above, or by themselves. They can be administered systemically, or can be targeted to a particular tissue. The therapeutic agents can be produced by a variety of means, including chemical synthesis; recombinant production; in vivo production (e.g., a transgenic animal, such as U.S. Pat. No. 4,873,316 to Meade et al.), for example, and can be isolated using standard means such as those described herein.

A combination of any of the above methods of treatment (e.g., administration of non-mutant LOXL1 polypeptide in conjunction with antisense therapy targeting mutant LOXL1 mRNA; administration of a first splicing variant encoded by LOXL1 in conjunction with antisense therapy targeting a second splicing encoded by LOXL1), can also be used.

### Computer-Implemented Aspects

As understood by those of ordinary skill in the art, the methods and information described herein may be implemented, in all or in part, as computer executable instructions on known computer readable media. For example, the methods described herein may be implemented in hardware. Alternatively, the method may be implemented in software stored in, for example, one or more memories or other computer readable medium and implemented on one or more processors. As is known, the processors may be associated with one or more controllers, calculation units and/or other units of a computer system, or implanted in firmware as desired. If implemented in software, the routines may be stored in any computer readable memory such as in RAM, ROM, flash memory, a magnetic disk, a laser disk, or other storage medium, as is also known. Likewise, this software may be delivered to a computing device via any known delivery method including, for example, over a communication channel such as a telephone line, the Internet, a wireless connection, etc., or via a transportable medium, such as a computer readable disk, flash drive, etc.

More generally, and as understood by those of ordinary skill in the art, the various steps described above may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

When implemented in software, the software may be stored in any known computer readable medium such as on a magnetic disk, an optical disk, or other storage medium, in a RAM or ROM or flash memory of a computer, processor, hard disk drive, optical disk drive, tape drive, etc. Likewise, the software may be delivered to a user or a computing system via any known delivery method including, for example, on a computer readable disk or other transportable computer storage mechanism.

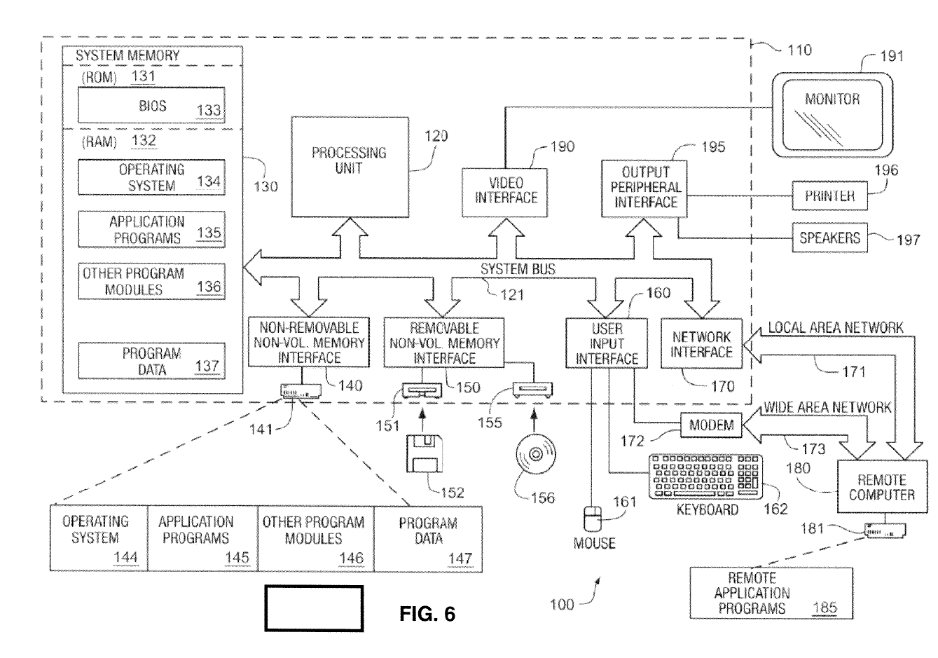

FIG. 6 illustrates an example of a suitable computing system environment 100 on which a system for the steps of the claimed method and apparatus may be implemented. The computing system environment 100 is only one example of a suitable computing environment and is not intended to suggest any limitation as to the scope of use or functionality of the method or apparatus of the claims. Neither should the computing environment 100 be interpreted as having any dependency or requirement relating to any one or combination of components illustrated in the exemplary operating environment 100.

The steps of the claimed method and system are operational with numerous other general purpose or special purpose computing system environments or configurations. Examples of well known computing systems, environments, and/or configurations that may be suitable for use with the methods or system of the claims include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The steps of the claimed method and system may be described in the general context of computer-executable instructions, such as program modules, being executed by a computer. Generally, program modules include routines, programs, objects, components, data structures, etc. that perform particular tasks or implement particular abstract data types. The methods and apparatus may also be practiced in distributed computing environments where tasks are performed by remote processing devices that are linked through a communications network. In both integrated and distributed computing environments, program modules may be located in both local and remote computer storage media including memory storage devices.

With reference to FIG. 6, an exemplary system for implementing the steps of the claimed method and system includes a general purpose computing device in the form of a computer 110. Components of computer 110 may include, but are not limited to, a processing unit 120, a system memory 130, and a system bus 121 that couples various system components including the system memory to the processing unit 120. The system bus 121 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computer 110 typically includes a variety of computer readable media. Computer readable media can be any available media that can be accessed by computer 110 and includes both volatile and nonvolatile media, removable and non-removable media. By way of example, and not limitation, computer readable media may comprise computer storage media and communication media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by computer 110. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The system memory 130 includes computer storage media in the form of volatile and/or nonvolatile memory such as read only memory (ROM) 131 and random access memory (RAM) 132. A basic input/output system 133 (BIOS), containing the basic routines that help to transfer information between elements within computer 110, such as during start-up, is typically stored in ROM 131. RAM 132 typically contains data and/or program modules that are immediately accessible to and/or presently being operated on by processing unit 120. By way of example, and not limitation, FIG. 6 illustrates operating system 134, application programs 135, other program modules 136, and program data 137.

The computer 110 may also include other removable/non-removable, volatile/nonvolatile computer storage media. By way of example only, FIG. 6 illustrates a hard disk drive 140 that reads from or writes to non-removable, nonvolatile magnetic media, a magnetic disk drive 151 that reads from or writes to a removable, nonvolatile magnetic disk 152, and an optical disk drive 155 that reads from or writes to a removable, nonvolatile optical disk 156 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 141 is typically connected to the system bus 121 through a non-removable memory interface such as interface 140, and magnetic disk drive 151 and optical disk drive 155 are typically connected to the system bus 121 by a removable memory interface, such as interface 150.

The drives and their associated computer storage media discussed above and illustrated in FIG. 6, provide storage of computer readable instructions, data structures, program modules and other data for the computer 110. In FIG. 6, for example, hard disk drive 141 is illustrated as storing operating system 144, application programs 145, other program modules 146, and program data 147. Note that these components can either be the same as or different from operating system 134, application programs 135, other program modules 136, and program data 137. Operating system 144, application programs 145, other program modules 146, and program data 147 are given different numbers here to illustrate that, at a minimum, they are different copies. A user may enter commands and information into the computer 20 through input devices such as a keyboard 162 and pointing device 161, commonly referred to as a mouse, trackball or touch pad. Other input devices (not shown) may include a microphone, joystick, game pad, satellite dish, scanner, or the like. These and other input devices are often connected to the processing unit 120 through a user input interface 160 that is coupled to the system bus, but may be connected by other interface and bus structures, such as a parallel port, game port or a universal serial bus (USB). A monitor 191 or other type of display device is also connected to the system bus 121 via an interface, such as a video interface 190. In addition to the monitor, computers may also include other peripheral output devices such as speakers 197 and printer 196, which may be connected through an output peripheral interface 190.

The computer 110 may operate in a networked environment using logical connections to one or more remote computers, such as a remote computer 180. The remote computer 180 may be a personal computer, a server, a router, a network PC, a peer device or other common network node, and typically includes many or all of the elements described above relative to the computer 110, although only a memory storage device 181 has been illustrated in FIG. 6. The logical connections depicted in FIG. 6 include a local area network (LAN) 171 and a wide area network (WAN) 173, but may also include other networks. Such networking environments are commonplace in offices, enterprise-wide computer networks, intranets and the Internet.

When used in a LAN networking environment, the computer 110 is connected to the LAN 171 through a network interface or adapter 170. When used in a WAN networking environment, the computer 110 typically includes a modem 172 or other means for establishing communications over the WAN 173, such as the Internet. The modem 172, which may be internal or external, may be connected to the system bus 121 via the user input interface 160, or other appropriate mechanism. In a networked environment, program modules depicted relative to the computer 110, or portions thereof, may be stored in the remote memory storage device. By way of example, and not limitation, FIG. 6 illustrates remote application programs 185 as residing on memory device 181. It will be appreciated that the network connections shown are exemplary and other means of establishing a communications link between the computers may be used.

Although the forgoing text sets forth a detailed description of numerous different embodiments of the invention, it should be understood that the scope of the invention is defined by the words of the claims set forth at the end of this patent. The detailed description is to be construed as exemplary only and does not describe every possibly embodiment of the invention because describing every possible embodiment would be impractical, if not impossible. Numerous alternative embodiments could be implemented, using either current technology or technology developed after the filing date of this patent, which would still fall within the scope of the claims defining the invention.

While the risk evaluation system and method, and other elements, have been described as preferably being implemented in software, they may be implemented in hardware, firmware, etc., and may be implemented by any other processor. Thus, the elements described herein may be implemented in a standard multi-purpose CPU or on specifically designed hardware or firmware such as an application-specific integrated circuit (ASIC) or other hard-wired device as desired, including, but not limited to, the computer 110 of FIG. 6. When implemented in software, the software routine may be stored in any computer readable memory such as on a magnetic disk, a laser disk, or other storage medium, in a RAM or ROM of a computer or processor, in any database, etc. Likewise, this software may be delivered to a user or a diagnostic system via any known or desired delivery method including, for example, on a computer readable disk or other transportable computer storage mechanism or over a communication channel such as a telephone line, the internet, wireless communication, etc. (which are viewed as being the same as or interchangeable with providing such software via a transportable storage medium).

Thus, many modifications and variations may be made in the techniques and structures described and illustrated herein without departing from the spirit and scope of the present invention. Accordingly, it should be understood that the methods and apparatus described herein are illustrative only and are not limiting upon the scope of the invention.

The present invention will now be exemplified by the following non-limiting examples.

### EXAMPLE 1

### Patient Cohort

Diagnosis of exfoliation syndrome (XFS) (also referred to as exfoliation syndrome (XFS), and exfoliation glaucoma (XFG) was done as previously described by Jonasson et al. (Eye 17: 747-753 (2003). Presence of XFS was confirmed by looking for the typical white, fluffy or granular material at the pupillary margin or on the anterior lens surface. Also, cental shield and/or peripheral band on the anterior lens capsule were considered to have definite XFS and if they also had POAG they were considered to have XFG. Diagnostic criteria for primary open angle glaucoma (POAG) were as described in Jonasson et al. (Eye 17: 747-753 (2003).

### Genotyping

A genome-wide scan of Icelandic individuals diagnosed with glaucoma, Primary Open Angle Glaucoma (POAG), XFS with glaucoma and XFS without glaucoma, as well as over 14,000 population controls, was performed using Infinium HumanHap300 SNP chips from Illumina for assaying approximately 317,000 single nucleotide polymorphisms (SNPs) on a single chip (Illumina, San Diego, Calif., USA). SNP genotyping for replication in other case-control cohorts was carried using the Centaurus platform (Nanogen).

### Statistical Methods for Association Analysis.

To test individual markers for association to disease phenotypes such as coronary artery disease or myocardial infarction, we use a likelihood ratio test to calculate a two-sided P-value for each allele of the markers. We calculate relative risk (RR) and population attributable risk (PAR) assuming a multiplicative model (C. T. Falk, P. Rubinstein, Ann Hum Genet. 51 (Pt 3), 227 (1987); J. D. Terwilliger, J. Ott, Hum Hered 42, 337 (1992)). To elucidate the linkage disequilibrium between markers in the region we used the CEPH Caucasian HapMap data. We calculated LD between pairs of SNPs using the standard definition of D′ (R. C. Lewontin, Genetics 50, 757 (1964)) and for the correlation coefficient r² (W. G. Hill, A. Robertson, Genetics 60, 615 (November, 1968). For the Icelandic cohort, to take into account that some of the individuals are related to each other, we obtained the null statistic of the test statistic either by simulating genotypes through the Icelandic genealogy or from the test statistic for all the 300,000 tested for association in the initial genome-wide association scan (citation). Model-free estimates of the genotype relative risk are generated as follows: RR of genotype G₁ compared to genotype G₀ was estimated by [n(G₁)/n(G₀)]/[m(G₁)/m(G₀)] where n and m denote genotype counts in patients and controls respectively. Results from different cohorts were combined using a Mantel-Hanezel model (citation) where cohorts are allowed to have different population frequencies for the alleles/genotypes but assume to have common relative risks.

### Results

### Genome-Wide Association Study

We successfully genotyped 165 Icelandic glaucoma patients, 78 patients diagnosed with Primary Open Angle Glaucoma (POAG), 60 patients diagnosed with XFS and glaucoma, and 55 patients diagnosed with XFS without glaucoma, as well as 14474 population control individuals without known history of XFS or glaucoma using the Illumina 330K chip. We performed a genome-wide scan for association to glaucoma and XFS, testing individually each of the SNPs on the chip that was successfully genotyped.

We found that one marker, rs2165241 on chromosome 15q24.1 showed significant association to glaucoma (Table 1; glaucoma combined), with an odds ratio of about 2.35.

Since it is well established that XFS is a major risk factor for the development of glaucoma, we decided to analyze phenotypical well-defined cohorts, i.e. (1) a cohort (glaucoma combined) comprising individuals with confirmed glaucoma, (2) a cohort (POAG) comprising individuals diagnosed with Primary Open Angle Glaucoma, (3) a cohort (XFS+G) comprising individuals diagnosed with XFS (some of which may also have glaucoma), and (4) a cohort (XFS) comprising individuals diagnosed with XFS in the absence of glaucoma. Analyzing these cohorts allows us to investigate the relative contribution of the various phenotypes to the observed signal.

As shown in Table 1, the OR of 2.35 observed in the glaucoma phenotype decreases to 1.37 in the POAG cohort. However, a much higher OR of 3.50 was observed in a cohort of XFS-glaucoma patients (XFG). This is indicative of the effect being strongest in this phenotypic cohort. The observed OR value of 3.18 in the XFS only group is close to being as strong as in the XFG group, indicating that the observed effect is mainly stemming from XFS and exerts its effect in glaucoma patients through the XFS-glaucoma phenotype.

The rs2165241 marker is located in a small region with strong linkage disequilibrium on chromosome 15q24.1, herein denoted LOXL1 LD block. Several other SNPs within the region were investigated for association to XFS, as shown in Table 2. These results indicate that this region on chromosome 15 encompassing the LOXL1 gene strongly associates with XFS.

Due to the fact that the at-risk T-allele of rs2165241 is quite common in the general population (470.3% allelic frequency) and the very high value for OR observed, the impact of the association is dramatic. Thus, the T allele of rs2165241 could account for more than 37% of XFS cases in the general population in individuals of European descent.

### Sequencing of LOXL1 Gene

Sequencing of the LOXL1 gene was performed, to investigate whether variants in exons, promoter region or 3′ untranslated region could be underlying the observed association to XFS and glaucoma. Samples from four populations were sequenced, 94 samples from each: Icelandic controls, HapMap CEU samples, Icelandic glaucoma patients, and Icelandic XFS patients. Approximately 1000 bp of 5′ sequence was assessed, as well as 200 bp flanking each exon and 300 bp of 3′ sequence.

Results of the sequencing are shown in Table 6. In addition to previously known SNPs, we identified several novel SNPs, several of which were found to be highly correlated with XFS in this sample. In particular, two SNPs, SG15S209 (rs8023330) and SG15S210 (rs12441130) were found to be highly correlated with rs2165241 (r² of 0.98 and 0.96, respectively), with estimated odds ratio (OR) values even higher than found for rs2165241.

TABLE 1
Association to XFS and Glaucoma on 15q24.1.
Shown is the association for the risk allele of the SNP rs2165241 to glaucoma in the Icelandic
case-control group. Results are shown for all glaucoma cases and for primary open angle
glaucoma (POAG) cases and pseudo-exfoliation glaucoma cases (XFG; individuals diagnosed with
XFS and glaucoma) separately. Also included are results for an independent group of pseudo-
exfoliation cases without the presence of glaucoma from the Reykjavik Eye Study (RES). Study
population includes the number of cases (n) and controls (m). The results include the OR, 95%
confidence intervals (CI) and P values calculated assuming the multiplicative model, and the
genotype specific OR for the heterozygous (0X) and homozygous carriers (XX) compared to the
risk for non-carriers (00). The P values and CI were adjusted for relatedness of the cases and
controls by simulations.

| Study population | Frequency | | | | Genotype specific OR | | |
|---|---|---|---|---|---|---|---|
| (n/m) | Control | Cases | OR (95% CI) | P | 00 | 0X (95% CI) | XX (95% CI) |
| Glaucoma Combined | 0.473 | 0.679 | 2.35 (1.87-2.97) | 5.13E−13 | 1 | 1.56 (0.96-2.55) | 4.55 (2.93-7.07) |
| (165/14474) | | | | | | | |
| POAG (78/14474) | 0.473 | 0.551 | 1.37 (0.99-1.88) | 0.055 | 1 | 1.18 (0.66-2.10) | 1.82 (0.98-3.38) |
| XFG (60/14474) | 0.473 | 0.758 | 3.50 (2.36-5.17) | 3.56E−10 | 1 | 4.23 (1.52-11.75) | 13.92 (5.91-32.83) |
| XFS only (55/14474) | 0.473 | 0.740 | 3.18 (2.12-4.76) | 1.92E−08 | 1 | 2.04 (0.80-5.18) | 7.76 (3.58-16.85) |

TABLE 2
Association to XFS for additional markers in the LOXL1 region.
Shown is the association to exfoliation syndrome (XFS) for ten
SNPs located in the LOXL1 gene, including rs2165241.
The association is calculated for the combined group of
115 XFS cases versus 14474 controls. The results include
for each SNP the frequency in cases and controls, OR,
95% CI and P value calculated assuming the multiplicative model.
Also included is the correlation r² between each of the
additional markers and the at-risk SNP rs2165241.

| | | | Frequency | | | |
|---|---|---|---|---|---|---|
| | Al- | | Con- | | | |
| SNP | lele | r^{2a} | trol | Cases | OR (95% CI) | P |
| rs2165241 | T | — | 0.473 | 0.750 | 3.34 (2.52-4.43) | 6.78E−17 |
| rs4337252 | G | 0.97 | 0.492 | 0.769 | 3.42 (2.57-4.57) | 6.25E−17 |
| rs2028386 | C | 0.96 | 0.489 | 0.768 | 3.45 (2.59-4.60) | 3.33E−17 |
| rs750460 | T | 0.77 | 0.457 | 0.728 | 3.18 (2.40-4.20) | 5.65E−16 |
| rs4243042 | T | 0.77 | 0.457 | 0.728 | 3.18 (2.40-4.20) | 5.24E−16 |
| rs12440667 | T | 0.75 | 0.461 | 0.729 | 3.15 (2.38-4.17) | 1.23E−15 |
| rs1992314 | C | 0.48 | 0.655 | 0.819 | 2.34 (1.72-3.19) | 7.88E−08 |
| rs893817 | A | 0.36 | 0.633 | 0.790 | 2.16 (1.60-2.91) | 4.92E−07 |
| rs4461027 | C | 0.31 | 0.392 | 0.536 | 1.78 (1.37-2.32) | 1.61E−05 |
| rs2304719 | C | 0.28 | 0.695 | 0.873 | 2.97 (2.11-4.19) | 5.63E−10 |

^{a}Correlation with the marker rs2165241 based on HapMap CEU data.

TABLE 3
Markers correlated with rs2165241.
All SNPs in the region 71.8 to 72.2 Mb on 15q24.1
(based on the HapMap v22 CEU dataset) that are correlated,
with r² ≧ 0.2, to at at-risk marker rs2165241 (indicated in bold).
Included in the table are two measure of correlation to rs2165241:
D′ and r², both estimated from the HapMap CEU dataset.

| SNP | Position^{a} | D′ | r² |
|---|---|---|---|
| rs4886467 | 71914630 | 0.56 | 0.28 |
| rs4261482 | 71916249 | 0.87 | 0.22 |
| rs4461027 | 71927365 | 0.61 | 0.31 |
| rs4886761 | 71931368 | 0.61 | 0.31 |
| rs2165241 | 71938019 | — | — |
| rs1992314 | 71939584 | 1.00 | 0.48 |
| rs4886776 | 71940813 | 1.00 | 0.48 |
| rs2028386 | 71942525 | 1.00 | 0.96 |
| rs4337252 | 71942582 | 1.00 | 0.97 |
| rs2028387 | 71942854 | 1.00 | 0.47 |
| rs4077284 | 71944208 | 0.81 | 0.36 |
| rs893816 | 71944281 | 1.00 | 0.48 |
| rs4886782 | 71944627 | 1.00 | 0.56 |
| rs893817 | 71944882 | 0.81 | 0.36 |
| rs893818 | 71945012 | 1.00 | 0.48 |
| rs893820 | 71945420 | 1.00 | 0.47 |
| rs12440667 | 71947256 | 0.92 | 0.75 |
| rs2304719 | 71951317 | 1.00 | 0.28 |
| rs750460 | 71957323 | 0.96 | 0.77 |
| rs4243042 | 71957441 | 0.96 | 0.77 |
| rs12437465 | 71959063 | 0.92 | 0.58 |

^{a}Basepair position on chromosome 16 in NCBI Build 34.
^{b}Correlation coefficients, D′ and r², with the SNP rs2165241.

TABLE 4
Polymorphic SNP markers within the LOXL1 LD block on chromosome
15.
Shown are marker names (public rs-names, the nucleotide base
change of the polymorphism, the position of the polymorphism
(Coordinates based on NCBI Build 34), the strand of the
polymorphism as listed (+ or −),
and the position of the polymorphism in SEQ ID NO: 84.

| | base | | | | Position in SEQ ID |
|---|---|---|---|---|---|
| marker | change | chr15 | position | strand | NO: 84 |
| rs4243038 | A/G | chr15 | 71928236 | + | 15 |
| rs7182614 | A/G | chr15 | 71928738 | + | 517 |
| rs28394669 | C/G | chr15 | 71928840 | + | 619 |
| rs28542042 | C/T | chr15 | 71929174 | + | 953 |
| rs28394570 | A/G | chr15 | 71929203 | + | 982 |
| rs12898282 | A/G | chr15 | 71929417 | + | 1196 |
| rs7167367 | A/G | chr15 | 71929689 | + | 1468 |
| rs35022996 | A/T | chr15 | 71930241 | + | 2020 |
| rs7173684 | A/G | chr15 | 71930700 | + | 2479 |
| rs7174531 | G/T | chr15 | 71930903 | + | 2682 |
| rs4886761 | C/T | chr15 | 71931368 | + | 3147 |
| rs8040846 | G/T | chr15 | 71932091 | + | 3870 |
| rs4886764 | C/T | chr15 | 71932241 | + | 4020 |
| rs12914565 | C/T | chr15 | 71932422 | + | 4201 |
| rs8026259 | C/T | chr15 | 71932576 | + | 4355 |
| rs28437367 | C/T | chr15 | 71932770 | + | 4549 |
| rs11852604 | A/G | chr15 | 71933334 | + | 5113 |
| rs6495084 | C/T | chr15 | 71933656 | + | 5435 |
| rs12708513 | C/G | chr15 | 71933995 | + | 5774 |
| rs16958477 | A/C | chr15 | 71934283 | + | 6062 |
| rs1048661 | G/T | chr15 | 71935363 | + | 7142 |
| rs3825942 | C/T | chr15 | 71935399 | − | 7178 |
| rs12906373 | C/T | chr15 | 71935877 | + | 7656 |
| rs1130133 | A/G | chr15 | 71936009 | + | 7788 |
| rs8023330 | A/G | chr15 | 71936416 | + | 8195 |
| rs28710821 | A/G | chr15 | 71936592 | + | 8371 |
| rs11637907 | C/T | chr15 | 71936633 | + | 8412 |
| rs1550436 | C/T | chr15 | 71936974 | + | 8753 |
| rs1550437 | C/T | chr15 | 71937115 | + | 8894 |
| rs6495085 | C/G | chr15 | 71937130 | + | 8909 |
| rs1550438 | C/T | chr15 | 71937207 | + | 8986 |
| rs6495086 | C/T | chr15 | 71937309 | + | 9088 |
| rs1550439 | A/T | chr15 | 71937456 | + | 9235 |
| rs8034403 | A/G | chr15 | 71937845 | + | 9624 |
| rs8034017 | A/G | chr15 | 71937860 | + | 9639 |
| rs2165241 | C/T | chr15 | 71938019 | + | 9798 |
| rs1078967 | A/G | chr15 | 71938804 | − | 10583 |
| rs1550433 | A/C | chr15 | 71939126 | + | 10905 |
| rs28588430 | C/G | chr15 | 71939247 | + | 11026 |
| rs28617339 | C/T | chr15 | 71939388 | + | 11167 |
| rs2415204 | A/C | chr15 | 71939440 | + | 11219 |
| rs28522673 | C/G | chr15 | 71939533 | + | 11312 |
| rs1992314 | C/G | chr15 | 71939584 | + | 11363 |
| rs1440101 | A/G | chr15 | 71940448 | + | 12227 |
| rs2289414 | A/G | chr15 | 71940617 | + | 12396 |
| rs4886776 | A/G | chr15 | 71940813 | + | 12592 |
| rs8041642 | A/G | chr15 | 71940923 | + | 12702 |
| rs8041685 | A/G | chr15 | 71941010 | + | 12789 |
| rs4886778 | A/C | chr15 | 71941205 | + | 12984 |
| rs8042039 | C/T | chr15 | 71941335 | + | 13114 |
| rs8027093 | C/T | chr15 | 71941484 | + | 13263 |
| rs8027022 | A/G | chr15 | 71941955 | + | 13734 |
| rs28603291 | C/T | chr15 | 71942287 | + | 14066 |
| rs4283182 | A/G | chr15 | 71942439 | + | 14218 |
| rs2028386 | C/G | chr15 | 71942525 | + | 14304 |
| rs4337252 | C/G | chr15 | 71942582 | + | 14361 |
| rs4243039 | A/G | chr15 | 71942719 | + | 14498 |
| rs2028387 | A/C | chr15 | 71942854 | + | 14633 |
| rs12594472 | A/T | chr15 | 71942957 | + | 14736 |
| rs2415205 | C/G | chr15 | 71943636 | + | 15415 |
| rs2899807 | A/T | chr15 | 71943637 | + | 15416 |
| rs28739347 | G/T | chr15 | 71943854 | + | 15633 |
| rs893815 | C/G | chr15 | 71943981 | + | 15760 |
| rs4077284 | A/G | chr15 | 71944208 | + | 15987 |
| rs893816 | C/T | chr15 | 71944281 | + | 16060 |
| rs4886782 | A/G | chr15 | 71944627 | + | 16406 |
| rs893817 | A/G | chr15 | 71944882 | + | 16661 |
| rs893818 | A/G | chr15 | 71945012 | + | 16791 |
| rs12917501 | A/G | chr15 | 71945220 | + | 16999 |
| rs4423374 | A/G | chr15 | 71945327 | + | 17106 |
| rs893819 | A/G | chr15 | 71945341 | + | 17120 |
| rs4243040 | A/G | chr15 | 71945380 | + | 17159 |
| rs893820 | C/T | chr15 | 71945420 | + | 17199 |
| rs4243041 | C/G | chr15 | 71945598 | + | 17377 |
| rs4886785 | A/G | chr15 | 71945608 | + | 17387 |
| rs16958494 | C/T | chr15 | 71947166 | + | 18945 |
| rs12440667 | C/T | chr15 | 71947256 | + | 19035 |
| rs28594928 | C/T | chr15 | 71947265 | + | 19044 |
| rs11855863 | C/T | chr15 | 71947320 | + | 19099 |
| rs9944214 | C/T | chr15 | 71947674 | + | 19453 |
| rs12594324 | C/T | chr15 | 71948182 | + | 19961 |
| rs12905253 | A/G | chr15 | 71948254 | + | 20033 |
| rs10851865 | A/G | chr15 | 71948314 | + | 20093 |
| rs9783739 | A/T | chr15 | 71949568 | + | 21347 |
| rs11638944 | C/G | chr15 | 71949899 | + | 21678 |
| rs8042181 | A/G | chr15 | 71949988 | + | 21767 |
| rs11072449 | A/G | chr15 | 71950301 | + | 22080 |
| rs12441130 | C/T | chr15 | 71950719 | + | 22498 |
| rs2304719 | C/T | chr15 | 71951317 | + | 23096 |
| rs11631579 | A/G | chr15 | 71951521 | + | 23300 |
| rs28758267 | A/G | chr15 | 71951664 | + | 23443 |
| rs12906558 | A/C | chr15 | 71951724 | + | 23503 |
| rs7173840 | G/T | chr15 | 71952189 | + | 23968 |
| rs7173508 | A/G | chr15 | 71952224 | + | 24003 |
| rs1001507 | G/T | chr15 | 71952770 | + | 24549 |
| rs11638420 | G/T | chr15 | 71952845 | + | 24624 |
| rs1530167 | C/T | chr15 | 71952901 | + | 24680 |
| rs1530168 | C/T | chr15 | 71952914 | + | 24693 |
| rs1530169 | C/T | chr15 | 71952937 | + | 24716 |
| rs10775207 | A/G | chr15 | 71953837 | + | 25616 |
| rs7163406 | A/G | chr15 | 71954089 | + | 25868 |
| rs35439299 | C/G | chr15 | 71954426 | + | 26205 |
| rs2304720 | C/T | chr15 | 71954551 | + | 26330 |
| rs28706550 | A/C | chr15 | 71954672 | + | 26451 |
| rs13329465 | G/T | chr15 | 71955016 | + | 26795 |
| rs11636165 | A/G | chr15 | 71955154 | + | 26933 |
| rs13329473 | C/T | chr15 | 71955342 | + | 27121 |
| rs4595753 | G/T | chr15 | 71955686 | + | 27465 |
| rs2304721 | A/C | chr15 | 71956171 | + | 27950 |
| rs4497636 | A/G | chr15 | 71956354 | + | 28133 |
| rs28578363 | C/T | chr15 | 71956919 | + | 28698 |
| rs893821 | C/T | chr15 | 71957052 | + | 28831 |
| rs4886796 | A/T | chr15 | 71957185 | + | 28964 |
| rs750460 | C/T | chr15 | 71957323 | − | 29102 |
| rs4243042 | A/T | chr15 | 71957441 | + | 29220 |
| rs2304722 | C/T | chr15 | 71957566 | + | 29345 |
| rs35203737 | A/C | chr15 | 71957708 | + | 29487 |
| rs1440102 | A/G | chr15 | 71957803 | + | 29582 |
| rs28566488 | C/G | chr15 | 71958285 | + | 30064 |
| rs12437465 | C/T | chr15 | 71959063 | + | 30842 |
| rs11072450 | A/G | chr15 | 71959561 | + | 31340 |
| rs12441138 | A/G | chr15 | 71959714 | + | 31493 |
| rs8818 | C/G | chr15 | 71960095 | + | 31874 |
| rs3522 | C/T | chr15 | 71960161 | + | 31940 |
| rs7168465 | C/T | chr15 | 71960239 | + | 32018 |
| rs7173049 | A/G | chr15 | 71960427 | + | 32206 |
| rs7175324 | G/T | chr15 | 71960824 | + | 32603 |
| rs12442139 | C/T | chr15 | 71961303 | + | 33082 |
| rs12442211 | A/G | chr15 | 71961492 | + | 33271 |
| rs12913399 | C/G | chr15 | 71961862 | + | 33641 |
| rs12102180 | A/C | chr15 | 71962242 | + | 34021 |
| rs28463653 | G/T | chr15 | 71962334 | + | 34113 |
| rs12899085 | A/G | chr15 | 71962739 | + | 34518 |
| rs4886494 | A/G | chr15 | 71963285 | + | 35064 |
| rs4886802 | C/T | chr15 | 71963288 | + | 35067 |
| rs11072451 | C/G | chr15 | 71963601 | + | 35380 |
| rs28563987 | A/G | chr15 | 71963685 | + | 35464 |
| rs2415206 | C/T | chr15 | 71963713 | + | 35492 |
| rs8031396 | A/G | chr15 | 71963745 | + | 35524 |
| rs11072452 | G/T | chr15 | 71963748 | + | 35527 |
| rs11631102 | A/G | chr15 | 71963752 | + | 35531 |
| rs8030957 | A/G | chr15 | 71963755 | + | 35534 |
| rs12904518 | A/C | chr15 | 71963761 | + | 35540 |
| rs12102015 | C/T | chr15 | 71964177 | + | 35956 |
| rs12101466 | A/G | chr15 | 71964205 | + | 35984 |
| rs12102019 | C/T | chr15 | 71964294 | + | 36073 |
| rs12101479 | C/G | chr15 | 71964365 | + | 36144 |
| rs28642047 | A/G | chr15 | 71964681 | + | 36460 |
| rs28433432 | A/C | chr15 | 71965447 | + | 37226 |
| rs28707234 | A/G | chr15 | 71965670 | + | 37449 |
| rs11072453 | A/T | chr15 | 71965673 | + | 37452 |
| rs4886803 | C/G | chr15 | 71965791 | + | 37570 |
| rs12907105 | C/G | chr15 | 71966244 | + | 38023 |

TABLE 5

Microsatellite markers in LOXL1 LD block on
chromosome 15.
Shown is the name of the marker, position (start
and end with respect to NCBI Build 34) of the
polymorphism, size of the amplimer and forward
and reverse primers for amplifying the
polymorphism

| | | | | | Fwd | Rev |
|---|---|---|---|---|---|---|
| Name | Chr | Start | End | Size | primer | primer |
| D15S818 | chr15 | 71946384 | 71946526 | 143 | TGTGCAT | GCTAAG |
| | | | | | CCTCTAT | ATGGCG |
| | | | | | GTCCCT | CCATTG |

TABLE 6
Polymorphic markers within exons and flanking intronic sequences in the
LOXL1 gene.

| | | | | | | | | | | | Location in |
|---|---|---|---|---|---|---|---|---|---|---|---|
| SNP | Pos | Var | rs Name | CEU | Glaucoma | Control | XFS | OR | P | r² | gene |
| SG15S202 | 71933656 | C/T | rs6495084 | 0.0398 | 0.0269 | 0.0055 | 0.0000 | | | | Promoter |
| SG15S203 | 71933995 | C/G | rs12708513 | 0.0393 | 0.0269 | 0.0055 | 0.0000 | | | | Promoter |
| SG15S204 | 71934283 | A/C | rs16958477 | 0.3764 | 0.4154 | 0.3611 | 0.5208 | 1.92 | 0.046 | 0.28 | Promoter |
| SG15S205 | 71934326 | C/G | | 0.0225 | 0.0000 | 0.0167 | 0.0000 | | | | Promoter |
| SG15S206 | 71934869 | A/G | | 0.0000 | 0.0040 | 0.0110 | 0.0000 | | | | 5′ UTR |
| SG15S178 | 71935363 | G/T | rs1048661 | 0.5511 | 0.7188 | 0.6404 | 0.8400 | 2.95 | 0.005 | 0.46 | Exon 1 |
| SG15S179 | 71935399 | A/G | rs3825942 | 0.1534 | 0.1181 | 0.1833 | 0.0000 | | | | Exon 1 |
| SG15S208 | 71935817 | C/T | | 0.0282 | 0.0126 | 0.0432 | 0.0000 | | | | Exon 1 |
| SG15S242 | 71935901 | G/T | | 0.0000 | 0.0405 | 0.0067 | 0.0833 | | | | Exon 1 |
| SG15S243 | 71936221 | A/C | | 0.0000 | 0.0242 | 0.0056 | 0.0455 | | | | Intron 1 |
| SG15S209 | 71936416 | A/G | rs8023330 | 0.3897 | 0.5273 | 0.4389 | 0.7391 | 3.62 | 0.00022 | 0.98 | Intron 1 |
| SG15S210 | 71950719 | C/T | rs12441130 | 0.3706 | 0.5615 | 0.4444 | 0.8000 | 5.00 | 4.44E−06 | 0.96 | Intron 1 |
| SG15S211 | 71951055 | C/T | | 0.0000 | 0.0038 | 0.0055 | 0.0000 | | | | Exon 2 |
| SG15S185 | 71951317 | C/T | rs2304719 | 0.7159 | 0.7752 | 0.6209 | 0.8800 | 4.48 | 0.00021 | 0.49 | Intron 2 |
| SG15S212 | 71951378 | A/G | | 0.0000 | 0.0039 | 0.0055 | 0.0200 | | | | Intron 2 |
| SG15S213 | 71954551 | C/T | rs2304720 | 0.7216 | 0.7756 | 0.6209 | 0.8696 | 4.07 | 0.00064 | 0.49 | Intron 2 |
| SG15S214 | 71955070 | A/G | | 0.0506 | 0.0440 | 0.0275 | 0.0652 | 2.47 | 0.25 | 0.02 | Intron 3 |
| SG15S215 | 71955124 | A/G | | 0.7360 | 0.8320 | 0.8500 | 0.9348 | 2.53 | 0.1 | 0.14 | Intron 3 |
| SG15S216 | 71955125 | C/T | | 0.0393 | 0.0280 | 0.0167 | 0.0217 | 1.31 | 0.82 | 0.01 | Intron 3 |
| SG15S217 | 71955189 | A/G | | 0.0000 | 0.0040 | 0.0000 | 0.0000 | | | | Intron 3 |
| SG15S218 | 71955295 | A/G | | 0.0000 | 0.0038 | 0.0110 | 0.0000 | | | | Exon 4 |
| SG15S219 | 71955430 | A/G | | 0.0805 | 0.0465 | 0.0714 | 0.0000 | | | | Intron 4 |
| SG15S220 | 71955686 | G/T | rs4595753 | 0.0393 | 0.0115 | 0.0055 | 0.0000 | | | | Intron 4 |
| SG15S221 | 71955688 | C/T | | 0.7360 | 0.8269 | 0.8516 | 0.9400 | 2.73 | 0.076 | 0.14 | Intron 4 |
| SG15S222 | 71956120 | A/G | | 0.0056 | 0.0000 | 0.0000 | 0.0000 | | | | Intron 5 |
| SG15S223 | 71956163 | C/G | | 0.0000 | 0.0039 | 0.0000 | 0.0000 | | | | Intron 5 |
| SG15S224 | 71956171 | A/C | rs2304721 | 0.0506 | 0.0440 | 0.0281 | 0.0600 | 2.21 | 0.31 | 0.02 | Intron 5 |
| SG15S225 | 71956354 | A/G | rs4497636 | 0.0291 | 0.0119 | 0.0057 | 0.0000 | | | | Intron 5 |
| SG15S180 | 71957323 | A/G | rs750460 | 0.3603 | 0.5391 | 0.4691 | 0.7708 | 3.81 | 0.00016 | 0.95 | Intron 5 |
| SG15S175 | 71957441 | A/T | rs4243042 | 0.3391 | 0.5385 | 0.4396 | 0.7600 | 4.04 | 4.14E−05 | 0.98 | Intron 5 |
| SG15S226 | 71957496 | C/T | | 0.0398 | 0.0269 | 0.0165 | 0.0200 | 1.22 | 0.87 | 0.01 | Intron 5 |
| SG15S227 | 71957566 | C/T | rs2304722 | 0.7360 | 0.8192 | 0.8462 | 0.9400 | 2.85 | 0.062 | 0.15 | Intron 5 |
| SG15S228 | 71957884 | A/G | | 0.0000 | 0.0273 | 0.0055 | 0.0400 | | | | Intron 6 |
| SG15S229 | 71960095 | C/G | rs8818 | 0.8483 | 0.8231 | 0.8462 | 0.8800 | 1.33 | 0.54 | 0.04 | 3′ UTR |
| SG15S230 | 71960130 | G/T | | 0.0112 | 0.0000 | 0.0000 | 0.0000 | | | | 3′ UTR |
| SG15S231 | 71960161 | C/T | rs3522 | 0.5375 | 0.4922 | 0.4667 | 0.5000 | 1.14 | 0.7 | 0.15 | 3′ UTR |
| SG15S234 | 71960409 | C/T | | 0.8295 | 0.8885 | 0.8462 | 0.8913 | 1.49 | 0.42 | 0.01 | downstream |
| SG15S235 | 71960427 | A/G | rs7173049 | 0.8125 | 0.8115 | 0.8407 | 0.9000 | 1.71 | 0.28 | 0.15 | downstream |
| SG15S236 | 71960695 | A/G | | 0.0114 | 0.0039 | 0.0167 | 0.0000 | | | | downstream |
| SG15S237 | 71960719 | A/G | | 0.9716 | 0.9805 | 0.9719 | 0.9783 | 1.30 | 0.81 | 0.02 | downstream |
| SG15S238 | 71960811 | C/T | | 0.0000 | 0.0000 | 0.0056 | 0.0000 | | | | downstream |
| SG15S239 | 71960824 | G/T | rs7175324 | 0.4034 | 0.4767 | 0.4663 | 0.5217 | 1.25 | 0.5 | 0.31 | downstream |

By sequencing all exons of the LOXL1 gene and their flanking sequences, as well as the 5′ promoter and 3′ regions in a total of 94 individuals from the HapMap Caucasian sample (CEU; see http://www.hapmap.org for details on population), 94 Icelandic control samples, 94 glaucoma patients and 94 patients with XFS, we identified several novel polymorphisms. Their position in NCBI Build 34, the type of amino acid change, the allelic frequency of the polymorphism, the location in the gene, the odds ratio (OR) of association to XFS, the p-value of the association and the correlation coefficient between the polymorphism and marker rs2165241 is indicated in the table.

TABLE 6a
Polymorphic markers within exons and flanking intronic sequences in the LOXL1 gene.

| | | | | | | | | | | | | Correlation r2 | | | | | |
|---|---|---|---|---|---|---|---|---|---|---|---|---|---|---|---|---|---|
| | | | Pos in | | | | | | | | | with coding | | | | Amino | |
| | | | SEQ | | | Frequency of at-risk allele | | | | | | mutations | | Location | codon | Acid | Type of |
| SNP | Pos | Strand | ID NO: 84 | Variation | rsName | CEU | Ctrl | Glaucoma | XFS | YRI | CHB/JPT | R141L | G153D | in gene | change | change | change |
| SG15S202 | 71933656 | + | 5435 | C/T | rs6495084 | 0.034 | 0.006 | 0.027 | 0 | NA | NA | 0.01 | 0 | Promoter | | | |
| SG15S273 | 71933903 | + | 5682 | C/T | NA | 0.991 | NA | NA | NA | NA | NA | 0.01 | 0 | Promoter | | | |
| SG15S203 | 71933995 | + | 5774 | C/G | rs12708513 | 0.966 | 0.994 | 0.973 | 1 | NA | NA | 0.01 | 0 | Promoter | | | |
| SG15S204 | 71934283 | + | 6062 | A/C | rs16958477 | 0.619 | 0.64 | 0.585 | 0.479 | NA | NA | 0.04 | 0.1 | Promoter | | | |
| SG15S205 | 71934326 | + | 6105 | C/G | NA | 0.975 | 0.983 | 1 | 1 | NA | NA | 0.01 | 0.06 | Promoter | | | |
| SG15S274 | 71934488 | + | 6267 | C/T | NA | 0 | 0 | 0.001 | 0.01 | NA | NA | 0 | 0 | Promoter | | | |
| SG15S206 | 71934869 | + | 6648 | A/G | NA | 0 | 0.011 | 0.004 | 0 | NA | NA | 0 | 0.03 | 5′ UTR | | | |
| SG15S262 | 71935229 | + | 7008 | C/G | NA | 0 | 0.003 | 0 | NA | 0 | 0 | 0 | 0 | Exon 1 | CAG->CAC | Q96H | conservative |
| SG15S263 | 71935232 | + | 7011 | A/G | NA | 0 | 0 | 0.001 | NA | 0 | 0 | 0 | 0 | Exon 1 | GCG->GCA | A97A | silent |
| SG15S178 | 71935363 | + | 7142 | G/T | rs1048661 | 0.559 | 0.651 | 0.74 | 0.848 | 0.853 | 0.471 | NA | 0.05 | Exon 1 | CGG->CTG | R141L | nonconservative |
| SG15S179 | 71935399 | + | 7178 | A/G | rs3825942 | 0.169 | 0.151 | 0.081 | 0 | 0.43 | 0.109 | 0.05 | NA | Exon 1 | GGC->GAC | G153D | nonconservative |
| SG15S264 | 71935416 | + | 7195 | G/T | NA | 0 | NA | NA | NA | 0.191 | 0 | 0 | 0 | Exon 1 | TCG->GCG | S159A | conservative |
| SG15S207 | 71935419 | + | 7198 | C/G | NA | 0 | 0.008 | 0.004 | 0 | 0 | 0 | 0 | 0.04 | Exon 1 | GCT->CCT | A160P | conservative |
| SG15S208 | 71935817 | + | 7596 | C/T | NA | 0.968 | 0.961 | 0.971 | 1 | 1 | 1 | 0.07 | 0 | Exon 1 | GAC->GAT | D292D | silent |
| SG15S242 | 71935901 | + | 7680 | G/T | NA | 1 | 0.986 | 0.978 | 0.938 | 0.964 | 0.696 | 0.01 | 0 | Exon 1 | GCG->GCT | A320A | silent |
| SG15S265 | 71936105 | + | 7884 | G/T | NA | 1 | NA | NA | NA | 1 | 0.963 | 0 | 0 | intron | | | |
| SG15S243 | 71936221 | + | 8000 | A/C | NA | 1 | 0.994 | 0.976 | 0.955 | NA | NA | 0.01 | 0 | intron | | | |
| SG15S209 | 71936416 | + | 8195 | A/G | rs8023330 | 0.625 | 0.562 | 0.473 | 0.261 | NA | NA | 0.48 | 0.15 | intron | | | |
| SG15S210 | 71950719 | + | 22498 | C/T | rs12441130 | 0.356 | 0.474 | 0.622 | 0.786 | NA | NA | 0.54 | 0.16 | intron | | | |
| SG15S266 | 71950798 | + | 22577 | C/T | NA | 1 | 1 | 0.998 | 1 | NA | NA | 0 | 0 | intron | | | |
| SG15S211 | 71951055 | + | 22834 | C/T | NA | 1 | 0.993 | 0.998 | 1 | NA | NA | 0 | 0 | Exon 2 | TCC->TCT | S382S | silent |
| SG15S185 | 71951317 | + | 23096 | C/T | rs2304719 | 0.712 | 0.701 | 0.807 | 0.917 | NA | NA | 0.09 | 0.39 | intron | | | |
| SG15S212 | 71951378 | + | 23157 | A/G | NA | 0 | 0.007 | 0.016 | 0.02 | NA | NA | 0.01 | 0 | intron | | | |
| SG15S272 | 71954406 | + | 26185 | C/G | NA | NA | NA | NA | NA | NA | NA | NA | NA | intron | | | |
| SG15S213 | 71954551 | + | 26330 | C/T | rs2304720 | 0.284 | 0.378 | 0.186 | 0.13 | NA | NA | 0.04 | 0.41 | intron | | | |
| SG15S214 | 71955070 | + | 26849 | A/G | NA | 0.051 | 0.045 | 0.053 | 0.06 | NA | NA | 0.02 | 0.01 | intron | | | |
| SG15S215 | 71955124 | + | 26903 | A/G | NA | 0.271 | 0.179 | 0.137 | 0.06 | NA | NA | 0.4 | 0.02 | intron | | | |
| SG15S216 | 71955125 | + | 26904 | C/T | NA | 0.966 | 0.981 | 0.98 | 0.98 | NA | NA | 0.04 | 0 | intron | | | |
| SG15S217 | 71955189 | + | 26968 | A/G | NA | 0 | 0 | 0.002 | 0 | NA | NA | 0 | 0.01 | intron | | | |
| SG15S218 | 71955295 | + | 27074 | A/G | NA | 0 | 0.007 | 0.002 | 0 | NA | NA | 0.01 | 0 | Exon 4 | GAG->AAG | E474K | conservative |
| SG15S219 | 71955430 | + | 27209 | A/G | NA | 0.086 | 0.059 | 0.036 | 0 | NA | NA | 0.03 | 0.33 | intron | | | |
| SG15S267 | 71955585 | + | 27364 | C/T | NA | 0 | 0.002 | 0 | 0 | NA | NA | 0 | 0.01 | intron | | | |
| SG15S220 | 71955686 | + | 27465 | G/T | rs4595753 | 0.034 | 0.011 | 0.011 | 0 | NA | NA | 0.01 | 0 | intron | | | |
| SG15S221 | 71955688 | + | 27467 | C/T | NA | 0.729 | 0.82 | 0.859 | 0.94 | NA | NA | 0.4 | 0.02 | intron | | | |
| SG15S222 | 71956120 | + | 27899 | A/G | NA | 0.008 | 0.004 | 0.002 | 0 | NA | NA | 0 | 0 | intron | | | |
| SG15S223 | 71956163 | + | 27942 | C/G | NA | 1 | 1 | 0.998 | 1 | NA | NA | 0 | 0 | intron | | | |
| SG15S224 | 71956171 | + | 27950 | A/C | rs2304721 | 0.051 | 0.044 | 0.052 | 0.06 | NA | NA | 0.03 | 0.01 | intron | | | |
| SG15S225 | 71956354 | + | 28133 | A/G | rs4497636 | 0.034 | 0.011 | 0.009 | 0 | NA | NA | 0.01 | 0 | intron | | | |
| SG15S180 | 71957323 | + | 29102 | A/G | rs750460 | 0.347 | 0.458 | 0.6 | 0.738 | NA | NA | 0.5 | 0.14 | intron | | | |
| SG15S175 | 71957441 | + | 29220 | A/T | rs4243042 | 0.653 | 0.541 | 0.4 | 0.262 | NA | NA | 0.5 | 0.14 | intron | | | |
| SG15S226 | 71957496 | + | 29275 | C/T | NA | 0.966 | 0.982 | 0.978 | 0.98 | NA | NA | 0.04 | 0 | intron | | | |
| SG15S227 | 71957566 | + | 29345 | C/T | rs2304722 | 0.271 | 0.182 | 0.145 | 0.06 | NA | NA | 0.39 | 0.01 | intron | | | |
| SG15S228 | 71957884 | + | 29663 | A/G | NA | 0 | 0.015 | 0.027 | 0.04 | NA | NA | 0.01 | 0 | intron | | | |
| SG15S268 | 71957932 | + | 29711 | A/G | NA | 0 | 0 | 0.002 | 0 | NA | NA | 0 | 0 | intron | | | |
| SG15S187 | 71959063 | + | 30842 | C/T | rs12437465 | 0.549 | 0.479 | 0.347 | 0.167 | NA | NA | 0.3 | 0.22 | intron | | | |
| SG15S269 | 71960015 | + | 31794 | A/G | NA | 0 | 0.003 | 0.002 | 0 | NA | NA | 0 | 0 | 3′ UTR | | | |
| SG15S229 | 71960095 | + | 31874 | C/G | rs8818 | 0.153 | 0.15 | 0.177 | 0.12 | NA | NA | 0.01 | 0.04 | 3′ UTR | | | |
| SG15S230 | 71960130 | + | 31909 | G/T | NA | 0.992 | 1 | 1 | 1 | NA | NA | 0 | 0.01 | 3′ UTR | | | |
| SG15S231 | 71960161 | + | 31940 | C/T | rs3522 | 0.472 | 0.5 | 0.554 | 0.52 | NA | NA | 0.14 | 0 | 3′ UTR | | | |
| SG15S234 | 71960409 | + | 32188 | C/T | NA | 0.847 | 0.839 | 0.887 | 0.88 | NA | NA | 0.06 | 0.01 | downstream | | | |
| SG15S235 | 71960427 | + | 32206 | A/G | rs7173049 | 0.814 | 0.823 | 0.825 | 0.9 | NA | NA | 0 | 0.18 | downstream | | | |
| SG15S271 | 71960454 | + | 32233 | A/G | NA | 0 | 0 | 0.002 | 0 | NA | NA | 0 | 0 | downstream | | | |
| SG15S236 | 71960695 | + | 32474 | A/G | NA | 0.992 | 0.983 | 0.995 | 1 | NA | NA | 0.01 | 0 | downstream | | | |
| SG15S237 | 71960719 | + | 32498 | A/G | NA | 0.025 | 0.028 | 0.023 | 0.022 | NA | NA | 0.06 | 0 | downstream | | | |
| DG15S1559 | 71960743 | + | 32522 | G indel* | NA | 0.008 | 0 | 0 | 0 | NA | NA | 0 | 0 | downstream | | | |
| SG15S238 | 71960811 | + | 32590 | C/T | NA | 1 | 1 | 1 | 1 | NA | NA | 0 | 0 | downstream | | | |
| SG15S239 | 71960824 | + | 32603 | G/T | rs7175324 | 0.576 | 0.534 | 0.472 | 0.478 | NA | NA | 0.15 | 0.02 | downstream | | | |

By sequencing all exons of the LOXL1 gene and their flanking sequences, as well as the 5′ promoter and 3′ regions in a total of 94 individuals from the HapMap CEU, JPT, CHB and YRI samples (CEU; see http://www.hapmap.org for details on these populations), 94 Icelandic control samples, 94 glaucoma patients and 94 patients with XFS, we identified several novel polymorphisms. Their position in NCBI Build 34, the type of amino acid change, the allelic frequency of the polymorphism, the location in the gene, the odds ratio (OR) of association to XFS, the p-value of the association and the correlation coefficient between the polymorphism and marker rs2165241 is indicated in the table.
*Single base insertion deletion polymorphism.

### EXAMPLE 2

### Common Sequence Variants in the LOXL1 Gene Confer Susceptibility to Exfoliation Glaucoma

Glaucoma is the second most common cause of blindness in the world (Resnikoff, S., et al., Bull World Health Organ 82, 844 (November, 2004)). Its pathophysiology is poorly understood and there is a compelling need for improved risk assessment and better treatment.

Glaucoma is a heterogeneous group of disorders that share a distinct optic nerve damage. In most populations open angle glaucoma (OAG), characterized by painless loss of vision, constitutes the majority of glaucoma cases and is defined as a progressive loss of optic disc neuroretinal rim tissue and consequent excavation of the optic disc with corresponding loss of visual field (Foster, P. J. et al., Br J Opthalmol 86, 238 (February, 2002); Jonasson, F. et al., Eye 17, 747 (August, 2003)). OAG may be divided into primary open angle glaucoma (POAG) and secondary glaucoma. POAG is without an identifiable cause of aqueous outflow resistance, whereas in secondary glaucoma the outflow resistance is of a known cause and in XFG it is considered to be due to the exfoliative material from which the syndrome derives its name. Exfoliation syndrome (XFS) is characterized by accumulation of abnormal microfibrillar deposits that line the aqueous bathed surfaces of the anterior segment of the eye. The prevalence of XFS increases with age and a number of articles have pointed to a geographical clustering of XFS although this condition is found worldwide (Ringvold, A. Acta Opthalmol Scand 77, 371 (1999)). XFS is the most common identifiable cause of secondary glaucoma in most populations and is characterized by rapid progression, high resistance to medical therapy, and a worse prognosis than in POAG (Schlotzer-Schrehardt, U. et al., Am J Opthalmol 141, 921 (2006)).

Family history is an important risk factor for both POAG and XFS which, together with ethnic differences in prevalence of POAG, points to a role of genetic factors in the risk of these conditions (Hewitt, A. W. et al., Clin Experiment Opthalmol 34, 472 (2006)). Three genes, MYOC (Stone, E. M. et al., Science 275, 668 (1997)), OPTN (Rezaie, T. et al., Science 295, 1077 (2002)), and WDR36 (Monemi, S. et. al., M.: Hum. Molec. Genet. 14: 725 (2005)), have been found to be mutated among POAG patients. However, mutations in these genes are of moderate frequency and thus explain only a small fraction of the POAG cases ((Hewitt, A. W. et al., Clin Experiment Opthalmol 34, 472 (2006)).

With the aim of identifying sequence variants that confer risk of glaucoma, we conducted a genome-wide association study on Icelandic patients with glaucoma, using the Illumina Hap300 chip. After quality filtering, 304,250 SNPs were tested for association to glaucoma in a sample of 195 cases and 14,474 population controls. The results were adjusted for relatedness between individuals and potential population stratification using the method of genomic control (Devlin, B. & Roeder, K. Biometrics 55, 997 (1999)). Specifically, the chi-square statistics were divided by an adjustment factor of 1.055.

### Results and Discussion

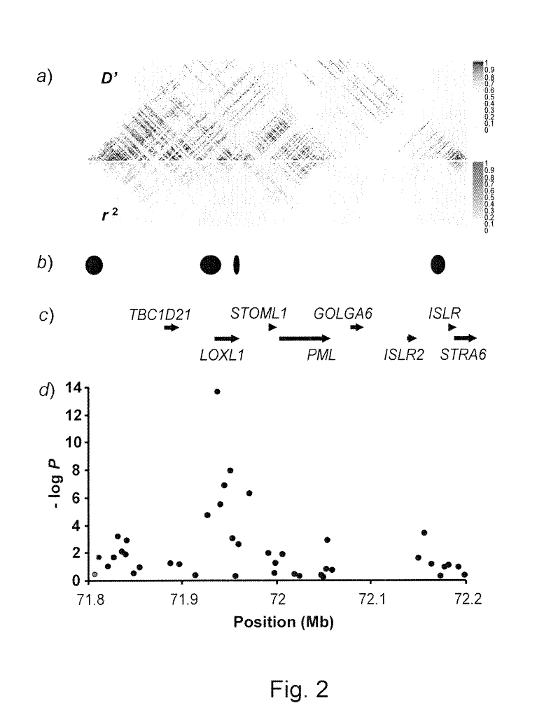

Overall, three SNPs achieved genome-wide significance (P<1.6×10⁻⁷) and are all located within a small region in strong linkage disequilibrium on chromosome 15q24.1 (FIG. 2). The strongest association to glaucoma was observed to allele T of rs2165241 (Table 7) with an odds ratio of 2.28 (P=2.0×10⁻¹⁴). Also genome-wide significant are allele C of rs2304719 (OR=2.07, P=1.2×10⁻⁸) and allele A of rs893817 (OR=1.85, P=1.4×10⁻⁷), but they are both substantially correlated with rs2165241, and are no longer significant (P>0.05) after adjusting for the effect of rs2165241.

The 195 glaucoma cases included 90 cases classified as POAG, 75 known XFG cases, and 30 cases that did not have a precise classification. Further analysis showed that the estimated effect of rs2165241 was weak and only marginally significant for POAG (OR=1.36, P=0.040), but very strong for XFG (OR=3.40, P=4.3×10⁻¹²) (Table 7). In an attempt to replicate the observed association, we genotyped rs2165241 in Swedish samples including 200 POAG cases, 199 XFG cases, and 198 controls. No association was seen with POAG (OR=0.83, P=0.18), but association similar to that in the Icelandic samples was observed for XFG (OR=3.78, P=3.1×10⁻¹⁷). Combining the results from the two sample sets using a Mantel-Haenszel model (Mantel, N. & Haenszel, W. J Natl Cancer Inst 22, 719 (1959)) gave an OR of 3.62 (P=1.0×10⁻²⁷) (Table 7).

To further explore the impact of the variant, we genotyped an additional 55 Icelandic XFS cases without glaucoma. Compared to the controls, the OR is 3.18 (P=1.9×10⁻⁸), and the frequency of rs2165241 T in XFS cases without glaucoma is similar to that in XFS cases with glaucoma (P>0.5). These results indicate that the susceptibility variant tagged by rs2165241 T is a major susceptibility variant for XFS and support the notion that the variant confers risk of glaucoma mainly through XFS.

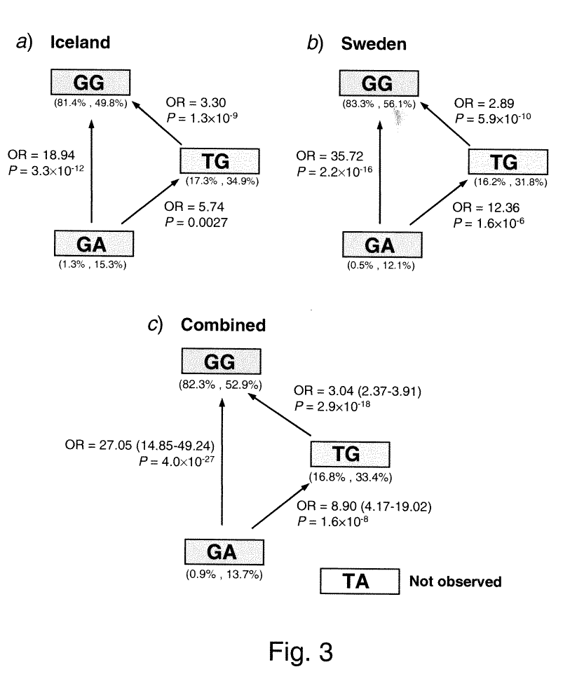

SNP rs2165241 is located in the first intron of the lysyl oxidase like protein 1 (LOXL1) gene. In an attempt to refine the observed association signal, we identified SNPs that are substantially correlated with rs2165241 (r²>0.2) based on the HapMap CEU data and are not part of the Illumina Hap300 chip (Table 3). Eight of those SNPs, in addition to the three best SNPs from the genome-wide scan, were successfully genotyped in all the Icelandic and Swedish XFG cases, in all the Swedish controls and in 647 of the Icelandic controls. Also genotyped were two known non-synonymous SNPs, rs1048661 (Arg→Leu, R141L) and rs3825942 (Gly→Leu, G135D), both located in the first exon of LOXL1. Rs1048661 was identified through the dbSNP database and rs3825942 is a HapMap SNP. Both non-synonymous SNPs showed strong association to XFG (combining Iceland and Sweden, OR=2.46, P=2.3×10⁻¹² for allele G of rs1048661, and OR=20.10, P=3.0×10⁻²¹ for allele G of rs3825942) (Table 7). Further analysis revealed that, while rs2165241 (P=1.0×10⁻²⁷) was more significant than rs1048661 and rs3825942 individually, it was no longer significant (P=0.71) after adjusting for both non-synonymous SNPs simultaneously (Tables 8, 9 and 10), the latter was also true for the other SNPs we typed. Results from investigating the joint effect of rs1048661 and rs3825942 are summarized in FIG. 3. The two SNPs are in substantial linkage disequilibrium (D′=1) and only three of the four possible haplotypes were detected in our samples. Among the three observed haplotypes, (G, A) had the lowest estimated risk. Combining results from Iceland and Sweden, relative to (G, A), (G, G) had an OR of 27.05 (P=4.0×10⁻²⁷) and (T, G) has an OR of 8.90 (P=1.6×10⁻⁸). Allele T of rs2165241 was strongly associated to XFG because it effectively tagged the high risk haplotype (G,G) (r²=0.9). Based on a multiplicative model for the risks of the two risk alleles, allele G of rs1048661 has relative risk of 3.04=27.05/8.90 compared to allele T, and allele G of rs3825942 has relative risk of 27.05 compared to allele A. It is interesting to note that the haplotype (T,A) that was not seen in our samples would be predicted to have even lower risk than (G, A). The three observed haplotypes did not show deviation from Hardy-Weinberg Equilibrium in either the cases or the controls, which is consistent with the model that the risks of the two haplotypes carried by an individual multiply. Under this model, the risk of individuals carrying two copies of the high risk haplotype (G,G) would have about 700 times the risk of those carrying two copies of (G, A), and about 2.47 fold higher than the population average risk. If the risk of the two higher risk haplotypes, (G,G) and (T, G), could be brought down to that of (G, A), it would eliminate more than 99% of the XFG cases. Hence the population attributable risk of the two higher risk haplotypes is over 99%. Sequencing of the seven exons of LOXL1 did not identify further variants associated with the disease (Table 11).

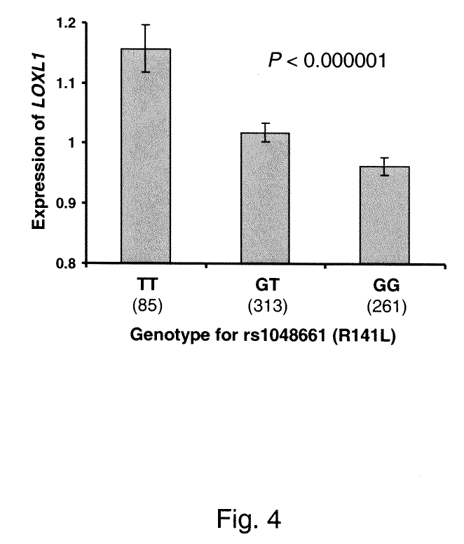

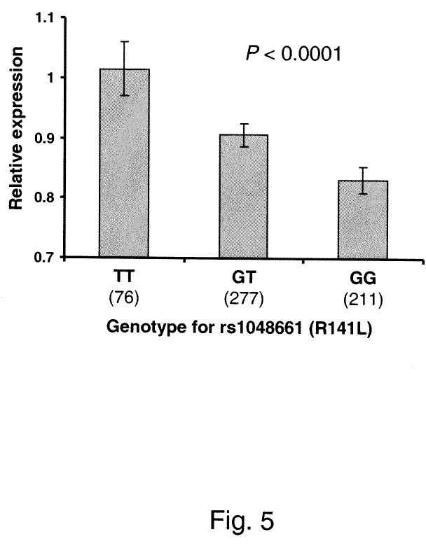

To determine if the risk variants could affect the mRNA expression of LOXL1 we analyzed its expression in adipose tissue from 659 individuals with genotype data for rs1048661 and rs3825942 (microarray expression data). The expression of LOXL1 was reduced by an estimated 7.7% with each copy carried of the risk G allele of rs1048661 (P=8.3×10⁻⁷); this effect was significant for both sexes and did not change if the expression was adjusted for the weight of the individuals (FIG. 4). In contrast, weak positive correlation was observed between the risk G allele of rs3825942 and expression of LOXL1 (P=0.034) and this effect disappeared altogether when the correlation was adjusted for the effect of rs1048661 (P=0.55). The result from the microarray expression data was confirmed with real-time PCR for a subset of 564 of the 659 individuals (FIG. 5).

The LOXL1 gene is a member of the lysyl oxidase family of proteins that catalyses oxidative deamination of lysine residues of tropoelastin which leads to their spontaneous cross-linking with consequential formation of elastin polymer fibers (Liu, X. et al., Nat Genet. 36, 178 (2004), Lucero, H. A. et al., Cell Mol Life Sci 63, 2304 (2006)). Elastogenesis also requires fibrillin-containing microfibrils that act as scaffolds that guide the cross-linking process and deposition of elastine (Thomassin, L. et al, J Biol Chem 280, 42848 (2005)). The lysyl oxidase family members are five and encode the prototypic LOX protein and LOX-like proteins 1 through 4 (LOXL1, LOXL2, LOXL3 and LOXL4). All the five LOX family members have similar exon structure consisting of seven exons, five of which (exons 2-6) exhibit strong homology and encode the C-terminal catalytic domain of these proteins. The sequence difference between the LOX genes resides mainly in exon 1 which encodes pro-peptide that is, following attachment of LOXL1 to the scaffolding structure, cleaved of for catalytic activation of the enzyme. Several studies have demonstrated that the LOXL1 pro-peptide binds to both tropoelastine and fibulin-5 and that these interactions are essential for directing the deposition of the enzyme onto elastic fibers (Liu, X. et al., Nat Genet. 36, 178 (2004), Thomassin, L. et al, J Biol Chem 280, 42848 (2005)).

The pathological process of XFS is characterized by chronic accumulation of abnormal fibrillar material in the anterior segment of the eye, leading to numerous clinical complications apart from secondary glaucoma development. Based on the analysis of the XFS material it has been proposed that XFS arises from abnormal aggregation of elastin microfibrillar components (elastic microfibrillopathy) produced by various intraocular cell types (Schlotzer-Schrehardt, U. et al., Am J Opthalmol 141, 921 (2006), Ritch, R. et al., Prog Retin Eye Res 22, 253 (2003)). Although a role for LOXL1 in the formation of the extracellular matrix of the eye has not been documented, LOXL1 expression is detected in ocular tissues like lamina cribrosa, lens epithelium, cornea, ciliary muscle and trabecular meshwork, all tissues that may be involved in extracellular matrix formation (Kirwan, R. P. et al., Mol Vis 11, 798 (2005), Netland, P. A. et al., Opthalmology 102, 878 (1995), Pena, J. D. et al., Exp Eye Res 67, 517 (1998)), (data accessible at NCBI GEO database). We demonstrate here the association of two coding SNPs, rs1048661 and rs3825942, with XFG that lead to amino acid change at position 141 (Arg to Leu) and 153 (Gly to Asp), respectively, both of which are located in the N-terminal pro-peptide. Based on the functional role of the pro-peptide these alterations could affect both the catalytic activity of the protein through modifications of pro-peptide cleavage and the binding to substrates like tropoelastine and fibulin-5. In addition, we demonstrated that the risk allele of rs1048661 associates with lower levels of the LOXL1 mRNA in adipose tissue. This effect could be mediated through its linkage disequilibrium to non-coding regulatory elements or through its own effect on mRNA stability or processing as previously documented for both synonymous and non-synonymous coding mutations in the genes like DRD1, MDR1 and OPRM1 (Duan, J. et. al., Hum Molec Genet. 12, 205 (2003), Wand, D. et. al., Pharmacogenet Genomics 15, 693 (2005), Zhang, H. et. al., Hum Molec Genet. 15, 807 (2006)). Assuming a similar regulatory network for LOXL1 expression in adipose and ocular tissues, these data suggest that inadequate levels of LOXL1 could predisposes to XFS.

In summary, we have demonstrated in two independent study groups that two non-synonymous changes in exon 1 of the LOXL1 gene on chromosome 15q24.1 associate to XFG. In Iceland and Sweden, the high risk haplotype is very common with a frequency that averages to about 50% in the general population. Approximately 25% of the individuals in the general population are homozygous for the haplotype with the highest risk and their risk of suffering XFG is estimated to be about 700 times greater than those carrying only the low risk haplotype, or about 2.47 times of the population average. Jointly, the two non-synonymous changes account for over 99% of all XFG cases. The product of the LOXL1 gene modifies elastin fibers that are a major constituent of the intraocular lesions in XFG. As to other forms of glaucoma, after removing the SNPs in the LOXL1 region, the genome-scan Q-Q plots for POAG and glaucoma overall cannot be distinguished from that resulting from random noise which might suggest that POAG is a more complex disease than XFG.

### Methods

Subjects from Iceland

Subjects with exfoliation syndrome (XFS) were recruited from the Reykjavik Eye Study (RES), a random sample from the National Population Census (Jonasson F., et al. Eye 17, 747-753 (2003)). All participants had maximal dilation of the pupils and XFS specifically looked for. The definition of XFS included those only found on slitlamp examination to have exfoliative material on the anterior lens capsule and no evidence of glaucomatous optic neuropathy nor glaucomatous visual field defect (Jonasson F., et al. Eye 17, 747-753 (2003); Foster P. J., et al. Br. J. Opthalmol. 86, 238-242 (2002); Wolfs R. C. V., et al. Invest. Opthalmol. Vis. Sci. 41, 3309-3321 (2000)). Subjects with exfoliation glaucoma (XFG) and primary open angle glaucoma (POAG) were recruited from the RES and from a list compiled by Icelandic ophthalmologists. The RES used the same definition for exfoliation as above and grading of stereoscopic fundus photographs to determine glaucomatous optic neuropathy (GON). The definition of GON included vertical cup to disc ratio and cup to disc asymmetry of ≧97.5^{th} percentile when accompanied with glaucomatous visual field defect (GVFD) and ≧99.5^{th} percentile when reliable GVFD could not be obtained. The diagnostic criteria used by the ophthalmologists included glaucomatous damage to the optic nerve head and/or glaucomatous damage to the visual field. Judging the optic nerve damage was done by the examining physician. Correspondingly, different perimetric methods were used and grading of the fields based on the examining physician's evaluation.

The 14474 controls used for the genome-wide association study were recruited as a part of various genetic programs at deCODE. The medical history of the controls is unknown. The breakdown of the control group into the various genetic programs is approximately (with the corresponding frequency of the T allele of rs2165241 in parenthesis): Type II diabetes 1200 (0.49), Schizophrenia 550 (0.46), Prostate cancer 1300 (0.48), Breast Cancer 1400 (0.48), Colon Cancer 900 (0.45), Addiction 2800 (0.47), Anxiety 900 (0.47), Infectious diseases 1500 (0.48), Population Controls 550 (0.46), Myocardial Infarction 1900 (0.48), Stroke 1500 (0.46), Longevity 1300 (0.46), Migraine 150 (0.49), Restless Leg Syndrome 150 (0.49). Note, some individuals have participated in more than one genetic program. Most importantly, no significant differences in frequencies are observed among the disease groups that make up the control group (P=0.65).

Ethical approval for the study was granted by the National Bioethics Committee and the Icelandic Data Protection Authority. All participants in the study signed informed consent. All personal identifiers associated with blood samples, clinical information and genealogy were encrypted by the Data Protection Authority, using a third-party encryption system in which the Data Protection Authority maintains the code.

Subjects from Sweden

Patients were recruited from the out-patient clinic at the Department of Opthalmology, University Hospital, Uppsala, and the Department of Opthalmology, Tierps Hospital, Tierp, located close to Uppsala. After obtaining informed consent, samples of peripheral blood were collected from 200 unrelated patients diagnosed with POAG and 200 unrelated patients with exfoliative glaucoma. Diagnostic criteria included increased IOP and glaucomatous damage to the optic nerve head and/or glaucomatous damage to the visual field. Diagnoses were obtained from the patients' medical records. The grading of the optic nerve head damage, therefore, had been performed by the patients' treating and examining physician. Correspondingly, many different perimetric methods for visual field testing were used, and the grading of the fields was based on the examining physicians' evaluation. At the two clinics involved in this study, dilation of the pupils and gonioscopy are standard procedures for the diagnosis of glaucoma. The presence of exfoliative material on, e.g. the iris or lens was needed for the diagnosis of exfoliation glaucoma. None of the patients in the POAG and the exfoliation glaucoma groups were related. An additional 200 samples were collected from control individuals matched for age, sex, and geographic and ethnic origin, in whom glaucoma had been excluded using IOP measurements and opthalmoscopy of the optic disc. The study was approved by the local Research Ethics Committee and performed according to the Declaration of Helsinki.

### Illumina Genome-Wide Genotyping

All Icelandic case- and control-samples were assayed with the Infinium HumanHap300 SNP chips (Illumina, San Diego, Calif., USA), containing 317,503 haplotype tagging SNPs derived from phase I of the International HapMap project. Of the SNPs assayed on the chip, 13,253 SNPs were excluded if they had (a) yield lower than 95% in cases or controls, (b) minor allele frequency less than 1% in the population, or (c) showed significant deviation from Hardy-Weinberg equilibrium in the controls (P<0.001). Any samples with a call rate below 98% were excluded from the analysis. The final analysis includes 304,250 SNPs.

### Single SNP Genotyping.

Single SNP genotyping for all samples was carried out at deCODE genetics in Reykjavik, Iceland applying the same platform to all populations studied. The genotyping was carried out using the Centaurus (Nanogen) platform (Kutyavin, I. V. et al. Nucleic Acids Research 34, e128 (2006)).

The quality of each Centaurus SNP assay was evaluated by genotyping each assay on the CEU samples and comparing the results with the HapMap data. Assays with >1.5% mismatch rate were excluded and a linkage disequilibrium (LD) test was used for markers known to be in LD. Key markers from the genome-wide analysis were re-genotyped on more than 10% of samples and a mismatch was observed in less than 0.5% of samples.

### Association Analysis

For association analysis we utilized a standard likelihood ratio statistic, implemented in the NEMO software (Gretarsdottir, S. et al. Nat Genet. 35, 131-8 (2003))

to calculate two-sided P values and odds ratio (OR) for each individual allele, assuming a multiplicative model for risk, i.e., that the risk of the two alleles a person carries multiply (Rice, J.A. Wadsworth Inc., Belmont, Calif., 1995)). Allelic frequencies, rather than carrier frequencies are presented for the markers, and P values are given after adjustment for the relatedness of the subjects. When estimating genotype specific OR (Table 7), genotype frequencies in the population were estimated assuming HWE.

In general, allele and haplotype frequencies are estimated by maximum likelihood and tests of differences between cases and controls are performed using a generalized likelihood ratio test⁶. This method is particularly useful in situations where there are some missing genotypes for the marker of interest and genotypes of another marker, which is in strong LD with the marker of interest, are used to provide some partial information. This was used in the association tests presented in Tables 7, 12, 13 and 14, to ensure that the comparison of the highly correlated markers was done using the same number of individuals. To handle uncertainties with phase and missing genotypes, maximum likelihood estimates, likelihood ratios and P values are computed directly for the observed data, and hence the loss of information due to uncertainty in phase and missing genotypes is automatically captured by the likelihood ratios.

Results from multiple case-control groups were combined using a Mantel-Haenszel model (Mantel, N. & Haenszel, W. J. Natl. Cancer Inst. 22, 719-748 (1959)) in which the groups were allowed to have different population frequencies for alleles, haplotypes and genotypes but were assumed to have common relative risks.

### Correction for Relatedness of the Subjects and Genomic Control

Some of the individuals in both the Icelandic patient and control groups are related to each other, causing the chi-square test statistic to have a mean >1 and median>0.675. We estimated the inflation factor for the genome-wide association by calculating the average of the 304,250 chi-square statistics, which was a method of genomic control (Devlin, B. & Roeder, K. Biometrics 55, 997-1004 (1999)) to adjust for both relatedness and potential population stratification. The inflation factor was estimated as 1.055 and the results presented from the genome-wide association and in Tables 7 and 12 are based on adjusting the chi-square statistics by dividing each of them by 1.055. For comparison we also used a previously described procedure where we simulated genotypes through the genealogy of 708,683 Icelanders to estimate the adjustment factor (Stefansson, H. et al. Nat Genet. 37, 129-37 (2005)). The corresponding adjustment factor was 1.030. As the adjustment factors from the simulation are smaller than estimated using the method of genomic control, we used the latter as a more conservative estimate. The adjustment factor for the subgroups of 75×FG cases, the 90 POAG cases and the 55 XFS cases without glaucoma, was estimated using simulation and were 1.016, 1.007 and 1.003, respectively.

### Sequencing of LOXL1

All seven exons of the LOXL1 gene were sequenced in 277 Icelandic control samples, 89 samples from the HapMap CEU population and 140 Icelandic glaucoma cases, including 25 cases with XFG. For part of exon 1, 180 samples from the HapMap YRI and CHB/JPT populations were also sequenced. PCR amplifications and sequencing reactions were set up on Zymark SciClone ALH300 robotic workstations and amplified on MIR Tetrads. PCR products were verified for correct length by agarose gel electrophoresis and purified using AMPure (Agencourt Bioscience). Purified products were sequenced using an ABI PRISM Fluorescent Dye Terminator system, repurified using CleanSEQ (Agencourt), and resolved on Applied Biosystems 3730 capillary sequencers. SNP calling from primary sequence data was carried out using deCODE Genetics Sequence Miner software. All LOXL1 variants identified by the automated systems were confirmed by manual inspection of primary signal traces.

### Correlation Between Genotype and Expression of LOXL1 in Adipose Tissue

Subcutaneous fat samples (5-10 cm3) were removed through a 3 cm incision at the bikini line (always from the same site to avoid site-specific variation) after local anesthesia using 10 ml of lidocaine-adrenalin (1%) from 659 individuals. Purification of the total RNA was performed with the RNeasy Mini Kit (QIAGEN GmbH, Hilden, Germany). Integrity of the total RNA was assessed through analysis on the Agilent 2100 Bioanalyzer (Agilent Technologies, Palo Alto, U.S., CA). Each labeled RNA sample including reference pools, was hybridized to a Human 25K array manufactured by Agilent Technologies. Array images were processed as described previously to obtain background noise, single-channel intensity and associated measurement error estimates (Monks, S. A. et al., Am J Hum Genet. 75, 1094-105 (2004)). Expression changes between two samples were quantified as mean logarithm (log₁₀) expression ratio (MLR), i.e. expression ratios compared to background corrected intensity values for the two channels for each spot on the arrays (Schadt, E. E. et. al. Nature 422, 297-302 (2003)). The hybridizations went through the standard QC process, i.e. signal to noise ratio, reproducibility and accuracy at spike-in compounds. The probe used to test the expression of LOXL1 resides in the 3′ un-translated region of the gene (position 71957636-71957696 in Build 34).

The correlation between MLR for LOXL1 and genotypes of the two non-synonymous SNPs, rs1048661 and rs3825942, was tested by regressing the MLR's on the number of copies of the at-risk G alleles. The effect of age and sex was taken into account by including an Age×Sex term as an explanatory variable in the regression analysis. To adjust for the weight of the individuals their body mass index (BMI) was included as an explanatory variable. All P-values were adjusted for relatedness of the individuals by simulating genotypes through the Icelandic genealogy as previously described.

### TaqMan Verification and Replication of DNA Microarray Results

Total RNA was converted to cDNA using the High Capacity cDNA Archive Kit (Applied Biosystems), primed with random hexamers. A TaqMan assay for the gene expression analysis of the LOXL1 gene was designed using Primer Express software (Applied Biosystems; forward primer: TGTGCTGCGGAGGAGAAGT, Reverse primer: ATCGTAGTCGGTGGCCTCAG and MGB probe: 6FAM-GGCCAGCACAGCC). Real-time PCR was carried out according to manufacturer's recommendations on an ABI Prism 7900HT Sequence Detection System. Quantification was performed using the ΔΔCt method (User Bulletin no. 2, Applied Biosystems 2001) using Human GUS (Applied Biosystems) for normalizing input cDNA.

LOXL1 Expression Data from the NCBI Geo Database.

The expression of LOXL1 in ocular tissue was verified by analyzing data for the following tissues available in the NCBI GEO database; lamina cribrosa (GEO accession number GDS1313), lens epithelium (GDS1327), cornea (GDS3023), ciliary muscle (GDS3359), and trabecular meshwork (GDS3359).

TABLE 7
Association between exfoliation glaucoma and rs2165241, rs1048661 and rs3825942.
Shown is the association of the risk alleles of the SNP rs2165241 and of the two non-synonymous SNPs, rs1048661 and rs3825942,
to glaucoma in the Icelandic discovery case-control group, the Swedish replication case-control group and the two groups combined.
Results are shown for all glaucoma cases and for primary open angle glaucoma (POAG) cases,
exfoliation glaucoma (XFG) and exfoliation without glaucoma separately. Study population includes
the number of individuals (n). The results include the OR, 95% CI and P values assuming the
multiplicative model. For the Icelandic case-control group, the P values and CI were adjusted
for relatedness as described in the methods. For the combined group we
calculated OR and P values using a Mantel-Haenszel model.

| Study | | | | | | | | | |
|---|---|---|---|---|---|---|---|---|---|
| population | rs2165241 T | | | rs1048661 (R141L) G | | | rs3825942 (G153D) G | | |
| (n) | Frq. | OR (95% CI) | P | Frq. | OR (95% CI) | P | Frq. | OR (95% CI) | P |
| Iceland | | | | | | | | | |
| Controls | 0.473 | | | 0.651 | | | 0.847 | | |
| (14474) | | | | | | | | | |
| Glaucoma | 0.672 | 2.28 (1.85-2.82) | 2.0 × 10⁻¹⁴ | 0.777 | 1.87 (1.49-2.35) | 7.4 × 10⁻⁸ | 0.936 | 2.66 (1.86-3.80) | 7.9 × 10⁻⁸ |
| Combined | | | | | | | | | |
| (195) | | | | | | | | | |
| POAG | 0.550 | 1.36 (1.01-1.83) | 0.04 | 0.711 | 1.32 (0.96-1.82) | 0.085 | 0.872 | 1.25 (0.81-1.91) | 0.32 |
| (90) | | | | | | | | | |
| XFG (75) | 0.753 | 3.40 (2.41-4.81) | 4.3 × 10⁻¹² | 0.827 | 2.56 (1.74-3.77) | 1.8 × 10⁻⁶ | 0.987 | 13.23 (5.59-31.29) | 4.1 × 10⁻⁹ |
| XF wout | 0.740 | 3.18 (2.12-4.76) | 1.9 × 10⁻⁸ | 0.789 | 2.02 (1.32-3.09) | 1.3 × 10⁻³ | 0.982 | 10.10 (4.02-25.36) | 8.5 × 10⁻⁷ |
| Glaucoma | | | | | | | | | |
| (55) | | | | | | | | | |
| Sweden | | | | | | | | | |
| Controls | 0.535 | | | 0.682 | | | 0.879 | | |
| (198) | | | | | | | | | |
| Glaucoma | 0.649 | 1.61 (1.26-2.05) | 0.00016 | 0.737 | 1.31 (1.00-1.70) | 0.048 | 0.929 | 1.79 (1.19-2.70) | 0.0052 |
| Combined | | | | | | | | | |
| (399) | | | | | | | | | |
| POAG | 0.488 | 0.83 (0.63-1.09) | 0.18 | 0.638 | 0.82 (0.61-1.10) | 0.19 | 0.863 | 0.87 (0.57-1.31) | 0.49 |
| (200) | | | | | | | | | |
| XFG | 0.813 | 3.78 (2.77-5.14) | 3.1 × 10⁻¹⁷ | 0.834 | 2.39 (1.72-3.34) | 2.7 × 10⁻⁷ | 0.995 | 27.28 (11.44-65.07) | 9.1 × 10⁻¹⁴ |
| (199) | | | | | | | | | |
| Combined | | | | | | | | | |
| Controls | | | | | | | | | |
| (14672) | | | | | | | | | |
| Glaucoma | | 1.96 (1.67-2.29) | 1.3 × 10⁻¹⁶ | | 1.59 (1.35-1.89) | 7.5 × 10⁻⁸ | | 2.20 (1.69-2.85) | 3.4 × 10⁻⁹ |
| Combined | | | | | | | | | |
| (594) | | | | | | | | | |
| POAG | | 1.04 (0.85-1.28) | 0.67 | | 1.02 (0.83-1.27) | 0.83 | | 1.04 (0.78-1.39) | 0.81 |
| (291) | | | | | | | | | |
| XFG | | 3.62 (2.87-4.55) | 1.0 × 10⁻²⁷ | | 2.46 (1.91-3.16) | 2.3 × 10⁻¹² | | 20.10 (10.80-37.41) | 3.0 × 10⁻²¹ |
| (274) | | | | | | | | | |

TABLE 8
Association between XFG and additional markers typed in LOXL1.
Association between exfoliation glaucoma (XFG) and thirteen SNPs, including rs2165241 and the two non-synonymous SNPs: rs1048661
(R141L) and rs3825942 (G153D), typed in LOXL1. The association is calculated for the Icelandic and the Swedish XFG cases and controls and for
the two populations combined using a Mantel-Haenszel model. Included is the correlation r² between all the markers and the haplotype that
carries the risk alleles of both of the exonic SNPs, rs1048661 (G) and rs3825942 (G), calculated for the Icelandic (IC) and the Swedish (SW)
control group, together with P values corresponding to a test of each of the SNPs for association to XFG conditional on the observed association
of the two non-synonymous SNPs, rs1048661 and rs3825942.

| | | | Combined (273/14672) | | | | | Iceland (75/14474) | | | |
|---|---|---|---|---|---|---|---|---|---|---|---|
| | | | r^{2a} | | | | | Frequency | | | |
| SNP | Allele | Position | IC | SW | OR (95% Cl) | P | P_{adj}^{b} | Control | Cases | OR (95% Cl) | P |
| rs4461027 | C | 71927365 | 0.41 | 0.26 | 1.69 (1.37-2.09) | 1.2 × 10⁻⁶ | 0.16 | 0.392 | 0.560 | 1.97 (1.43-2.72) | 0.000036 |
| rs1048661 | G | 71935363 | 0.53 | 0.60 | 2.46 (1.91-3.16) | 2.3 × 10⁻¹² | — | 0.651 | 0.827 | 2.56 (1.74-3.77) | 1.8 × 10⁻⁶ |
| rs3825942 | G | 71935399 | 0.18 | 0.18 | 20.1 (10.8-37.4) | 3.0 × 10⁻²¹ | — | 0.847 | 0.987 | 13.2 (5.6-31.3) | 4.1 × 10⁻⁹ |
| rs2165241 | T | 71938019 | 0.88 | 0.90 | 3.62 (2.87-4.55) | 1.0 × 10⁻²⁷ | 0.71 | 0.473 | 0.753 | 3.40 (2.41-4.80) | 4.3 × 10⁻¹² |
| rs1992314 | C | 71939584 | 0.53 | 0.60 | 2.44 (1.90-3.15) | 3.9 × 10⁻¹² | 0.54 | 0.654 | 0.827 | 2.53 (1.72-3.72) | 2.6 × 10⁻⁶ |
| rs2028386 | C | 71942525 | 0.95 | 0.98 | 3.73 (2.95-4.71) | 3.9 × 10⁻²⁸ | 0.26 | 0.490 | 0.780 | 3.69 (2.60-5.25) | 3.4 × 10⁻¹³ |
| rs4337252 | G | 71942582 | 0.95 | 0.98 | 3.71 (2.94-4.70) | 5.7 × 10⁻²⁸ | 0.26 | 0.491 | 0.780 | 3.67 (2.58-5.22) | 4.5 × 10⁻¹³ |
| rs4886782 | A | 71944627 | 0.48 | 0.46 | 2.19 (1.76-2.71) | 8.2 × 10⁻¹³ | 1.00 | 0.327 | 0.547 | 2.47 (1.79-3.43) | 5.1 × 10⁻⁸ |
| rs893817 | A | 71944882 | 0.46 | 0.47 | 2.14 (1.69-2.71) | 3.5 × 10⁻¹⁰ | 0.65 | 0.633 | 0.807 | 2.42 (1.66-3.52) | 3.8 × 10⁻⁶ |
| rs12440667 | T | 71947256 | 0.84 | 0.78 | 3.35 (2.67-4.19) | 6.1 × 10⁻²⁶ | 0.34 | 0.461 | 0.740 | 3.33 (2.37-4.69) | 5.2 × 10⁻¹² |
| rs2304719 | C | 71951317 | 0.38 | 0.39 | 4.00 (2.96-5.41) | 2.2 × 10⁻¹⁹ | 0.87 | 0.695 | 0.887 | 3.43 (2.22-5.30) | 2.7 × 10⁻⁸ |
| rs750460 | T | 71957323 | 0.84 | 0.78 | 3.25 (2.60-4.07) | 4.5 × 10⁻²⁵ | 0.54 | 0.457 | 0.740 | 3.37 (2.40-4.74) | 2.7 × 10⁻¹² |
| rs4243042 | T | 71957441 | 0.84 | 0.78 | 3.25 (2.60-4.07) | 4.6 × 10⁻²⁵ | 0.54 | 0.456 | 0.740 | 3.38 (2.40-4.75) | 2.5 × 10⁻¹² |

| | | | Sweden (199/198) | | | |
|---|---|---|---|---|---|---|
| | | | Frequency | | | |
| SNP | Allele | Position | Control | Cases | OR (95% Cl) | P |
| rs4461027 | C | 71927365 | 0.441 | 0.543 | 1.51 (1.14-1.99) | 0.0041 |
| rs1048661 | G | 71935363 | 0.682 | 0.834 | 2.39 (1.72-3.34) | 2.7 × 10⁻⁷ |
| rs3825942 | G | 71935399 | 0.879 | 0.995 | 27.3 (11.4-65.1) | 9.1 × 10⁻¹⁴ |
| rs2165241 | T | 71938019 | 0.535 | 0.813 | 3.78 (2.77-5.14) | 3.1 × 10⁻¹⁷ |
| rs1992314 | C | 71939584 | 0.682 | 0.837 | 2.39 (1.72-3.34) | 2.7 × 10⁻⁷ |
| rs2028386 | C | 71942525 | 0.556 | 0.824 | 3.75 (2.74-5.12) | 1.2 × 10⁻¹⁶ |
| rs4337252 | G | 71942582 | 0.556 | 0.824 | 3.75 (2.74-5.12) | 1.2 × 10⁻¹⁶ |
| rs4886782 | A | 71944627 | 0.371 | 0.540 | 1.99 (1.50-2.64) | 1.6 × 10⁻⁶ |
| rs893817 | A | 71944882 | 0.636 | 0.776 | 1.98 (1.46-2.70) | 0.000014 |
| rs12440667 | T | 71947256 | 0.497 | 0.769 | 3.36 (2.50-4.52) | 1.2 × 10⁻¹⁵ |
| rs2304719 | C | 71951317 | 0.747 | 0.930 | 4.46 (2.96-6.73) | 8.3 × 10⁻¹³ |
| rs750460 | T | 71957323 | 0.497 | 0.759 | 3.18 (2.37-4.27) | 1.6 × 10⁻¹⁴ |
| rs4243042 | T | 71957441 | 0.497 | 0.759 | 3.18 (2.37-4.27) | 1.6 × 10⁻¹⁴ |

^{a}Correlation with the at-risk haplotype rs1048661 (G) rs3825942 (G) in Icelandic (IC) and Swedish (SW) controls.
^{b}Adjusted P values when the association is tested conditional on the observed association to the two SNPs, rs1048661 and rs3825942.

TABLE 9
Conditional association between XFG and additional markers typed in LOXL1.
Association between exfoliation glaucoma (XFG) and thirteen SNPs, including rs2165241 and the two non-synonymous SNPs
rs1048661 and rs3825942, typed in LOXL1. The association is calculated for the combined Icelandic and Swedish
case-control groups and for each SNP the association is tested conditional on the observed association to each
of the other SNPs.

| | | | Adjusted P values | | | | | | |
|---|---|---|---|---|---|---|---|---|---|
| SNP | Allele | Position | rs4461027 | rs1048661 | rs3825942 | rs2165241 | rs1992314 | rs2028386 | rs4337252 |
| rs4461027 | C | 71927365 | — | 0.033 | 0.0019 | 0.52 | 0.031 | 0.25 | 0.26 |
| rs1048661 | G | 71935363 | 4.7 × 10⁻⁸ | — | 4.5 × 10⁻¹⁸ | 0.008 | 0.27 | 0.00036 | 0.00046 |
| rs3825942 | G | 71935399 | 4.5 × 10⁻¹⁸ | 6.8 × 10⁻²⁷ | — | 1.5 × 10⁻⁹ | 6.3 × 10⁻²⁷ | 3.9 × 10⁻⁹ | 2.5 × 10⁻⁹ |
| rs2165241 | T | 71938019 | 8.2 × 10⁻²³ | 1.0 × 10⁻¹⁸ | 2.2 × 10⁻¹⁶ | — | 4.1 × 10⁻¹⁹ | 0.15 | 0.12 |
| rs1992314 | C | 71939584 | 6.5 × 10⁻⁸ | 0.48 | 5.9 × 10⁻¹⁸ | 0.0043 | — | 0.00015 | 0.00017 |
| rs2028386 | C | 71942525 | 3.7 × 10⁻²³ | 4.0 × 10⁻²⁰ | 4.2 × 10⁻¹⁶ | 0.098 | 1.2 × 10⁻²⁰ | — | 0.25 |
| rs4337252 | G | 71942582 | 6.2 × 10⁻²³ | 8.3 × 10⁻²⁰ | 4.4 × 10⁻¹⁶ | 0.14 | 2.2 × 10⁻²⁰ | 0.54 | — |
| rs4886782 | A | 71944627 | 2.8 × 10⁻⁸ | 0.000029 | 3.0 × 10⁻⁷ | 0.89 | 0.000027 | 0.85 | 0.8 |
| rs893817 | A | 71944882 | 6.7 × 10⁻⁶ | 0.97 | 1.1 × 10⁻¹⁵ | 0.099 | 0.97 | 0.026 | 0.028 |
| rs12440667 | T | 71947256 | 4.6 × 10⁻²¹ | 1.9 × 10⁻¹⁵ | 2.0 × 10⁻¹⁵ | 0.34 | 1.2 × 10⁻¹⁵ | 0.11 | 0.088 |
| rs2304719 | C | 71951317 | 8.8 × 10⁻¹⁵ | 7.7 × 10⁻¹⁴ | 2.3 × 10⁻⁶ | 0.0059 | 7.2 × 10⁻¹⁴ | 0.014 | 0.012 |
| rs750460 | T | 71957323 | 4.6 × 10⁻²⁰ | 2.0 × 10⁻¹⁴ | 1.2 × 10⁻¹⁴ | 0.63 | 1.3 × 10⁻¹⁴ | 0.23 | 0.19 |
| rs4243042 | T | 71957441 | 4.8 × 10⁻²⁰ | 1.8 × 10⁻¹⁴ | 1.2 × 10⁻¹⁴ | 0.64 | 1.4 × 10⁻¹⁴ | 0.23 | 0.19 |

| | | | Adjusted P values | | | | | |
|---|---|---|---|---|---|---|---|---|
| SNP | Allele | Position | rs4886782 | rs893817 | rs12440667 | rs2304719 | rs750460 | rs4243042 |
| rs4461027 | C | 71927365 | 0.075 | 0.035 | 0.28 | 0.11 | 0.35 | 0.35 |
| rs1048661 | G | 71935363 | 0.0001 | 0.0021 | 0.32 | 1.2 × 10⁻⁶ | 0.53 | 0.57 |
| rs3825942 | G | 71935399 | 1.2 × 10⁻¹⁵ | 1.4 × 10⁻²⁶ | 1.4 × 10⁻¹⁰ | 3.9 × 10⁻⁸ | 1.1 × 10⁻¹⁰ | 1.0 × 10⁻¹⁰ |
| rs2165241 | T | 71938019 | 1.2 × 10⁻¹⁶ | 7.7 × 10⁻²⁰ | 0.0019 | 1.0 × 10⁻¹¹ | 0.00029 | 0.00027 |
| rs1992314 | C | 71939584 | 0.00013 | 0.0031 | 0.27 | 1.6 × 10⁻⁶ | 0.44 | 0.44 |
| rs2028386 | C | 71942525 | 8.4 × 10⁻¹⁷ | 1.7 × 10⁻²⁰ | 0.00054 | 1.6 × 10⁻¹¹ | 0.00011 | 0.000099 |
| rs4337252 | G | 71942582 | 1.3 × 10⁻¹⁶ | 3.1 × 10⁻²⁰ | 0.00073 | 2.2 × 10⁻¹¹ | 0.00015 | 0.00014 |
| rs4886782 | A | 71944627 | — | 0.000013 | 0.66 | 0.00016 | 0.83 | 0.82 |
| rs893817 | A | 71944882 | 0.0072 | — | 0.0018 | 5.9 × 10⁻⁶ | 0.0068 | 0.0065 |
| rs12440667 | T | 71947256 | 9.5 × 10⁻¹⁵ | 1.9 × 10⁻¹⁹ | — | 6.5 × 10⁻¹¹ | 0.037 | 0.036 |
| rs2304719 | C | 71951317 | 3.0 × 10⁻¹¹ | 3.0 × 10⁻¹⁵ | 0.00036 | — | 0.00022 | 0.00021 |
| rs750460 | T | 71957323 | 8.2 × 10⁻¹⁴ | 5.0 × 10⁻¹⁸ | 0.6 | 3.3 × 10⁻¹⁰ | — | 1 |
| rs4243042 | T | 71957441 | 8.9 × 10⁻¹⁴ | 5.3 × 10⁻¹⁸ | 0.61 | 3.5 × 10⁻¹⁰ | 0.74 | — |

TABLE 10
Pair-wise correlation for SNPs typed in and around the LOXL1 gene.
Pair-wise correlation, D′ (lower left corner) and r² (upper right corner), for the thirteen
SNPs in LOXL1 that were tested for association to XFG. The correlation is estimated for
control individuals from the Icelandic and the Swedish study groups, respectively.

| | r² | | | | | | |
|---|---|---|---|---|---|---|---|
| D′ | rs4461027 | rs1048661 | rs3825942 | rs2165241 | rs1992314 | rs2028386 | rs4337252 |
| Iceland | | | | | | | |
| rs4461027 | — | 0.23 | 0.05 | 0.35 | 0.22 | 0.39 | 0.39 |
| rs1048661 | 0.82 | — | 0.13 | 0.45 | 0.98 | 0.49 | 0.48 |
| rs3825942 | 0.71 | 1.00 | — | 0.14 | 0.13 | 0.14 | 0.15 |
| rs2165241 | 0.69 | 0.98 | 1.00 | — | 0.46 | 0.93 | 0.92 |
| rs1992314 | 0.83 | 1.00 | 1.00 | 1.00 | — | 0.49 | 0.49 |
| rs2028386 | 0.76 | 0.98 | 1.00 | 0.99 | 1.00 | — | 0.99 |
| rs4337252 | 0.76 | 0.98 | 1.00 | 1.00 | 1.00 | 1.00 | — |
| rs4886782 | 0.92 | 0.98 | 1.00 | 1.00 | 1.00 | 1.00 | 1.00 |
| rs893817 | 0.80 | 0.99 | 0.89 | 0.94 | 1.00 | 0.94 | 0.94 |
| rs12440667 | 0.68 | 0.98 | 1.00 | 0.98 | 0.99 | 1.00 | 0.99 |
| rs2304719 | 0.80 | 0.20 | 0.95 | 1.00 | 0.20 | 1.00 | 0.99 |
| rs750460 | 0.69 | 0.98 | 1.00 | 1.00 | 1.00 | 1.00 | 1.00 |
| rs4243042 | 0.69 | 0.98 | 1.00 | 1.00 | 1.00 | 1.00 | 1.00 |
| Sweden | | | | | | | |
| rs4461027 | — | 0.11 | 0.07 | 0.21 | 0.11 | 0.27 | 0.27 |
| rs1048661 | 0.55 | — | 0.06 | 0.54 | 1.00 | 0.58 | 0.58 |
| rs3825942 | 0.80 | 1.00 | — | 0.16 | 0.06 | 0.17 | 0.17 |
| rs2165241 | 0.54 | 1.00 | 1.00 | — | 0.54 | 0.92 | 0.92 |
| rs1992314 | 0.55 | 1.00 | 1.00 | 1.00 | — | 0.58 | 0.58 |
| rs2028386 | 0.64 | 1.00 | 1.00 | 1.00 | 1.00 | — | 1.00 |
| rs4337252 | 0.64 | 1.00 | 1.00 | 1.00 | 1.00 | 1.00 | — |
| rs4886782 | 0.89 | 1.00 | 1.00 | 1.00 | 1.00 | 1.00 | 1.00 |
| rs893817 | 0.56 | 1.00 | 1.00 | 0.78 | 1.00 | 0.79 | 0.79 |
| rs12440667 | 0.56 | 1.00 | 1.00 | 1.00 | 1.00 | 1.00 | 1.00 |
| rs2304719 | 0.82 | 0.31 | 0.97 | 1.00 | 0.31 | 1.00 | 1.00 |
| rs750460 | 0.56 | 1.00 | 1.00 | 1.00 | 1.00 | 1.00 | 1.00 |
| rs4243042 | 0.56 | 1.00 | 1.00 | 1.00 | 1.00 | 1.00 | 1.00 |

| | r² | | | | | |
|---|---|---|---|---|---|---|
| D′ | rs4886782 | rs893817 | rs12440667 | rs2304719 | rs750460 | rs4243042 |
| Iceland | | | | | | |
| rs4461027 | 0.63 | 0.23 | 0.34 | 0.19 | 0.35 | 0.36 |
| rs1048661 | 0.25 | 0.93 | 0.44 | 0.03 | 0.44 | 0.44 |
| rs3825942 | 0.07 | 0.10 | 0.13 | 0.30 | 0.14 | 0.13 |
| rs2165241 | 0.55 | 0.43 | 0.96 | 0.41 | 0.97 | 0.97 |
| rs1992314 | 0.24 | 0.94 | 0.45 | 0.04 | 0.44 | 0.44 |
| rs2028386 | 0.51 | 0.46 | 0.92 | 0.43 | 0.91 | 0.91 |
| rs4337252 | 0.51 | 0.46 | 0.90 | 0.44 | 0.90 | 0.90 |
| rs4886782 | — | 0.26 | 0.55 | 0.22 | 0.56 | 0.56 |
| rs893817 | 1.00 | — | 0.44 | 0.03 | 0.43 | 0.43 |
| rs12440667 | 1.00 | 0.96 | — | 0.39 | 0.97 | 0.98 |
| rs2304719 | 1.00 | 0.20 | 0.99 | — | 0.39 | 0.39 |
| rs750460 | 0.99 | 0.95 | 1.00 | 1.00 | — | 1.00 |
| rs4243042 | 0.99 | 0.96 | 1.00 | 1.00 | 1.00 | — |
| Sweden | | | | | | |
| rs4461027 | 0.60 | 0.14 | 0.25 | 0.18 | 0.25 | 0.25 |
| rs1048661 | 0.28 | 0.82 | 0.46 | 0.07 | 0.46 | 0.46 |
| rs3825942 | 0.08 | 0.08 | 0.14 | 0.39 | 0.14 | 0.14 |
| rs2165241 | 0.51 | 0.40 | 0.86 | 0.39 | 0.86 | 0.86 |
| rs1992314 | 0.28 | 0.82 | 0.46 | 0.07 | 0.46 | 0.46 |
| rs2028386 | 0.47 | 0.44 | 0.79 | 0.42 | 0.79 | 0.79 |
| rs4337252 | 0.47 | 0.44 | 0.79 | 0.42 | 0.79 | 0.79 |
| rs4886782 | — | 0.34 | 0.60 | 0.20 | 0.60 | 0.60 |
| rs893817 | 1.00 | — | 0.55 | 0.04 | 0.55 | 0.55 |
| rs12440667 | 1.00 | 0.98 | — | 0.33 | 1.00 | 1.00 |
| rs2304719 | 1.00 | 0.27 | 1.00 | — | 0.33 | 0.33 |
| rs750460 | 1.00 | 0.98 | 1.00 | 1.00 | — | 1.00 |
| rs4243042 | 1.00 | 0.98 | 1.00 | 1.00 | 1.00 | — |

TABLE 11
Exonic variants in the LOXL1 gene.
SNPs identified in exons of LOXL1 (NCBI accession 005576) through the sequencing of 89 CEU HapMap samples, 277 Icelandic control samples
and 140 glaucoma cases, including 25 with XFG. For part of exon 1, 90 YRI and 90 CHB/JPT HapMap samples were also sequenced. Included is
the position in NCBI Build 34, the variation, public name if known (from dbSNP version 125), frequency in the different groups, location in the
gene, the amino acid base change (NCBI accession NP005567) and the effect (based on Blosum 45 amino acid similarity matrix).

| | | | Frequency of allele D | | | | | | | | NCBI34 reference |
|---|---|---|---|---|---|---|---|---|---|---|---|
| | | | HapMap | | | | | | | | assembly |
| | Variation | Public | populations^{b} | | | Iceland | | Location in | | Triplet | nucleotide |
| Position^{a} | D/d | name | CEU | YRI | CHB/JPT | Controls | XFG | gene | Location in protein^{c} | codon | (plus strand) |
| 71935363 | G/T | rs1048661 | 0.559 | 0.853 | 0.470 | 0.629 | 0.827 | Exon 1 | Arg->Leu (R141L) | CGG->CTG | G |
| | | | | | | | | | non-conservative | | |
| 71935399 | G/A | rs3825942 | 0.831 | 0.570 | 0.890 | 0.828 | 0.987 | Exon 1 | Gly->Asp (G153D) | GGC->GAC | G |
| | | | | | | | | | non-conservative | | |
| 71935416 | T/G | | 1.000 | 0.801 | 1.000 | 1.000 | 1.000 | Exon 1 | Ser->Ala (S159A) | TCG->GCG | T |
| | | | | | | | | | conservative | | |
| 71935419 | G/C | | 1.000 | 1.000 | 1.000 | 0.989 | 1.000 | Exon 1 | Ala->Pro (A160P) | GCT->CCT | G |
| | | | | | | | | | conservative | | |
| 71935817 | C/T | | 0.968 | 1.000 | 1.000 | 0.965 | 0.975 | Exon 1 | Asp->Asp (D292D) | GAC->GAT | C |
| | | | | | | | | | silent | | |
| 71935901 | G/T | | 1.000 | 0.963 | 0.690 | 0.987 | 0.958 | Exon 1 | Ala->Ala (A320A) | GCG->GCT | G |
| | | | | | | | | | silent | | |
| 71951055 | C/T | | 1.000 | — | — | 0.993 | 1.000 | Exon 2 | Ser->Ser (S382S) | TCC->TCT | C |
| | | | | | | | | | silent | | |
| 71955295 | G/A | | 1.000 | — | — | 0.993 | 1.000 | Exon 4 | Glu->Lys (E474K) | GAG->AAG | G |
| | | | | | | | | | conservative | | |

^{a}Position in NCBI Build 34.
^{b}Frequency in the 60 parents for the CEU and YRI HapMap groups. Only part of exon 1 was sequenced for YRI and CHP/JPT.
^{c}NP_005567.

TABLE 12
Association between glaucoma and variants in 15q24.1
Results for 41 SNPs on the Illumina Hap300 chip tested for association to glaucoma in
the interval 71.8 to 72.2 Mb on chromosome 15q24.1 (NCBI build 34) in the
genome-wide association study. The results are based on 195 glaucoma cases
(including both POAG and XFG) and 14474 controls from the Icelandic discovery
case-control group. Also included are the corresponding results after adjusting
for the observed association to rs1048661
and rs3825942.

| | | | | | | | Adjusted for | |
|---|---|---|---|---|---|---|---|---|
| | | | | | | | rs1048661 and | |
| | | | Frequency | | | | rs3825942 | |
| SNP | All | Pos^{a} | Cases | Controls | OR | P^{b} | OR^{c} | P^{c} |
| rs1963477 | A | 71807689 | 0.092 | 0.079 | 1.18 | 0.36 | 0.92 | 0.59 |
| rs4886630 | C | 71811557 | 0.949 | 0.918 | 1.66 | 0.021 | 1.21 | 1.00 |
| rs896588 | A | 71821310 | 0.238 | 0.204 | 1.23 | 0.10 | 1.17 | 0.23 |
| rs921535 | T | 71827160 | 0.874 | 0.831 | 1.42 | 0.021 | 0.90 | 0.54 |
| rs999742 | C | 71831334 | 0.823 | 0.749 | 1.56 | 0.00071 | 0.99 | 0.86 |
| rs8026593 | G | 71836202 | 0.987 | 0.963 | 2.99 | 0.0043 | 1.45 | 0.43 |
| rs4886425 | G | 71840360 | 0.900 | 0.857 | 1.50 | 0.014 | 0.99 | 1.00 |
| rs10519014 | A | 71841435 | 0.164 | 0.108 | 1.61 | 0.0014 | 1.14 | 0.38 |
| rs8030745 | T | 71848908 | 0.912 | 0.896 | 1.21 | 0.29 | 0.91 | 0.64 |
| rs1452388 | C | 71854895 | 0.175 | 0.146 | 1.25 | 0.12 | 1.07 | 0.63 |
| rs4445847 | T | 71887830 | 0.544 | 0.494 | 1.22 | 0.059 | 0.75 | 0.015 |
| rs13638 | T | 71897355 | 0.887 | 0.854 | 1.34 | 0.065 | 0.88 | 0.49 |
| rs4886467 | T | 71914630 | 0.431 | 0.411 | 1.08 | 0.45 | 0.70 | 0.0016 |
| rs4461027 | C | 71927365 | 0.503 | 0.392 | 1.57 | 2.0 × 10⁻⁵ | 0.86 | 0.22 |
| rs2165241 | T | 71938019 | 0.672 | 0.473 | 2.28 | 2.0 × 10⁻¹⁴ | 0.87 | 0.65 |
| rs8041685 | G | 71941010 | 0.921 | 0.839 | 2.23 | 3.5 × 10⁻⁶ | 0.61 | 1.00 |
| rs893817 | A | 71944882 | 0.762 | 0.633 | 1.85 | 1.4 × 10⁻⁷ | 0.85 | 0.73 |
| rs2304719 | C | 71951317 | 0.826 | 0.695 | 2.07 | 1.2 × 10⁻⁸ | 0.87 | 0.50 |
| rs1530169 | C | 71952937 | 0.836 | 0.765 | 1.56 | 0.001 | 1.02 | 0.90 |
| rs2304721 | C | 71956171 | 0.946 | 0.939 | 1.14 | 0.55 | 0.99 | 0.95 |
| rs3522 | C | 71960161 | 0.601 | 0.522 | 1.37 | 0.0027 | 0.97 | 0.80 |
| rs10152898 | T | 71970938 | 0.269 | 0.165 | 1.87 | 5.1 × 10⁻⁷ | 1.58 | 0.00023 |
| rs2507 | G | 71991493 | 0.338 | 0.278 | 1.33 | 0.011 | 1.32 | 0.0253 |
| rs734854 | T | 71997665 | 0.367 | 0.341 | 1.12 | 0.295 | 0.92 | 0.44 |
| rs3816197 | T | 71998697 | 0.349 | 0.302 | 1.24 | 0.06 | 1.33 | 0.01 |
| rs3784563 | C | 72006810 | 0.749 | 0.690 | 1.34 | 0.013 | 1.13 | 0.33 |
| rs11072463 | C | 72019166 | 0.354 | 0.331 | 1.11 | 0.35 | 0.89 | 0.31 |
| rs4886844 | T | 72024757 | 0.426 | 0.408 | 1.07 | 0.51 | 0.83 | 0.09 |
| rs3784556 | G | 72048005 | 0.915 | 0.904 | 1.15 | 0.45 | 1.09 | 0.27 |
| rs876383 | G | 72049230 | 0.569 | 0.556 | 1.06 | 0.60 | 1.20 | 0.086 |
| rs5742915 | T | 72052450 | 0.572 | 0.535 | 1.16 | 0.15 | 1.24 | 0.049 |
| rs1036673 | C | 72054279 | 0.372 | 0.293 | 1.43 | 0.0013 | 1.23 | 0.062 |
| rs723434 | T | 72059147 | 0.844 | 0.818 | 1.20 | 0.20 | 0.96 | 0.83 |
| rs8038527 | A | 72150013 | 0.787 | 0.737 | 1.32 | 0.026 | 1.04 | 0.78 |
| rs8033129 | C | 72156236 | 0.879 | 0.811 | 1.70 | 0.00041 | 1.23 | 0.20 |
| rs11635599 | C | 72163813 | 0.223 | 0.186 | 1.26 | 0.076 | 1.03 | 0.84 |
| rs12442054 | G | 72173841 | 0.910 | 0.901 | 1.11 | 0.56 | 1.23 | 0.28 |
| rs4886574 | C | 72177537 | 0.446 | 0.405 | 1.18 | 0.12 | 1.08 | 0.48 |
| rs923118 | T | 72182088 | 0.272 | 0.233 | 1.23 | 0.085 | 1.12 | 0.34 |
| rs351237 | C | 72193056 | 0.431 | 0.391 | 1.18 | 0.12 | 1.07 | 0.53 |
| rs351230 | C | 72198814 | 0.890 | 0.877 | 1.13 | 0.47 | 1.18 | 0.30 |

^{a}Basepair position in NCBI Build 34.
^{b}P value adjusted for relatedness and population stratification using the method genomic control.
^{c}Odds ratio (OR) and P value adjusted for the observed association to the two exonic SNPs, rs1048661 and rs3825942.

TABLE 13
Conditional association between XFG and additional markers typed in LOXL1.
Association between exfoliation glaucoma (XFG) and thirteen SNPs,
including rs2165241 and the two non-synonymous SNPs rs1048661 and rs3825942, typed in LOXL1.
The association is calculated for the combined Icelandic and Swedish case-control groups and for each SNP the
association is tested conditional on the observed association to each of the other SNPs.

| | | | Adjusted P values | | | | | | |
|---|---|---|---|---|---|---|---|---|---|
| SNP | Allele | Position | rs4461027 | rs1048661 | rs3825942 | rs2165241 | rs1992314 | rs2028386 | rs4337252 |
| rs4461027 | C | 71927365 | — | 0.033 | 0.0019 | 0.52 | 0.031 | 0.25 | 0.26 |
| rs1048661 | G | 71935363 | 4.7 × 10⁻⁸ | — | 4.5 × 10⁻¹⁸ | 0.008 | 0.27 | 0.00036 | 0.00046 |
| rs3825942 | G | 71935399 | 4.5 × 10⁻¹⁸ | 6.8 × 10⁻²⁷ | — | 1.5 × 10⁻⁹ | 6.3 × 10⁻²⁷ | 3.9 × 10⁻⁹ | 2.5 × 10⁻⁹ |
| rs2165241 | T | 71938019 | 8.2 × 10⁻²³ | 1.0 × 10⁻¹⁸ | 2.2 × 10⁻¹⁶ | — | 4.1 × 10⁻¹⁹ | 0.15 | 0.12 |
| rs1992314 | C | 71939584 | 6.5 × 10⁻⁸ | 0.48 | 5.9 × 10⁻¹⁸ | 0.0043 | — | 0.00015 | 0.00017 |
| rs2028386 | C | 71942525 | 3.7 × 10⁻²³ | 4.0 × 10⁻²⁰ | 4.2 × 10⁻¹⁶ | 0.098 | 1.2 × 10⁻²⁰ | — | 0.25 |
| rs4337252 | G | 71942582 | 6.2 × 10⁻²³ | 8.3 × 10⁻²⁰ | 4.4 × 10⁻¹⁶ | 0.14 | 2.2 × 10⁻²⁰ | 0.54 | — |
| rs4886782 | A | 71944627 | 2.8 × 10⁻⁸ | 0.000029 | 3.0 × 10⁻⁷ | 0.89 | 0.000027 | 0.85 | 0.8 |
| rs893817 | A | 71944882 | 6.7 × 10⁻⁶ | 0.97 | 1.1 × 10⁻¹⁵ | 0.099 | 0.97 | 0.026 | 0.028 |
| rs12440667 | T | 71947256 | 4.6 × 10⁻²¹ | 1.9 × 10⁻¹⁵ | 2.0 × 10⁻¹⁵ | 0.34 | 1.2 × 10⁻¹⁵ | 0.11 | 0.088 |
| rs2304719 | C | 71951317 | 8.8 × 10⁻¹⁵ | 7.7 × 10⁻¹⁴ | 2.3 × 10⁻⁶ | 0.0059 | 7.2 × 10⁻¹⁴ | 0.014 | 0.012 |
| rs750460 | T | 71957323 | 4.6 × 10⁻²⁰ | 2.0 × 10⁻¹⁴ | 1.2 × 10⁻¹⁴ | 0.63 | 1.3 × 10⁻¹⁴ | 0.23 | 0.19 |
| rs4243042 | T | 71957441 | 4.8 × 10⁻²⁰ | 1.8 × 10⁻¹⁴ | 1.2 × 10⁻¹⁴ | 0.64 | 1.4 × 10⁻¹⁴ | 0.23 | 0.19 |

| | | | Adjusted P values | | | | | |
|---|---|---|---|---|---|---|---|---|
| SNP | Allele | Position | rs4886782 | rs893817 | rs12440667 | rs2304719 | rs750460 | rs4243042 |
| rs4461027 | C | 71927365 | 0.075 | 0.035 | 0.28 | 0.11 | 0.35 | 0.35 |
| rs1048661 | G | 71935363 | 0.0001 | 0.0021 | 0.32 | 1.2 × 10⁻⁶ | 0.53 | 0.57 |
| rs3825942 | G | 71935399 | 1.2 × 10⁻¹⁵ | 1.4 × 10⁻²⁶ | 1.4 × 10⁻¹⁰ | 3.9 × 10⁻⁸ | 1.1 × 10⁻¹⁰ | 1.0 × 10⁻¹⁰ |
| rs2165241 | T | 71938019 | 1.2 × 10⁻¹⁶ | 7.7 × 10⁻²⁰ | 0.0019 | 1.0 × 10⁻¹¹ | 0.00029 | 0.00027 |
| rs1992314 | C | 71939584 | 0.00013 | 0.0031 | 0.27 | 1.6 × 10⁻⁶ | 0.44 | 0.44 |
| rs2028386 | C | 71942525 | 8.4 × 10⁻¹⁷ | 1.7 × 10⁻²⁰ | 0.00054 | 1.6 × 10⁻¹¹ | 0.00011 | 0.000099 |
| rs4337252 | G | 71942582 | 1.3 × 10⁻¹⁶ | 3.1 × 10⁻²⁰ | 0.00073 | 2.2 × 10⁻¹¹ | 0.00015 | 0.00014 |
| rs4886782 | A | 71944627 | — | 0.000013 | 0.66 | 0.00016 | 0.83 | 0.82 |
| rs893817 | A | 71944882 | 0.0072 | — | 0.0018 | 5.9 × 10⁻⁶ | 0.0068 | 0.0065 |
| rs12440667 | T | 71947256 | 9.5 × 10⁻¹⁵ | 1.9 × 10⁻¹⁹ | — | 6.5 × 10⁻¹¹ | 0.037 | 0.036 |
| rs2304719 | C | 71951317 | 3.0 × 10⁻¹¹ | 3.0 × 10⁻¹⁵ | 0.00036 | — | 0.00022 | 0.00021 |
| rs750460 | T | 71957323 | 8.2 × 10⁻¹⁴ | 5.0 × 10⁻¹⁸ | 0.6 | 3.3 × 10⁻¹⁰ | — | 1 |
| rs4243042 | T | 71957441 | 8.9 × 10⁻¹⁴ | 5.3 × 10⁻¹⁸ | 0.61 | 3.5 × 10⁻¹⁰ | 0.74 | — |

TABLE 14
Sequence identification for markers provided in sequence listing

| Seq ID | SNP/Marker |
|---|---|
| 1 | rs1963477 |
| 2 | rs488663 |
| 3 | rs896588 |
| 4 | rs921535 |
| 5 | rs999742 |
| 6 | rs8026593 |
| 7 | rs4886425 |
| 8 | rs10519014 |
| 9 | rs8030745 |
| 10 | rs1452388 |
| 11 | rs4445847 |
| 12 | rs13638 |
| 13 | rs4886467 |
| 14 | rs4461027 |
| 15 | rs2165241 |
| 16 | rs8041685 |
| 17 | rs893817 |
| 18 | rs2304719 |
| 19 | rs1530169 |
| 20 | rs2304721 |
| 21 | rs3522 |
| 22 | rs10152898 |
| 23 | rs2507 |
| 24 | rs734854 |
| 25 | rs3816197 |
| 26 | rs378456 |
| 27 | rs11072463 |
| 28 | rs4886844 |
| 29 | rs3784556 |
| 30 | rs876383 |
| 31 | rs5742915 |
| 32 | rs1036673 |
| 33 | rs723434 |
| 34 | rs8038527 |
| 35 | rs8033129 |
| 36 | rs11635599 |
| 37 | rs12442054 |
| 38 | rs4886574 |
| 39 | rs923118 |
| 40 | rs351237 |
| 41 | rs351230 |
| 42 | rs4261482 |
| 43 | rs4886761 |
| 44 | rs1992314 |
| 45 | rs4886776 |
| 46 | rs2028386 |
| 47 | rs4337252 |
| 48 | rs2028387 |
| 49 | rs4077284 |
| 50 | rs893816 |
| 51 | rs4886782 |
| 52 | rs893818 |
| 53 | rs893820 |
| 54 | rs12440667 |
| 55 | rs750460 |
| 56 | rs4243042 |
| 57 | rs12437465 |
| 58 | DG15818 |
| 59 | SG5S205 |
| 60 | SG5S206 |
| 61 | SG5S208 |
| 62 | SG5S209 |
| 63 | SG5S210 |
| 64 | SG5S211 |
| 65 | SG5S212 |
| 66 | SG5S214 |
| 67 | SG5S215 |
| 68 | SG5S216 |
| 69 | SG5S217 |
| 70 | SG5S218 |
| 71 | SG5S219 |
| 72 | SG5S221 |
| 73 | SG5S222 |
| 74 | SG5S223 |
| 75 | SG5S226 |
| 76 | SG5S228 |
| 77 | SG5S230 |
| 78 | SG5S234 |
| 79 | SG5S236 |
| 80 | SG5S237 |
| 81 | SG5S238 |
| 82 | SG5S242 |
| 83 | SG5S243 |
| 84 | LD block C15 |
| 85 | LOXL1 |
| 86 | rs4886725 |
| 87 | rs12915956 |
| 88 | rs896590 |
| 89 | rs12438872 |
| 90 | rs893821 |
| 91 | rs2304722 |
| 92 | rs4886623 |
| 93 | rs2167648 |
| 94 | rs1584738 |
| 95 | rs1901570 |
| 96 | rs4886660 |
| 97 | rs4886421 |
| 98 | rs4886663 |
| 99 | rs4886664 |
| 100 | rs4145873 |
| 101 | rs4145874 |
| 102 | rs1452389 |
| 103 | rs1078967 |
| 104 | rs8041642 |
| 105 | rs8042039 |
| 106 | rs1048661 |
| 107 | rs3825942 |
| 108 | SG15S187 |
| 109 | SG15S207 |
| 110 | SG15S262 |
| 111 | SG15S263 |
| 112 | SG15S264 |
| 113 | SG1SS265 |
| 114 | SG15S266 |
| 115 | SG15S267 |
| 116 | SG15S268 |
| 117 | SG15S269 |
| 118 | SG15S271 |
| 119 | SG15S272 |
| 120 | SG15S273 |
| 121 | G indel |
| 122 | rs8035197 |
| 123 | rs8035210 |
| 124 | rs8034178 |
| 125 | rs8034355 |
| 126 | rs4776620 |
| 127 | rs6495030 |
| 128 | rs2680346 |
| 129 | rs7170655 |
| 130 | rs16958177 |
| 131 | rs16958188 |
| 132 | rs1548099 |
| 133 | rs7402796 |
| 134 | rs896598 |
| 135 | rs4887140 |
| 136 | rs7174255 |
| 137 | rs4886647 |
| 138 | rs4886415 |
| 139 | rs4886416 |
| 140 | rs2198294 |
| 141 | rs2061803 |
| 142 | rs2167648 |
| 143 | rs1584738 |
| 144 | rs1901570 |
| 145 | rs4577028 |
| 146 | rs1349642 |
| 147 | rs921541 |
| 148 | rs744616 |
| 149 | rs2084851 |
| 150 | rs7170291 |
| 151 | rs7496005 |
| 152 | rs12440507 |
| 153 | rs11855607 |
| 154 | rs4558370 |
| 155 | rs4464030 |
| 156 | rs4283181 |
| 157 | rs11634339 |
| 158 | rs1440101 |
| 159 | rs2289414 |
| 160 | rs12594472 |
| 161 | rs16958494 |
| 162 | rs12441130 |
| 163 | rs12441138 |
| 164 | rs12904518 |
| 165 | rs12102015 |
| 166 | rs12101466 |
| 167 | rs8042532 |
| 168 | rs4886809 |
| 169 | rs2289413 |
| 170 | rs4993000 |
| 171 | rs2301273 |
| 172 | rs11072464 |
| 173 | rs3825941 |
| 174 | rs743582 |
| 175 | rs10851869 |
| 176 | rs1550435 |
| 177 | rs4887066 |
| 178 | rs6495089 |
| 179 | rs1385650 |
| 180 | rs10910 |
| 181 | rs2959010 |
| 182 | rs2277603 |
| 183 | rs16969393 |
| 184 | rs12439113 |
| 185 | rs6495122 |
| 186 | rs2290573 |
| 187 | rs12487 |
| 188 | rs11630918 |
| 189 | rs7497393 |
| 190 | rs1127796 |
| 191 | rs11856413 |
| 192 | rs11072512 |
| 193 | rs1133323 |
| 194 | rs1133322 |
| 195 | rs11072513 |
| 196 | rs11072514 |
| 197 | rs2415251 |

### EXAMPLE 3

### Determination of LOXL1 Variants on Endogenous Processing of LOXL1

The pro-sequence of LOXL1 is required for proper deposition of the protein in the ECM. The main extracellular protease responsible for cleavage of the pro-sequence is bone morphogenetic protein-1 (BMP-1), also referred to as procollagen-C-proteinase (PCP) (3,9). Although a rigorously defined consensus sequence for BMP-1 cleavage is not established, the enzyme shows preference for A or G at the P-site and D at the P′-site. For example, BMP-1 mediated cleavage of human LOX occurs at the G168-D169 bond. The corresponding cleavage site in LOXL1 has however not been identified, although G-D pairs at sites 135 and 304 have been mentioned as plausible candidates.

Interestingly, the G153D variant (GA haplotype) reveals what may be a cryptic protelytic cleavage site for BMP-1. It is possible that individuals carrying the GA haplotype are protected against XFG due to a more efficient and proper proteolytic processing of ProLOXL1, compared to the other haplotypes. Efficient processing of the enzyme could lead to an increase in overall total activity and or deposition of the enzyme in the ECM, which could be beneficial in abrogating the harmful accumulation of abnormal fibrillar material observed in XFG.

The following experiments are designed to investigate the processing of LOXL1 in vivo and develop strategies for designing LOXL1 with optimized proteolytic cleavage.

### Experimental Design:

### A) Expression of LOX and LOXL1 Variants as Substrates for BMP-1.

In order to determine if the G153D variant does in fact introduce a new BMP-1 cleavage site, the processing reaction with BMP-1 needs to be studied in a controlled in-vitro environment. Variants of LOXL1 will be produced in E. coli in enough quantities to use as substrates for BMP-1. LOX will also be produced to use as a control.

The following variants of LOXL1 will be made:
i) R141L
ii) G153D
iii) R141L, G153D

The constructs containing a smt3 tag (11 kDa) for increased solubilization and a His-tag for purification will be expressed in E. coli.

### B) In-Vitro Processing of Lox and LOXL1.

Assays for in-vitro processing of LOXL1 variants will be performed using a similar approach as was used to identify the LOX cleavage site (Panchenko, M. V. et al. J Biol Chem 271:7113-19 (1996); Uzel, M. I. et al. J Biol Chem 276:22537-43 (2001)). LOXL1 (or LOX) smt3 fusion proteins will be incubated with human, recombinant BMP-1 (R&D Systems) and the proteolytic reaction will be monitored by detection of protein fragments by immunostaining after separation by SDS-PAGE. Immunostaining of gels can be performed with either commercial LOX/LOXL1 antibodies (Santa Cruz Biotechnology) for C-terminal fragments or smt3 antibody (AbCam) for N-terminal fragments. Quantification and kinetics of the processing reaction will be performed. N-terminal sequencing of isolated protein bands will be done to accurately determine the site(s) of cleavage for all variants.

### C) ECM Deposition and Processing of LOXL1 Variants in RFL-6 Cells.

Elegant experiments by Thommasin et al. have shown that the pro-sequence of LOX and LOXL1 is required for targeting and deposition onto elestic fibers in the ECM (Thomassin, L. Et al J Biol Chem 280:42848-55 (2005)). They studied this process in rat fetal lung fibroblasts (RFL-6, ATCC # CCL-192), mainly because this cell line produces an elaborate fibrillar matrix containing various ECM proteins, including tropoelastin and fibrillin-1 and -2. They were able to show the co-localization of LOX/LOXL1 onto elastic fibers by transient transfection of several LOX/LOXL1 constructs containing a C-terminal V5 epitope tag.

LOXL1 variants will be cloned into a pcDNA4-V5/H is vector (Invitrogen) and used for lipofectamine transfection of RFL-6 cells as described (Thomassin, L. Et al J Biol Chem 280:42848-55 (2005)). Targeting of LOXL1 variants to the ECM will be assessed with immunofluorescence microscopy using a monoclonal Anti-V5 antibody (Invitrogen) and co-localization with elastin will be measured using polyclonal AB's to tropoelastin (AbCam). Proteloytic processing of V5-epitope tagged LOXL1 variants will be assessed in media and cell layer of RFL-6 cells as described (Thomassin, L. Et al J Biol Chem 280:42848-55 (2005)).

### D) Activity of LOXL1 Variants in RFL-6 Cells.

Thomassin et al. did not assess enzymatic activity of LOXL1 in transfected RFL-6 cells. A sensitive enzymatic assay suitable for measuring LOXL1 activity in cell extracts will be set up (Palamakumbura, A. H. & Trackman, P. C. Anal Biochem 300:245-51 (2002)). This fluorometric assay detects the formation of peroxide via its HRP mediated reaction with Amplex Red (Molecular Probes). The produced fluorophore (resorufin) can be excited in the far visible region (>500 nm), which eliminates various interference such as fluorescence quenching. Measurements of enzyme activity coupled with quantitation of protein products in both media and cell layer should allow for quantitative assessment of the effects of different LOXL1 variants. Either 1,5 diaminopentane or tropoelastin can be used as substrates for LOXL1. To ensure specificity, control reactions with BAPN, a known specific and irreversible LOX/LOXL inhibitor will be conducted. Background activity in media and cell layer from non-transfected cells will also be quantified.

Another possibility is to use a V5 antibody to pull down (IP) specific LOXL1 variants expressed and assess their activity. This approach would probably only be attainable with protein found in the media.

### E) Expression and Activity of Endogenous LOXL1 in a Human Lens Cell-Line.

Exfoliation material in XFG is mainly associated with the anterior surface of the lens (Schlötzer-Gerhardt, U. Naumann, G. O. H. Am J Opthalmol 141:921-37 (2006)). The endogenous expression of LOXL1 in a human, lens cell-line will be examined in order to study the function of LOXL1 under conditions related to XFG. This cell-line (B-3, ATCC# CRL-11421) is of epithelial morphology and has been immortalized with SV-40 virus. LOX/LOXL activity will also be assessed in both media and cell layer of these cells as described above for the RFL-6 cells.

### F) Transfection and Studies of LOXL1 Variants in B-3 Cells.

Transfection (stable or transient) of the same LOXL1 variant constructs as used for the RFL-6 cells will be performed with the human B-3 lens cells. Similar experiments as described in parts C and D will be conducted to probe the effect of the site-specific mutations in LOXL1 in the B-3 cells. The focus will be on processing and LOX/LOXL1 activity in these cells.

G) Design of an Optimal Cleavage site in LOXL1.

Based on results with the D153 variant, an “optimal” BMP-1 cleavage site will be designed in LOXL1. A series of target onstructs will be prepared and expressed in E. coli using the approach described in part A. The smt3 tagged LOXL1 proteins will be measured for BMP-1 activity in an in-vitro processing assay as described in part B. The best BMP-1 substrates will subsequently be assessed in other cellular assay environments as described above for the natural variants.

1. A method of determining a susceptibility to at least one ocular condition selected from exfoliation syndrome and glaucoma in a human individual, the method comprising:
obtaining nucleic acid sequence data about a human individual identifying at least one allele of at least one polymorphic marker associated with the human LOXL1 gene, wherein different alleles of the at least one polymorphic marker are associated with different susceptibilities to the at least one condition in humans, and
determining a susceptibility to at least one condition selected from exfoliation syndrome and glaucoma from the nucleic acid sequence data.

2. The method of claim 1, comprising obtaining nucleic acid sequence data about at least two polymorphic markers associated with the LOXL1 gene.

3. The method of claim 1, wherein determination of a susceptibility comprises comparing the nucleic acid sequence data to a database containing correlation data between polymorphic markers of the human LOXL1 gene and susceptibility to the at least one condition.

4. The method of claim 3, wherein the database comprises at least one risk measure of susceptibility to the at least one condition for the polymorphic markers of the LOXL1 gene.

5. The method of claim 3, wherein the database comprises a look-up table containing at least one risk measure of the at least one condition for the polymorphic markers.

6. The method of a claim 1, wherein obtaining nucleic acid sequence data comprises obtaining a biological sample from the human individual and analyzing sequence of the at least one polymorphic marker in nucleic acid in the sample.

7. The method of claim 6, wherein analyzing sequence of the at least one polymorphic marker comprises determining the presence or absence of at least one allele of the at least one polymorphic marker.

8. The method of claim 1, wherein the obtaining nucleic acid sequence data comprises obtaining nucleic acid sequence information from a preexisting record.

9. The method of claim 1, further comprising reporting the susceptibility to at least one entity selected from the group consisting of the individual, a guardian of the individual, a genetic service provider, a physician, a medical organization, and a medical insurer.

10. The method of claim 1, wherein the at least one polymorphic marker is selected from the group consisting of the markers listed in Table 16, and markers in linkage disequilibrium therewith.

11. (canceled)

12. The method of claim 10, wherein linkage disequilibrium is defined by numerical values of r² of at least 0.2 and/or values of |D′| of at least 0.8.

13. The method of claim 1, wherein the at least one polymorphic marker is selected from the group consisting of rs4886725 (SEQ ID NO:86), rs12915956 (SEQ ID NO:87), rs896590 (SEQ ID NO:88), rs12438872 (SEQ ID NO:89), rs4261482 (SEQ ID NO:42), rs2165241 (SEQ ID NO:15), rs1992314 (SEQ ID NO:44), rs4886776 (SEQ ID NO:45), rs2028386 (SEQ ID NO:46), rs4337252 (SEQ ID NO:47), rs2028387 (SEQ ID NO:48), rs4077284 (SEQ ID NO:49), rs893816 (SEQ ID NO:50), rs4886782 (SEQ ID NO:51), rs893817 (SEQ ID NO:17), rs893818 (SEQ ID NO:52), rs893820 (SEQ ID NO:53), rs12440667 (SEQ ID NO:54), rs1530169 (SEQ ID NO:19), rs893821 (SEQ ID NO:90), rs750460 (SEQ ID NO:55), rs4243042 (SEQ ID NO:56), rs2304722 (SEQ ID NO:91), rs4886623 (SEQ ID NO:92), rs2167648 (SEQ ID NO:93), rs1584738 (SEQ ID NO:94), rs1901570 (SEQ ID NO:95), rs8026593 (SEQ ID NO:6), rs4886660 (SEQ ID NO:96), rs4886421 (SEQ ID NO:97), rs4886663 (SEQ ID NO:98), rs4886664 (SEQ ID NO:99), rs4145873 (SEQ ID NO:100), rs4145874 (SEQ ID NO:101), rs1452389 (SEQ ID NO:102), rs1078967 (SEQ ID NO:103), rs8041642 (SEQ ID NO:104), rs8041685 (SEQ ID NO:16), rs8042039 (SEQ ID NO:105), rs2304719 (SEQ ID NO:18), rs12437465 (SEQ ID NO:57), rs1048661 (SEQ ID NO:106), and rs3825942 (SEQ ID NO: 107).

14. (canceled)

15. A method of determining a susceptibility to at least one ocular condition selected from exfoliation syndrome and glaucoma in a human individual, the method comprising:
obtaining nucleic acid sequence data about a human individual identifying both alleles of at least two polymorphic markers associated with the human LOXL1 gene,
determine the identity of at least one haplotype based on the sequence data, and
determining a susceptibility to at least one condition selected from exfoliation syndrome and glaucoma from the haplotype data.

16. The method according to claim 1, wherein the at least one allele or haplotype is indicative of increased susceptibility to exfoliation syndrome and/or glaucoma.

17. The method according to claim 16, wherein the increased susceptibility is characterized by a relative risk or an odds ratio of at least 1.5, including at least 2.0, at least 2.5, at least 3.0, at least 3.5, and at least 4.0.

18. The method according to claim 16, wherein the at least one allele is rs2165241 allele T, rs1048661 allele G or rs3825942 allele G.

19. The method according to claims 15, wherein the presence of
a. The haplotype characterized by the presence of allele G in marker rs1048661 (SEQ ID NO: 106) and allele G in marker rs3825942 (SEQ ID NO: 107); and/or
b. The haplotype characterized by the presence of allele T in marker rs1048661 (SEQ ID NO: 106) and allele G in marker rs3825942 (SEQ ID NO: 107) is indicative of increased susceptibility of exfoliation syndrome or glaucoma.

20. The method of claim 15, wherein the presence of the at least one allele or haplotype is indicative of decreased susceptibility to exfoliation syndrome and/or glaucoma.

21. The method of claim 17, wherein the presence of rs1048661 allele T and/or rs3825942 allele A is indicative of decreased susceptibility to symptoms associated with exfoliation syndrome and/or glaucoma.

22. The method of claim 15, wherein the presence of the haplotype characterized by the presence of allele G in marker rs1048661 (SEQ ID NO: 106) and allele A in marker rs3825942 (SEQ ID NO: 107) is indicative of a decreased susceptibility of developing exfoliation syndrome or glaucoma.

23. The method of claim 20, wherein the decreased susceptibility is characterized by an odds ratio or relative risk of less than 0.7, including less than 0.6, less than 0.5, less than 0.4, less than 0.35, less than 0.3, and less than 0.25.

21-42. (canceled)

43. A method of diagnosing a susceptibility to at least one ocular condition selected from exfoliation syndrome and glaucoma in a human individual, the method comprising:
obtaining LOXL1 amino acid sequence data about at least one encoded LOXL1 protein of a human individual,
identifying at least one polymorphic site associated with the LOXL1 amino acid sequence, wherein different amino acids of the at least one polymorphic site are associated with different susceptibilities to the at least one condition in humans, and
diagnosing susceptibility to at least one condition selected from exfoliation syndrome and glaucoma from the amino acid sequence data.

44. The method of claim 43, wherein determination of the presence of an Arginine at position 141 and/or a Glycine at position 153 in the LOXL1 protein as set forth in SEQ ID NO:85 is indicative of an increased susceptibility to the at least one condition.

45. (canceled)

46. The method of claim 43, wherein determination of the presence of an Leucine at position 141 and/or an Aspartic acid at position 153 in the LOXL1 protein as set forth in SEQ ID NO:85 is indicative of an decreased susceptibility to the at least one condition.

47. (canceled)

48. A method of diagnosing a susceptibility of symptoms associated with exfoliation syndrome and/or glaucoma in an individual, the method comprising determining the identity of at least one allele of at least one polymorphic marker in a nucleic acid sample obtained from the individual, wherein the at least one marker is selected from the group of markers located within the LOXL1 LD block, wherein susceptibility of symptoms associated with exfoliation syndrome and/or glaucoma is correlated with the identity of the at least one allele.

49. The method of claim 48, comprising determining both alleles of the at least one polymorphic marker.

50. The method of claim 49, further comprising assessing the identity of at least one haplotype in the individual, wherein the presence of the at least one haplotype is indicative of a susceptibility to symptoms associated with exfoliation syndrome and/or glaucoma.

51. The method of claim 48, wherein the at least one polymorphic marker is selected from the group of markers listed in Table 4, and markers in linkage disequilibrium therewith.

52-67. (canceled)

68. A method of identification of a marker for use in assessing susceptibility to exfoliation syndrome and/or glaucoma, the method comprising
a. identifying at least one polymorphism associated with the LOXL1 gene; and
b. determining the genotype status of a sample of individuals diagnosed with, or having a susceptibility to, exfoliation syndrome and/or glaucoma, and a control sample;
wherein a significant difference in frequency of at least one allele in at least one polymorphism in individuals diagnosed with, or having a susceptibility to, exfoliation syndrome, as compared with the frequency of the at least one allele in the control sample is indicative of the at least one polymorphism being useful for assessing susceptibility to exfoliation syndrome and/or glaucoma.

69. (canceled)

70. The method of claim 68, wherein an increase in frequency of the at least one allele in the at least one polymorphism in individuals diagnosed with, or having a susceptibility to, exfoliation syndrome, as compared with the frequency of the at least one allele in the control sample is indicative of the at least one polymorphism being useful for assessing increased susceptibility to exfoliation syndrome and/or glaucoma, or
wherein decrease in frequency of the at least one allele in the at least one polymorphism in individuals diagnosed with, or having a susceptibility to, exfoliation syndrome, as compared with the frequency of the at least one allele in the control sample is indicative of the at least one polymorphism being useful for assessing decreased susceptibility to, or protection against, exfoliation syndrome and/or glaucoma.

71. (canceled)

72. A method of genotyping a nucleic acid sample obtained from a human individual at risk for, or diagnosed with, exfoliation syndrome and/or glaucoma, comprising determining the identity of at least one allele of at least one polymorphic marker in the sample, wherein the marker is selected from the group consisting of rs4886725 (SEQ ID NO:86), rs12915956 (SEQ ID NO:87), rs896590 (SEQ ID NO:88), rs12438872 (SEQ ID NO:89), rs4261482 (SEQ ID NO:42), rs2165241 (SEQ ID NO:15), rs1992314 (SEQ ID NO:44), rs4886776 (SEQ ID NO:45), rs2028386 (SEQ ID NO:46), rs4337252 (SEQ ID NO:47), rs2028387 (SEQ ID NO:48), rs4077284 (SEQ ID NO:49), rs893816 (SEQ ID NO:50), rs4886782 (SEQ ID NO:51), rs893817 (SEQ ID NO:17), rs893818 (SEQ ID NO:52), rs893820 (SEQ ID NO:53), rs12440667 (SEQ ID NO:54), rs1530169 (SEQ ID NO:19), rs893821 (SEQ ID NO:90), rs750460 (SEQ ID NO:55), rs4243042 (SEQ ID NO:56), rs2304722 (SEQ ID NO:91), rs4886623 (SEQ ID NO:92), rs2167648 (SEQ ID NO:93), rs1584738 (SEQ ID NO:94), rs1901570 (SEQ ID NO:95), rs8026593 (SEQ ID NO:6), rs4886660 (SEQ ID NO:96), rs4886421 (SEQ ID NO:97), rs4886663 (SEQ ID NO:98), rs4886664 (SEQ ID NO:99), rs4145873 (SEQ ID NO:100), rs4145874 (SEQ ID NO:101), rs1452389 (SEQ ID NO:102), rs1078967 (SEQ ID NO:103), rs8041642 (SEQ ID NO:104), rs8041685 (SEQ ID NO:16), rs8042039 (SEQ ID NO:105), rs2304719 (SEQ ID NO:18), rs12437465 (SEQ ID NO:57), rs1048661 (SEQ ID NO:106), and rs3825942 (SEQ ID NO: 107), and markers in linkage disequilibrium therewith, and wherein the identity of the at least one allele of the at least one polymorphic marker is indicative of a susceptibility of exfoliation syndrome and/or glaucoma.

73-74. (canceled)

75. The method of a claim 72, wherein the sample is a blood sample or a buccal swab.

76. (canceled)

77. The method of a claim 72, wherein genotyping is performed using a process selected from allele-specific probe hybridization, allele-specific primer extension, allele-specific amplification, nucleic acid sequencing, 5′-exonuclease digestion, molecular beacon assay, oligonucleotide ligation assay, size analysis, and single-stranded conformation analysis.

78-84. (canceled)

85. The method of claim 1, further comprising assessing at least one biomarker in a sample from the individual.

86. The method of claim 85, wherein the sample is a blood sample or tear fluid.

87. (canceled)

88. The method of claim 85, wherein the biomarker is LOXL1 protein.

89. The method of claim 1, further comprising analyzing non-genetic information to make overall risk assessment, diagnosis, or prognosis of the individual.

90. The method of claim 89, wherein the non-genetic information is selected from age, gender, ethnicity, socioeconomic status, previous disease diagnosis, medical history of subject, family history of exfoliation syndrome and/or glaucoma, biochemical measurements, and clinical measurements.

91. The method of a claim 1, further comprising analyzing expression levels of LOXL1 in a sample from the individual.

92-94. (canceled)

95. The method of claim 91, wherein expression is determined by measuring LOXL1 protein levels in the sample.

96. The method of a claim 91, wherein expression is determined by measuring LOXL1 mRNA levels in the sample.

97. The method according to claim 91, wherein a decreased expression level of LOXL1 is indicative of increased susceptibility to exfoliation syndrome or glaucoma.

98. A method of assessing an individual for probability of response to a therapeutic agent for preventing and/or ameliorating symptoms associated with exfoliation syndrome and/or glaucoma, comprising: determining the identity of at least one allele of at least one polymorphic marker in a nucleic acid sample obtained from the individual, wherein the at least one polymorphic marker is selected from polymorphic markers associated with the human LOXL1 gene, wherein the identity of the at least one allele of the at least one marker is indicative of a probability of a positive response to the therapeutic agent.

99-102. (canceled)

103. The method of claim 98, wherein the therapeutic agent is selected from prostaglandin analogs, prostamides, α2 adrenergic agonists, carbonic anhydrase inhibitors, β-blockers, cholinergic agonists.

104. The method of claim 98, wherein the therapeutic agent is selected from latanoprost, travoprost, unoprostone, bimatoprost, brimonidine, apraclonidine, dorzolamide, brinzolamide, acetazolamide, methazolamide, betaxolol, carteolol, levobunalol, metipranolol, timolol, pilocarpine, carbachol, echothiphate and epinephrine.

105. A method of predicting prognosis of an individual experiencing symptoms associated with, or an individual diagnosed with, exfoliation syndrome and/or glaucoma, the method comprising
obtaining nucleic acid sequence data about a human individual identifying at least one allele of at least one polymorphic marker associated with the human LOXL1 gene, wherein different alleles of the at least one polymorphic marker are associated with different susceptibilities to the at least one condition in humans, and
predicting prognosis of the individual from the nucleic acid sequence data.

106-111. (canceled)

112. A method of predicting treatment outcome of an individual undergoing treatment for exfoliation syndrome and/or glaucoma, the method comprising
obtaining nucleic acid sequence data about a human individual identifying at least one allele of at least one polymorphic marker associated with the human LOXL1 gene, wherein different alleles of the at least one polymorphic marker are associated with different susceptibilities to the at least one condition in humans, and
predicting treatment outcome of the individual from the nucleic acid sequence data.

113-116. (canceled)

117. A kit for assessing susceptibility to symptoms associated with exfoliation syndrome and/or glaucoma of a human individual, the kit comprising reagents for selectively detecting at least one allele of at least one polymorphic marker in the genome of the individual, wherein the polymorphic marker is selected from the group consisting of the polymorphic markers listed in Table 4, Table 6 and Table 6a, and markers in linkage disequilibrium therewith, and wherein the presence of the at least one allele is indicative of a susceptibility to symptoms associated with exfoliation syndrome and/or glaucoma.

118. The kit of claim 117, wherein the at least one polymorphic marker is selected from the group consisting of the group consisting of rs4886725 (SEQ ID NO:86), rs12915956 (SEQ ID NO:87), rs896590 (SEQ ID NO:88), rs12438872 (SEQ ID NO:89), rs4261482 (SEQ ID NO:42), rs2165241 (SEQ ID NO:15), rs1992314 (SEQ ID NO:44), rs4886776 (SEQ ID NO:45), rs2028386 (SEQ ID NO:46), rs4337252 (SEQ ID NO:47), rs2028387 (SEQ ID NO:48), rs4077284 (SEQ ID NO:49), rs893816 (SEQ ID NO:50), rs4886782 (SEQ ID NO:51), rs893817 (SEQ ID NO:17), rs893818 (SEQ ID NO:52), rs893820 (SEQ ID NO:53), rs12440667 (SEQ ID NO:54), rs1530169 (SEQ ID NO:19), rs893821 (SEQ ID NO:90), rs750460 (SEQ ID NO:55), rs4243042 (SEQ ID NO:56), rs2304722 (SEQ ID NO:91), rs4886623 (SEQ ID NO:92), rs2167648 (SEQ ID NO:93), rs1584738 (SEQ ID NO:94), rs1901570 (SEQ ID NO:95), rs8026593 (SEQ ID NO:6), rs4886660 (SEQ ID NO:96), rs4886421 (SEQ ID NO:97), rs4886663 (SEQ ID NO:98), rs4886664 (SEQ ID NO:99), rs4145873 (SEQ ID NO:100), rs4145874 (SEQ ID NO:101), rs1452389 (SEQ ID NO:102), rs1078967 (SEQ ID NO:103), rs8041642 (SEQ ID NO:104), rs8041685 (SEQ ID NO:16), rs8042039 (SEQ ID NO:105), rs2304719 (SEQ ID NO:18), rs12437465 (SEQ ID NO:57), rs1048661 (SEQ ID NO:106), and rs3825942 (SEQ ID NO: 107), and markers in linkage disequilibrium therewith.

119-122. (canceled)

123. The kit of claim 117, wherein the reagents comprise at least one pair of oligonucleotides that hybridize to opposite strands of a genomic nucleic acid segment obtained from the subject, wherein each oligonucleotide primer pair is designed to selectively amplify a fragment of the genome of the individual that includes one polymorphic marker, and wherein the fragment is at least 30 base pairs in size.

124. The kit of claim 123, wherein the at least one oligonucleotide is completely complementary to the genome of the individual.

125. The kit of claim 123, wherein the oligonucleotide is about 18 to about 50 nucleotides in length.

126-130. (canceled)

131. A computer-readable medium having computer executable instructions for determining susceptibility to exfoliation syndrome and/or glaucoma, the computer readable medium comprising:
data indicative of at least one polymorphic marker;
a routine stored on the computer readable medium and adapted to be executed by a processor to determine risk of developing exfoliation syndrome and/or glaucoma for the at least one polymorphic marker;
wherein the at least one polymorphic marker is associated with the LOXL1 gene.

132. The computer readable medium of claim 131, wherein the computer readable medium contains data indicative of at least two polymorphic markers.

133. The computer readable medium of claim 131, wherein the at least one polymorphic marker is selected from rs4886725 (SEQ ID NO:86), rs12915956 (SEQ ID NO:87), rs896590 (SEQ ID NO:88), rs12438872 (SEQ ID NO:89), rs4261482 (SEQ ID NO:42), rs2165241 (SEQ ID NO:15), rs1992314 (SEQ ID NO:44), rs4886776 (SEQ ID NO:45), rs2028386 (SEQ ID NO:46), rs4337252 (SEQ ID NO:47), rs2028387 (SEQ ID NO:48), rs4077284 (SEQ ID NO:49), rs893816 (SEQ ID NO:50), rs4886782 (SEQ ID NO:51), rs893817 (SEQ ID NO:17), rs893818 (SEQ ID NO:52), rs893820 (SEQ ID NO:53), rs12440667 (SEQ ID NO:54), rs1530169 (SEQ ID NO:19), rs893821 (SEQ ID NO:90), rs750460 (SEQ ID NO:55), rs4243042 (SEQ ID NO:56), rs2304722 (SEQ ID NO:91), rs4886623 (SEQ ID NO:92), rs2167648 (SEQ ID NO:93), rs1584738 (SEQ ID NO:94), rs1901570 (SEQ ID NO:95), rs8026593 (SEQ ID NO:6), rs4886660 (SEQ ID NO:96), rs4886421 (SEQ ID NO:97), rs4886663 (SEQ ID NO:98), rs4886664 (SEQ ID NO:99), rs4145873 (SEQ ID NO:100), rs4145874 (SEQ ID NO:101), rs1452389 (SEQ ID NO:102), rs1078967 (SEQ ID NO:103), rs8041642 (SEQ ID NO:104), rs8041685 (SEQ ID NO:16), rs8042039 (SEQ ID NO:105), rs2304719 (SEQ ID NO:18), rs12437465 (SEQ ID NO:57), rs1048661 (SEQ ID NO:106), and rs3825942 (SEQ ID NO: 107), and markers in linkage disequilibrium therewith.

134-135. (canceled)

136. An apparatus for determining a genetic indicator for exfoliation syndrome or glaucoma in a human individual, comprising:
a processor
a computer readable memory having computer executable instructions adapted to be executed on the processor to analyze marker and/or haplotype information for at least one human individual with respect to at least one polymorphic marker associated with the LOXL1 gene, and
generate an output based on the marker or haplotype information, wherein the output comprises a risk measure of the at least one marker or haplotype as a genetic indicator of exfoliation syndrome or glaucoma for the human individual.

137. The apparatus according to claim 136, wherein the computer readable memory further comprises data indicative of the frequency of at least one allele of at least one polymorphic marker or at least one haplotype in a plurality of individuals diagnosed with, or presenting symptoms associated with, exfoliation syndrome or glaucoma, and data indicative of the frequency of at the least one allele of at least one polymorphic marker or at least one haplotype in a plurality of reference individuals, and wherein a risk measure is based on a comparison of the at least one marker and/or haplotype status for the human individual to the data indicative of the frequency of the at least one marker and/or haplotype information for the plurality of individuals diagnosed with exfoliation syndrome or glaucoma.

138. The apparatus according to claim 136, wherein the computer readable memory further comprises data indicative of the risk of developing exfoliation syndrome and/or glaucoma associated with at least one allele of at least one polymorphic marker or at least one haplotype, and wherein a risk measure for the human individual is based on a comparison of the at least one marker and/or haplotype status for the human individual to the risk associated with the at least one allele of the at least one polymorphic marker or the at least one haplotype.

139. The apparatus according to claim 138, wherein the computer readable memory further comprises data indicative of the frequency of at least one allele of at least one polymorphic marker or at least one haplotype in a plurality of individuals diagnosed with, or presenting symptoms associated with, exfoliation syndrome and/or glaucoma, and data indicative of the frequency of at the least one allele of at least one polymorphic marker or at least one haplotype in a plurality of reference individuals, and wherein risk of developing exfoliation syndrome and/or glaucoma is based on a comparison of the frequency of the at least one allele or haplotype in individuals diagnosed with, or presenting symptoms associated with, exfoliation syndrome and/or glaucoma, and reference individuals.

140. The apparatus according to claim 137, wherein the at least one marker or haplotype comprises markers selected from rs4886725 (SEQ ID NO:86), rs12915956 (SEQ ID NO:87), rs896590 (SEQ ID NO:88), rs12438872 (SEQ ID NO:89), rs4261482 (SEQ ID NO:42), rs2165241 (SEQ ID NO:15), rs1992314 (SEQ ID NO:44), rs4886776 (SEQ ID NO:45), rs2028386 (SEQ ID NO:46), rs4337252 (SEQ ID NO:47), rs2028387 (SEQ ID NO:48), rs4077284 (SEQ ID NO:49), rs893816 (SEQ ID NO:50), rs4886782 (SEQ ID NO:51), rs893817 (SEQ ID NO:17), rs893818 (SEQ ID NO:52), rs893820 (SEQ ID NO:53), rs12440667 (SEQ ID NO:54), rs1530169 (SEQ ID NO:19), rs893821 (SEQ ID NO:90), rs750460 (SEQ ID NO:55), rs4243042 (SEQ ID NO:56), rs2304722 (SEQ ID NO:91), rs4886623 (SEQ ID NO:92), rs2167648 (SEQ ID NO:93), rs1584738 (SEQ ID NO:94), rs1901570 (SEQ ID NO:95), rs8026593 (SEQ ID NO:6), rs4886660 (SEQ ID NO:96), rs4886421 (SEQ ID NO:97), rs4886663 (SEQ ID NO:98), rs4886664 (SEQ ID NO:99), rs4145873 (SEQ ID NO:100), rs4145874 (SEQ ID NO:101), rs1452389 (SEQ ID NO:102), rs1078967 (SEQ ID NO:103), rs8041642 (SEQ ID NO:104), rs8041685 (SEQ ID NO:16), rs8042039 (SEQ ID NO:105), rs2304719 (SEQ ID NO:18), rs12437465 (SEQ ID NO:57), rs1048661 (SEQ ID NO:106), and rs3825942 (SEQ ID NO: 107), and markers in linkage disequilibrium therewith and generate an output based on the marker or haplotype information, wherein the output comprises a risk measure of the at least one marker or haplotype as a genetic indicator of exfoliation syndrome or glaucoma for the human individual.

141. (canceled)

142. The apparatus according to claim 137, wherein the at least one haplotype is selected from
the haplotype characterized by the presence of allele G in marker rs1048661 (SEQ ID NO:106) and allele G in marker rs3825942 (SEQ ID NO: 107); and
the haplotype characterized by the presence of allele T in marker rs1048661 (SEQ ID NO: 106) and allele G in marker rs3825942 (SEQ ID NO: 107).

143. (canceled)

144. A pharmaceutical composition for the treatment of symptoms associated with glaucoma or exfoliation syndrome in an individual in need thereof, comprising a polypeptide encoded by a human LOXL1 gene, or fragments thereof, and pharmacologically acceptable carriers and/or excipients.

145. The pharmaceutical composition according to claim 144, wherein the polypeptide is characterized by the presence of a Leucine at position 141 in SEQ ID NO: 85.

146. The pharmaceutical composition according to claim 144, wherein the polypeptide is characterized by the presence of an Aspartic Acid at position 153 in SEQ ID NO:85.

147. The pharmaceutical composition according to claim 144, wherein the polypeptide is characterized by the presence of a Leucine at position 141 in SEQ ID NO: 85 and an Aspartic Acid at position 153 in SEQ ID NO:85.

148. An assay for screening compounds for preventing or ameliorating symptoms associated with exfoliation syndrome and/or glaucoma, comprising
(i) administering a test compound to an animal having symptoms associated with exfoliation syndrome and/or glaucoma, or a cell population isolated therefrom;
(ii) determining the level of gene expression of LOXL1 in a sample from the animal or in the cell population isolated therefrom;
(iii) determining the level of gene expression of LOXL1 in a sample from at least one control animal that does not have symptoms associated with exfoliation syndrome or glaucoma, or in a cell population isolated therefrom, in the absence of the compound;
(iv) comparing the expression levels obtained in (ii) and (iii);
wherein a test compound that provides expression levels that are similar in a treated animal and the at least one control animal are identified as candidates for drugs for preventing, or ameliorating symptoms associated with, exfoliation syndrome or glaucoma.

149. The method according to claim 148, wherein the animal is a human individual.

150. The method according to claim 149, further comprising determining the genotype of the human individual for markers rs1048661 (SEQ ID NO: 106) or rs3825942 (SEQ ID NO: 107), or markers in linkage disequilibrium therewith, prior to administration of the test compound, wherein the genotype status of the human individual is used for determining whether the animal is suitable for screening the test compound.

151. The method of claim 150, wherein the genotype of the human individual for markers rs1048661 (SEQ ID NO:106) or rs3825942 (SEQ ID NO:107) is determined, and wherein the presence of allele G at marker rs1048661 and/or allele G at marker rs3825942 is a measure of the human individual being suitable for screening the test compound.

152. A method for treating a human individual for symptoms associated with an ocular condition selected from exfoliation syndrome and glaucoma, comprising administering a compound identified according to the method of claim 148.

153. A method for treating a human individual for symptoms associated with an ocular condition selected from exfoliation syndrome and glaucoma, comprising expressing a human LOXL1 gene in vivo in an amount sufficient to treat the condition.

154. The method of claim 153, wherein the LOXL1 gene has a nucleotide sequence as set forth in SEQ ID NO:84, that contains at least one polymorphic site.

155. The method of claim 154, wherein the nucleotide sequence is characterized by the presence of a T at position 7142 in SEQ ID NO:84.

156. The method of claim 155, wherein the nucleotide sequence is characterized by the presence of an A at position 7178 in SEQ ID NO:84.

157. The method of claim 154, wherein the nucleotide sequence is characterized by the presence of a T at position 7142 and an A at position 7178 in SEQ ID NO:84.

158. The method according to claim 153, comprising
(a) administering to the human individual a vector comprising a human LOXL1 gene; and
(b) allowing LOXL1 protein to be expressed in an amount sufficient to treat the symptoms associated with exfoliation syndrome or glaucoma.

159. The method according to claim 158, wherein said vector is selected from an adenoviral vector and a lentiviral vector.

160. The method according to claim 158, wherein said vector is a replication-defective viral vector.

161. The method according to claim 158, wherein said vector is administered by a method selected from topical administration, intraocular administration, parenteral administration, intranasal administration, intratracheal administration, intrabronchial administration and subcutaneous administration.

162. A method of treating an ocular condition selected from exfoliation syndrome and glaucoma, comprising administering an agent that regulates the expression, activity or physical state of the LOXL1 gene or its encoding RNA or protein.

163-172. (canceled)

173. A method of preventing or ameliorating symptoms associated with glaucoma or exfoliation syndrome, the method comprising administering to an individual in need thereof a composition comprising a LOXL1 polypeptide in a therapeutically effective amount.

174. The method according to claim 173, wherein the LOXL1 polypeptide is characterized by the amino acid sequence set forth in SEQ ID NO:85.

175. The method according to claim 174, wherein the polypeptide is characterized by the amino acid sequence as set forth in SEQ ID NO:85 having a a Leucine in position 141 or the amino acid sequence as set forth in SEQ ID NO:85 having an Aspartic Acid in position 153 or the amino acid sequence as set forth in SEQ ID NO:85 having a Leucine in position 141 and an Aspartic Acid in position 153.

176-179. (canceled)

180. A method of determining whether a human individual is at risk for developing elevated intraocular pressure, exfoliation syndrome and/or glaucoma as a complication of being treated with a glucocorticoid therapeutic agent, the method comprising
determining the presence or absence of at least one allele of at least one polymorphic marker in the individual, wherein the at least one polymorphic marker is associated with the LOXL1 gene, and wherein determination of the presence of the at least one allele is indicative of an increased risk of developing elevated intraocular pressure and/or glaucoma as a complication of being treated with a glucocorticoid therapeutic agent.

181-187. (canceled)

188. A method of prophylaxis therapy for an ocular condition selected from glaucoma and exfoliation syndrome, comprising:
selecting a human subject at risk for an ocular condition selected from glaucoma and exfoliation syndrome;
administering to the subject a therapeutically effective amount of a composition comprising a therapeutic agent for glaucoma, elevated intraocular pressure or exfoliation syndrome,
wherein the selecting comprises determining a LOXL1 variant for the human subject, and selecting for prophylaxis therapy a human subject with a LOXL1 variant that correlates with an increased risk for the ocular condition.

189. The method of claim 188, wherein the selecting comprises determining the presence or absence of a genotype or haplotype in the LOXL1 gene that correlates with increased risk of the ocular condition.

190-203. (canceled)


SEQUENCE LISTING (197 sequences)

SEQ ID NO: 1 - DNA, 601 bp, Homo sapiens
agggctgcagagtgagctaaggtctggaggtgaggatgactctgaaggccttcctggagagcaggctgaggaggtaatgcctgacttggtgggggaggggtgctgaggacccgcaggccactgtggaattggacccatctggctgaggcaaaggcgctggcagttatgttgcactaagtgggccaagaggtgacctctgctttggtctgactttggccacagacctaagaatctggatacagttgggctagaagggccttggcagactgcacagctcaacctgtcttgtgcaggtgggaartgggagatccagggaggagggacttgcctgcaccctccccacctcctgcttgggtgtgtggccctcccgctgggtgggaagagcaggcaggaaacattgtctgaggtttccaaccacacgggaggaaccctgaaaagacattcctgagagaggcataaactaagacctagaaaagacagaggctgacctcacagcccctgacttcctgcttcaacctcccttcccagcccaggccagtctcttccccatggtcgcacctttgctcagcagttcttcccagcgggaatgactgcccccact

SEQ ID NO: 2 - DNA, 601 bp, Homo sapiens
caagtgctgctgcctccttcatgaagccttcctgattgcccagggtaaatggaactccttgatctgttagcttctttgctggaccctctagattgatttctgattgatttttgtctgctccagggtcatttggagtgatggttgcctggtatttgaataaagagctcccctatcagaacattcaggttcaaatcttagtgcctctgttcctactgtatgaccttgggcaaccaacctacttaacctttctgagtctcactttcctcaactggcagatgaggggcattttgaggattaaacmaggggtattttgaggattatggtcgggtttcaaaccataccttctttcctgctttctttttctccacatcacttagcaccatctcacatcttatgcttcgttttgttcagtgcctgtccgccctcactccaggagggcaggggtgcctgtgcattgctccctgccacatacctggcacagagaggcggctccatggagcggaccggatggatggtgcaggatgagcaccaggctccgcatcagggcacagtaaatgttggctctgtgccctttcatgtctggaatccccagtttggcatt

SEQ ID NO: 3 - DNA, 601 bp, Homo sapiens
agcctgggtgattccacttccccaagtcagctgttgcaccccaccctctcttccctttctaagctgaccaatttctctctccccttaaaaatagtttaaaaaaagtgtttcctaaatcctggagcactggaatttggtgattgttcatggagatttgggccagctgtttcctatgctggggctctagcagggcagtgataagggaggggagaggaactcagacccagggagggagggaggcccttggtgggctcagcagcagaccaccagagaggacactgtgtgagtccctaccatgttrcatttcacgctggggacctaggctcactcaggagctgtgatgccaggagaacaaatcgtggcaacacgctccacttggcttttggacttaaaagcgtaacccaaagggcaactgacaatttatgaatgactctttctggctgcctaggcttggttccaggccccttgtgtgtgtccctgtgcgtgtgcatatggttatgtgtatgcgtacttggcccaatgtctgcctctgagctcacatgtgcacacaagcatgtatacatgccaaggcttggctacatctgcctctgtcttctgctca

SEQ ID NO: 4 - DNA, 601 bp, Homo sapiens
tctcttggtagctgctgctgcttctttcttatcttcctcctggaccctggaaatccctgtaattctgctgtcaatctcttcttcccttttttagtttcttttccccactatctccctcatctccgaacacccacacccagccagctccacatgtgctgcacttttcttggaagcagctctcacggctgtcccctgtcaaggctgccaccctggttcagatgcccagtggcctctgtcttaaattgctacagaaatctccaatggctcctatttctttcttgaaaccactgccaaaatattytcccagaataccatttctccttgtctctccctaggtcaaaaactgagcaccattcccggtgccttgagtgcaaaccattcctcttagtactcagaccctccgttctccgtctgggtcccccaagttagagcaaggaagagattggcatatgacctgtcagaggtagggcgctgcatacagataagggggcttgggagttggggctagcttagagagggtagggacttccccagtgagtgttggaggaggcattgctgaaaccagaggcagaattgtagctggaaggaactgggaaacacc

SEQ ID NO: 5 - DNA, 601 bp, Homo sapiens
gccaattaaacctcttttcttataaattacttagcctcagctatttctttataccaatgcaaaaaaaaaaataaaaaaaataagaataaaaattaaaaaaacagcctaacacactaccatagaagaattcctcagtggggcaacaagatgtgatgttagggttaactatagttcctgtaatccctctagatcttggtttaagaattcagccttgaagctgaaaaggaaagacaattgaggatggccaatgacaggaagtagtgcccagtaccaaggacagcgctgctccctggacagcaaygttgatttaggaagcaacctgtttccagaataatagaaaaggcctgtgctctgcgtcaggaaggaggtctttgtatctccttccatgagttggaagagaaaaaatgtcatggatattctggaagctccttgaatccctggatacttgactcctcaggcagacctctggtgagattggtgagaatgaaactttttcctaaaagatctaaaaataagccacatgacatgccaatatatctaggtggcatgactaaaggatgcctggaaggggtctgggaagtcactttagctccttgtgttt

SEQ ID NO: 6 - DNA, 601 bp, Homo sapiens
gcaatgtttgttgagtgaaaacctttccctgggggttggcctttgtgtggcttctccaactccagccttagagcaatgctagatcctctaaaatcccataagattagtgggagactataggctaaaaagggggataaagccaagtagatgcattactaattaattcagcagacatctagtgaatactttcagtgccaagcactgtgctaagtgatgggaccacagaggtgattcgggcatattcccaatctcaagaaattcactgtggaatagagaaatgaagttatctaagctgcttgcrtttccctcattctctggccgatatctaacctagtatctgacacatagtcaatgtttaataaacagttactgaacaaataaatgaataaatgagcaagtgagggaacaaatgctatggagggtacacataaggctgtgtcagagtatggggaagaaaagagtgctgggaatatcctttcatcatctctcatttactgaggcatatgctggttgttggggattcagagagaaggggcgcttgtcccctgttcttgaagatcctgcagcccaggggacagaaaaaatcaatgtttacaagaca

SEQ ID NO: 7 - DNA, 601 bp, Homo sapiens
gagacactttatgaagtggtccttcctaggggatcccccattagaggatgagaacagatgtccatggaaggcagaggagctactttccatgacctgtgggcatcaatggctctgttcccaaaggattgaggaggggatccacagggccacagaagaacgtaagtcacagtgactgctcagtggccatatatggaacattcattcatcagcaagcacttatagggtgcctacctcatgccagggaccctgcacatgtaactcatgtttccttaagctagggaacaatgaggtacaatcatarcaattgccatcaattcattgaccctttccctacttaccattgaaggaccaaccccatgtaaagtcaccccacttccacacacacagtgatttttggaatttccatgggtatctccattgtacacagggataaatattttctgttattaaatggggggtgtgctgtgtccttgctctcccttcctgagaccctcacagtgatgctcactgtctgtgaagtcataggaggggaccccgctgctgaagccttttatgagaaagccatattccctctgctcccaaagctgccagtccacaagtg

SEQ ID NO: 8 - DNA, 601 bp, Homo sapiens
ttttctaatgaaaaataaaaacagacacctttgcagggaagaagaccaaccttactcttagagccagggcattatgccacaaagtggccttgtgcaaccccagggcttggctgtgtgactggaagagtcagccctgacggaagcttccttgctatgctttcagcttacctccactcttcatcttacccaagcacagctatgaccatgcccctctccggtcaaaagccttcagaggccttcagacatctgctgggtgaaatctgaatgggcttcttggcatcaaagggctgccatactctgrttctaagccatcttcactcctatcacagattacagccacacctagaaacccactctttcccatacttgccccttgctgccaatcctcaggacctttgcacaacatttgttctgtgtggagcactccacacttccccatctccacctgtagaaactcactctttccctgggggacctgggattcaaggaggaccatggccatgggggtctgacaccacatctgccaatgctctcctctctaattttctctctcctgtctccctgtgccctcattccctccgccactcctttggcttcattt

SEQ ID NO: 9 - DNA, 601 bp, Homo sapiens
ttggtgcaggctggagaagtcagtgcaggattcttagaggaggcagagtttgagcctagcctttgcaggatgaagaagagtagcttaaaagattaaagaggagcatgcatcttgggtgggtttaatgcacaagtaaatatgtagaggtgagaaagagtattcaggaagaattttctgaactttaggatagaactaaggttgggctggaaaatagatccttgggtgatcccagcagatccaaggccactctggggtcaaacatttctctatgagaagagcttgtgcccggggatgacttacygtccccaagtggacagaaccatcagcaggaggtcagtcccagatctgaatggccaagggcttaggcttcccccgactcttgggtctgtggaacagctgtcctaaaccccacctccccaagccacagaaaatgaagccagattggaataaaggatgcccttctgctatgctgatggcgagggatcaatgtcattctgcacccttcccccaccacggccaagaggtggcaagacgcgtgcaatgggcaaggagacctgtgctccatgtactgtccttccaccaggagctggaccacgggctc

SEQ ID NO: 10 - DNA, 601 bp, Homo sapiens
atcaaggtgctggtggggctgtgttccctgcaggaggctccagggaagactctgtttccttgacacctctggcttctagaggctgctacattccttggcaaagcctccagtggccaactgagtctttctaacattgtgtcactctgacatggactgttcttctgcctccttctcccacctttagggacccttatgattacattgggccaaactttataacctctctattttaaaaagtcggctgatgggcaacctaaattcagtctgcaatctcattttctctgtgccacatcatttaacytattcacaggattcagggattagaatatggacatttggccggggaatggggggtattattctacctctcacaccgagtaaaagggactgtagtgctcccacatctacacacggggctcaggctcccatttgggagctggagcttggtgttctcattttcgtttgccgccttgggcaggtccttttcctcctggaccccagcatccccttctgtatttaaacaagtgggctagatgactgctgtattcattagtgtaaagttgagctctgaaccatagagacccagcaagaactcagttgc

SEQ ID NO: 11 - DNA, 601 bp, Homo sapiens
ctaatttttgttttttgtttttttttagtagatatggggttttatcatgttggccaggctggtctcgaactcctgacctcaagtgatctgccagcttgggccttccaaagtgctgggattacaggcgtgagccaccatgcctggcctgttttgccttctaatactttactactcatgtggtttgtggactagcagcatcacctgttactcaggagcttgttagaaatgcatcatcttgggccccaccccaaaccctctgaatcaactctgcacctgcagaagatccttaagtgatttgtaygccaatttaattttgagtcacatattcagatgtatactaatgggaagttgggagtgtgtatatttacatgtctactcagctctgtccatcagcacatctttatgtctgcaatgtggcatttcaagatattcttctctccagcagaagagagtatcccaggaagaaaggaatcttctcttattccaacaggaaccctcactcctgttgttgttgttgttgttgttgttgttgttagagatattattattattatgtgctctgttgcccaggctggagtgcagtggtgagatctcggctgac

SEQ ID NO: 12 - DNA, 601 bp, Homo sapiens
atccttattaaatgtgctcagttttgtcctccacaggggtctggaagcctttcagagctggttctactctaaccatttgtatccagatatggatccaccgcagccttggaaaaccctccatcaaggaggagccactgacccagtctcttccagcaagcaccctccctagtctcagaatgtgtctctcagccagccagcctctcttcctatcgtacgaccctgtctcagccctcatgtgcgtcccagcccctaaggtcaaagacatagcatccaccacaaaccattattccagaggactgtytatattgcagctgttttatctactccctcactccagtgcccctggccttcatcctgcccatcgaaggcatcagtccactgcgggctttggtgtcctcagaggaagaaatcctttaatgtctgaggaacctagggaggaggcaggtgggaggtgcacagacattggtctgtccctttcattcttcggttcttctaagcagcgacattctcctgagcctctgggtgcccagacccactgggatggacacagggaatgggggatgagggccctgctctcaagtagtacagcctgctcagggag

SEQ ID NO: 13 - DNA, 601 bp, Homo sapiens
aatttatactctccactgccttcccaggaagctgagttctggtcacaggtgaccctttgtgctgccatcttgaaggagctgggtaggcaaattagggacaggaggggctagggaagtgggagacgtatgtgggaggaggtcggggagaggctggagagaactatggaagaaggatttcatgtgtctccgtggggtatgagaccatcttctgaatcttcccacccaaattgtcagtatactcaaccttgctccctgatgtctttgagtgaatggaatctggagtggagagggtggtggcagkttggaaagaaggatgagggaccagccccactggttacatgtggagaggacaaggtacacggaggaggaaggcaggcctggcagagctcagatatctgtgggaggaggaggcaggttcttgcctctctcttcctctgggaagcagcttgtttgggaaatccaagcccaagtccatccagggcaagtgcctcgattttcccttgagaggaagaaaagggctttcctgacccaggaggaggagaagactgaggtcaagtcaatcaaagggacaaagctaacccctatgccatcaagaatgtat

SEQ ID NO: 14 - DNA, 601 bp, Homo sapiens
agaggcagatatgaagatttcttcaagggtgcttattacagcatcatttataatagcaaataaagtacccaaaaaaatcaacaaaggaggtgattcattcagttatgatttgtccacataagtcagtcattaaaaatctggttaaagaaaactcgtttccgatccagccaggtgctgagacaggcagaactttccagagcttctccttggaagctgccagctgctgtgatgtcctatttcttgatccaggtggtaatccagaggtgtgctcacgttgggatcattcatcaagtggtacacytaggatttgtggacttttgtgtgtgtgtatgctatactcatgtttttaaaagttatttttggccgggcacggtggctcacgcctgtaagcccagcactttgggaggccgaggtgggcagatcatgaggtcaggagatcaagaccatcctggctaacatggtgaaatcctgtctcacctaaaaaaatacaaaaaaattagccgtgcgtgatggcaggtgcctgtagtcccagctacttgggaggctgaggcaggagaatggcatgaacctgggaggcggagcttgcagtgagccgagatcg

SEQ ID NO: 15 - DNA, 601 bp, Homo sapiens
atcccactttaagaacctgcaggagctcaggaaaaatcagatggcaggtccttctgtggtgtgatgaaggatgcatccacctgctctggtccttaccaggtacttgcagcatccctgcagaagggcgcattatagccatgcatcagctgctctagggccccttggagaataggatgttttacctagagggtgctaaacttttgcagtggcttttcttagaatgcaagacctcagcatgaggctcagcctgatgggccccacctctgggcagagaaaactgagctctcaaatgccacaataytggcagaggcatgcctgggacttggcatctgggcaccaagtcagtgggtcttctagggcctgtctttgggctgttagttcttaaggaagccagttgattgagaggggaatgggacagggcaaggccatgtctgaagtgggggtgcttggtgcttagcagacctctggccactaccctgagccccacagttcattttgggtcaaaaacatttctcccctgaaggtagcatcctaggtgccaagctgagtgtactcacatgcatcaagtccaggcccttccctggggcagtctaccaggagg

SEQ ID NO: 16 - DNA, 601 bp, Homo sapiens
agagtctctccccagtgtggggttagggtgtagcctggtcatggtcattgcagattccagaacgtttgtgtaacaaacactcatatggtgcatactaagtgccggacactgttcttggagttttgcagttagaatcacttaatccctgtgacaaccctgggattgttatccccattttgcagatgaggaaactgtggccctgagaagttaagcgacttgccctggaggcagaatcacagatcagcatccctccttccttccccgaagttgtctcagattgttccttcagaaagaaaaagartcttcaggcttagaaagctgtgtcggatcagacaagctgtggaaacggagggttctgggccctcctcctgcctccctgtttctcctttgagagtcatcagtgtctgaacaaacaagtggagtcctaagcagcctcattccaggcagacttcctgtctcctctcttatcagccacttgcaaggcaccttctctctcccctgctctcaaactggaccatccctgcaagaagagacagctcctgttttcaataataagaataagaatatctgatatttattgaacatacactatgctaagcat

SEQ ID NO: 17 - DNA, 601 bp, Homo sapiens
tgctctgcagagctttctgcttctctgccacctaggggctcctctggagtctggctgcgctagccaaaacatcccgctcagcacatatttcacaagcaactgaccagttcctcctggactttgtttatcctccagccctgggctcaagagattagggctcttttgcattccactttccatcatttgcccctcatgaggaggaggcttggagaggaaagattccagagacaaggtttactgaacccacaaaaaagaggaagccaagagggaaacaggtgaggtgtggacgagcaatgggaargcagccacagggctctcagcgccaggcagcctccctccaggaaccgctgggcaggagggaacagatttctaatgaagcaggaaggatttaggtcagaaggtgggagaatttcctgactgctaaagtatcgtctgtaggggttgtgagagggctcgggctcttgcccaaggctggggaatgaactgaacagctcctaaaaagtattttaatctccaagtgtttatcgatctatccaacctgtgttcaggggccagctagttcagtctccctcatcatccagatggggaaagtgagcctcag

SEQ ID NO: 18 - DNA, 601 bp, Homo sapiens
cctgacttggtcccagaccccaactatgtgcaagcatccacttatgtgcagagagcccacctgtactccctgcgctgtgctgcggaggagaagtgtctggccaggtaaggagctgaggcagaagtgtagagtgttgggatagtccccgggagtcacccagaacccagcctagctgtggctgccagctaggctgtctgcaagctgatgaccctggccaagtgccccagcctcccaggaccctgaccgtttcatcagtgagccttgcctgccccgagggagtcaggaggggaggggagcaagygggggcccagcatttctgtatactgtacctcactatacaaggaactccacttttactgtggcacttcatctttatatgagccctgaaggcatgggttaccccctcaggaaactgaggagcaatgaaaggaactcaaaccttttggatctgagctgtttaatgagaaggttccttttgggagcccaggcttccagtggtcatacagggacccaaggaagtcctattgggtggatgtacctgcaggatagagcccaggagtatggagccatgtccacagaggacagttgtggctttggctgg

SEQ ID NO: 19 - DNA, 601 bp, Homo sapiens
gctgctggaggtcagaggagggagggatcagcaagggctgaggcctcagaggaggtgggcgtgggagttcagacctggagaatcctgcaaaactggagccctggggcaggagggccctctgagatcatttcaagtatgggccgcaggctggtgccagttcacatgctgcaggcagaacccagagaagaaaagggaccaattccaagtctcaccaccagtttgtggccaagctgggcctagaacctagacccttatggtttcccacggtgtgctgagccgaacagaagggcaggatttaacygaacagagggaaggaggatgttttgtttcaacccagacatagacagattccaagcctcccccagacaaggctgagggcctttcatcctggcagacagtctgagccgcctgttgtccagacattctctcaaagagagcgctaatccctacgactggccaccgctcccctctggccttaggatcggaccttaatgagtgccagggaagagcccctgaggccaacagagttagctgtcagccgtcagctaaagaggggaggggccttcagccaccccttcctgttcctaccacacatgtgtgt

SEQ ID NO: 20 - DNA, 601 bp, Homo sapiens
caggggaaggtgggccagaaactcctgaaggtgggcgtggggtggctctgggaaacaagcagcatcacagccgctcctcttgtccctttcccagggcctgagcccaggctgctatgacacctacaatgcggacatcgactgccagtggatcgacataaccgacgtgcagcctgggaactacatcctcaaggtgggcctctgggtctggggctttccctccaacctgatgttcatgtccaatgtccccccgttcctgaaagaagcttcagcctctgggcctgttcccttctccccagctgcmgagcagaggcagcatctgaggcatcacttgaggtttaggcttcccagccgaagatttaggtctcaggctgacatagcctctctttccataaagaacccagggaacctttgccccagctgtggtaatgccagaggcagtggtgatactgtctccctaagtgacagaggaccagggagagcaagaaagacttggagagccatgtggtctatcttcctgtcttcggacagggcagactttgtatcatttgggagacagatgagaggccctcagctccttatagggacccctgcaaagccaagg

SEQ ID NO: 21 - DNA, 601 bp, Homo sapiens
gagggaaatgtaggcccatgcctgccctgccacgagggatctgggctgtgggagggacgccctggctgaggaggccacgggggcctactttgcagcccctcattgacccactgtctttccttcctcagatcctgatctccgggagggacagatggccaatctctccccttccaaagcaggccctgctccccgggcagcctcccgccgaggggcccagcccccaacccacaggcacggaggggcatccctccctgccggcctcagggagcgaacgtggatgaaaaccacagggattccggaygccagaccccattttatacttcacttttctctacagtgttgttttgttgttgttggtttttattttttatactttggccataccacagagctagattgcccaggtctgggctgaataaaacaaggtttttctactctgtggctctgcatgcggcctgctggctggctggccagccacagctagtttgggcctgggggatgtccttaggcatcatccttcccctcgccaaatgtggaaagggcagccaccacccggtcaggaccacagtgaccatgagggcgctgagcccatcgtggaagc

SEQ ID NO: 22 - DNA, 601 bp, Homo sapiens
attgttgtattgttatttttttcaatattttttatctgtgtgttcactgtgcagttggttgaatccgtggatgtggaacccgtagataccgagggctgactgtatttcattctggtttaaattttcatttccctagtgactaacaatgttgagcatgtgttcatgtggttgtttgccaggatatctcttctttgatgaaatttctgttcagatcctttgcccatttttaattggcttgtttgtctcattattgagttggaagaattctttatctattctggatacaagttattcatcagtkatatgttttgcaaatattttttcccaatctgtcgtttgtcttctcattttttactagtaccttctaaagagcggaagtttttaattttgataagagccaatttatcggttttttttccttttatgggctgtgttttttgtcctatctaaatcttaacttaaacaatgtcacaaagattttctgtaagcactgcaataaatggtatcccatgagtttttaggactaagttctaatttttgattttgcccaaatttctatctaagggatctagggagtcatgccctacaaatcctaaattct

SEQ ID NO: 23 - DNA, 601 bp, Homo sapiens
ccagccccacccttgcttccaggggctttgagacactcaggagaaggagcttcaaatattgtgagggtctgtctgtgtcagccaccaggatgtcaatccctgcctgggctggcagcatggaccttgtggaccctcctagcccaccccatgcccccaggagccccgcttggcttcttctcttttatacattgtaaacaccacatttatttgtctgaggcttgcaaacctctggtaagaagcacagaacgcagggctctccttcatgctgccctgggccccagctcgccaggcatgcaggacrgagctggcagatgagtcagaaatctttgggagcagcgccagggaagcagcaggccctgctccttcccatgcccaatcccgccagcacgggcctggactgcagccaggaaggtgggccagccaggatccccaggtaacttcgccacagggacctcttcttggagctgccttcccttccctccccatccctctccaatccccgcagagcttagagcctgggccacatgcttggtccctcacacaaggaccaaaccagctgctgtcaggctgtttctgtgggtcctgtctctccagtcaggaa

SEQ ID NO: 24 - DNA, 601 bp, Homo sapiens
gcggatccggcccaggcggaacacaatcatccgctcgtaggtgggcacaatctgtccacaatggcaagggaaagagttgaaagtggtcagcctaggctggccaatgctgtctgcctaggtcccttcctcacttctctcctcctcacctgggtcacagttaggtatccctagcactgtttccagatgagaaaaccaaggctcagggaagctaagtgactgctcaaaaattgcatagccctaagtgaaatagtgaagatttgaatgcagttctgaataactccaaaggccatgtgtcttaacyactgcactgtattgacttgatccttagagaaacttttcatctctgtggtctgaagttagacatgactcataccaggaaactgtctgtccttggattagttaggagaaaaggctcggctgccctaacagcacccccggggaaagggctttgtcaactttaggtccctggacctcctagagaagtagtgatgtccaaattgagaaatgaaggcccagagaggcaaagaaacctgttcaaggtcacatggcatttgtttctgtggcagttttagaactaggatataaggccatttaaggaccc

SEQ ID NO: 25 - DNA, 601 bp, Homo sapiens
aaggggtaataatatttagggaaaggggacagaggataggcaaatttattcatcaatgccaaagctctcagtgtggcccaaccaccctgggggctgacccagccagccttaccttcagggcaaaccagccagaaatggggaaggtgaccaacagcagcaagaaccccaggaaactgatgaggccatgacagaggcaggagggccagctctggggtacatctgcaggtgagagcagaggtggtcaactggacctgcacgacagctgccaagcggagtgaacttgggcaagtgtaccttacgyccaccttcccctcgagaggctccttccttctcagggctctcaatctggctccttgcctaatattcccgtatctttgggcagctgcaagttagggtctgttccctcctcccatacatcaacactcatttgtcccaagtccaacaaccctcacaaggccttattctctgctaggcaacagccattcaatctcagaatccagtccgcgtgatgatgctctcctcactggactgagtaggggtcaatcctctcgtttaatttatattgcctacccatacctggcatggagccaggagcacctgg

SEQ ID NO: 26 - DNA, 601 bp, Homo sapiens
agatagcgctgcctggtgttaatggtttcaacttttcagacagaaactaactacttgtctctgatcctagaatccagccttactgagattccgtgtcagtgctctcggaaaattccactaaattctaaagggcagtttggtaacatgtatcaaatgtagaattcccgtccccctacacccagcagtggcacttctaaaaatttatcctaaagaagttactggataaagatgcaaagatgattctgcagcattatttattacaggaggaaactggaaacttactatccacctgcgtggggamtggcagcataaattgtggtcatcaacacggacactgcatatctgcagaccacatttagagttgcaaggccccagaagctccttgtcctcagctgtgtgactctttggatctttgctcctctcgccctctacctggtacttggatgcaccaccccaccctgtgccggcctactcacctggtcagcgtaggggtgcggtggttggggttggagcagaagatgttgttggtcttgcgggtgccgtccaggaactcacgcaccgactggttgcgcagctctgctaggggccgggcctcgtgctt

SEQ ID NO: 27 - DNA, 601 bp, Homo sapiens
ttgaatttgtggagagtttcagcctatggataagttgcatgtgtgtgcagacttgtactctatggcaagaacccagaggtcccagaagatacgttcagatcaagtgctgtggcagttttcagaagggaaaacttgtatttatgtgggagggtcagggaaaccctgatattttagcatttgggctacaccttgaagggtttatttgtctctataagttgcatctaaaacaaactgtataaattagtgagtttttttagttagtagcttaagcaaaagctggattgatggctgatgtaagtgmataggggtcaagctggcctctgacctgaccaattagatattcagattgtgtcaccaggaggtactttctctgtatttcttgtgacctggctttcttccaagactggtccattcacagaagggtttactcctcatgggtgccagatgattgcaacagctccagaggttacaccctcagccccaaattcaaggggaaaagagtgtgaatctctcccagcattccaagctaaatctcagtgtgtctttgattgggtcaggttcctctccctgcaagggtggctcaggctaggggggttctcag

SEQ ID NO: 28 - DNA, 601 bp, Homo sapiens
aaataaataaaataaaatacactactcaggaggctgaggtgagaagatcacatgagcccaagaattcaagtccagcctgggcaacatagcaagaccctgtctctgaaaaaaaaattaacagtgtggctatttcactgaataatcataaagggataaatgaaacaatacatcccagagacgagaaagagctttgttgtaatacctagatgggatgttctttgagtgttccccattgcttcctatttaaagtccaaactcctttacttagaattgattatagaaagtttaggaaaatctaaayaggtgaaaaggagggatgagagaaagaggaaactactatcactctagaccttttcctttcctttttgctatctctgtgatcatctaaaacaaaatgaacaacatttagtgttgtataacttgcttttcttataaatagtcatgaagatctttcaaaaaaatatacagttttgaattttttaggcagtggcatcttttttttttttttttttttttgagatggagtctcgctctgtcgcccaggctggagtgcagtggtgcgatcttggctcactgcaagctccgcctcccgggttcatgc

SEQ ID NO: 29 - DNA, 601 bp, Homo sapiens
ggacattgtttctttgaatgcaaaactgtgttcaggctgagcctcgtgagagccaggattttttcaggatatacatgcaacttggatacacctgcaatctggctcatgagctacatccatctcactccccttcctcatagatttcacggcctgagcaggtcaagtgcattctaactcaacacagcaggtctaacagtcgtcccaagcatgctcagaggaagtggctggattggcattgtaatcacactccaaaagtccaggaggtacatcagggaagatccagcctctcccctcctctgckgtattacaccctggtatgaaaatgtcacctgcattcaagactgccacatctataaacaaatgagcagcattcctctaccacagacatgcaatgaatcccccgattcagtggttcttgactttggttaggcattcatagctttaagaaaaaactgaggcccaggctgcacccaagactgattcgattagaatctcagggcgtaggacctaggaatctgtagttgggaaaactccccagtgtacagccagcttgagaacaattgtccttatcactgattcattccaggtttgctcatcagcc

SEQ ID NO: 30 - DNA, 601 bp, Homo sapiens
gtccagcctctcctccattaacaagcttggtggaaagggggtgtccttttattaaaggagacagtagaggtttcttagagaaggcagctcgagtaagtttaggacaaaggacatttccctggccccgggacaatggaaggctctaaaaacgaggttctagtggctgccataaatgaccctgctgtaacgatgggaggggtggcctaaagagacagtactgtggctctccagggagctctcctcttggaagagcagatctgaaggcgggaagactggttctaaggccgaggacattgccccragctttgtggaggcactcaggaacacaaaggggcatttccagttgggtttggagcaggaagaagaacgaaaaagagaaagtcctgtgtgccatgtcagtgggtggcagcatggactcagcctcagctattggcaccaaggatctgaaccagggaggacccagaaggggagcccagcccctccaccagcttgtgggagagcgctgctgcccagtgaagcctggagagaggaaacaactggattctcccaaagcgggagaaggtggagtctccagccacccagctacaagctaggaagacgc

SEQ ID NO: 31 - DNA, 601 bp, Homo sapiens
cccagctggtgaacaaagtgtttggagggcagccgagcctccccttctgactgctatcccaccacaaccaggatcagagctgcatttgggggctgatgtgtggctactgccagcagaccccaaggtgaggtctctagatggtgagtcagcagccctagaggaccccctgctcccaccccagagctctctgtgaccctgggtcctcaccctgcccttctctcctgcccagcccagaagattagccagctggctgcggtgaaccgggaaagcaagttccgcgtggtcatccagcctgaagccytcttcagcatctactccaaggccgtgtccctggaggtggggctgcagcacttcctcagctttctgagctccatgcgccgccctatcttggcctgctacaagctgtgggggcctggcctcccaaacttcttccgggccctggaggacattaacaggctgtgggaattccaggaggccatctcgggcttcctggctgccctgcctctcatccgggagcgtgtgcccggggccagcagcttcaaactcaagaacctggcccagacctacctggcgagaaacatgagcgagcgcagcgccatgg

SEQ ID NO: 32 - DNA, 601 bp, Homo sapiens
agcatcccctgggagcttcttagaaatgcagacctgtgggctctaccacaggcctctggaatcagaatcttcagggtggggcccagcagtctgtgttttaacaggttctcccggtgatgctggtgcatgctcaagtttgagaaccgctgctctcatagactgctcctccaaggggaagcagtgtggaacagcagagaaagtcccgtccctatctctgactgtgcagtgagtgtggcctaggaacaggtcccttcctaagctctagtgtccccatctgtaaaatgggctgattggcctccayggtcagagcggccatctggctgtgccatcctgcatttttaggaatggaaagcaggcctctgaggcagtggacaggaagacttctcatccttgaattctagctcccattccaaactgttagtccccaaccttgtggtcacatcagaggtcaacagcagaacagaggcaggtcagggttgtccgctctgattagcagaacgacagccccttcctcctcctccctccttcccatccctactcattagctccctgctcctgggatgcttcccaagcagcccaggcctctagcctccatggctga

SEQ ID NO: 33 - DNA, 601 bp, Homo sapiens
agagacgtccaaacttcctgttttttgtttttgtttgtttggttgtttcattttgtttttgtttgtttgcttttagtatggggagaatctgttgaaattctagactcttctctcaacagagtctccacaggtatttgctttattcaggtccagcgggtctggagctgacatctggattcagagacaaaaataagtgacggtactggccccattcctcccctagccccaggctcagcctgtgcctgtgcctgggaccaggaccaaggccactacaggcgagtgtgttctcagaaacctggayctccattcccttccatctccctccatctggcccacctagtctccacccacatagaggtgcagcccgcatcccacccactccttcccgtgaccacttcttacctacattgtaacggggtggggccttaccctaaatcatgcattctcaacagaggcattatcaccccacagaggatgaaaaggcaggggaggaagggcagttgaccatcttggctattaccatggtttctgaccctccaaaggctcacagtccatacatatgtccatgtaaggaagaagtaaaaccatctctattcgcaga

SEQ ID NO: 34 - DNA, 601 bp, Homo sapiens
aaatactaataataccaatagtggctaacatttattgaactcttactatatttcaggcacatactatggatcttacaagcattattttgtcattccttatagtgatcctgtgaggtaatattattattctaatttacaggcaaagaaaccaaagagcacagagggttagtagtttgttgaagatcacacagctggcaatttgcagggttgaagtttgtccttttgttatctgactccaatgccagagatcttagtcaccaagccaaacctcctcctgaagcctctacaaaccagtgaagtrtagaataacatattttctatatggttaaatttgcggctaattacagagtttgtctagaaaaaggatttctcttgctgtttctcttatttctcaaaatagcatttttttttttcctgattataaaagtagtgcaggccaggcgcagtggctcatgcctataatcccagcactttggcaggccaaggtgggcaggtcacttgtgatcaggagtttgagaccagcttggccaacatggtggtctcaaactactatttcatgtctctactaaaaatacgaagaaattaggcaggtgttgtggtg

SEQ ID NO: 35 - DNA, 601 bp, Homo sapiens
gaggtggggccaagattcatttttttcccatgtgaatatccagttgacctaacaccttttattgaaaagattatcttttcccccactgtaagtaaggccttagcacctttgtcatcagtgaggtgaccatatatgtatgcattgtttttccccttgcagtccggactcggcttctgtcatcacatctctttgtctgactctctttgctgtgtctctcttccacttttaaggatcctagtgatcacattgagtccacccagataagccaggataatttccccattttaaagccagctgaatmacaactttaattctgtctgaagcctgaattctcttttgccatgtaacctaacatattctcaggtttgggggatttttttaccattgatttttgagagttctttatgtattctagatactagagagttctttatatattcttcttttctttttttttcactcttgttgcccaggctggagtgcaatggcatgatctcagctcactgcaacctccacctcccaggttcaaggaattctgcctcagcctcccgagtagctgggattacaggtgcacgccaccacgcctgcctaattttgtatt

SEQ ID NO: 36 - DNA, 601 bp, Homo sapiens
gtctccagctctggctgagctgtcagctgacagcaaccacatgagtgctcccaacaagatcagcagaagaaccacacagataccagcctacaggactatgaaaaatgatcaatcatcttcatcttaaaccattaagttttagagtggtttgtcatgtagcaatagataactgaacacttgtaaatcatctaactggattttcaaaaacccaatttgaatgtctttcctttaatttataacaacttattcataatttcttatacttaatgatatgtttgggttgattcctgacactttgagytttctatcggccatgcttttttgcctcccacccccatttgtgccctttgttagataggtcaggttttatttatggtatttttcttcttacagtcaggaagttataaatcctatttccattcttcgggcttttctattcaagaaacaaaggaatatatctgtttcttcatctttctttggcgattgggtgtgcttcgggtttaattaggttgaattttacccatcagaaggaagctactgttcgtggcctagaaaacgataaagtatagagtaaggcaatcaaaatcaccacaggaaagga

SEQ ID NO: 37 - DNA, 601 bp, Homo sapiens
aaagggcttccagttgcttgaaaagcaaaaataaaaagaagaagctcatatgtcaataaggtaagtcattaagaaagtaggtgcttttaataccaggtaatgtcagcataagaatatgtatacaaaggtgggttatttttagtattggttgaaggcacattattcttttgacaattagaataaagaaagcaattagaaactccaggaaaatgaaaagctgtgcagaaaaagctgtagcccaaatataaagcaaatgaaatgtggcatgtctttgagcagctgatggagcataagaaaattraatacatttgaccttgatgctaaggaccccttctgagcagcctgggcatcatgacattggatcagaaaggaaagaaatgtaattcttggatattccttggctcagcggtgacagtgaactgtactcacacaggcatgagaatgtaatattggtttattgattttttcagagtcaatttatggacaaaatgcagctgttataataatgacttcagaatagtatctaaatgttatcatcatttaaaatgtaaaagtaagactattaaagacttgagagatagaggagagatagaagtcggtt

SEQ ID NO: 38 - DNA, 601 bp, Homo sapiens
ctggaaggtcaaggttattgttcattcattcctttgttcagcaactatttgttaagcacctactaagtggcaggccctttgctgggcactagggggatagcagtggacagaacgaagtacccacctgtgtggagctcacattcagattgcggagatagacaggaaacacactaacagagatgtcaggggaggggagggaagaaggacaagtaaggcaaggtgaggagacagcaagggacaaagactgtttatatagagtggcctgggaaggcctttctggggagctggcatttgaacaaayctcaaaagacaagaggaagggagccctgcaggtgtggacagactttgtccagttagaggaaatagtaggagcagcagccccaaggtgggagcagggcacacgtggggctgcacacagtgagcagggccagagcaaaggggggtgaggccagggaaagtggccaggttgcccaccaaggctcacctgggttcctaggtcagagatcctgagaagaggggagaagccctcatgcccaagtggtgggaaggggtggaggagaatggggaggaaatgggagtgaggccaccccaggtaaggtca

SEQ ID NO: 39 - DNA, 601 bp, Homo sapiens
gctgctaaaagtgagtgagagagagagaaaaaacacaacccaaaaaaatttggcatctcttcccccctcaagtttctggtgtcacttatgaaacacaggtccttgttgctgcagagaagcagttgttttgctggaaggagggagtgcgcgggctgccccgggctcctccctgccgcctcctctcagtggatggttccaggcaccctgtctggggcagggagggcacaggcctgcacatcgaaggtggggtgggaccaggctgcccctcgccccagcatccaagtcctcccttgggcgcccktggccctgcagactctcagggctaaggtcctctgttgctttttggttccaccttagaagaggctccgcttgactaagagtagcttgaaggtaagccagtggggaggagggctccagggccagcggcgggagcgggaggcctgttggacataggggctggttccctcttggtccatccctgctggtctgaggtgcgtgggacaatccctagcttggagccgtccagggggcatctgcttcttccacaacccacaactgaggccccagaaatcccagctgcgtttgggctgagcctctggcc

SEQ ID NO: 40 - DNA, 601 bp, Homo sapiens
ggtggggccaagaggcttccacacagagttcagtttggggcagggggaactccccacaaggcaccaccctggcaggggttctgaggcctccaggagactctccagaccctgacctctcactttgctcattctctgcccaccagtgtgctacatctcagccttggtcttgtcctgcttactcaccttcctggtcctgatgcgctcactggtgacacacaggtgagtgacagactctggctcacccagcgccgaggtggggaaggagtgggagtcctgtgggcctgcggggaggccctgggcytggagggagggaagggctctgccggcccgggctgtcatggagcccacctgtgcgtggctgcgctgcagtgggagaaggtggggctgggtcctgcccttgggccttcctgtctggttggggaaacgtctgtgagtgtgagggacatagggcaccaccaactcagggcagctagtggagcttgccccctgagtaggggccaccgggtgggctggatgagagggaaggtggcctggggaagctgcacctgcccgggaatggaaggagataggaggtgaggactgtgtggacgagaacagaggc

SEQ ID NO: 41 - DNA, 601 bp, Homo sapiens
ggctcccgggcctgagtggtgctgttcaccatactccttcctttgaggactacaggattccatctcccgctgaagctggtgctttcagctacactgacagggacggccatttaccaggtatgtccctcaccctgtactgccatctgcctctggagccctgccctcaacccccaagcctggagcagttccggcccaacccagctctccaggtgctccatccagaaccctgaccaggtgctgcaggccatcttgggagttctggcctacccagaagggccggggtgttcactcttggactcaygaagtgactcggagcattaattttggaggccacataaacacgaatttaaatcctagctccactatttacctcctgtgtggccttgggcaagttatataacctctctgaacctgttagctcatctgtaaaaaggagaaaatagaataattttcctctttgagtaattgggaagatttagcaagacaacatgtttgataaactctgcctcaccacctccgaaaaaaaaaaaaccccgaaaaaaccaacaacaacaattaaaatcctgggagatgggtatgattattctccactttacagatg

SEQ ID NO: 42 - DNA, 601 bp, Homo sapiens
ttggggtatagacgtgaaattgttttctaacttccctgagcctcactgtcttcatctgtacaatgggactactattgcctggctcacaggactgctagtacggtcaaccaggagcctagatggaaagcttctagcccaggcctgtaacagtgggagggctgggtagctgtccattcctctccctatgtggaaagttattctttacctatcaaacagtagaacagaggctagaattataatgcctgcccccagaaagtggctttattgagatgtacaaaatattctacattaatttttattycattctcacatttattgagatgaacaaaatcagtgttatggattgttatagacacagtatctgtccctgtgccctgaccctgactgtggggctgtcaaatgtgcttcctggggaaccagatacaccagacacaccacacacacacataaacacacacaggaacacatgaagccaagcaaattcagagacaatttgatgagtgtaacacacacacaggagggtatatgcacatttgtgagtgcactgcaggcccatggctggatgaagcatgggaaatgggggaatccacagggcgcacgt

SEQ ID NO: 43 - DNA, 601 bp, Homo sapiens
gccccctcctcaggccccctcctcggctacctcggccctggggactgggtcccccaccccaccgttgcgccagatgtggctgcaggggaggggtaggcacggggcccagctggcagtggtcactcggtggtcctctccctgcagagtcagcactttctgtgccagctcgcccagcctcctcactcagcacttcctccagcccgctttccagaggcagcagctgcctgggttgggtcttggattcccagtgctgccccaaacccatctacagcgcagcctggaagatggagggtttgggtgytctggggccttccctcttggcatccccactatcacagaaaattccgacccaccatgggcctcctagtacgtccctctgcaaagcaagggaggaatcccacagctcctgacccctctctgctccccagacccgctgctggtcaggcctgctgcttcctggttcccccatggactttccagaactcagccttccatgacagggtcacaggaccctggctgctccgtgggagccccggcaacacagagctgagcacgtggcaggggcttaataaagtcatgcgcaatcaacaggctctttcat

SEQ ID NO: 44 - DNA, 601 bp, Homo sapiens
ctcccaaggccttaccctcgcaccccccgcctgactcaccggctctggcaatcctgtgaaaggaacatggaacaggcagccctgggcatcctcccagggcatcttggatggctgtgctagccttgcctgacgtgtgtcctctatccctgacttcacatggccaatcagaggccaggagagaaccgcagctgcctcctccctggtcccttgctcccagtctctccctcttcagtctgtcctgcatgtggggcagataaacccctgcagtctgaccaggttactcctctgctcaagaagattstgtagctcctcagggcctttaggacaaaatctagtgtctcagcccagcactccagagttagaggcacctccttcctgtcctttcttcctccttttctcaagcctccacagcccccatgggaccatgctgctcgtgggcctagatgttacacctccctagacacatgcaagggtgtttctagggtacctccttctctgtccagcctcctgcacccaagaggccagacctgtctggaccctcagtccctgagagcagccctggctgcctgctaccacctctcctgcctgagcccctagctca

SEQ ID NO: 45 - DNA, 601 bp, Homo sapiens
tgtgactctctatcctgagttcttaacaaccatgcatttaaaaaaaaaatttatatattattgcttatcttgtacacctcatttattcactcattcatttggtcattcattcattcaacagatatttgagtgctagattctgggcactggttgtgacagggtgagatgggacagtgagtgtgaaagactttggaagcagagtctctccccagtgtggggttagggtgtagcctggtcatggtcattgcagattccagaacgtttgtgtaacaaacactcatatggtgcatactaagtgccrgacactgttcttggagttttgcagttagaatcacttaatccctgtgacaaccctgggattgttatccccattttgcagatgaggaaactgtggccctgagaagttaagcgacttgccctggaggcagaatcacagatcagcatccctccttccttccccgaagttgtctcagattgttccttcagaaagaaaaagagtcttcaggcttagaaagctgtgtcggatcagacaagctgtggaaacggagggttctgggccctcctcctgcctccctgtttctcctttgagagtcatcagtgt

SEQ ID NO: 46 - DNA, 601 bp, Homo sapiens
ggatgggaaagcgaggccccagaggcctttgccatccaaacctcaagagacaacccatccttccccctgcttgtgggccagctggaaccacaggttttccaaacccaacgcaaatctttctgcaaaagaaaggactctgggaatcgattccagaccctgcttgacaatgaagtctgtgctcacacaggcagtgccaccagacgttttatggagtatgggcattggctcacgcacttgccaaggacatacaggtgctaggccctgagctgggcgtggagaatttcaggggcatcccagaacsctccctggcctcactgacgtctcagcctggcagtgagaaaacagacacctggggtggagagtggcccaagtccatgtctgtctttgccagctctcagcacccactgtgcacctgggagacgtgcaccagaagctgagtacaggcaaaagataataccactcacccctgccctcatgggctcttgcctggctgcagaaggcagatatcatctcgtgaggaaatagggcagatatcatctcatgaggaaatagtcatttaacaaatatttattgagcgacttactgtatgctgggcaccgtt

SEQ ID NO: 47 - DNA, 601 bp, Homo sapiens
tccttccccctgcttgtgggccagctggaaccacaggttttccaaacccaacgcaaatctttctgcaaaagaaaggactctgggaatcgattccagaccctgcttgacaatgaagtctgtgctcacacaggcagtgccaccagacgttttatggagtatgggcattggctcacgcacttgccaaggacatacaggtgctaggccctgagctgggcgtggagaatttcaggggcatcccagaaccctccctggcctcactgacgtctcagcctggcagtgagaaaacagacacctggggtgsagagtggcccaagtccatgtctgtctttgccagctctcagcacccactgtgcacctgggagacgtgcaccagaagctgagtacaggcaaaagataataccactcacccctgccctcatgggctcttgcctggctgcagaaggcagatatcatctcgtgaggaaatagggcagatatcatctcatgaggaaatagtcatttaacaaatatttattgagcgacttactgtatgctgggcaccgttctagagctgggtattcattcgtgaaaagaaagagacaaaattcttgcccttgtggat

SEQ ID NO: 48 - DNA, 601 bp, Homo sapiens
ggcagtgagaaaacagacacctggggtggagagtggcccaagtccatgtctgtctttgccagctctcagcacccactgtgcacctgggagacgtgcaccagaagctgagtacaggcaaaagataataccactcacccctgccctcatgggctcttgcctggctgcagaaggcagatatcatctcgtgaggaaatagggcagatatcatctcatgaggaaatagtcatttaacaaatatttattgagcgacttactgtatgctgggcaccgttctagagctgggtattcattcgtgaaaagmaagagacaaaattcttgcccttgtggatcttctattttcatgtataaacataataaatatgtaaaaaaaatagaaggtactaagtgctgtggaaaaatagagataggtaaggaagatcgggagtgtaggagagggacaggttgcaaaggtggtcaggcctcatgaaggtgacacttgagcaaagacttgaggcatatgagtgagccatgcagctgtctgcaggaagagcattccagataggaagacagccagtgcaaggccctgaggcaggagttccctggggttttcaggtgtgctcca

SEQ ID NO: 49 - DNA, 601 bp, Homo sapiens
cgcaatcaagcacacacttacaaatcccttctgtgagtcaagcccttctgctgctgtctctccccattggtggggtggacaggtgagtgctgtaaaagggtgcagggtggtggtggaggggtgttcaggactgacagggtgggctttggagctaaagactgtgtgcctctgcataggtagagctggtgtgtgtgtgtgtgtgtgtgtgtgtgtgtgtgtgtgtgtacgcgcatgcacatgcatagctgggaattccaggacaagggcaccatggagtcagggggtaaaaaaggttggggtrtgctgccagagacctgcctactgatccttaggattaagagttctggaaggctaggtaaggtcaaaccacagacggctttagctgccactctgagacttctaggcttgattctgtgggcaatggggagccattgaagaccctgggcctggcttgatggctgtcctagctctcaggcttgagctgggttacatcttcattatggggcaaagtggaatctgcagccaccttgcctgcctgctcaccaagcaggtgtggcactgccaggccagtgctgacaagttaatttcctgattatgagat

SEQ ID NO: 50 - DNA, 601 bp, Homo sapiens
ggtggacaggtgagtgctgtaaaagggtgcagggtggtggtggaggggtgttcaggactgacagggtgggctttggagctaaagactgtgtgcctctgcataggtagagctggtgtgtgtgtgtgtgtgtgtgtgtgtgtgtgtgtgtgtgtacgcgcatgcacatgcatagctgggaattccaggacaagggcaccatggagtcagggggtaaaaaaggttggggtatgctgccagagacctgcctactgatccttaggattaagagttctggaaggctaggtaaggtcaaaccacagayggctttagctgccactctgagacttctaggcttgattctgtgggcaatggggagccattgaagaccctgggcctggcttgatggctgtcctagctctcaggcttgagctgggttacatcttcattatggggcaaagtggaatctgcagccaccttgcctgcctgctcaccaagcaggtgtggcactgccaggccagtgctgacaagttaatttcctgattatgagataatcatttcctagggaatcgggcagccagctcccgccgtggaggcctctctggctcatctttattcaaggccc

SEQ ID NO: 51 - DNA, 601 bp, Homo sapiens
aatggggagccattgaagaccctgggcctggcttgatggctgtcctagctctcaggcttgagctgggttacatcttcattatggggcaaagtggaatctgcagccaccttgcctgcctgctcaccaagcaggtgtggcactgccaggccagtgctgacaagttaatttcctgattatgagataatcatttcctagggaatcgggcagccagctcccgccgtggaggcctctctggctcatctttattcaaggccctgctctgcagagctttctgcttctctgccacctaggggctcctctrgagtctggctgcgctagccaaaacatcccgctcagcacatatttcacaagcaactgaccagttcctcctggactttgtttatcctccagccctgggctcaagagattagggctcttttgcattccactttccatcatttgcccctcatgaggaggaggcttggagaggaaagattccagagacaaggtttactgaacccacaaaaaagaggaagccaagagggaaacaggtgaggtgtggacgagcaatgggaaggcagccacagggctctcagcgccaggcagcctccctccaggaacc

SEQ ID NO: 52 - DNA, 601 bp, Homo sapiens
tccagccctgggctcaagagattagggctcttttgcattccactttccatcatttgcccctcatgaggaggaggcttggagaggaaagattccagagacaaggtttactgaacccacaaaaaagaggaagccaagagggaaacaggtgaggtgtggacgagcaatgggaaggcagccacagggctctcagcgccaggcagcctccctccaggaaccgctgggcaggagggaacagatttctaatgaagcaggaaggatttaggtcagaaggtgggagaatttcctgactgctaaagtatcrtctgtaggggttgtgagagggctcgggctcttgcccaaggctggggaatgaactgaacagctcctaaaaagtattttaatctccaagtgtttatcgatctatccaacctgtgttcaggggccagctagttcagtctccctcatcatccagatggggaaagtgagcctcagaaaggggactggggagactggacaagacagtgaactcacctctggagcttcccccctgagaaggccccagctgcatcccaccagggaaggggcagatggtctttgtcctccggttcccgagggcgggcac

SEQ ID NO: 53 - DNA, 601 bp, Homo sapiens
ctgtgttcaggggccagctagttcagtctccctcatcatccagatggggaaagtgagcctcagaaaggggactggggagactggacaagacagtgaactcacctctggagcttcccccctgagaaggccccagctgcatcccaccagggaaggggcagatggtctttgtcctccggttcccgagggcgggcacgggagccgtcagcaactccagacccagcattgagcaggaggacagggtgggcttgtcccgagcacacaggccccaagagtcagtgtgacaggttcctcctactgtgcycctgcacactcccacagaggaagctccaggggcagagccaggatatagaacattggttctgtcttctcggctccgtgtcttcactgtcctggaggtttgaagggcatgctatagctaaaaggcttaccccacatcagtggctggctgtctccctgtggaatgaatggccctgggaccgtattccctcgcacgcagctcctccccagccgagcatcctggctttggccatctcaagggtcttcgctgccccgtgactcagcatccctgccatcctggcccaggctctgtgcagcctgcatc

SEQ ID NO: 54 - DNA, 601 bp, Homo sapiens
ttgagcccaggagatcaaggctgcagttagccatgttcacgccactgcattcctgcctggacaacagagcaagaccctgtctcaaagaaaaaagaaaaggtggaggtggcgtggggagctggatatgtaccttcaccaggtccaggatcttcctttaaaatgcatactttttgaggtgagggctgatcacacagtctgataataactggacttcacccaacattggccactctgcttggagggcctgttgtaccctacaagccaaaaattgcagaccagcctccttactcctacactgatygtgtccactagcccccaactcaatcaacccttctttatccccttccaacatcacataatcccccagccccaagccctgcagaggggccaagagcccaagcctgcaggcctggctgctgacgaaatgtcccctgggagagctgcccagagaaggagccattgttccttcctggaaaaacataccagctatgggagggggtctgctattccctgggacctcggcaaggatcctgagggtgaggcgggggggcctggcctgaactcctgaggctgctcctttccagcagcttccccgagctgc

SEQ ID NO: 55 - DNA, 601 bp, Homo sapiens
cgtgcacctgaggaagaagagaaacagggagaaagggagtctagtcaggggggctgcacctcaactgctcaccagagaaggtggtaaagaggcagacagacagacagagaggtgacagagagagggagaacagagacacactgctagagagacacagaggggaaagaagaggcagctggtaatccaagaaaacatcatctaatcccccttatcagaagattggatgtttccctagagctagagcctatgccaggtgggccccgcatggggcatttggggtcagagaggttggatcctgccygagggaatgcagtaatgaggagctgagccaggattcaacacctggtctccccaaatccaaggctcaaagtcattccctatgcatccctgtctaggtgactgatcgagggcctgtctgatcccctgctctggagccccacgtgcccaacattcactgagatgatggcccaagtatgatctgtgctttttgtctgaaaaggagctgttgggatacaacacttttaaatgttggattaactagggtagggccagctttatttgcagaacatctaagaacagatgcagggatgcaactttcaaa

SEQ ID NO: 56 - DNA, 601 bp, Homo sapiens
cacagatcatacttgggccatcatctcagtgaatgttgggcacgtggggctccagagcaggggatcagacaggccctcgatcagtcacctagacagggatgcatagggaatgactttgagccttggatttggggagaccaggtgttgaatcctggctcagctcctcattactgcattccctcgggcaggatccaacctctctgaccccaaatgccccatgcggggcccacctggcataggctctagctctagggaaacatccaatcttctgataagggggattagatgatgttttcttggwttaccagctgcctcttctttcccctctgtgtctctctagcagtgtgtctctgttctccctctctctgtcacctctctgtctgtctgtctgcctctttaccaccttctctggtgagcagttgaggtgcagcccccctgactagactccctttctccctgtttctcttcttcctcaggtgcacgtgaacccaaagtatattgttttggagtctgacttcaccaacaacgtggtgagatgcaacattcactacacaggtcgctacgtttctgcaacaaactgcaaaattgtccagtaagagtt

SEQ ID NO: 57 - DNA, 601 bp, Homo sapiens
gtgtgtgaccagggtcagggctcagtctgtgaccagagtcagggcccagtgtgtagccaggctcagggctcagtgtgtgaccaggatcagggctcagtgtgtgaccaggatcagggcttagtgtgtggccagggtcagggctcagtctgtgaccagagccaggctcatttagagacagccctctcacacttggttgctaccatcaacctaactgttcacagcatgctgccaccacatcatgaatattaaaaatgggaacagcagcctccagttcagtgcagagattaggttcctcatgagyagaatgagatcagaattccccagggcacacagcatgtttgaaaggacgaactcctgtcccatcactggcagtttggctctttagcaggcctgggtttccagcacattcctgctcctcagggcctgtcttagccattcaagacccagggtcacctaagtggtagaagccattctagggaaggaggaggaaggaagaagggggtatccaggaaacaaaggaggctgctgggggctcggggaggcagggaatccctcctgctgctgggaaagggggatgggaacagaaacagcatcccacgtg

SEQ ID NO: 58 - DNA, 143 bp, Homo sapiens
gctaagatggcgccattgcagtccagcctgggcaacagagctagactccgtatctatctatctatctatctatctatctatctatctatctaggtagatagatagaacaggaggccagctagaagggacatagaggatgcaca

SEQ ID NO: 59 - DNA, 603 bp, Homo sapiens
caagagctgcggtgcacctgggacctggaattagagagtggtccctgttcagcatctccccgaggaggcccaccaacaaagagggtgtgtcttttttttttttttcttcctattgagggtgtgggataatggtggaaggaacatgcaaagagggtgtgtcttaattagcactggctttagggaacaaggaaaagggagaacccgggagtacgggaaggaggctggggcagacaggagtcagaggcccattccagcccaaacgagaagccagtgagcaaggtggagaccagggatgctgtgascaaagcagagaggaatgggcggggtggtgctgacaccccagccccgttctgcctgccagagccccacttaccaggcccgagtccccagaggtcccctcctactccctgctcgattcccttcctcagaggcaggtctgtggcttggctgggaactccagggactgagggagcactgcagctgtgggaccggcgcatagctaaaagccggcgggccatagggccccgcggaggaggccccagcaggcggaccaggaggccgaagcctcccgacgctcccagcctgttgcttattcattcagag

SEQ ID NO: 60 - DNA, 603 bp, Homo sapiens
aggcggaccaggaggccgaagcctcccgacgctcccagcctgttgcttattcattcagagtgggaaagcgccagccgagcggccagccagtgcggggctggccatgtaaggcccacaggcggtcctgcccgcccggtgccctgcggagagcctcgtgcagccctgggcaccgcccctgccctgccctgaccccttggccttgaaatgctgtcatcggaggagccgtcccgctcgggacaaggccagcatggacaaagctagagctggggcaagcaaggagccttcctgtcctcgaggccgtrggaagagaagcacgcccagggggccactcctgagagcctctctgtccaccaggcctctgcagaggggtcaccatggctctggcccgaggcagccggcagctgggggccctggtgtggggcgcctgcctgtgcgtgctggtgcacgggcagcaggcgcagcccgggcagggctcggaccccgcccgctggcggcagctgatccagtgggagaacaacgggcaggtgtacagcttgctcaactcgggctcagagtacgtgccggccggacctcagcgctccgagagtagctcccgggtgctgc

SEQ ID NO: 61 - DNA, 603 bp, Homo sapiens
accccgcgtcgcggacctacgaccagggtttcgtgtactaccggcccgcgggcggcggcgtgggcgcgggggcggcggccgtggcctcggcgggggtcatctacccctaccagccccgggcgcgctacgaggagtacggcggcggcgaagagctgcccgagtacccgcctcagggcttctacccggcccccgagaggccctacgtgccgccgccgccgccgccccccgacggcctggaccgccgctactcgcacagtctgtacagcgagggcacccccggcttcgagcaggcctaccctgaycccggtcccgaggcggcgcaggcccatggcggagacccacgcctgggctggtacccgccctacgccaacccgccgcccgaggcgtacgggccgccgcgcgcgctggagccgccctacctgccggtgcgcagctccgacacgcccccgccgggtggggagcggaacggcgcgcagcagggccgcctcagcgtgggcagcgtgtaccggcccaaccagaacggccgcggtgagtacggccccggcgcccctccggccgcgcgtacccctggccactggaaactgctccgggccccccgggccc

SEQ ID NO: 62 - DNA, 603 bp, Homo sapiens
gcccctccttagaacttcctggaggcctccccggagctgctaagcacggagcagcgccccctcccgacttcccccgacccagccaacagcacccccctggcatctcacctcccagcttaacccgcacccaggcatcatcagtgccaggggttggccaggcaggtgtgggctctgatcctggctttggtggagggactagccgagctttgggaggaggctcgccttctgcacttggcttgggtcatctgaggacaaaaggaaggagcccctcatccaccttctccctcatttcaatgagggcrtctccttccccccttagagatgttctatgccagctcacagggtgaggcaaggagcatcagaaacctgagtcttcagggccagaaaggccttaagggatgcccctggaggtgtggcaggctgcccattactgggcatgtgtgaggttggcttgagtgcgcaggaggttagaggggagcgtgtgctccaggcaggagtccggacttcctcaggggagtctgcaaggctggggcaagagagaccagtcaggcactggcctgggtgcagaatttcaggggctaccaaaaaacacagtagttgaga

SEQ ID NO: 63 - DNA, 603 bp, Homo sapiens
cttctagcccatcccttcctgcccaccctgactcctgccagcctgcatccacctggctctgcccatgaaccttcagggacacactccaggccagaagcccagggctctcttggtggtttacaaagaatgctgactgcagggatgagaggaacggagggtgtctgggggctgctgtccagaggcctggggagtaaggactcgagggagtgcctctgtcaggaaattacatggttcactcattcctgcctccaggtctttgttcatgctgttttccctgccgtggacaccctttcccgctctcygattctctaaatcctgccccatctcccagatcttgttcatgtccaagcttttccaggaagtcttagcagctcccacaccgcagagctcgagatgtaagctcttgctgcctgacttccctgaccccacacctgggcccaggacctaacacccaaggagaatggcaaagggttttggggtatgagaggatgtttggggtgtgaggaggtctctgggcattagcagtggccaacctgatgctctcaatgtcatgctcttcctccctccccccttctctcccactgcccactccaggtctccctg

SEQ ID NO: 64 - DNA, 603 bp, Homo sapiens
gttcatgtccaagcttttccaggaagtcttagcagctcccacaccgcagagctcgagatgtaagctcttgctgcctgacttccctgaccccacacctgggcccaggacctaacacccaaggagaatggcaaagggttttggggtatgagaggatgtttggggtgtgaggaggtctctgggcattagcagtggccaacctgatgctctcaatgtcatgctcttcctccctccccccttctctcccactgcccactccaggtctccctgacttggtcccagaccccaactatgtgcaagcatcyacttatgtgcagagagcccacctgtactccctgcgctgtgctgcggaggagaagtgtctggccaggtaaggagctgaggcagaagtgtagagtgttgggatagtccccgggagtcacccagaacccagcctagctgtggctgccagctaggctgtctgcaagctgatgaccctggccaagtgccccagcctcccaggaccctgaccgtttcatcagtgagccttgcctgccccgagggagtcaggaggggaggggagcaagcgggggcccagcatttctgtatactgtacctcactataca

SEQ ID NO: 65 - DNA, 603 bp, Homo sapiens
ctgtactccctgcgctgtgctgcggaggagaagtgtctggccaggtaaggagctgaggcagaagtgtagagtgttgggatagtccccgggagtcacccagaacccagcctagctgtggctgccagctaggctgtctgcaagctgatgaccctggccaagtgccccagcctcccaggaccctgaccgtttcatcagtgagccttgcctgccccgagggagtcaggaggggaggggagcaagcgggggcccagcatttctgtatactgtacctcactatacaaggaactccacttttactgtgrcacttcatctttatatgagccctgaaggcatgggttaccccctcaggaaactgaggagcaatgaaaggaactcaaaccttttggatctgagctgtttaatgagaaggttccttttgggagcccaggcttccagtggtcatacagggacccaaggaagtcctattgggtggatgtacctgcaggatagagcccaggagtatggagccatgtccacagaggacagttgtggctttggctggtccaggctgcctctgcctagtcacagcaggggcagtgagcccgggggacaaagaagtttcag

SEQ ID NO: 66 - DNA, 603 bp, Homo sapiens
ttggtgcctgagctcccctcccaccatgagcactctgggagacacagatactggattagagggcccggggaggtgttacctgccaactcttttcactggggcaagtgtttctcagagaagccttagccaaagctaggctggaagatggatatagaaggattggtctcaatcagggatggaaatgtctagaatacagggcattgcccagtgaggccattacaggtgggagcccaaaaaggaagtgaggggtctagagcatgtgctgtcctggagtgacccagctaggtggggagcacagaggrgccaccccgcagtactcaggagacaggagaacctagaccagccaggaggctgtgccacagcagggtcacctgagcccatctcaggatggggacaggatgagaggcccagggaagactaggccctcttctttctccttctctcctctgcccctaggcattaccacagcatggacgagttcagccactacgacctactggatgcagccacaggcaagaaggtggccgagggccacaaggccagtttctgcctggaggacagcacctgtgacttcggcaacctcaagcgctatgcatgcacctc

SEQ ID NO: 67 - DNA, 603 bp, Homo sapiens
attagagggcccggggaggtgttacctgccaactcttttcactggggcaagtgtttctcagagaagccttagccaaagctaggctggaagatggatatagaaggattggtctcaatcagggatggaaatgtctagaatacagggcattgcccagtgaggccattacaggtgggagcccaaaaaggaagtgaggggtctagagcatgtgctgtcctggagtgacccagctaggtggggagcacagaggggccaccccgcagtactcaggagacaggagaacctagaccagccaggaggctgtrccacagcagggtcacctgagcccatctcaggatggggacaggatgagaggcccagggaagactaggccctcttctttctccttctctcctctgcccctaggcattaccacagcatggacgagttcagccactacgacctactggatgcagccacaggcaagaaggtggccgagggccacaaggccagtttctgcctggaggacagcacctgtgacttcggcaacctcaagcgctatgcatgcacctctcatacccaggttgggctggagagatggggtttggggcatgggaggataaggag

SEQ ID NO: 68 - DNA, 603 bp, Homo sapiens
ttagagggcccggggaggtgttacctgccaactcttttcactggggcaagtgtttctcagagaagccttagccaaagctaggctggaagatggatatagaaggattggtctcaatcagggatggaaatgtctagaatacagggcattgcccagtgaggccattacaggtgggagcccaaaaaggaagtgaggggtctagagcatgtgctgtcctggagtgacccagctaggtggggagcacagaggggccaccccgcagtactcaggagacaggagaacctagaccagccaggaggctgtgycacagcagggtcacctgagcccatctcaggatggggacaggatgagaggcccagggaagactaggccctcttctttctccttctctcctctgcccctaggcattaccacagcatggacgagttcagccactacgacctactggatgcagccacaggcaagaaggtggccgagggccacaaggccagtttctgcctggaggacagcacctgtgacttcggcaacctcaagcgctatgcatgcacctctcatacccaggttgggctggagagatggggtttggggcatgggaggataaggagt

SEQ ID NO: 69 - DNA, 603 bp, Homo sapiens
gccttagccaaagctaggctggaagatggatatagaaggattggtctcaatcagggatggaaatgtctagaatacagggcattgcccagtgaggccattacaggtgggagcccaaaaaggaagtgaggggtctagagcatgtgctgtcctggagtgacccagctaggtggggagcacagaggggccaccccgcagtactcaggagacaggagaacctagaccagccaggaggctgtgccacagcagggtcacctgagcccatctcaggatggggacaggatgagaggcccagggaagactargccctcttctttctccttctctcctctgcccctaggcattaccacagcatggacgagttcagccactacgacctactggatgcagccacaggcaagaaggtggccgagggccacaaggccagtttctgcctggaggacagcacctgtgacttcggcaacctcaagcgctatgcatgcacctctcatacccaggttgggctggagagatggggtttggggcatgggaggataaggagttggggaggcaaagagcgaggcccgctgaggcccggcaagtgccaaggcttctggccactcagct

SEQ ID NO: 70 - DNA, 603 bp, Homo sapiens
ggagcccaaaaaggaagtgaggggtctagagcatgtgctgtcctggagtgacccagctaggtggggagcacagaggggccaccccgcagtactcaggagacaggagaacctagaccagccaggaggctgtgccacagcagggtcacctgagcccatctcaggatggggacaggatgagaggcccagggaagactaggccctcttctttctccttctctcctctgcccctaggcattaccacagcatggacgagttcagccactacgacctactggatgcagccacaggcaagaaggtggccragggccacaaggccagtttctgcctggaggacagcacctgtgacttcggcaacctcaagcgctatgcatgcacctctcatacccaggttgggctggagagatggggtttggggcatgggaggataaggagttggggaggcaaagagcgaggcccgctgaggcccggcaagtgccaaggcttctggccactcagctctgctcacagtgaaggtcttctcaccagtcctcaggctgccacactgccctgcagggactgttccctccctgccccagcccctttcccatgttattccaggtgatc

SEQ ID NO: 71 - DNA, 603 bp, Homo sapiens
agcagggtcacctgagcccatctcaggatggggacaggatgagaggcccagggaagactaggccctcttctttctccttctctcctctgcccctaggcattaccacagcatggacgagttcagccactacgacctactggatgcagccacaggcaagaaggtggccgagggccacaaggccagtttctgcctggaggacagcacctgtgacttcggcaacctcaagcgctatgcatgcacctctcatacccaggttgggctggagagatggggtttggggcatgggaggataaggagttggrgaggcaaagagcgaggcccgctgaggcccggcaagtgccaaggcttctggccactcagctctgctcacagtgaaggtcttctcaccagtcctcaggctgccacactgccctgcagggactgttccctccctgccccagcccctttcccatgttattccaggtgatctgctcgtggagagaaggaaacatcgcaacagtctggagagcaacacgtcctattggcctgttcacccacccatatccctctttccatcagccaccctaaatatccacaaactgtccatctgtcctgtctcttttt

SEQ ID NO: 72 - DNA, 603 bp, Homo sapiens
gctggagagatggggtttggggcatgggaggataaggagttggggaggcaaagagcgaggcccgctgaggcccggcaagtgccaaggcttctggccactcagctctgctcacagtgaaggtcttctcaccagtcctcaggctgccacactgccctgcagggactgttccctccctgccccagcccctttcccatgttattccaggtgatctgctcgtggagagaaggaaacatcgcaacagtctggagagcaacacgtcctattggcctgttcacccacccatatccctctttccatcagcyaccctaaatatccacaaactgtccatctgtcctgtctctttttatccattctgccatccatctcgtccctcccacctggctattagatatctgtctttcctctggtccatctagccagtggctttaggaaggtgtggacccaccctgagcagttgccctgggccagggtcaggggagaaacccaggggaaggtgggccagaaactcctgaaggtgggcgtggggtggctctgggaaacaagcagcatcacagccgctcctcttgtccctttcccagggcctgagcccaggctgctatgaca

SEQ ID NO: 73 - DNA, 603 bp, Homo sapiens
ggtgtggacccaccctgagcagttgccctgggccagggtcaggggagaaacccaggggaaggtgggccagaaactcctgaaggtgggcgtggggtggctctgggaaacaagcagcatcacagccgctcctcttgtccctttcccagggcctgagcccaggctgctatgacacctacaatgcggacatcgactgccagtggatcgacataaccgacgtgcagcctgggaactacatcctcaaggtgggcctctgggtctggggctttccctccaacctgatgttcatgtccaatgtccccccrttcctgaaagaagcttcagcctctgggcctgttcccttctccccagctgccgagcagaggcagcatctgaggcatcacttgaggtttaggcttcccagccgaagatttaggtctcaggctgacatagcctctctttccataaagaacccagggaacctttgccccagctgtggtaatgccagaggcagtggtgatactgtctccctaagtgacagaggaccagggagagcaagaaagacttggagagccatgtggtctatcttcctgtcttcggacagggcagactttgtatcatttggga

SEQ ID NO: 74 - DNA, 603 bp, Homo sapiens
ggagaaacccaggggaaggtgggccagaaactcctgaaggtgggcgtggggtggctctgggaaacaagcagcatcacagccgctcctcttgtccctttcccagggcctgagcccaggctgctatgacacctacaatgcggacatcgactgccagtggatcgacataaccgacgtgcagcctgggaactacatcctcaaggtgggcctctgggtctggggctttccctccaacctgatgttcatgtccaatgtccccccgttcctgaaagaagcttcagcctctgggcctgttcccttctccscagctgccgagcagaggcagcatctgaggcatcacttgaggtttaggcttcccagccgaagatttaggtctcaggctgacatagcctctctttccataaagaacccagggaacctttgccccagctgtggtaatgccagaggcagtggtgatactgtctccctaagtgacagaggaccagggagagcaagaaagacttggagagccatgtggtctatcttcctgtcttcggacagggcagactttgtatcatttgggagacagatgagaggccctcagctccttatagggacccctgcaaa

SEQ ID NO: 75 - DNA, 603 bp, Homo sapiens
gagcaggggatcagacaggccctcgatcagtcacctagacagggatgcatagggaatgactttgagccttggatttggggagaccaggtgttgaatcctggctcagctcctcattactgcattccctcgggcaggatccaacctctctgaccccaaatgccccatgcggggcccacctggcataggctctagctctagggaaacatccaatcttctgataagggggattagatgatgttttcttggattaccagctgcctcttctttcccctctgtgtctctctagcagtgtgtctctgttytccctctctctgtcacctctctgtctgtctgtctgcctctttaccaccttctctggtgagcagttgaggtgcagcccccctgactagactccctttctccctgtttctcttcttcctcaggtgcacgtgaacccaaagtatattgttttggagtctgacttcaccaacaacgtggtgagatgcaacattcactacacaggtcgctacgtttctgcaacaaactgcaaaattgtccagtaagagtttgcccaccacccttcctggctccgtccctttcctgcctggggagcaggcaaggcca

SEQ ID NO: 76 - DNA, 603 bp, Homo sapiens
gactccctttctccctgtttctcttcttcctcaggtgcacgtgaacccaaagtatattgttttggagtctgacttcaccaacaacgtggtgagatgcaacattcactacacaggtcgctacgtttctgcaacaaactgcaaaattgtccagtaagagtttgcccaccacccttcctggctccgtccctttcctgcctggggagcaggcaaggccacctgagatacctaagatacctgcactttaggtcaccttgatggccgaacgcagctgcagcagggcccatcaaaaagagccttgtccrtgccaaccccaagctgagtgtttacggtggggctcaggccgtagggtgttgatctggggctgcatcctgtggctccctgagatgtcccaagtgaaaaccagcagaaggcagagtcatttgatgcaaggccatgagctctgcagtcccagttccacagcttcctcgctatgtgacattgggttacttccctgagctactttcctcaactataaaataggtatttccacataggctggcaatgaggatctacaggcctgatgtctgcatgttcacagtggcacacagtgaacagcagctgtga

SEQ ID NO: 77 - DNA, 603 bp, Homo sapiens
gagctgggctgagggggtcggaactttctcttgagggaaatgtaggcccatgcctgccctgccacgagggatctgggctgtgggagggacgccctggctgaggaggccacgggggcctactttgcagcccctcattgacccactgtctttccttcctcagatcctgatctccgggagggacagatggccaatctctccccttccaaagcaggccctgctccccgggcagcctcccgccgaggggcccagcccccaacccacaggcacggaggggcatccctccctgccggcctcagggagckaacgtggatgaaaaccacagggattccggacgccagaccccattttatacttcacttttctctacagtgttgttttgttgttgttggtttttattttttatactttggccataccacagagctagattgcccaggtctgggctgaataaaacaaggtttttctactctgtggctctgcatgcggcctgctggctggctggccagccacagctagtttgggcctgggggatgtccttaggcatcatccttcccctcgccaaatgtggaaagggcagccaccacccggtcaggaccacagtga

SEQ ID NO: 78 - DNA, 603 bp, Homo sapiens
ctccctgccggcctcagggagcgaacgtggatgaaaaccacagggattccggacgccagaccccattttatacttcacttttctctacagtgttgttttgttgttgttggtttttattttttatactttggccataccacagagctagattgcccaggtctgggctgaataaaacaaggtttttctactctgtggctctgcatgcggcctgctggctggctggccagccacagctagtttgggcctgggggatgtccttaggcatcatccttcccctcgccaaatgtggaaagggcagccaycacccggtcaggaccacagtgaccatgagggcgctgagcccatcgtggaagcctgtggggttgagcccttggccaagcctgtcgcatgggaatgggaccacatccgactaggggagggccctgcagcggggtgggcggcaccagcctgctctccctgctcagcccctgctcctatctgggctcgtgtgcccactgctgccccttctcagccttcagcctcacactcccaccggtccccctcctggccctctcactctcacaccgttcactctccaccagccacccactcccttacaattgc

SEQ ID NO: 79 - DNA, 603 bp, Homo sapiens
tggaaagggcagccaccacccggtcaggaccacagtgaccatgagggcgctgagcccatcgtggaagcctgtggggttgagcccttggccaagcctgtcgcatgggaatgggaccacatccgactaggggagggccctgcagcggggtgggcggcaccagcctgctctccctgctcagcccctgctcctatctgggctcgtgtgcccactgctgccccttctcagccttcagcctcacactcccaccggtccccctcctggccctctcactctcacaccgttcactctccaccagccacccrctcccttacaattgcaggctcccgaactcaagtctcctgaaggccaggtgctgtgctagggcacacgcaggagcatcgtagcttctgccctcagggagcgcataaccacagggaacggggagcacgcgttgcccagtgaggcagcacctaggggttctggggagccttcacatgggagagagcctccttccaggcagggaggggagaagaggattcttggcagagagacagcattgagcagagatcatgaggtggggctgcaggccgacagggtgtgagtgggagatggagggaggccccg

SEQ ID NO: 80 - DNA, 603 bp, Homo sapiens
caggaccacagtgaccatgagggcgctgagcccatcgtggaagcctgtggggttgagcccttggccaagcctgtcgcatgggaatgggaccacatccgactaggggagggccctgcagcggggtgggcggcaccagcctgctctccctgctcagcccctgctcctatctgggctcgtgtgcccactgctgccccttctcagccttcagcctcacactcccaccggtccccctcctggccctctcactctcacaccgttcactctccaccagccacccactcccttacaattgcaggctcccraactcaagtctcctgaaggccaggtgctgtgctagggcacacgcaggagcatcgtagcttctgccctcagggagcgcataaccacagggaacggggagcacgcgttgcccagtgaggcagcacctaggggttctggggagccttcacatgggagagagcctccttccaggcagggaggggagaagaggattcttggcagagagacagcattgagcagagatcatgaggtggggctgcaggccgacagggtgtgagtgggagatggagggaggccccgcagccctgcagcccccgtcccctg

SEQ ID NO: 81 - DNA, 603 bp, Homo sapiens
catccgactaggggagggccctgcagcggggtgggcggcaccagcctgctctccctgctcagcccctgctcctatctgggctcgtgtgcccactgctgccccttctcagccttcagcctcacactcccaccggtccccctcctggccctctcactctcacaccgttcactctccaccagccacccactcccttacaattgcaggctcccgaactcaagtctcctgaaggccaggtgctgtgctagggcacacgcaggagcatcgtagcttctgccctcagggagcgcataaccacagggaayggggagcacgcgttgcccagtgaggcagcacctaggggttctggggagccttcacatgggagagagcctccttccaggcagggaggggagaagaggattcttggcagagagacagcattgagcagagatcatgaggtggggctgcaggccgacagggtgtgagtgggagatggagggaggccccgcagccctgcagcccccgtcccctgccctggctgtggcatcccagaggtcctggacactggaatggaccaggaagaagtgagagggtggtgcctctcctcctcgaggccctgaacaa

SEQ ID NO: 82 - DNA, 603 bp, Homo sapiens
cctcggcgggggtcatctacccctaccagccccgggcgcgctacgaggagtacggcggcggcgaagagctgcccgagtacccgcctcagggcttctacccggcccccgagaggccctacgtgccgccgccgccgccgccccccgacggcctggaccgccgctactcgcacagtctgtacagcgagggcacccccggcttcgagcaggcctaccctgaccccggtcccgaggcggcgcaggcccatggcggagacccacgcctgggctggtacccgccctacgccaacccgccgcccgaggcktacgggccgccgcgcgcgctggagccgccctacctgccggtgcgcagctccgacacgcccccgccgggtggggagcggaacggcgcgcagcagggccgcctcagcgtgggcagcgtgtaccggcccaaccagaacggccgcggtgagtacggccccggcgcccctccggccgcgcgtacccctggccactggaaactgctccgggccccccgggcccctccttagaacttcctggaggcctccccggagctgctaagcacggagcagcgccccctcccgacttcccccgacccagccaaca

SEQ ID NO: 83 - DNA, 603 bp, Homo sapiens
ctggagccgccctacctgccggtgcgcagctccgacacgcccccgccgggtggggagcggaacggcgcgcagcagggccgcctcagcgtgggcagcgtgtaccggcccaaccagaacggccgcggtgagtacggccccggcgcccctccggccgcgcgtacccctggccactggaaactgctccgggccccccgggcccctccttagaacttcctggaggcctccccggagctgctaagcacggagcagcgccccctcccgacttcccccgacccagccaacagcacccccctggcatctcmcctcccagcttaacccgcacccaggcatcatcagtgccaggggttggccaggcaggtgtgggctctgatcctggctttggtggagggactagccgagctttgggaggaggctcgccttctgcacttggcttgggtcatctgaggacaaaaggaaggagcccctcatccaccttctccctcatttcaatgagggcatctccttccccccttagagatgttctatgccagctcacagggtgaggcaaggagcatcagaaacctgagtcttcagggccagaaaggccttaagggatgcccctgg

SEQ ID NO: 84 - DNA, 38486 bp, Homo sapiens
ggcagttaccttgaaacaaggaaatgttcatcaactgttattacacaaacaaagcagcttgtaaaatgacataggcactatgatcctaattgtttttttaaatgagtgggggggtggagcctaaaacaatggtttttctctaggttacagaatgaattactttttcttttttactctgtattctaaatttcctataattgacctatttcttttgtaatctgagattaaaaattgtattacaaaaaaacttaagggagagattgctgatatctcatctttcatcaccctgtggccctttcctttccagaacccagctgggcaaaactagtctccactggccccaagggcctgctccttctgttacaggaaagggttcccgatccaggccccaagagaggcttcttagatctcacacaagaaagaatatggggcaagtccacagagtaaagtgaaagcaagtttattaagaaagtgaaggaataaatgaatggcgactccataagcagaacagccccaatggctgctggttagctgtcatctatgattatcttttgattgtatattaaagaagggatggattattcatgagttttctgggcaagaggtagggagttcccagaactgagagttccccttccttttagctcacatagggtaacttccggatgttgccacagcatttgtaaactgtcatgcactggtgggagtgtcttttagcatgctaatgtgatatgatagtgtacaatggggagtgaggacaaccagaggtcatgatcatcaccatcttggtttgggctggcttttttttttttgagacaagagtttcactcttgttgcccaggctggagtacaatgacgtgatctcggctcactgcaacctctgcctcctgggttcaagtgattctcctgcctcagcctcccgagtagctgggattacaggtgcgcaccaccacgcctggctaattttttgtatttctagtagagacagggttttaccatggccaggctggtcttgaactcctgagctctggtgatctgcccgtctcggcctcccacagtgctgggattataggtatgagccaccatgcccagccatctggctttttttttttttaattgcattctgttttatcagtggagtgcttgtgacctgtatcttgggcctcctgtctcatcctgtgactaccaaggcctagcctcctgggaatgcagcccagtaagtctcaggctcattttacccagtccccattcaagatggagcccctctggttggaatacctctgtcacttccatcaccacatccagtctcaccccacaccacctccccttctccagctccactaccaacccccaccagggggaaatgagtaaactgcatgagctctgagaaagcagaaaaaaagccacctcatgaaggtgaaccaaggactgagtcgctgacagacagagatggatgggtaaatgggcagacagacagacagacatggagactgaaatagaaatgcctcccaaaggtcacagagagtcagtgccacgtcttcacctccacccaggactatcacttatacacccccaccagcctgggcacccactggaaacttggcaggtctggctgctctctgtgatgactgcagtcagcacagggcctggtattccacagatacccagggaacatgctgagtgagtgagtgagtggatgaatgaaggaatcgttcattggtagaagcatcagcagatagagatgacaggagggagtgtgaaagagagttgaggaagaacagaaacagatggagacagatggaggctgcaagtcacagacttggccacagatagaaaatgaacgaataaggtggagggaggttactgcagaaaatttaaaagctatagaaagccggtcgtggtcatgtgcacctgtagtcccagaggctgaggcaggaggatggcttgagcccaggagttcaaggctgctgtgaatccactgcactccagcctggggaacatagcaagactatgcttttttaaaaaaaacaaaaacaaacaaagaaaaagctagagaaacccagagagatcccagtgaaagttaaatttacagacaaaaagaaaaggagagagagagcacaagccctcccttaacttgtcaggatgttagcagagagaaccaggggttcctgccttagtttccctcctagatttgcaggtctctgctggactgtgtcccaagggcctggcgccctgcttggttttgtcagttctctccctcgggcaggggacagtttcctcttacccccacagcatgccagctcagggccctcccacccagaaggccccagacaactatgcagaatccccccgccaaggcaggtccctgggagaagatctctctccccagctggtcctcattcctgcaatgacaatcggctcctgtctccactggctctgggctcagggcccccgggttggataggggttggggctggaagtggaggtagagacaggagcctgcttgccccagactctggggaggggtgaaagtcagagaggggagaagctctaaggggctgaggcggcttcccgcctccacagagcccaacctgaatgcccagagtactgtatcagcttggttatttccaaatcccctgggagccgtaggggaggggacccagagagctgggaggaaaaccgactgggaagtagccagagccacagaattctcctgggtcctagagaagggtgggttccttccacccccaaccccattagtccctcgaaaaggctctgacctcacctcccccacttccggagtggagttccagccctgaagaaacacttccaaacccatggctctggccccctcctcaggccccctcctcggctacctcggccctggggactgggtcccccaccccaccgttgcgccagatgtggctgcaggggaggggtaggcacggggcccagctggcagtggtcactcggtggtcctctccctgcagagtcagcactttctgtgccagctcgcccagcctcctcactcagcacttcctccagcccgctttccagaggcagcagctgcctgggttgggtcttggattcccagtgctgccccaaacccatctacagcgcagcctggaagatggagggtttgggtgctctggggccttccctcttggcatccccactatcacagaaaattccgacccaccatgggcctcctagtacgtccctctgcaaagcaagggaggaatcccacagctcctgacccctctctgctccccagacccgctgctggtcaggcctgctgcttcctggttcccccatggactttccagaactcagccttccatgacagggtcacaggaccctggctgctccgtgggagccccggcaacacagagctgagcacgtggcaggggcttaataaagtcatgcgcaatcaacaggctctttcatccttctatcaccacccctccgagggtcccttgacaccggttcctagtgccaggaactggcaacaagcttcaagcacatgcctgggtcttaaaaatgtgggtgtgaatcccagcctgcagggacaggcggagagagaagctgcccttcaccccagacagctctggctcagctcagccttcgagtccacgccctcatttgaccccagctctgcaccctccataacctgcccaggtctcttttaggaccccaccctgcaggtttcaacctaaggatgccaggacacctccagccaccaaggcaaggccttgcccagcatgaggagaggtgatggaaggcaggctcccctgggctcacagaactgctgggcctgtcagccactgcaggagacctaaacccaagtagaatgaggtcagaaggagggagtagagaggaagggagaggacagctctggtggaggggccacctctggcccccctgcacaaggttcaccctgagcaaggaaagatgctgaagacagataaaaacaggaaagacagaggaagtgggagtgggggtcaaccccaagagataaatccctcccttctctcacaagtcaagaggaaagaaggaaaatagtggccacatgtctggctctgtgctggggacttttcataatcctctcattccatcctctcaaatatgccacaagaaaggtatgattacccccaggtcacagacgggactctgaagctctgagagcttaaaaagcttccccggggagtggctgggccaggccaggtccctagctcaagtcatggtgtggacccccaggtctccatctcagcagggatggctggcaggagcgcagtcctggccaggggagtctgtgcagaggcccaggctatgttcagagcagagtttattcaaatagagcctaacaggaaacttggctcctctgactcactctgattcgaccttattaagaaaaaaagagagaggagcaggagccagcatcgggtaaggtttcacgccaagagctggctctgaggcccgctgagtggaatggcatgtgccctgatccccccacatccaaagccttgaggcagcccctgccctgctgtccgagtcaaggcgaggggtcctgccttcacgttagggcaactgagttcccttcctcacagccatcctctgtcctccctccactcctcttcccttccctccctgcctaggggtaccctgaggcctgtttccatttctccccctcctctgctgcagcagctgcccatctggctggcgggagggccctacaggaccccagggattccaagcagctgaggccaacactgcagggggcaagcaggagggagggaaggcttaaccctccaggtcccggccctcagtaagccctgcctcagcatcttgcttggtgtcagtcacaccagtggctctttggaggaatcttgtctggagtctgagatggaaaccccatgctggggagctgaggtgtcagcgttgacaagttgctggccaggaggtattggaagccccctacctctgggctggattctgggcatttaaagggtagagacatgctggagtccaagcctcagtcctgaagaacagtgcaatgggtgaacaaatgctgcctggcaaggatggggttgggaggtctatagttcccaaagaatgtccactctagttgcctcctctctaggtgggctccaggcatttccaaaataaagaatatacaggagccaaattaaggacaggtccttcataccttgtttataagcccaagagggcaatttctgcccttcggttcccaggaacacggttggagaaggcagcccaggggaggccagaagagcagtatttggagtgtgatttcttggatgccaaagcgttcaaacttctgggaccccctcctatccctgccattcccagaaggaacagagaatctcccaaagcactctcaggagccatctggcctaatcttccattggtaagccgtggcacctggatcagatagagtaagggactaagccacacagcaacaggaccaggccaggtctccggaacccccttctgttgttcaatgcttactggcttctctgcctcacagtgacccctgtcccatatcaaagacagcccccagtttcattttatccatgtgtacactcaagttattcccaggctatgcagagccaagagatgtaggacagaaaccatacgtgatgtctgggaagttgatctctcccaggatctcacaagtgcttttcagctcagggatacaccccactcttcagactgggaaagtaagcccctgaggtgtgccacgagggaaaaggtgcagctccagtgcctctcttcgcaggcccaagagctgcggtgcacctgggacctggaattagagagtggtccctgttcagcatctccccgaggaggcccaccaacaaagagggtgtgtcttttttttttttttcttcctattgagggtgtgggataatggtggaaggaacatgcaaagagggtgtgtcttaattagcactggctttagggaacaaggaaaagggagaacccgggagtacgggaaggaggctggggcagacaggagtcagaggcccattccagcccaaacgagaagccagtgagcaaggtggagaccagggatgctgtgaccaaagcagagaggaatgggcggggtggtgctgacaccccagccccgttctgcctgccagagccccacttaccaggcccgagtccccagaggtcccctcctactccctgctcgattcccttcctcagaggcaggtctgtggcttggctgggaactccagggactgagggagcactgcagctgtgggaccggcgcatagctaaaagccggcgggccatagggccccgcggaggaggccccagcaggcggaccaggaggccgaagcctcccgacgctcccagcctgttgcttattcattcagagtgggaaagcgccagccgagcggccagccagtgcggggctggccatgtaaggcccacaggcggtcctgcccgcccggtgccctgcggagagcctcgtgcagccctgggcaccgcccctgccctgccctgaccccttggccttgaaatgctgtcatcggaggagccgtcccgctcgggacaaggccagcatggacaaagctagagctggggcaagcaaggagccttcctgtcctcgaggccgtgggaagagaagcacgcccagggggccactcctgagagcctctctgtccaccaggcctctgcagaggggtcaccatggctctggcccgaggcagccggcagctgggggccctggtgtggggcgcctgcctgtgcgtgctggtgcacgggcagcaggcgcagcccgggcagggctcggaccccgcccgctggcggcagctgatccagtgggagaacaacgggcaggtgtacagcttgctcaactcgggctcagagtacgtgccggccggacctcagcgctccgagagtagctcccgggtgctgctggccggcgcgccccaggcccagcagcggcgcagccacgggagcccccggcgtcggcaggcgccgtccctgcccctgccggggcgcgtgggctcggacaccgtgcgcggccaggcgcggcacccattcggctttggccaggtgcccgacaactggcgcgaggtggccgtcggggacagcacgggcatggcccgggcccgcacctccgtctcccagcaacggcacgggggctccgcctcctcggtctcggcttcggccttcgccagcacctaccgccagcagccctcctacccgcagcagttcccctacccgcaggcgcccttcgtcagccagtacgagaactacgaccccgcgtcgcggacctacgaccagggtttcgtgtactaccggcccgcgggcggcggcgtgggcgcgggggcggcggccgtggcctcggcgggggtcatctacccctaccagccccgggcgcgctacgaggagtacggcggcggcgaagagctgcccgagtacccgcctcagggcttctacccggcccccgagaggccctacgtgccgccgccgccgccgccccccgacggcctggaccgccgctactcgcacagtctgtacagcgagggcacccccggcttcgagcaggcctaccctgaccccggtcccgaggcggcgcaggcccatggcggagacccacgcctgggctggtacccgccctacgccaacccgccgcccgaggcgtacgggccgccgcgcgcgctggagccgccctacctgccggtgcgcagctccgacacgcccccgccgggtggggagcggaacggcgcgcagcagggccgcctcagcgtgggcagcgtgtaccggcccaaccagaacggccgcggtgagtacggccccggcgcccctccggccgcgcgtacccctggccactggaaactgctccgggccccccgggcccctccttagaacttcctggaggcctccccggagctgctaagcacggagcagcgccccctcccgacttcccccgacccagccaacagcacccccctggcatctcacctcccagcttaacccgcacccaggcatcatcagtgccaggggttggccaggcaggtgtgggctctgatcctggctttggtggagggactagccgagctttgggaggaggctcgccttctgcacttggcttgggtcatctgaggacaaaaggaaggagcccctcatccaccttctccctcatttcaatgagggcatctccttccccccttagagatgttctatgccagctcacagggtgaggcaaggagcatcagaaacctgagtcttcagggccagaaaggccttaagggatgcccctggaggtgtggcaggctgcccattactgggcatgtgtgaggttggcttgagtgcgcaggaggttagaggggagcgtgtgctccaggcaggagtccggacttcctcaggggagtctgcaaggctggggcaagagagaccagtcaggcactggcctgggtgcagaatttcaggggctaccaaaaaacacagtagttgagatgagtaatattgagatgtatttttttaagtcaaaattaatgcaaaaaaaaaaaaaaaaaaaaaaaacctgtgataaaggaaatatccagattttaaatagagtcagtaatagtggtatgccgagccatattggagcctgaggctaactgaaaaatcagcaacacggatcctgtctttatttgatatttggatattttgttcattacagatagttttgcattaatttttattttaaaaactattgtgttaaatattacttatctccgttactgagtttttttggcaccccctaaaattttgcaccccgaggaaaaaaaaaaaaaaacacctcactggcctcattcccctcaccctagtcctggccctggcggagagccccagctgtggacaggactccctctggcttttcagggggtgaacatacgtcctcctcttgggaaggcccaagccctctgcacatgaggacacagtggaaaggcagcttccacacatgctgttagcagacagggctactgtgacaagggctgactggtgggtgtggccagtgctggacggccatcagccattctctgctgtcctagaatccccctccctgctaccctctccatacaggcagcttttgggccaaggttttctactcagtccccaatacttatgcagtccaactctgccttgcctctttcaagctgtcgtcagtttcctctacccataattctctgctccccattgaggttgctgtgcctggcccaccaagtaagggctgactgagccctccagtgcctcagagtataggtgctagtgccccagcctcctcctcagcctctgtcagcagagggatgatgctttgtctccagccaagctgacctcagtgaggggctgctgctcatggggtgcctgtctccctgggtactgctggaggccaagactaggggagaggagcgtctccactgggaacactcactctatcacatccaaggtgtcgcttttgagttgtgtgctgaatcccactttaagaacctgcaggagctcaggaaaaatcagatggcaggtccttctgtggtgtgatgaaggatgcatccacctgctctggtccttaccaggtacttgcagcatccctgcagaagggcgcattatagccatgcatcagctgctctagggccccttggagaataggatgttttacctagagggtgctaaacttttgcagtggcttttcttagaatgcaagacctcagcatgaggctcagcctgatgggccccacctctgggcagagaaaactgagctctcaaatgccacaatattggcagaggcatgcctgggacttggcatctgggcaccaagtcagtgggtcttctagggcctgtctttgggctgttagttcttaaggaagccagttgattgagaggggaatgggacagggcaaggccatgtctgaagtgggggtgcttggtgcttagcagacctctggccactaccctgagccccacagttcattttgggtcaaaaacatttctcccctgaaggtagcatcctaggtgccaagctgagtgtactcacatgcatcaagtccaggcccttccctggggcagtctaccaggaggggcatctcagtgctgtgattaggcaagcattggtggggccaggggctttgtgagcccagaggagggccctccctgtcaggggctagggctcagggagacttccacaggaagcaatgtctaagcattgctgcaagggatggtcatcagcaccatccaggtgccccttcccctaatcattgagggtgtggggtgggagtgtgttttttgctggggaggggattgcaggctagtttagggctcacaaagaggccagagagggagcagtagtctctcccatcattggttcctccctccccaccctgctaggccagaccgtggaaaatcccagcccagggaaggccaagccttgtgcttgagatcagacccccaggcctggtgtgggagcccaaggcggattctccgagactgacttcattcctcctggccacacacccaggggtctgaggctgggcgctggcagcctgtgagtgtgtgtgggcgtggacgtgtcctcacaacaccaagcacatgtgagcaccacccacctccctccatgggtgggtggtttccaagcgcctgtcctgagcccagtttcttcctcgggtacctgtgccctcttccttcctaccctgggcactgagactgcccaaggcttcctgcagtaccattctctccaactctgaaacatgaaactgaaaagaaactagaggaaacagaggcccagagagggtcacatactttgcctggctctcatagcaaatccttgaagagaggcctggagggtggccagccacttcccaggcccagctgggtctcctgcactgtgtcggctccctcgatgtgaccactcctgctattaccaaccaggccctcctctcctgggatcctcgcctcagctcctgcctccctggagtttcagcttgtctgagctgggggtcttagtgccacctaagaagccccacagccccagaagaccaaagctcccctcccaaggccttaccctcgcaccccccgcctgactcaccggctctggcaatcctgtgaaaggaacatggaacaggcagccctgggcatcctcccagggcatcttggatggctgtgctagccttgcctgacgtgtgtcctctatccctgacttcacatggccaatcagaggccaggagagaaccgcagctgcctcctccctggtcccttgctcccagtctctccctcttcagtctgtcctgcatgtggggcagataaacccctgcagtctgaccaggttactcctctgctcaagaagattctgtagctcctcagggcctttaggacaaaatctagtgtctcagcccagcactccagagttagaggcacctccttcctgtcctttcttcctccttttctcaagcctccacagcccccatgggaccatgctgctcgtgggcctagatgttacacctccctagacacatgcaagggtgtttctagggtacctccttctctgtccagcctcctgcacccaagaggccagacctgtctggaccctcagtccctgagagcagccctggctgcctgctaccacctctcctgcctgagcccctagctcacctgggcctgctccaccccacctccacaggctgcctgcccacctctgccctccaggtcactcgccttcccctgcagagtttggagtagatgcagggaaggaccaaagcaggcttcacccaacaagcttccagagttagagcccagaggcagggataataataccaacttggccatttgctgaagaccacttcctgggcttactgacttcaatgtgcagtttcattcatcctcacaacctcctaatgaggtgcagattatggtcctcctggtgtagatgagaaaacaggcctagggaggtggcacagaagggtacagccgagcttgaattcaaactgaacctgtgctctctgcatcacagtgagatgacccaggaggatatcatgttcagcctgcgggcagggtggggcagggcatgagcatggacccctctgccagctttgcctctgtggtcctcacctcccacatctgcatccagaggaagaattatgcttgcatttgctgagtgcgttatacatcatctcctttaattcccaggtcctagccaggtactgttatttgtcccgtgttacagatgtggtgacgagctcacttgctgacttttagtagtggaaccaggatttgagtctgtgtgactctctatcctgagttcttaacaaccatgcatttaaaaaaaaaatttatatattattgcttatcttgtacacctcatttattcactcattcatttggtcattcattcattcaacagatatttgagtgctagattctgggcactggttgtgacagggtgagatgggacagtgagtgtgaaagactttggaagcagagtctctccccagtgtggggttagggtgtagcctggtcatggtcattgcagattccagaacgtttgtgtaacaaacactcatatggtgcatactaagtgccggacactgttcttggagttttgcagttagaatcacttaatccctgtgacaaccctgggattgttatccccattttgcagatgaggaaactgtggccctgagaagttaagcgacttgccctggaggcagaatcacagatcagcatccctccttccttccccgaagttgtctcagattgttccttcagaaagaaaaagagtcttcaggcttagaaagctgtgtcggatcagacaagctgtggaaacggagggttctgggccctcctcctgcctccctgtttctcctttgagagtcatcagtgtctgaacaaacaagtggagtcctaagcagcctcattccaggcagacttcctgtctcctctcttatcagccacttgcaaggcaccttctctctcccctgctctcaaactggaccatccctgcaagaagagacagctcctgttttcaataataagaataagaatatctgatatttattgaacatacactatgctaagcattttatttttctcatttaattcccacaacagccttgtgagataaatagtattatcagctctattttatagatgaggaaactgaggctcggagaaattaaccaactcacccaaggtctcagggttaatgagtggccaagcttagacctaggctgactgtccaatccagtgggccctggcctcactctgttccgtctcttagggacgggtgttttatattctgtgtcacctgtctctgtgttcactgttcccctgaggtgtaaacacctgaaaaaaaccaaatttgggtttttgcctcaagtttgcgggccccacagagttgctggggatggtggcagagcagggttgggagccattagtaccattatgggcaccaggttactgctagacttggcctgggagctctggctttttgtcctctctccttcctcctaacctcaggggtcctcactgcagcatcctctgctctcagaaggtgtacccctctccagtagctccctagtagctcctgagtccattctacctcctttctaagctgggaacacatggaagacttcccaggacagtggctgacatgggcaggagaggaaaggggcagggagggagctggggtccgcccaggcggcccacacctccttccaattgctgtgcccagcattcctgcagaagagcagtcagggttgggatgctggaaagagaggtttagacttgcccctcccctggccctgacctagccgcctcatttccacaggcagctggaccaagggccttctttttgtcgggactcatgatcacagggtgagggttgggtctgggtccctctccctcttgctgatgggcacccagaaaccaggcattgcagtaccacttggaaatggggagaggggtgtcagaatgccaaagcaggaagagacacaggatgggaaagcgaggccccagaggcctttgccatccaaacctcaagagacaacccatccttccccctgcttgtgggccagctggaaccacaggttttccaaacccaacgcaaatctttctgcaaaagaaaggactctgggaatcgattccagaccctgcttgacaatgaagtctgtgctcacacaggcagtgccaccagacgttttatggagtatgggcattggctcacgcacttgccaaggacatacaggtgctaggccctgagctgggcgtggagaatttcaggggcatcccagaaccctccctggcctcactgacgtctcagcctggcagtgagaaaacagacacctggggtggagagtggcccaagtccatgtctgtctttgccagctctcagcacccactgtgcacctgggagacgtgcaccagaagctgagtacaggcaaaagataataccactcacccctgccctcatgggctcttgcctggctgcagaaggcagatatcatctcgtgaggaaatagggcagatatcatctcatgaggaaatagtcatttaacaaatatttattgagcgacttactgtatgctgggcaccgttctagagctgggtattcattcgtgaaaagaaagagacaaaattcttgcccttgtggatcttctattttcatgtataaacataataaatatgtaaaaaaaatagaaggtactaagtgctgtggaaaaatagagataggtaaggaagatcgggagtgtaggagagggacaggttgcaaaggtggtcaggcctcatgaaggtgacacttgagcaaagacttgaggcatatgagtgagccatgcagctgtctgcaggaagagcattccagataggaagacagccagtgcaaggccctgaggcaggagttccctggggttttcaggtgtgctccagtgtggttagaacagagtagagtggaaatggaggtataacaggggacaggtcagagaggtaatggggtccgggccatggggagcttgtcggccattgtaaggggtttgccttttattttctgtgcatcccttttactcagcgtttgagctgagtagaagcatgggtccctgactgctaaacagggcccctgactgctgtgccaggagtggacagtggagagtcagccaggggaaccgacaggaggctgttccagagagtaggaagagtggtagcaatggaggaagccagctttagggtatgttcttgagatagagctgaggggatttcctgctgggttggatatggggtatgaaagaaagagaggtgtcagggatcacacagtggtttatggctggtgtggctggaaggatgcaattgccttgagctgagatgggaaggctgtgggcagcgtaagttttgtaagaaatatgaaaatttcaggtcactgtgcagggcctgagagggcaaggagggaacccctggagtaactgaagcatcaagggaagcttctagaggtggggtagggggcaccaggcagggcctgcaagggcggggagggctcaggggcaggggttgggttcacgctgggaggaaatgtgcattaggtgtgcagagaggcagcagaggcttggcctctgagcagggagctgacctccgcattggttagcattgtctgcctccccctcagcccagcctgcctcctgggcagctggcgcaatcaagcacacacttacaaatcccttctgtgagtcaagcccttctgctgctgtctctccccattggtggggtggacaggtgagtgctgtaaaagggtgcagggtggtggtggaggggtgttcaggactgacagggtgggctttggagctaaagactgtgtgcctctgcataggtagagctggtgtgtgtgtgtgtgtgtgtgtgtgtgtgtgtgtgtgtgtacgcgcatgcacatgcatagctgggaattccaggacaagggcaccatggagtcagggggtaaaaaaggttggggtatgctgccagagacctgcctactgatccttaggattaagagttctggaaggctaggtaaggtcaaaccacagacggctttagctgccactctgagacttctaggcttgattctgtgggcaatggggagccattgaagaccctgggcctggcttgatggctgtcctagctctcaggcttgagctgggttacatcttcattatggggcaaagtggaatctgcagccaccttgcctgcctgctcaccaagcaggtgtggcactgccaggccagtgctgacaagttaatttcctgattatgagataatcatttcctagggaatcgggcagccagctcccgccgtggaggcctctctggctcatctttattcaaggccctgctctgcagagctttctgcttctctgccacctaggggctcctctggagtctggctgcgctagccaaaacatcccgctcagcacatatttcacaagcaactgaccagttcctcctggactttgtttatcctccagccctgggctcaagagattagggctcttttgcattccactttccatcatttgcccctcatgaggaggaggcttggagaggaaagattccagagacaaggtttactgaacccacaaaaaagaggaagccaagagggaaacaggtgaggtgtggacgagcaatgggaaggcagccacagggctctcagcgccaggcagcctccctccaggaaccgctgggcaggagggaacagatttctaatgaagcaggaaggatttaggtcagaaggtgggagaatttcctgactgctaaagtatcgtctgtaggggttgtgagagggctcgggctcttgcccaaggctggggaatgaactgaacagctcctaaaaagtattttaatctccaagtgtttatcgatctatccaacctgtgttcaggggccagctagttcagtctccctcatcatccagatggggaaagtgagcctcagaaaggggactggggagactggacaagacagtgaactcacctctggagcttcccccctgagaaggccccagctgcatcccaccagggaaggggcagatggtctttgtcctccggttcccgagggcgggcacgggagccgtcagcaactccagacccagcattgagcaggaggacagggtgggcttgtcccgagcacacaggccccaagagtcagtgtgacaggttcctcctactgtgctcctgcacactcccacagaggaagctccaggggcagagccaggatatagaacattggttctgtcttctcggctccgtgtcttcactgtcctggaggtttgaagggcatgctatagctaaaaggcttaccccacatcagtggctggctgtctccctgtggaatgaatggccctgggaccgtattccctcgcacgcagctcctccccagccgagcatcctggctttggccatctcaagggtcttcgctgccccgtgactcagcatccctgccatcctggcccaggctctgtgcagcctgcatctgaggaagaatgcccacagaggccggcacagggcctcaggccccgagtcaggtggaggacaattagaagtggaagaatggaggccatgaagcctgaagtggggctgggtcaacccaaggccacacagaaggcctatgacacagtagggcaagaccccaggctcctgtctccctcctgggagcaagaaattgatggtggccagggttttcctgggtagtctgagcaagcccatccacccagtctttgggtggtgagacccgccccagcctacccccaccagtggtgctgggggcttgggagaagactgagcagggctgtaggtgacccagggagctggctgcgtaccttcatcaggcccaggaggggcacgtacggccgttcactgtagcgtgtttgtggagcggggagttagaggtgacctggaggtctaaaacaggaggctggccgggtgcggtcactcacccctgtaatcccagcactttgagaggccaaggcgggtggaacacttgaggtcaggagttcgagaccaggctggccaacatggtgaaaccccatctctactaaaaatacaaaatttagccagagcacacacctgtaatcccagctgctcaggaggctgaggcaggagaattgcttgaacccgggaggtggaggttgcagtgagctaagatggcgccattgcagtccagcctgggcaacagagctagactccgtatctatctatctatctatctatctatctatctatctatctaggtagatagatagaacaggaggccagctagaagggacatagaggatgcacatcgtggaatcctatgtagcagtgacaggcagtgaactagatagaccttaaaagtttacatggactgggctcggtggctcaccccataacctcagcactttgggaggctaaggtgggcagatcacctgaggtcaggagttcgagaccagcctggccaacatggtgaaaccctgtctctactgaaaatacaaaaattagccaggcatggtggcatgcgcctgtggtcctagctacttgggaagctgagatggaaggatcacttgaacccgggaagcggatgttgcagtgagccaagatcgcaccactgccctccagcctgggctgatggtttcccctgtatttggagtaagcaaaaagacctcatttgactgggtgcggtggttcacgcctgcaatcccaggactttgggaggctgaggcaggaggatcacttgagcccaggagatcaaggctgcagttagccatgttcacgccactgcattcctgcctggacaacagagcaagaccctgtctcaaagaaaaaagaaaaggtggaggtggcgtggggagctggatatgtaccttcaccaggtccaggatcttcctttaaaatgcatactttttgaggtgagggctgatcacacagtctgataataactggacttcacccaacattggccactctgcttggagggcctgttgtaccctacaagccaaaaattgcagaccagcctccttactcctacactgatcgtgtccactagcccccaactcaatcaacccttctttatccccttccaacatcacataatcccccagccccaagccctgcagaggggccaagagcccaagcctgcaggcctggctgctgacgaaatgtcccctgggagagctgcccagagaaggagccattgttccttcctggaaaaacataccagctatgggagggggtctgctattccctgggacctcggcaaggatcctgagggtgaggcgggggggcctggcctgaactcctgaggctgctcctttccagcagcttccccgagctgctgcctcttgtgtcacccccatggccatgagggtgtgacagaggtggagctcagagagggagtcacaggaggccctggtgattgttagagatgtagaacaaagaaacttacaaaataatggtaagagtgattccatttaagttctagaaaaggcataactaatctacagtgattagaagcagagcagtggctgcctggggtggcggaggcggggagaggacttacagggaaggggccagagggtgacagaaacaatccacagcttgattgtggtggaagccacacggctttatacatttgttagaacttatcaaactgtacacctaaaatctgtgcatttcactgtgtatagagtacagctcaaaaagtttatttttataaaaagagtgacaggaggagtcctgggagggcaaggagacctaggatctcctcctggggtaccccagctccctgtgtgaccctgtgcagatttatgggcctgagcctcagtttcctccggtgcttccaaagacattatgtgccttggtgtcttgggggcaggacatggaaaacactttgggttgcctagaacagtcattaaagaggctctcctgacagagcctgaggggctttggaccagtcagtcaccctcaaaacaaaagttgctccactgagggcgagtaggactcagggcctgcccatagaatgccatctggagcggaggagacacacacaggttagtatggtgaaatctaagtgtagatcaatacagatggggtggtgagagaggagaattttgtgcagtctgagatatcattgcaatcaggggagacttcccgcaacaagaggcccctgcctgagcctgagattcctcaacagtaagaggaagaagatgatcatctgtagcgagggtcaaggctcagattgtgaccagcagccccaagctgtttgtccttagtctctcttccaattctccacatgcagtactctacaaatgcccctcctccataactctggcacttataaccctactccactcagttttaatcagacttgatgatatcacagcaattcctgtttttttttttttcctttagctgaagagctttagtccatcatcaggggacaaacagaaatgagactgagattcagaattgctgataggcatcagacctccgtctgtgaccaggatcagggctcggtgtgtgaccagcatcgggactcagactgggatcaggaacagggcttggtgtgtgatcagggtcaaggctcagggtgtgcccagggccagggttcagtatgtgaccaaggtcagcgcttaatgtgaccagggtcagaggtcagtctgtgaccaaggtcagggtgcagactgtgatcaggcacagggctcagtgtgtgtacatagtcagggctgggagaggccaggaacagcattggccctcactggccccacctggcctcttcctcccgctcctccaacttccctgggttgcatagccagctcatgaaatagcctagataggaagggtcaggccacaaaagctttgtggagccatctgtgcagtatttcttgaaactgggcctggggtgggggcagcaagctgtctgatgtcaaatgctgggtctcctgagtcttaaggcccagctaccccaccctatacgcagctgagggaaaggcactgggtgatggggaaggggcccatccctctcatcctcacccctcccccagaggcttccagggtccagagagtgagaaccagctcccagctggcttgagggcctatgggtcccttgggaaatgactgcaaacattcctaggggctcaagtaacagggcctaggctggccaagaagtcccccaccagccaggagccacctcatcctcacccacccttgtactccaagcttcctcatcattccaagtaggtgagatcaccctggctgcctggaccaggctcccccatgaggcccaggccccagggagagcaaagctagggtctgggaactcagggtggtgtggggtgggggatatgctggcagacacttctgccgatggcttagaagccagccttcccctccttggcctcatcttccagacctaaaagctgatgagagggtcttggcagcaggaaaggggtgggaaatccctccatgggactcaagtcctcccaccttgcctgctctcacccttcctgaatccctcaagagcagaggacttcaaagaaactaagaggagcttacagcctcatcttatcacttacctgacagaatccctgaggccagagaggagacggaattagaggccccagaaccactcccctttaagtcagtggctgggctgggatagagcccagggccctggagtcccccctgcccacacccactgctgcccgcacccaccccagcccacaccccatcacgaacagaggagatattccctcttcattccaatgcagaatggagtctggacacctaagtttggtttctgcttattcctagtaactcctgctcagactcctgcatgcccagggaggaaggtggaggggcaggaacaggacggacaggccccgggctctggcacatcctggggaacaagggaccacaaggacgggggcagtctccagacttcccctgggcgcttgaccccaggccttgcaggggagagagccagggcctccctcaggttggtggccggaggttgcagacagaggagggtgtggtcaggtctagggtgatgatggcaggaagtggtggcaggtgatggggggcgggggtatggggaggagtggctgcaccaggcagaaaggaatcctgagaaggtcaggctgggtgcccaggctgagaatcctgccctgtcagcagcctgctctggggaagtgttttccttctagcccatcccttcctgcccaccctgactcctgccagcctgcatccacctggctctgcccatgaaccttcagggacacactccaggccagaagcccagggctctcttggtggtttacaaagaatgctgactgcagggatgagaggaacggagggtgtctgggggctgctgtccagaggcctggggagtaaggactcgagggagtgcctctgtcaggaaattacatggttcactcattcctgcctccaggtctttgttcatgctgttttccctgccgtggacaccctttcccgctctctgattctctaaatcctgccccatctcccagatcttgttcatgtccaagcttttccaggaagtcttagcagctcccacaccgcagagctcgagatgtaagctcttgctgcctgacttccctgaccccacacctgggcccaggacctaacacccaaggagaatggcaaagggttttggggtatgagaggatgtttggggtgtgaggaggtctctgggcattagcagtggccaacctgatgctctcaatgtcatgctcttcctccctccccccttctctcccactgcccactccaggtctccctgacttggtcccagaccccaactatgtgcaagcatccacttatgtgcagagagcccacctgtactccctgcgctgtgctgcggaggagaagtgtctggccaggtaaggagctgaggcagaagtgtagagtgttgggatagtccccgggagtcacccagaacccagcctagctgtggctgccagctaggctgtctgcaagctgatgaccctggccaagtgccccagcctcccaggaccctgaccgtttcatcagtgagccttgcctgccccgagggagtcaggaggggaggggagcaagcgggggcccagcatttctgtatactgtacctcactatacaaggaactccacttttactgtggcacttcatctttatatgagccctgaaggcatgggttaccccctcaggaaactgaggagcaatgaaaggaactcaaaccttttggatctgagctgtttaatgagaaggttccttttgggagcccaggcttccagtggtcatacagggacccaaggaagtcctattgggtggatgtacctgcaggatagagcccaggagtatggagccatgtccacagaggacagttgtggctttggctggtccaggctgcctctgcctagtcacagcaggggcagtgagcccgggggacaaagaagtttcagttatatgttgcactggaaagaggggaaggagaccagtttccatgaacctagaaggtgagcacagctggctcgccattattcatgggtgatcctgcctctcgcagtgtttgcttctaatttataagcagttcatattctgctggaacataggtgctttgcggatacctccctgggccaaaataattagttaagattatgttatctgcttcttttgttaatatccctttatgttgaaagcacctatttttcttaattacctgctttttggacatttgaactctctgtttagtttgactttttactatctaggaaactctttagaatctcaacattttctcatttctagaacttctagttcactgccactctaagtagctgatcataaacccttatctgtctgtaacaagataaagagcttgaactagaatgtaactcaactcactccttcattgagaaagtcttgctttaaaaaaagaaaaagtatccgctgaactaagcagagtgattaggaatgttgtcgatgtacattctggtgcaagttcccaatcccatcccagactggcatcagtgtgggaactgcccctggtccagggaccgcaggctgagtaccactcagccagcctgtctgggcagttcttgtcttgtttcagttgtcttggggaagagagcataatggtcctgctggaattcctccagcaatgaggaacttacagcctccatgacagcccttccagtcttaagagaattagtcaaagtcaaactattaacatcactgcctcttcatgaaggatcctgagtgcccggacctgcgctcccagctggggagaggaggcagcctgtcctctgtcctggagggtctcctggtacagctggagagaacagccctcacctgtaccccagtgacagaaccaagacacgagtgggaaaagagccagaaggactggttcacaggccagggctgctggaggtcagaggagggagggatcagcaagggctgaggcctcagaggaggtgggcgtgggagttcagacctggagaatcctgcaaaactggagccctggggcaggagggccctctgagatcatttcaagtatgggccgcaggctggtgccagttcacatgctgcaggcagaacccagagaagaaaagggaccaattccaagtctcaccaccagtttgtggccaagctgggcctagaacctagacccttatggtttcccacggtgtgctgagccgaacagaagggcaggatttaactgaacagagggaaggaggatgttttgtttcaacccagacatagacagattccaagcctcccccagacaaggctgagggcctttcatcctggcagacagtctgagccgcctgttgtccagacattctctcaaagagagcgctaatccctacgactggccaccgctcccctctggccttaggatcggaccttaatgagtgccagggaagagcccctgaggccaacagagttagctgtcagccgtcagctaaagaggggaggggccttcagccaccccttcctgttcctaccacacatgtgtgtgcatgtgtgtgtatgtgtgcatgtgtgtgtgtctcccctcactgcagcccctcgttcctctttccacccttttccttttgtcacaaggcctacctcagttggaaatctggagtcagcaggtgacttgcgagcaggcaattcataccagacccatgttccctgaatggaaaagggcaggttgccagcctgaagccatgcaatcagggagaggtggtctggacccagtgcaagatgtagatggtatcgggctgtgctgcagtcagtgctgttagagctcacaggcaatccggatcctctatgtagagcaatcagggcagacttcctggaggaggggacttttgagttgaccctaggatactgggtgagttttggaattctgggtcaagcaaacagcatgggcaaaagtatgggtcagggaaaaagttcagcctgtttggaaaataggctttgaagcaggaacccagggtgtgtgggggagtggaagggcagatgggactagaaaagtgaagaggctttgaccagaggacttcacgtctgtttttcaggcccacacccaagacatggcatactttggtcaacaattagggctctgtcttgttgtcagagacttatcagaatctcctggataggacttttttttttcttttttttagaaagggggtctcactctgttgcccagactggagtgcagcagcacaattatagctcattgcagcctcaaattcctgggctcaagggatcagtctccaagtagctgggactacaggcataagccaccttacctggctaattttttaattttttaaaaatttctttaattaaattaattttttaaattttaatttctcactatgttgcccaggctggtctcaaactatgggcctcaagtgatcctcctgcctcgacctccaaaaagctctgtaattacaggcatgagccaaagtgcctggcccccgcaatagggctgtttttcagtggcccttcagagattgctatgtgctcctgcacctaggggctgaccccagctgagtcacctgcaggtatcagggacactgccatgcttggtaccgctttagaaagatgctgttggcagctggcggccacatggtggaggccagaaaggaggggacagccaggatttcacggggtcagaagacagaggaagaggcctgagcctccgccacagttcgaggagacagaggagaagatcttcagggacaaggagtggggctgcagaaccagtggccaagggctgggggtggggctgagggggcccatgctgggttctggtgtcactgtgccccaaccccccctcatctcccccgccgtccctgcagcacagcctatgcccctgaggccaccgactacgatgtgcgggtgctactgcgcttcccccagcgcgtgaagaaccagggcacagcagacttcctccccaaccggccacggcacacctgggagtggcacagctgccaccagtgagtggggaggggctgggcccgtcctcttccacttctcctctgggccagggaccttggtgcctgagctcccctcccaccatgagcactctgggagacacagatactggattagagggcccggggaggtgttacctgccaactcttttcactggggcaagtgtttctcagagaagccttagccaaagctaggctggaagatggatatagaaggattggtctcaatcagggatggaaatgtctagaatacagggcattgcccagtgaggccattacaggtgggagcccaaaaaggaagtgaggggtctagagcatgtgctgtcctggagtgacccagctaggtggggagcacagaggggccaccccgcagtactcaggagacaggagaacctagaccagccaggaggctgtgccacagcagggtcacctgagcccatctcaggatggggacaggatgagaggcccagggaagactaggccctcttctttctccttctctcctctgcccctaggcattaccacagcatggacgagttcagccactacgacctactggatgcagccacaggcaagaaggtggccgagggccacaaggccagtttctgcctggaggacagcacctgtgacttcggcaacctcaagcgctatgcatgcacctctcatacccaggttgggctggagagatggggtttggggcatgggaggataaggagttggggaggcaaagagcgaggcccgctgaggcccggcaagtgccaaggcttctggccactcagctctgctcacagtgaaggtcttctcaccagtcctcaggctgccacactgccctgcagggactgttccctccctgccccagcccctttcccatgttattccaggtgatctgctcgtggagagaaggaaacatcgcaacagtctggagagcaacacgtcctattggcctgttcacccacccatatccctctttccatcagccaccctaaatatccacaaactgtccatctgtcctgtctctttttatccattctgccatccatctcgtccctcccacctggctattagatatctgtctttcctctggtccatctagccagtggctttaggaaggtgtggacccaccctgagcagttgccctgggccagggtcaggggagaaacccaggggaaggtgggccagaaactcctgaaggtgggcgtggggtggctctgggaaacaagcagcatcacagccgctcctcttgtccctttcccagggcctgagcccaggctgctatgacacctacaatgcggacatcgactgccagtggatcgacataaccgacgtgcagcctgggaactacatcctcaaggtgggcctctgggtctggggctttccctccaacctgatgttcatgtccaatgtccccccgttcctgaaagaagcttcagcctctgggcctgttcccttctccccagctgccgagcagaggcagcatctgaggcatcacttgaggtttaggcttcccagccgaagatttaggtctcaggctgacatagcctctctttccataaagaacccagggaacctttgccccagctgtggtaatgccagaggcagtggtgatactgtctccctaagtgacagaggaccagggagagcaagaaagacttggagagccatgtggtctatcttcctgtcttcggacagggcagactttgtatcatttgggagacagatgagaggccctcagctccttatagggacccctgcaaagccaagggctcctgtgactaagacccttttagcctgaacccctcctgctctaatggacactcactccatcacctcttcccttggttgagggaaaagtagaaaagcatattaacaacacacatccagacttggtacatccgtgtaggccaggttgtcatgggcccacctggggaagccatgtctcagcctcctccacacaggtataaaatgagtagggatcattccaccagttcatctggacccactcagaagcagaaaagcagctcagtaaaggcagagcagtgaagccaggtttgcaggcggaggccttccctgcagaaagtcagcaggtggagagatgcatccggcagcggttctctgcagttccctcctgagtgctcttcagcaggctccaagcttgctcctccagggcctggggttgtccagtatattttttcttttcctcattcatccactcaatcaacatgtaggaagtcccctctgagttctaggcactagacacacaaaccacataagcaatgcagttcacagtccctgccctttgggagtgtaccaggatttgaaagttgcatccctgcatctgttcttagatgttctgcaaataaagctggccctaccctagttaatccaacatttaaaagtgttgtatcccaacagctccttttcagacaaaaagcacagatcatacttgggccatcatctcagtgaatgttgggcacgtggggctccagagcaggggatcagacaggccctcgatcagtcacctagacagggatgcatagggaatgactttgagccttggatttggggagaccaggtgttgaatcctggctcagctcctcattactgcattccctcgggcaggatccaacctctctgaccccaaatgccccatgcggggcccacctggcataggctctagctctagggaaacatccaatcttctgataagggggattagatgatgttttcttggattaccagctgcctcttctttcccctctgtgtctctctagcagtgtgtctctgttctccctctctctgtcacctctctgtctgtctgtctgcctctttaccaccttctctggtgagcagttgaggtgcagcccccctgactagactccctttctccctgtttctcttcttcctcaggtgcacgtgaacccaaagtatattgttttggagtctgacttcaccaacaacgtggtgagatgcaacattcactacacaggtcgctacgtttctgcaacaaactgcaaaattgtccagtaagagtttgcccaccacccttcctggctccgtccctttcctgcctggggagcaggcaaggccacctgagatacctaagatacctgcactttaggtcaccttgatggccgaacgcagctgcagcagggcccatcaaaaagagccttgtccgtgccaaccccaagctgagtgtttacggtggggctcaggccgtagggtgttgatctggggctgcatcctgtggctccctgagatgtcccaagtgaaaaccagcagaaggcagagtcatttgatgcaaggccatgagctctgcagtcccagttccacagcttcctcgctatgtgacattgggttacttccctgagctactttcctcaactataaaataggtatttccacataggctggcaatgaggatctacaggcctgatgtctgcatgttcacagtggcacacagtgaacagcagctgtgatcattagtgtccttttcaagtggactggattaaacagtcacagattagaagtgattctggttcaaaggagccatcttcagtccctaggcccccattgtagaagaagtgcctaaaatctgggcttgggattttggcacatgctgctgggtagggtcctcagtctttctcctgattccccatgtgaagtagtttgcagagggaccccttgcttacaagctcacaaggaaaccccaccacactccagatcgaagcaacccttttccattaaaaaaaaaaaaaaaaattagtcctagagctttcagctttagttcatcaactgggagaaaggtggtagagatcttaaggcccagagaggtatctttccctaaaagtgtgtacttattaatggatggcaggtatgagagattaagcctatgactatggtcagggtgtggtgtatggccagggtcaggattcagtctgtgacaagggttagggctcagtgtgtgacaagggttaggactcaatgtgtgaccaggatcaaggctcagtgtgtgaccaggatcagggttcagtgtatacctagggtcagggcccagtgtgtgaccagggtcagggctcagtctgtgaccagagtcagggcccagtgtgtagccaggctcagggctcagtgtgtgaccaggatcagggctcagtgtgtgaccaggatcagggcttagtgtgtggccagggtcagggctcagtctgtgaccagagccaggctcatttagagacagccctctcacacttggttgctaccatcaacctaactgttcacagcatgctgccaccacatcatgaatattaaaaatgggaacagcagcctccagttcagtgcagagattaggttcctcatgagtagaatgagatcagaattccccagggcacacagcatgtttgaaaggacgaactcctgtcccatcactggcagtttggctctttagcaggcctgggtttccagcacattcctgctcctcagggcctgtcttagccattcaagacccagggtcacctaagtggtagaagccattctagggaaggaggaggaaggaagaagggggtatccaggaaacaaaggaggctgctgggggctcggggaggcagggaatccctcctgctgctgggaaagggggatgggaacagaaacagcatcccacgtgccaggcgcctggagcagccaggagggctgtcggcagggcagatggagaagcagcctgcgcggccctcccagctccccgggggcctgtgggcagcagggggagccaaagcccttccttccaagagctctttacataagtgcttggaaaggccgggccttattagagtgtctcagtctctgtgaccctgaccgcaggcaggcagctgggccagcgtgcacaggaagctggacagagcgttcccagaaaggccacgggactgacgcctgtgggtttccatcgtgtttctggaggaaaaaggaggcttaaaaaaaaaaaagtactgagttagaaaaaacaagaaagtatttccagacagtagcagctctgctaatggcctagaaaacaatctgggactagggcatggggaggggaatgtgctgagactgtgccctgggtgggtgggagtccatcaaacccctccttttggagctgggctgagggggtcggaactttctcttgagggaaatgtaggcccatgcctgccctgccacgagggatctgggctgtgggagggacgccctggctgaggaggccacgggggcctactttgcagcccctcattgacccactgtctttccttcctcagatcctgatctccgggagggacagatggccaatctctccccttccaaagcaggccctgctccccgggcagcctcccgccgaggggcccagcccccaacccacaggcacggaggggcatccctccctgccggcctcagggagcgaacgtggatgaaaaccacagggattccggacgccagaccccattttatacttcacttttctctacagtgttgttttgttgttgttggtttttattttttatactttggccataccacagagctagattgcccaggtctgggctgaataaaacaaggtttttctactctgtggctctgcatgcggcctgctggctggctggccagccacagctagtttgggcctgggggatgtccttaggcatcatccttcccctcgccaaatgtggaaagggcagccaccacccggtcaggaccacagtgaccatgagggcgctgagcccatcgtggaagcctgtggggttgagcccttggccaagcctgtcgcatgggaatgggaccacatccgactaggggagggccctgcagcggggtgggcggcaccagcctgctctccctgctcagcccctgctcctatctgggctcgtgtgcccactgctgccccttctcagccttcagcctcacactcccaccggtccccctcctggccctctcactctcacaccgttcactctccaccagccacccactcccttacaattgcaggctcccgaactcaagtctcctgaaggccaggtgctgtgctagggcacacgcaggagcatcgtagcttctgccctcagggagcgcataaccacagggaacggggagcacgcgttgcccagtgaggcagcacctaggggttctggggagccttcacatgggagagagcctccttccaggcagggaggggagaagaggattcttggcagagagacagcattgagcagagatcatgaggtggggctgcaggccgacagggtgtgagtgggagatggagggaggccccgcagccctgcagcccccgtcccctgccctggctgtggcatcccagaggtcctggacactggaatggaccaggaagaagtgagagggtggtgcctctcctcctcgaggccctgaacaacgagccctcgggatgcctccagacccacctgcttccctgtgggggcaacaccagtgagaaagggaaagagactttgtccaagtaccagggctctgggttccagggccaatcctgctcactgctgtgtgactgtgggcaagtcagacttcatctccgggcctcagtttcctcatgagtaagatgggaatgccctcctgcctggctcccacactgacatggagagttgatgagctactgctgcaggggaggtgccgggggagctacagggtgtcagcacccacccctccagatcttggtgagccccagcccatttgcagaggtcctccaagaaggtgtcccctgcttttggctctgatgcgatccttgcctcagggtccctggtagcaaggaggttttctggtgcagtagagagggagggcttcttggagccatgctatgcatccaggagggttctggcccagtcccctggagcccttgggccactcaccagcccaggcagcagggtggagtgggtgtggctgcagccctccctcggcccttcctctgcaggtctggctgggatttaactctgctgacagggtaggtgccgcagctctgccactctccctctggagctggaccaggacgggtgagaggagtacaggcacatgcctgtcatccagggctgccattctgctgcatcccctgccggtgtgtgcttcccaagatgctgtccttattgcagccccaccccctctccttcctagatactcccaggagtgagcttgggagggggtcagacaaggtcaaccaattccagactgtcaggaggcccgtatgaggtagagacaaaagcccccatttctcaggtcagaaaactgaagcctgtctgagaccacaggcaagttcaaggcagacacagtgctggagctcacgtctctaagccctaggccagggccaaatgccgggtgtgtgcagttttctggggagatctgtaccctcagcagatgagctatggccctgaacacagaattggatggaaggagagagtaagggtggcttttccaaaaagcccacacatcacaaagtgtggggaggtggggagcacacaaggagactcccagacacagtgctcccgcgtgcccatgaacctttggcctgaagttcagattctaggggggctggtggatgtccccaagcctgccatcagagtgaggacacctgggccagtcctcacatgctgggaggactctgagattagatctctggggaggggcttcagatacccctttgccccccaaaaagcagaggaggctttcacccaaaggagaagagagatagagagagaagagagaagacagccgtgacgcgcagggaccagggcagccctcaccgcttcctggctgacctgtcagggccctatcagtgcagcttcacggcactgcccagcagtgacggcccctcgggaaaggagatgcagctgctcccacctcagggggcagggtttcctcaggtattttgactccatgtagaatggcagagtctccctgagaatggggagagaaaaactgtcaagacggcaggggcccagcaggcgctgagctgaggagggcggctgggagaggagtgctctcccagctcggcctcagcctcttgaggtctgtctggctgtcactgcatggaggaacagcctgcttgttccttgttcttgctgtcacctggctcgggcccctccctagatagagctcacctgttgggctcaccccaccaagccaggcccagggaggggtgtgtgaggcccactggaggccaggtcacgtgacgcagggactcaggcccactggtcccttcacccttccgtttgccccttcaaggccagctgaaaccccgatttctcgaagccttccttaaccatcccctctccccttcaactggggtgcactgtgtggcctcattcaggactctcccacccccaccctgccacggacacacaggggcatgggaggaggcacccgagccttgcatgctggacaaaggttacataggtgccatctcagccaggtgcagtggctcacgcctgtaattccagcgctgtgggaggtcgaggcgggcagatcacttggggtcaggagttcgagaccaactggccaacacggcaaaaccccatctctgctaaaaatacaaaaattagccaggcatggtggcacgcgcctgtaatcccagctattcgggaggctgaggcaggagaatcgcttgaacccaggaggcgaaggttgcagtgagccaagattgtgccactgcactccagcctgggcgatagagtaagactctacctcaaaaaaaaaaaaaaaagaaaagcaaaacaacaacaacaaaaaaaccataggtgccatcttcactcctgacccagcacctccttctgggctgcacagaccaagagggtctctgctggtacactgggcactggtgtgcaggaatccacctgggctgtaagggcctcaagtcacctgcccagcatccctccctgtcctcttcttgctcagccccagggctggggagctcactgcttctcgagacatttgctgttagaaacaccttgaggtgaaacagacccccaccccctgccccgcagccttcccttgggctggtcctcggggtctactaagtaaatgactctggtggatgtgactggacagcaggaaggacagagacagcagaggcgacacctggcagggggcacagctgtggccctgactctgtccttcagtgggcctcagtctccctatctgtcctgagggggctgagtgtcttgctagtccttacctggcagagaagaaggggcaggggaagagaagagagggaggggaggaaatgggaggtagaaggaggctgggtagggagtgataggcaaagtccagcctgggggaaggtgggcaagcagcagggggactgcctcagaaggggagcagggagccagcagaggcactgctagggctgcagcctggtgagggctggggcgttatctggcttggaggatgccacagagagaggcctgaagtgggggtactggacctcaaaccagggctggcctgaaacccttggaagggcctgctaatagagacagaggtgccctgggcccttggcagggcggggtgggctctgaaagccagcccagacctgacccctctgcggcctaccatcttggccacagccttgaccttctccctccccctgcacctcagatgcccacaccccctgtgcattacactgtgcgctcccacccagggcagcagccccctgcagggagggcccaggctaaaatcccacccattctgtttcctctccttccactagaatcccacctgttttgagaagttggcagggcctgtgttagggaagcacctgataaccaacgtgagaatggtggcgctggtcccatgtcctgtctcacttttagatgaagagcttcttcatcctcaaaaaaaatgactcagaagagagccctttaatagactgctcggctaggggtggagggctcgtaaggatatttacagatccttgaaaacagagcttccagcatttaaccctgaagagagctggagcctgggccccaactccttaccccacctgagcctcctctctgcactggaggcctctgagcctgactccgcgcaccccacgctggcccacaagccaaggctccttctgatgccccctgaggctgctggcattggccagggctccatcagcacctccttgggtgggggggattaccgctttccctacttactccagccttggttagggggcccatttcccagctcctgccaggctctccacccatttgtggtcaaggcaagagagaccacacccccttgaatgtccaggcactgagtccctgccggctactcatacggatgggccctccccagccattcctcaccacggctgcgtccggatccacaccacaaccacagcattgttggaaagcaactgcacacctccctgcaacttcccctcgcatcccccagactgagtcatgcaggcggtgcccgtgggcagacctggccagggttggcaccagctggccagagagaatgagaggcactgaccacacccaggaacctcgctaaagcccatgcataagggtcaggagagcagccagatgctgggacccaagggtgcctgtgacacacaccaccctgaaagttgcagctgagcttttctgagaaggatggcaaagtcctggaaaagctttaacgacaaaactcaacatatggaaggcagaggaagcaggtaaggattctggtctccttttatgtgaaatagctttaaaaaaaactttttcaaaatcacgtttaatatgcgttgagccccctggctaccaggtgggaaagtcaaagtcctgaatcatttactcccaccctagacaccttgaacttcagaacttcagagaggagtcctgtggtctttagtcctcctggtcaccgctccctggctgattcactctcctccaggggcggtgtctccacttcctgcaaagcttgggcacatgggcagccatgtccctcgaggttcgccacctccccagggcactgccaggaggcactgtcctcgttactagcagagccagagccaccaccacaaccatagccaccacctccccagcccagggaactctcccagcctctttcctcggcctgtcttcactcccttctaactactcactcttttcctcttcctctctggggcaaccttccatcatccctgccatgtgccttggttcaaggaatcagctttggattctggcccttaagatgaggagtcacaagtacctcctaggaatgagaaagaggaaaagacctagcaggggaatgccaataatattagtgacactcactaatttcttcaacacttagccatgggccaggccctgctctaaatgctctacaaaggttaagcccatttaatcctcacaagaactctatgagggagacactgtcattcccattttgtagatggacaaactgaggcacagagaagtcatgtgacttgccctagttccgtcagctagtgaatgggagagccagaattccaacacaggcacatttctggagacagggcataggttcctccagatcctcaaaggcatgtgggaccaggaaagaagccataggtctgtgctctgaaacatggcaagaacactgttcaaaccatcccatcatctccttcctccacgagtcctctagaatcatgtgtggctcctggaaaccgcagcttgaaaaggattggtgagaagcctaattcctcctagaagtagtgtggtagtgtaattcagacttagattgttctttatgggtgagagagagggcaaattacaatagataaactggctgggtgccgtggctcatgcctgtaatctcagcactttgggagcctgaggtgggtggatcgtttgagcccaggagtctgagatcagcctgggaaacatggtgaaaccctgtctctacaaaatacacaaaaattagcggggcttggtggtgcacgcctatagtctcagctatctgggaggctgaggtgggaggattgcttgagctcaggaagtggaggttgcag

SEQ ID NO: 85 - protein, 574 aa, Homo sapiens
MALARGSRQLGALVWGACLCVLVHGQQAQPGQGSDPARWRQLIQWENNGQVYSLLNSGSEYVPAGPQRSESSSRVLLAGAPQAQQRRSHGSPRRRQAPSLPLPGRVGSDTVRGQARHPFGFGQVPDNWREVAVGDSTGMARARTSVSQQRHGGSASSVSASAFASTYRQQPSYPQQFPYPQAPFVSQYENYDPASRTYDQGFVYYRPAGGGVGAGAAAVASAGVIYPYQPRARYEEYGGGEELPEYPPQGFYPAPERPYVPPPPPPPDGLDRRYSHSLYSEGTPGFEQAYPDPGPEAAQAHGGDPRLGWYPPYANPPPEAYGPPRALEPPYLPVRSSDTPPPGGERNGAQQGRLSVGSVYRPNQNGRGLPDLVPDPNYVQASTYVQRAHLYSLRCAAEEKCLASTAYAPEATDYDVRVLLRFPQRVKNQGTADFLPNRPRHTWEWHSCHQHYHSMDEFSHYDLLDAATGKKVAEGHKASFCLEDSTCDFGNLKRYACTSHTQGLSPGCYDTYNADIDCQWIDITDVQPGNYILKVHVNPKYIVLESDFTNNVVRCNIHYTGRYVSATNCKIVQS

SEQ ID NO: 86 - DNA, 599 bp, Homo sapiens
gacccctggtctatgtcaaaaatcccatggaatccacaagaaagccactacgactactaagtgagttttactaggttgcaggaatacaagattgatatatagaagtacattgtatttctattagaaatttaaattttaaaatgatactttttacagtagcatctgaaatatgaaatacttatggataaacttgacaaaatatatgaatgacctagacactgaaaactacaagacactgctgagagaaattttaaaagacctacataaacagagaaatatattgtgttcacaggtcagagractcaataatgtcatcaattctcccaaactggcttacagattcaatacagtctccaccaaaatcacagctgcctttattatagaaatggacaaattgactaaaatccttatggaaatgcatttctaaagttaaagcatttccttttatagttaaggatcccgggacacagcccagctttgcctgttcccttctgcttcctgccacattgcatgctgagtgttgcagtttctgaatgagtgtaggttgggagaacatgatgacaatcagaggggcagaaagcttgctctgatgagtggca

SEQ ID NO: 87 - DNA, 599 bp, Homo sapiens
gagcatccagacagaatgtggaaaagattcacgtgtcggggccagccagagtgacccggggtgggacgggggtgtgagggctgggctagggagctcagagaggttcaggagctctgacaagagcccacctgtgttcatttcctgaggctgccttagcaagtaccacaaactgggtggcttccgacaacagaaacctgtcttctcgcagttctggagccagaagcccaaaatcaaggtgttggctccttccaaaggctctagggtagaacccttccttacctcccagcttgtgtggctccyggtgttcctcagctggtggctgcctcactccattccctgtccccatggtggtgagaccttctgctttctgtgtgttataagaacacatgtcattggatttggggtccacctggaaagtcttacggatctcgtccggagatccttgacttagtcacatctacaaagactcttttcccccacatggttacattcacaggtcctaggggttaggacatggatatgtctttttggaggccaccatgcggccagcaataccaactaatcatattctaataatgaggacaaaacaccgcctcact

SEQ ID NO: 88 - DNA, 599 bp, Homo sapiens
tcaatgccctcccaggacctggcccaacctaagccaagggtccaggctctcccacccagcccctcctccccaaccccctcttcgccacagagtaccagcagggcttccatgagatgatgatgctcttccagctgatggtggagcacgaccacgagaccttctggcttttccagttcttcctgcagaaaacggtgagggcaaggcctgagctcagggacgcccctccccaaccccccactactccccaaacaactctttgaggatgggcaggcattggccaccccagccattccttccagscttctgggtctcagaggagcctgggagggtgacgctaaacccagtggttcaaagtagtggtggtggggaggcactgaagtggagaagggggatggagcaaagctctgggtccacctctcaacaaaccttgaaagagcatggctggccaggcccagggaaggtgccctctcctggcattttcttccccaagcacggttctcctccctcctcgctgcttcttcctcaatggctccctgtggcctccagaataaaatccaataaccttagccagtcattcagggctgttcccttcacataac

SEQ ID NO: 89 - DNA, 599 bp, Homo sapiens
cagggacgcccctccccaaccccccactactccccaaacaactctttgaggatgggcaggcattggccaccccagccattccttccaggcttctgggtctcagaggagcctgggagggtgacgctaaacccagtggttcaaagtagtggtggtggggaggcactgaagtggagaagggggatggagcaaagctctgggtccacctctcaacaaaccttgaaagagcatggctggccaggcccagggaaggtgccctctcctggcattttcttccccaagcacggttctcctccctcctcrctgcttcttcctcaatggctccctgtggcctccagaataaaatccaataaccttagccagtcattcagggctgttcccttcacataacctgaattccaggcacaccaggggactcaatagtccccaggggctccctcactgtccaagttgaggggttttgaaggtgcagctccctggaacacctccctggaaagcctttcctcatgatctgcgagaggctgcccagaaccctccctcatccctccctccaccccaccatccctgcacatgtgcttgctgcccttcccttgtcccagttg

SEQ ID NO: 90 - DNA, 599 bp, Homo sapiens
caggtttgcaggcggaggccttccctgcagaaagtcagcaggtggagagatgcatccggcagcggttctctgcagttccctcctgagtgctcttcagcaggctccaagcttgctcctccagggcctggggttgtccagtatattttttcttttcctcattcatccactcaatcaacatgtaggaagtcccctctgagttctaggcactagacacacaaaccacataagcaatgcagttcacagtccctgccctttgggagtgtaccaggatttgaaagttgcatccctgcatctgttctyagatgttctgcaaataaagctggccctaccctagttaatccaacatttaaaagtgttgtatcccaacagctccttttcagacaaaaagcacagatcatacttgggccatcatctcagtgaatgttgggcacgtggggctccagagcaggggatcagacaggccctcgatcagtcacctagacagggatgcatagggaatgactttgagccttggatttggggagaccaggtgttgaatcctggctcagctcctcattactgcattccctcgggcaggatccaacctctctgaccccaaa

SEQ ID NO: 91 - DNA, 599 bp, Homo sapiens
atttggggagaccaggtgttgaatcctggctcagctcctcattactgcattccctcgggcaggatccaacctctctgaccccaaatgccccatgcggggcccacctggcataggctctagctctagggaaacatccaatcttctgataagggggattagatgatgttttcttggattaccagctgcctcttctttcccctctgtgtctctctagcagtgtgtctctgttctccctctctctgtcacctctctgtctgtctgtctgcctctttaccaccttctctggtgagcagttgaggygcagcccccctgactagactccctttctccctgtttctcttcttcctcaggtgcacgtgaacccaaagtatattgttttggagtctgacttcaccaacaacgtggtgagatgcaacattcactacacaggtcgctacgtttctgcaacaaactgcaaaattgtccagtaagagtttgcccaccacccttcctggctccgtccctttcctgcctggggagcaggcaaggccacctgagatacctaagatacctgcactttaggtcaccttgatggccgaacgcagctgcagcagggccca

SEQ ID NO: 92 - DNA, 599 bp, Homo sapiens
tgtgggctctgtaatctccatgactttatccacccttcccctcagggtcttacgggacatcgctatctttcaatctctccaagacttatttagccagagtctttaccacactcctacctgagtttatccagcttctcttgcatgcctccggtgacaggctgctcactatccacagggtcacttcttatcctaccagggctggtcaaaacctgtgtctctttaggtcagcagtgcctggtgccagaggtcctatcagtagacccaaagctgggtgaacaaaacaaaggctgggcaactcaytgtcacctgggcatgtgaccagcctttgctctccactctggctaattcttttgtctgggggccacatcgcctgcctgccctccctccttctgtgcgattttcttcttcttctctctttcctccccccacttccttgtatctaaggattgcatgagtcacttcactttgagctgccagatgtcggatgcagttgggcttggagtggaagccaattttttccctttactcaaaaagagcttggggctgatgaaagcccccaacactgtcatcctggatttttcaggacggtaggatggctg

SEQ ID NO: 93 - DNA, 599 bp, Homo sapiens
accccctagcttaggatccatgcaatcctgaaagacacaatcccaaatgtttaaatcctgaaagctgaattctgggaaagggattagtgcagtttgggttatagcagcatagttgcatcatattaattgcatcctgttaagcggaactattatcttcttattatatttgttggtttaagaagatgcatgtgggagccaggttgacaaggggtagacttggggagttaattttaggtgtcaatttgaccagattaaggaaaacctagaaacctggtaaagcattgttttgggtgtgtctgyggggtgtttccagaggagattagtgtgtgagttcagtggactaggtggaaaagatctgccctcagtgttagcaggcaccatcctcttgcccaggggcccaaagagaacacatacagagggtggactccgctctctgagagctgggacaggcttttcttctgctgccttggacatcagaactccaggcccaccagcctttgactccaggcccaccaagattgacaccagcggcctcccaagtcctgaggctttctgccttggactgagagttaccccatctgcttccctggttctgaggt

SEQ ID NO: 94 - DNA, 599 bp, Homo sapiens
accagattaaggaaaacctagaaacctggtaaagcattgttttgggtgtgtctgtggggtgtttccagaggagattagtgtgtgagttcagtggactaggtggaaaagatctgccctcagtgttagcaggcaccatcctcttgcccaggggcccaaagagaacacatacagagggtggactccgctctctgagagctgggacaggcttttcttctgctgccttggacatcagaactccaggcccaccagcctttgactccaggcccaccaagattgacaccagcggcctcccaagtcctsaggctttctgccttggactgagagttaccccatctgcttccctggttctgaggtctttggacataggctgagccatgctactgacatcccagggtctttagcttgtagacagactgccaagggacttatcagccaccatagttttgtgagccaattcccctagtaaatcacctctcatatgtctatatacatatcctattggttctgtctctctggagaaccctgattaatataaatttggtcttggggaagctgaatattatttcttcttactgtattccttacaacacaacagaaga

SEQ ID NO: 95 - DNA, 599 bp, Homo sapiens
gccatcattgcctgtgttgttgtgtaaaggtgtcaatgaagtgttctgtcatgtttttatatgtttcccaaataatccattttaaaaatgtaagtaaatattttttaaagcattaaatttttttcagagttatattttcagggttttcatctttcaagaatgtgaatgtggggattttagactttagggattttgatattttaggattttgacatccaggattatggcatttgggatgtgtctttcaggactatggcccggaccatttaggaaggggtcttttcccagctttctcagttsctgggtaaactggcttcctgacctaaacgggtcctaggtccatctcatctctttaaattcaaactgctctcaaatacaaactcagtggtcctctctggcctcttcccacagatgtttctcaaacccattcatctaaggctgagcacacagcctacttagcttccccgagcaaagcctctagtctagacaaggccacagttaatacaggagtcacccctatgagacatacttctttataatctttctcagtgtgttaggactttttcagatgcaaataacagaaacctgatccaaattag

SEQ ID NO: 96 - DNA, 599 bp, Homo sapiens
ggatggcagaactgggagatactcttagcagtcagtccacacatccccccacctacagccccatggctaataagccatgtgtttcttccattttatcccatttaatcccgatccccagcagagcctaatgaggcagatgttggtgcccattatgcaatgataatgagggggcaaggactccctgggataccactgacaatgacttcccttagagggtactaaggcagctatgggaagatgcttttgaggatcaggtggggagtgtgcagggaggcccagtctcaggaagacggggcagtycctcagtgcctgtgttggtccctaggactcctggggagggctgcaccactcaccaaagcaacactgagagccacagaatgctaatcagactcagacatcaggatctcagacaataactgcgatactgaaaatacacaaaataaccaggcagcctttgtaattaacttccgctcccttccagcagtcatttggagttaatggtttcttaattcagtgctcatttggtgactttaatttccaattaattttgccgattttcctccgaggtgctcatggtgtgatagaaaattcctatatgg

SEQ ID NO: 97 - DNA, 599 bp, Homo sapiens
cagtccacacatccccccacctacagccccatggctaataagccatgtgtttcttccattttatcccatttaatcccgatccccagcagagcctaatgaggcagatgttggtgcccattatgcaatgataatgagggggcaaggactccctgggataccactgacaatgacttcccttagagggtactaaggcagctatgggaagatgcttttgaggatcaggtggggagtgtgcagggaggcccagtctcaggaagacggggcagtccctcagtgcctgtgttggtccctaggactccyggggagggctgcaccactcaccaaagcaacactgagagccacagaatgctaatcagactcagacatcaggatctcagacaataactgcgatactgaaaatacacaaaataaccaggcagcctttgtaattaacttccgctcccttccagcagtcatttggagttaatggtttcttaattcagtgctcatttggtgactttaatttccaattaattttgccgattttcctccgaggtgctcatggtgtgatagaaaattcctatatggtgaaatccccaccacagggacctggagtgggt

SEQ ID NO: 98 - DNA, 599 bp, Homo sapiens
caagctgccaccagaagaccagactcctgcctgagtgtcaggaatcagcatctcctgggttcaggaggtccttggggagttcaggggtccatgagagactcctcccctaagagggggctggtgagagctgataaagtggtgcaggggttgaaggtacccctgaatttttctagctctggagagttttgagtcatagtggatggggcaggagaggaagcccagctctcctggaagcatcagggaaggggtcaggaccaagcacagtgccctcccttccatcaagcagaggcatttaagagrtcttggtccagatcccctttgctgggccaatgccctcattgcccagctgctgcagatgtcagctgctatcatctcacagcctcccccttctctagagttgcctttggccagtgagattcacctcttctggaaagttacattacccatggctcgggacatcctgtacacaaggactgagtggtatagacagaaggctgcatcccttctccttaatctccctcccttttgcctcaaggtgagtctgccactgtgatgagctagtgttccagagtggtgcagccagacccagatgaggccac

SEQ ID NO: 99 - DNA, 599 bp, Homo sapiens
tggggagttcaggggtccatgagagactcctcccctaagagggggctggtgagagctgataaagtggtgcaggggttgaaggtacccctgaatttttctagctctggagagttttgagtcatagtggatggggcaggagaggaagcccagctctcctggaagcatcagggaaggggtcaggaccaagcacagtgccctcccttccatcaagcagaggcatttaagaggtcttggtccagatcccctttgctgggccaatgccctcattgcccagctgctgcagatgtcagctgctatcakctcacagcctcccccttctctagagttgcctttggccagtgagattcacctcttctggaaagttacattacccatggctcgggacatcctgtacacaaggactgagtggtatagacagaaggctgcatcccttctccttaatctccctcccttttgcctcaaggtgagtctgccactgtgatgagctagtgttccagagtggtgcagccagacccagatgaggccacatccttgcttaagtccttccctccctttctgctgccagaactccctcagtaaatcccatgcacctgaatctc

SEQ ID NO: 100 - DNA, 599 bp, Homo sapiens
ttgaattacatcatccatgcctttagggaagttgctggaggtcagtggttccatcttgggcagcataatggttaagggcacagagtctggaactagagtccccgggttcaagttcctgttctgcacttacagtctatgctgcccctgggaaaattccttaatcttttgctgtctgttttcttatttcaaaatagggacactaacaatgcttagcttgtagggttgctatgaggatgacatggatttctttgagtaaaggacttagaacagcctctggcacactgcaagaggaggacaagmgtttaatatataaactattggtgtcatttgggttccccagaagctaagcccaagacagagattcaggtgcatgggatttactgagggagttctggcagcagaaagggagggaaggacttaagcaaggatgtggcctcatctgggtctggctgcaccactctggaacactagctcatcacagtggcagactcaccttgaggcaaaagggagggagattaaggagaagggatgcagccttctgtctataccactcagtccttgtgtacaggatgtcccgagccatgggtaatgtaactttc

SEQ ID NO: 101 - DNA, 599 bp, Homo sapiens
aaaatcaaaaaatacaaaattagctggccatggtggcaggcacctataatcccaactactcaagaggctgaggcaggagaattgcttgaaccctgggggcggaggttgcagtgagctgagattgcaccacttcattccagcctgggcgacagagtgagactctgtctcaaaaaaagagtgcttggttatcatggggccagagctccaggattccagacccccctgttcctcagtccttctgaaaaaggacctggagaaaagctctccctcacactttagtggctcaggctcttgaattayatcatccatgcctttagggaagttgctggaggtcagtggttccatcttgggcagcataatggttaagggcacagagtctggaactagagtccccgggttcaagttcctgttctgcacttacagtctatgctgcccctgggaaaattccttaatcttttgctgtctgttttcttatttcaaaatagggacactaacaatgcttagcttgtagggttgctatgaggatgacatggatttctttgagtaaaggacttagaacagcctctggcacactgcaagaggaggacaagagtttaata

SEQ ID NO: 102 - DNA, 599 bp, Homo sapiens
attcagtctgcaatctcattttctctgtgccacatcatttaacctattcacaggattcagggattagaatatggacatttggccggggaatggggggtattattctacctctcacaccgagtaaaagggactgtagtgctcccacatctacacacggggctcaggctcccatttgggagctggagcttggtgttctcattttcgtttgccgccttgggcaggtccttttcctcctggaccccagcatccccttctgtatttaaacaagtgggctagatgactgctgtattcattagtgtraagttgagctctgaaccatagagacccagcaagaactcagttgctttaaaaaagacagtctgatacatcaatccaagatgaacattctgggtgtacaaaggctgtgctccatgtggtcattcagggatggaggtgattttcatctcatccctctgttgtaactcagggacttgctactcaaagtgtggtccctggaccgacagcatcagcatcaccttggagcacactagaaatggaaattcttagctctcacccacagagatattgaatcagagattcttgggctggggcccagccat

SEQ ID NO: 103 - DNA, 599 bp, Homo sapiens
gggcctgggaagtggctggccaccctccaggcctctcttcaaggatttgctatgagagccaggcaaagtatgtgaccctctctgggcctctgtttcctctagtttcttttcagtttcatgtttcagagttggagagaatggtactgcaggaagccttgggcagtctcagtgcccagggtaggaaggaagagggcacaggtacccgaggaagaaactgggctcaggacaggcgcttggaaaccacccacccatggagggaggtgggtggtgctcacatgtgcttggtgttgtgaggacacrtccacgcccacacacactcacaggctgccagcgcccagcctcagacccctgggtgtgtggccaggaggaatgaagtcagtctcggagaatccgccttgggctcccacaccaggcctgggggtctgatctcaagcacaaggcttggccttccctgggctgggattttccacggtctggcctagcagggtggggagggaggaaccaatgatgggagagactactgctccctctctggcctctttgtgagccctaaactagcctgcaatcccctccccagcaaaaaacacactcccacccca

SEQ ID NO: 104 - DNA, 599 bp, Homo sapiens
cattcaacagatatttgagtgctagattctgggcactggttgtgacagggtgagatgggacagtgagtgtgaaagactttggaagcagagtctctccccagtgtggggttagggtgtagcctggtcatggtcattgcagattccagaacgtttgtgtaacaaacactcatatggtgcatactaagtgccggacactgttcttggagttttgcagttagaatcacttaatccctgtgacaaccctgggattgttatccccattttgcagatgaggaaactgtggccctgagaagttaagcracttgccctggaggcagaatcacagatcagcatccctccttccttccccgaagttgtctcagattgttccttcagaaagaaaaagagtcttcaggcttagaaagctgtgtcggatcagacaagctgtggaaacggagggttctgggccctcctcctgcctccctgtttctcctttgagagtcatcagtgtctgaacaaacaagtggagtcctaagcagcctcattccaggcagacttcctgtctcctctcttatcagccacttgcaaggcaccttctctctcccctgctctcaaactgg

SEQ ID NO: 105 - DNA, 599 bp, Homo sapiens
gatcagacaagctgtggaaacggagggttctgggccctcctcctgcctccctgtttctcctttgagagtcatcagtgtctgaacaaacaagtggagtcctaagcagcctcattccaggcagacttcctgtctcctctcttatcagccacttgcaaggcaccttctctctcccctgctctcaaactggaccatccctgcaagaagagacagctcctgttttcaataataagaataagaatatctgatatttattgaacatacactatgctaagcattttatttttctcatttaattcccayaacagccttgtgagataaatagtattatcagctctattttatagatgaggaaactgaggctcggagaaattaaccaactcacccaaggtctcagggttaatgagtggccaagcttagacctaggctgactgtccaatccagtgggccctggcctcactctgttccgtctcttagggacgggtgttttatattctgtgtcacctgtctctgtgttcactgttcccctgaggtgtaaacacctgaaaaaaaccaaatttgggtttttgcctcaagtttgcgggccccacagagttgctggg

SEQ ID NO: 106 - DNA, 599 bp, Homo sapiens
gctgatccagtgggagaacaacgggcaggtgtacagcttgctcaactcgggctcagagtacgtgccggccggacctcagcgctccgagagtagctcccgggtgctgctggccggcgcgccccaggcccagcagcggcgcagccacgggagcccccggcgtcggcaggcgccgtccctgcccctgccggggcgcgtgggctcggacaccgtgcgcggccaggcgcggcacccattcggctttggccaggtgcccgacaactggcgcgaggtggccgtcggggacagcacgggcatggccckggcccgcacctccgtctcccagcaacggcacgggggctccgcctcctcggtctcggcttcggccttcgccagcacctaccgccagcagccctcctacccgcagcagttcccctacccgcaggcgcccttcgtcagccagtacgagaactacgaccccgcgtcgcggacctacgaccagggtttcgtgtactaccggcccgcgggcggcggcgtgggcgcgggggcggcggccgtggcctcggcgggggtcatctacccctaccagccccgggcgcgctacgaggagtacggcggcggcg

SEQ ID NO: 107 - DNA, 599 bp, Homo sapiens
ggtagaagccctgaggcgggtactcgggcagctcttcgccgccgccgtactcctcgtagcgcgcccggggctggtaggggtagatgacccccgccgaggccacggccgccgcccccgcgcccacgccgccgcccgcgggccggtagtacacgaaaccctggtcgtaggtccgcgacgcggggtcgtagttctcgtactggctgacgaagggcgcctgcgggtaggggaactgctgcgggtaggagggctgctggcggtaggtgctggcgaaggccgaagccgagaccgaggaggcggagyccccgtgccgttgctgggagacggaggtgcgggcccgggccatgcccgtgctgtccccgacggccacctcgcgccagttgtcgggcacctggccaaagccgaatgggtgccgcgcctggccgcgcacggtgtccgagcccacgcgccccggcaggggcagggacggcgcctgccgacgccgggggctcccgtggctgcgccgctgctgggcctggggcgcgccggccagcagcacccgggagctactctcggagcgctgaggtccggccggcacgtactctgagcccgagttgagcaag

SEQ ID NO: 108 - DNA, 601 bp, Homo sapiens; identical to SEQ ID NO: 56

SEQ ID NO: 109 - DNA, 603 bp, Homo sapiens
agagtacgtgccggccggacctcagcgctccgagagtagctcccgggtgctgctggccggcgcgccccaggcccagcagcggcgcagccacgggagcccccggcgtcggcaggcgccgtccctgcccctgccggggcgcgtgggctcggacaccgtgcgcggccaggcgcggcacccattcggctttggccaggtgcccgacaactggcgcgaggtggccgtcggggacagcacgggcatggcccgggcccgcacctccgtctcccagcaacggcacgggggctccgcctcctcggtctcgscttcggccttcgccagcacctaccgccagcagccctcctacccgcagcagttcccctacccgcaggcgcccttcgtcagccagtacgagaactacgaccccgcgtcgcggacctacgaccagggtttcgtgtactaccggcccgcgggcggcggcgtgggcgcgggggcggcggccgtggcctcggcgggggtcatctacccctaccagccccgggcgcgctacgaggagtacggcggcggcgaagagctgcccgagtacccgcctcagggcttctacccggcccccgagaggccctacgt

SEQ ID NO: 110 - DNA, 603 bp, Homo sapiens
gcagaggggtcaccatggctctggcccgaggcagccggcagctgggggccctggtgtggggcgcctgcctgtgcgtgctggtgcacgggcagcaggcgcagcccgggcagggctcggaccccgcccgctggcggcagctgatccagtgggagaacaacgggcaggtgtacagcttgctcaactcgggctcagagtacgtgccggccggacctcagcgctccgagagtagctcccgggtgctgctggccggcgcgccccaggcccagcagcggcgcagccacgggagcccccggcgtcggcasgcgccgtccctgcccctgccggggcgcgtgggctcggacaccgtgcgcggccaggcgcggcacccattcggctttggccaggtgcccgacaactggcgcgaggtggccgtcggggacagcacgggcatggcccgggcccgcacctccgtctcccagcaacggcacgggggctccgcctcctcggtctcggcttcggccttcgccagcacctaccgccagcagccctcctacccgcagcagttcccctacccgcaggcgcccttcgtcagccagtacgagaactacgaccccgcgtcgcgga

SEQ ID NO: 111 - DNA, 603 bp, Homo sapiens
gaggggtcaccatggctctggcccgaggcagccggcagctgggggccctggtgtggggcgcctgcctgtgcgtgctggtgcacgggcagcaggcgcagcccgggcagggctcggaccccgcccgctggcggcagctgatccagtgggagaacaacgggcaggtgtacagcttgctcaactcgggctcagagtacgtgccggccggacctcagcgctccgagagtagctcccgggtgctgctggccggcgcgccccaggcccagcagcggcgcagccacgggagcccccggcgtcggcaggcrccgtccctgcccctgccggggcgcgtgggctcggacaccgtgcgcggccaggcgcggcacccattcggctttggccaggtgcccgacaactggcgcgaggtggccgtcggggacagcacgggcatggcccgggcccgcacctccgtctcccagcaacggcacgggggctccgcctcctcggtctcggcttcggccttcgccagcacctaccgccagcagccctcctacccgcagcagttcccctacccgcaggcgcccttcgtcagccagtacgagaactacgaccccgcgtcgcggacct

SEQ ID NO: 112 - DNA, 603 bp, Homo sapiens
ctcagagtacgtgccggccggacctcagcgctccgagagtagctcccgggtgctgctggccggcgcgccccaggcccagcagcggcgcagccacgggagcccccggcgtcggcaggcgccgtccctgcccctgccggggcgcgtgggctcggacaccgtgcgcggccaggcgcggcacccattcggctttggccaggtgcccgacaactggcgcgaggtggccgtcggggacagcacgggcatggcccgggcccgcacctccgtctcccagcaacggcacgggggctccgcctcctcggtckcggcttcggccttcgccagcacctaccgccagcagccctcctacccgcagcagttcccctacccgcaggcgcccttcgtcagccagtacgagaactacgaccccgcgtcgcggacctacgaccagggtttcgtgtactaccggcccgcgggcggcggcgtgggcgcgggggcggcggccgtggcctcggcgggggtcatctacccctaccagccccgggcgcgctacgaggagtacggcggcggcgaagagctgcccgagtacccgcctcagggcttctacccggcccccgagaggcccta

SEQ ID NO: 113 - DNA, 603 bp, Homo sapiens
aggcctaccctgaccccggtcccgaggcggcgcaggcccatggcggagacccacgcctgggctggtacccgccctacgccaacccgccgcccgaggcgtacgggccgccgcgcgcgctggagccgccctacctgccggtgcgcagctccgacacgcccccgccgggtggggagcggaacggcgcgcagcagggccgcctcagcgtgggcagcgtgtaccggcccaaccagaacggccgcggtgagtacggccccggcgcccctccggccgcgcgtacccctggccactggaaactgctccgkgccccccgggcccctccttagaacttcctggaggcctccccggagctgctaagcacggagcagcgccccctcccgacttcccccgacccagccaacagcacccccctggcatctcacctcccagcttaacccgcacccaggcatcatcagtgccaggggttggccaggcaggtgtgggctctgatcctggctttggtggagggactagccgagctttgggaggaggctcgccttctgcacttggcttgggtcatctgaggacaaaaggaaggagcccctcatccaccttctccctcatttc

SEQ ID NO: 114 - DNA, 603 bp, Homo sapiens
cacactccaggccagaagcccagggctctcttggtggtttacaaagaatgctgactgcagggatgagaggaacggagggtgtctgggggctgctgtccagaggcctggggagtaaggactcgagggagtgcctctgtcaggaaattacatggttcactcattcctgcctccaggtctttgttcatgctgttttccctgccgtggacaccctttcccgctctctgattctctaaatcctgccccatctcccagatcttgttcatgtccaagcttttccaggaagtcttagcagctcccacacygcagagctcgagatgtaagctcttgctgcctgacttccctgaccccacacctgggcccaggacctaacacccaaggagaatggcaaagggttttggggtatgagaggatgtttggggtgtgaggaggtctctgggcattagcagtggccaacctgatgctctcaatgtcatgctcttcctccctccccccttctctcccactgcccactccaggtctccctgacttggtcccagaccccaactatgtgcaagcatccacttatgtgcagagagcccacctgtactccctgcgctgtgctgc

SEQ ID NO: 115 - DNA, 603 bp, Homo sapiens
agaaggtggccgagggccacaaggccagtttctgcctggaggacagcacctgtgacttcggcaacctcaagcgctatgcatgcacctctcatacccaggttgggctggagagatggggtttggggcatgggaggataaggagttggggaggcaaagagcgaggcccgctgaggcccggcaagtgccaaggcttctggccactcagctctgctcacagtgaaggtcttctcaccagtcctcaggctgccacactgccctgcagggactgttccctccctgccccagcccctttcccatgttaytccaggtgatctgctcgtggagagaaggaaacatcgcaacagtctggagagcaacacgtcctattggcctgttcacccacccatatccctctttccatcagccaccctaaatatccacaaactgtccatctgtcctgtctctttttatccattctgccatccatctcgtccctcccacctggctattagatatctgtctttcctctggtccatctagccagtggctttaggaaggtgtggacccaccctgagcagttgccctgggccagggtcaggggagaaacccaggggaaggtgggcc

SEQ ID NO: 116 - DNA, 603 bp, Homo sapiens
aaagtatattgttttggagtctgacttcaccaacaacgtggtgagatgcaacattcactacacaggtcgctacgtttctgcaacaaactgcaaaattgtccagtaagagtttgcccaccacccttcctggctccgtccctttcctgcctggggagcaggcaaggccacctgagatacctaagatacctgcactttaggtcaccttgatggccgaacgcagctgcagcagggcccatcaaaaagagccttgtccgtgccaaccccaagctgagtgtttacggtggggctcaggccgtagggtrttgatctggggctgcatcctgtggctccctgagatgtcccaagtgaaaaccagcagaaggcagagtcatttgatgcaaggccatgagctctgcagtcccagttccacagcttcctcgctatgtgacattgggttacttccctgagctactttcctcaactataaaataggtatttccacataggctggcaatgaggatctacaggcctgatgtctgcatgttcacagtggcacacagtgaacagcagctgtgatcattagtgtccttttcaagtggactggattaaacagtcacagattag

SEQ ID NO: 117 - DNA, 603 bp, Homo sapiens
gacagtagcagctctgctaatggcctagaaaacaatctgggactagggcatggggaggggaatgtgctgagactgtgccctgggtgggtgggagtccatcaaacccctccttttggagctgggctgagggggtcggaactttctcttgagggaaatgtaggcccatgcctgccctgccacgagggatctgggctgtgggagggacgccctggctgaggaggccacgggggcctactttgcagcccctcattgacccactgtctttccttcctcagatcctgatctccgggagggacagatgrccaatctctccccttccaaagcaggccctgctccccgggcagcctcccgccgaggggcccagcccccaacccacaggcacggaggggcatccctccctgccggcctcagggagcgaacgtggatgaaaaccacagggattccggacgccagaccccattttatacttcacttttctctacagtgttgttttgttgttgttggtttttattttttatactttggccataccacagagctagattgcccaggtctgggctgaataaaacaaggtttttctactctgtggctctgcatgcggcc

SEQ ID NO: 118 - DNA, 603 bp, Homo sapiens
attccggacgccagaccccattttatacttcacttttctctacagtgttgttttgttgttgttggtttttattttttatactttggccataccacagagctagattgcccaggtctgggctgaataaaacaaggtttttctactctgtggctctgcatgcggcctgctggctggctggccagccacagctagtttgggcctgggggatgtccttaggcatcatccttcccctcgccaaatgtggaaagggcagccaccacccggtcaggaccacagtgaccatgagggcgctgagcccatcrtggaagcctgtggggttgagcccttggccaagcctgtcgcatgggaatgggaccacatccgactaggggagggccctgcagcggggtgggcggcaccagcctgctctccctgctcagcccctgctcctatctgggctcgtgtgcccactgctgccccttctcagccttcagcctcacactcccaccggtccccctcctggccctctcactctcacaccgttcactctccaccagccacccactcccttacaattgcaggctcccgaactcaagtctcctgaaggccaggtgctgtgctagg

SEQ ID NO: 119 - DNA, 603 bp, Homo sapiens
tctcaaactatgggcctcaagtgatcctcctgcctcgacctccaaaaagctctgtaattacaggcatgagccaaagtgcctggcccccgcaatagggctgtttttcagtggcccttcagagattgctatgtgctcctgcacctaggggctgaccccagctgagtcacctgcaggtatcagggacactgccatgcttggtaccgctttagaaagatgctgttggcagctggcggccacatggtggaggccagaaaggaggggacagccaggatttcacggggtcagaagacagaggaagaggsctgagcctccgccacagttcgaggagacagaggagaagatcttcagggacaaggagtggggctgcagaaccagtggccaagggctgggggtggggctgagggggcccatgctgggttctggtgtcactgtgccccaaccccccctcatctcccccgccgtccctgcagcacagcctatgcccctgaggccaccgactacgatgtgcgggtgctactgcgcttcccccagcgcgtgaagaaccagggcacagcagacttcctccccaaccggccacggcacacctgggagtggcacagctgc

SEQ ID NO: 120 - DNA, 603 bp, Homo sapiens
gaatctcccaaagcactctcaggagccatctggcctaatcttccattggtaagccgtggcacctggatcagatagagtaagggactaagccacacagcaacaggaccaggccaggtctccggaacccccttctgttgttcaatgcttactggcttctctgcctcacagtgacccctgtcccatatcaaagacagcccccagtttcattttatccatgtgtacactcaagttattcccaggctatgcagagccaagagatgtaggacagaaaccatacgtgatgtctgggaagttgatctctyccaggatctcacaagtgcttttcagctcagggatacaccccactcttcagactgggaaagtaagcccctgaggtgtgccacgagggaaaaggtgcagctccagtgcctctcttcgcaggcccaagagctgcggtgcacctgggacctggaattagagagtggtccctgttcagcatctccccgaggaggcccaccaacaaagagggtgtgtcttttttttttttttcttcctattgagggtgtgggataatggtggaaggaacatgcaaagagggtgtgtcttaattagcactggctttag

SEQ ID NO: 121 - DNA, 601 bp, Homo sapiens
  misc_feature (301)..(301): n is either G or nothing
ctgagcccatcgtggaagcctgtggggttgagcccttggccaagcctgtcgcatgggaatgggaccacatccgactaggggagggccctgcagcggggtgggcggcaccagcctgctctccctgctcagcccctgctcctatctgggctcgtgtgcccactgctgccccttctcagccttcagcctcacactcccaccggtccccctcctggccctctcactctcacaccgttcactctccaccagccacccactcccttacaattgcaggctcccgaactcaagtctcctgaaggccagntgctgtgctagggcacacgcaggagcatcgtagcttctgccctcagggagcgcataaccacagggaacggggagcacgcgttgcccagtgaggcagcacctaggggttctggggagccttcacatgggagagagcctccttccaggcagggaggggagaagaggattcttggcagagagacagcattgagcagagatcatgaggtggggctgcaggccgacagggtgtgagtgggagatggagggaggccccgcagccctgcagcccccgtcccctgccctggctgtggcatcccagagg

SEQ ID NO: 122 - DNA, 599 bp, Homo sapiens
cagtggcgtaatctcagctcactgcaagctccgcctcctgggttcacgccattctccttcctcagcctcccaagtagctgggactacaggcgcccgccaccgcgcccggctagtttttgcatttttagtagagacggggtttcaccttgttagccaggatggtctcggtctcctgacctcgtgatccgcccacctcagcctcccaaagtgctgggattacaggcgtgagccaccgcgcccggcggttcaaggcatttaaggagcaccatttaaccattgcctatcagtgggcatgtcagyggcaccctaactgagtgttgctgtttccaaacaatgtggcatgaatgcatctaaagggcaggttatcaatggaaaagcagaccaccacctgggattcactggtataaaataaatgtctcatgcaaatcaaaatgatatgcatttccaaacgtatgtaaatgcataaaaaaggtcttgaaaggccacagcatggactgatgacaggggtcctctcttggggaggactagggttgtaataagtatggagaagcattggaggaaagtgtggtttgtctgtgtggtttcaattttttttttca

SEQ ID NO: 123 - DNA, 599 bp, Homo sapiens
gctcactgcaagctccgcctcctgggttcacgccattctccttcctcagcctcccaagtagctgggactacaggcgcccgccaccgcgcccggctagtttttgcatttttagtagagacggggtttcaccttgttagccaggatggtctcggtctcctgacctcgtgatccgcccacctcagcctcccaaagtgctgggattacaggcgtgagccaccgcgcccggcggttcaaggcatttaaggagcaccatttaaccattgcctatcagtgggcatgtcagtggcaccctaactgagygttgctgtttccaaacaatgtggcatgaatgcatctaaagggcaggttatcaatggaaaagcagaccaccacctgggattcactggtataaaataaatgtctcatgcaaatcaaaatgatatgcatttccaaacgtatgtaaatgcataaaaaaggtcttgaaaggccacagcatggactgatgacaggggtcctctcttggggaggactagggttgtaataagtatggagaagcattggaggaaagtgtggtttgtctgtgtggtttcaattttttttttcagcaagtatgttttcag

SEQ ID NO: 124 - DNA, 599 bp, Homo sapiens
ccgcgcccggctagtttttgcatttttagtagagacggggtttcaccttgttagccaggatggtctcggtctcctgacctcgtgatccgcccacctcagcctcccaaagtgctgggattacaggcgtgagccaccgcgcccggcggttcaaggcatttaaggagcaccatttaaccattgcctatcagtgggcatgtcagtggcaccctaactgagtgttgctgtttccaaacaatgtggcatgaatgcatctaaagggcaggttatcaatggaaaagcagaccaccacctgggattcaytggtataaaataaatgtctcatgcaaatcaaaatgatatgcatttccaaacgtatgtaaatgcataaaaaaggtcttgaaaggccacagcatggactgatgacaggggtcctctcttggggaggactagggttgtaataagtatggagaagcattggaggaaagtgtggtttgtctgtgtggtttcaattttttttttcagcaagtatgttttcaggtacgtcttaggtacattttaaaaacttccatacatctccctgttcttcatgttgctggaggtgcagcccaggatggctgagt

SEQ ID NO: 125 - DNA, 599 bp, Homo sapiens
agcaccatttaaccattgcctatcagtgggcatgtcagtggcaccctaactgagtgttgctgtttccaaacaatgtggcatgaatgcatctaaagggcaggttatcaatggaaaagcagaccaccacctgggattcactggtataaaataaatgtctcatgcaaatcaaaatgatatgcatttccaaacgtatgtaaatgcataaaaaaggtcttgaaaggccacagcatggactgatgacaggggtcctctcttggggaggactagggttgtaataagtatggagaagcattggaggaragtgtggtttgtctgtgtggtttcaattttttttttcagcaagtatgttttcaggtacgtcttaggtacattttaaaaacttccatacatctccctgttcttcatgttgctggaggtgcagcccaggatggctgagtggggtggatggctctgttggttaggacatttcataacaggctctttggttatgagatagtctaaatgaaagaggcttgaaaccaaagagggctagcttactctgaaaaggacaccagttttgtgctggcaaccaggcagagagagtctccaagaccctggaa

SEQ ID NO: 126 - DNA, 599 bp, Homo sapiens
taatatccaggatggcagaaattctacagtgggttgtttcctgctcagaattgccctatagtccccttagtaatccctaaactcttagaaaaagtctgctgcatggaaaaccttatttgagtggagctttaggaaacaaaggagaatgtgaaatgattaagggcaggtgacaaaactcaaagggagcagaaattcgaggtgatgtaaccaaggcattgtgaaacccctgggggtccctcggaaatcctgggatgggcagtagcttttccgtggtacatgggatggaggcttcagcatgtwtaatcggccagttgaagagagggactccaaaagactgaagatcctgaggctatgtgagttaatgtaagtaaaaacatgccaattgatttaaaaaaaatcagaaacctataaagaagtaaaaagtgctagcatagcgtttccatccatttggtgtaatacattcactcattaaatactaaactcacgcactgccccctccaactttcccctctgcaattgcatcttatggtctagcttttaaagtaatgcagtacatggagtcagaagcccggagatccataaaccatccagacatctaa

SEQ ID NO: 127 - DNA, 599 bp, Homo sapiens
gccttgccgtgaatggccaaacatatgctgttattatcaggaaattctgtattcctcccgtaagcatcctctcccccatcaaaagctttatttgcctaggaatcaccatgtgctacaagtctttttggttagttttgcttagggagttggacattccagtcagcttgaatactatagtagttaggctatagacaaacaaaccaaaatacatggtgattataaaatgaaaatttcagacacttatttctcttaaacataaaagtttagatgttaacaatagttggcaaggtacatccctcmatgtgtggcttccacctctgggtccaaggcgtaggacccatttgttactctttcctaaccagaaacgaaaagaaaaggggctgggaaaagggacccaattctttttagctaagaccccaaagtggaacacatcctacaacttctgctcacatctcgttggacaaaaacatagtcacaagaacatatctacctatgagaaaggctgggagatgtagtctccagctggactaatgtgccctgctaacagctggtggtgcatttctgttacagaaagtaagaaagagggaagggatacaggg

SEQ ID NO: 128 - DNA, 599 bp, Homo sapiens
acgtggcgtatttatttagactgcatcacattacactctcgcagttcacagacatttccaggaggttgttgctccaccacttcattcaaaactaggttcacggttttcatgttttcctctctacatctctcctactaatttcccttttaatgttcgaacatgattctgatgaagtctttcagttaaaaattgtgagattaaacttgtactctctaactccgaagtgaaagagattttaggttcaatattaggaataaaattctactatcaggactgttgtttttgtttttcatcagtagmatgggctgccttggaagttatgaagttacccatggctaccgctatacaaaagtggcttttgactagcagctttgcctcgccactgcttcctttctcccatccagtccttgaggtctgcaagctgtttcagtatacatcgcagtgaagagtgtaacaaacagattagctacctgacttgcaattttttggctttggaatttttggttcaggattttatactgtcctagctaaatgaattgacaaaacagcaaggtataacttacaatgaagaagtttatcaaccttcccccgcaggatta

SEQ ID NO: 129 - DNA, 599 bp, Homo sapiens
tttttttgcttgggcaaatgtgtgtgtgtgtgtgtgtgtgtttgtatgtgtatgtgtgtgtcccctttgagaatctgcaaccatagatctttacttgacataggcttgctatttgaatttatactccttgatggtccagaaacccaaggtaagaaattaatgtaaaatttatccctggctaagtgatacccatggaatgctcagcaaatgtgcatgaaaaaataaaaaaaattatatgactttaaacacccaataaaaggagaaatgatttttgataaagccaaatgatgttacataatkcagccattaaaatgatgcttagaaccgggagcttgggggagagagataaagagaagttgtttaatacttgcagagattccgttctgcaagatggaaaagttctggagatctgttccacaggaatgtgaatatacttaacactactgaagtggatactcaataatggctaagatggtgaatttagtgttatgtgtttttttaccgcacaggaaaaaagagaaacaaagagttcttaatggtgaggagaaatgcttatggtagaatgctagagagaagggggacacaaaattgtatagacc

SEQ ID NO: 130 - DNA, 599 bp, Homo sapiens
ccaaagtggttgtagatggggtaaatctcttccttgtgctggaacccgtgcattgccctccacaccccacccctcctttccccttacagctctagaggtcacttgggccattcctctccacaccgccaagcaaatttccactaattaaaactggaatttaaccactctagcagccttctgtcctgtccatacagtctccagggctcaacaatggggtctacccactaccagatgtggtcgtctccagcgtggaggagagaaaccattcttaatgggcagcttgttaacatccccaggacrtgactggttcggatagacccacttccagcaaagtgcagctgcctgtgaggctctgctccagccccaccctcctgtcctgcctctgcgatgacatatggggcaggtactatccctctctggaccttgcgaactgaccctgggagcagccacaagaatgccgccctgaacccttgttcctgagctcaggacatttcctatgcccaatatgacagggaggcagagagcccttgagagagaaggaaacaagacggtggtggtgcatattctggcaggccctggggaaggggcttcacacatat

SEQ ID NO: 131 - DNA, 599 bp, Homo sapiens
atggggaaacgagggaatctgatactgatgcatttagctaaaccttgcttttagggcacaccactgggccttcagaatatgcaagcctatggggcaaagatacaagaaaaattcacttactgacatcttgctatgtcccattcagtacttgaaaaatggcaattactcagtgaatgagttatccatgtggctggcactacagtggattgatgaaatagcaacaagggctatttgatttgaagattcccttattaaaaagcatgaaatcatccctcagccctgcaatgatgctctaaattyaaaagcaaacaaagcaacaagaaaccaaaaccagaaaaaaaaaaaaaaccaccaaaacaaaactgggtgagtgcttcgcaaagtccagcagaggcctgctgcctcaaagcaccagagggtcaaagtggcctgtgcctgccttcactgccccacgacccagaatgaaacttcctccctttcaggaagcctttgggagtcttgaccctccaactctgaatctggagagggagttaggtcccatggacaagggtgggccaagcaaaggcaagacgtgtgtttattcaatgtacattcactga

SEQ ID NO: 132 - DNA, 599 bp, Homo sapiens
gggttcggggcaggccaatggaggagtcaggttgcatcagaattccagctagctcccgtgtaacatgttacaggaaggtgcagctccagcatcccacccctctgccctgtgggagaaagaaaagggcactgcaaatgcattgcaggagggacgtttaacccgcttgcaagatccaattatcttcaagggcttagcatatctatgttacttaccaatgtgctaattgtgtgctgttcttatctattcataagagtgggtccctatcatgatggtctactggataactgttgctgagctatsataaagctattctcccagtatcagactagacaggataccctggcttcctcccccacagttaaagaggatggagtggctctgagtggggctctggggactcaaaagtcccttgcagaaggtgctcttatcttttccattttgcaccaatgtgcacctctactttaacaagggctctctccaccatctcctatatggttcccccagccacctttgtgaagttccaccagtatctcatgtccttcattcccacttctaggtgacccagctctattccatttcaagtgaaattccatgtgaaa

SEQ ID NO: 133 - DNA, 599 bp, Homo sapiens
ggcccctcaaccattgcccaggccaggagtggggaaaccacttggctgaggcctccagaagagctggaggaatcaggacatctggattccagacttagctttgacattgttctgtgtgtcttttggaagatcacagaacctctctggacctcaacctcattcacctccaagtgggtatgacaatcccagaccaccaagtgagtatgacaatcccagccaaccttatgagagcttcatgaaggacagtgagaagggcagcaaaaggactctgcaaagcactggggatgaaaatgacaccayctcccatctctgagcacctaccatgcaccatgctttcgagtgctcacctccacccatgagatcattatctcctgtgtctacagatgaggagtcttgggcactagtccctggaaagctgagaatctccccctgcggccccctgcaaaactgcacaccatcatgaagtgctgctgtgactgctgggcttgggcaggcggcagggatagggccctaccctccaatctggctacttcttcttgtgcttctcccaggatgatgccttcctgccccaaaggaacctcagagaaactcctatctcc

SEQ ID NO: 134 - DNA, 599 bp, Homo sapiens
tggccaagttactcaacgtctctgtgcctaagttcatcatcaaaggagaattctagtaatacctacttgatagggctgttgtgaggatttaatggcttaatgcgctttggacagcatatggaacaaggcagactaccaacatctacaatgcaccgttgggggctaacatgtgatcatgcccccgcaccaggcctaagagcagtgggaattagttctgcaccaaaggaatgaccctctgatgtgggtccagtgactgtggcagaatttccagtgcctgtggcagaataatgggacatgccrggcctttcaaaacctagcacatggacagtacctattttgcccaaagttagggctgtctccagaaagaacagaaccagaagaagttctaaaaaccaactggctatgaggcccacaggcaaatctggcccaacctattaggtgagacaggtcacaccacttgatgaatgacatagtgaggatgtgaaagggaagccatgtgcccaagggtatgcagcttctctgtggggggcctgggtcagttcacaatccccactgctcttccctggtgcctcgtttcctgggccaatggtgagggcagg

SEQ ID NO: 135 - DNA, 599 bp, Homo sapiens
cacccctgagctgaatctcaaaggaggagtaggagtgactgtcagtgtggggggttggggatcagcattccaggcagggaggagggtgggagcacaggctcagaagcagaaatgggaggaggggagtttaagttaactggggccttttagataccattttgactgcagaccacagagagactcagagaccatggttaggagcttagattttatcccggggcggagggagctttgatattgggaggaacctgggcctgcttgccttctcaggaaacccctctggggcattgtgagtgggtktgaggaggtgccaccgagatagagcagttaggaggctatttcaccgttggccagaagacagtggagtctatgaatctatgctcatgagagaggctggggcttgagagtaacattttgtttgtttttttttttgaaccatcattagaggggtgagatgaacagggacgctgcgtagaagtaacaaaagcagtagttctgggctggaacctcagaaaagagtccatgagtgagagggaggagtgattagaaagataggagaagaaccagaaagtggagagttatgtaggcccagtaggagg

SEQ ID NO: 136 - DNA, 599 bp, Homo sapiens
catcatttgttactacaagggccctaaaaggtagtgcctcctagactggagaagggacctttgagagcccctagtgaagtctctatgcagaactcagggtcccctgacggatatccactcacctctcccaagatggggagatcaccctgcgcatatgctctgtccaagtccagtctgtacgacgctggggtcagggatgaatgggacagtctatgcccctgaagctctaagcctgacagggagacagacctcagacggggagcacacaccatagcagaatggagcctggacagtgtggargtgccacccccaaccataagagccagcccccatcccctgatcctgagctgcctccatataaacttttgggggatgagggggaagactaattctggagacccagggtctatccggcctggctgcctgccccagccctgggaaagagcctggcccatctctgagcagatgagccagcctgggccaggtacaacaaacactgtttgctggtcttccccgggtgttgaccactagccctggctactgttccagcagcagggcctgcctccagactatttagggaggttctggaaaaacagcaa

SEQ ID NO: 137 - DNA, 599 bp, Homo sapiens
ggatgggaagggacttgccagcacccctgctcccctggccagcataggagtggctgctgcaggaacggagggcccagagaggggccttgggcccagcaaaatagctccaaggccacacaggtcaaagccctgggttaccttgttcccccacgcagcagcagggctggcgaagcaggaggagaaggcagggcaggactggtgccctccaaacctcagccagcaccccaggcccaggcctgcctctgctggtcccaacattagccacatgggttgtaactcaagctacacagcatcctgggycatctctcctcttttggggacagttgtgtgacgtcctgttcctgaccatgtgtcaagccacctgaaggcagcggtgctgtcccaccccaacgtggtattcccctctgctcccctgcccttggcgggaggaccatgcccagagaatgagggttgggaggactcaagtggtctgggcagctgccgggaggaggcatgtgaaagacagaaggactttggaaagggaggcagactcaggggagggatcccaggagaagggagatgcaccctgagcagaaggaaagatgaggccatggaaatgc

SEQ ID NO: 138 - DNA, 599 bp, Homo sapiens
ggtctgggcagctgccgggaggaggcatgtgaaagacagaaggactttggaaagggaggcagactcaggggagggatcccaggagaagggagatgcaccctgagcagaaggaaagatgaggccatggaaatgcctggtgtttgtggaggccaggtggccttcatcctaggggagggagcaggaaggactcctgtgcagggccttgagtgatcagccatgagaagaggatttgtcttcagggtagagaaggatcaccctggggccttcctgagctggtggggacactccctgcacattcgytcccgccactctctagggctgccagctggcctcccttcctggtgctgccatcactcctcctctctcctgaggactccgatactgggctcaggctccagctcctttctacggggtgagtgaaaccaaatgggcgggtttgctggcaaggggcaggaggcaccagggcacggtagggtggagaggaggccagttccaaaggctggggtctgggctccaaccacactctgcattcactgcctcctccctgagtaaaccaggcactctcctgggcctcaggctcctcctgacaaccagcagga

SEQ ID NO: 139 - DNA, 599 bp, Homo sapiens
ggcgggtttgctggcaaggggcaggaggcaccagggcacggtagggtggagaggaggccagttccaaaggctggggtctgggctccaaccacactctgcattcactgcctcctccctgagtaaaccaggcactctcctgggcctcaggctcctcctgacaaccagcaggatgccagcttcttttgggactgcaccagcgtggggctgtggcacagggccgggtgtgagcagctggtctgccctgcagctgctgcagagagcagctcccgtcctgggggcaggggccatgtggtctgtatytctcagtgtgtctccacctctgcttctctgtccattttcctctgtatctcttggtagctgctgctgcttctttcttatcttcctcctggaccctggaaatccctgtaattctgctgtcaatctcttcttcccttttttagtttcttttccccactatctccctcatctccgaacacccacacccagccagctccacatgtgctgcacttttcttggaagcagctctcacggctgtcccctgtcaaggctgccaccctggttcagatgcccagtggcctctgtcttaaattgctacagaa

SEQ ID NO: 140 - DNA, 599 bp, Homo sapiens
ttgccagaatatactaggcgtactgaaccttttcaccaaagaaacactaaaatatttacattcgacaattatcatctgtggtcatggtgatgttagcccctagttttgaggtaatggcctcccccagcttcagaacatgctctaagcctgtttggaacatatctcaggctagggacagcttgagactaagtcagcccaggcaatacagtgatgcttctcatctgtttggtcagtgctgagtggagacagagtggatcagacgatcctaggagaccccttcctcatactgctctggttctrtccctgaagtgatttctgaagatgttggccatcaggcataaaaactcaaattgggccaggaacagtggcttaatgcttgtaatcccagcactttgggaggccaaggcagccaatctcttgagcccaggagttcgagaccagcctggcaacatggcaaaaccctgtctctacaaacaatacaaaatttaactgggtgtggtagggaatgcctgtaatcccaggggaagtggtgtatgtaagggtggaagtgtagataggggtagccaggcagggatgggcttgggagaggagacaagcaa

SEQ ID NO: 141 - DNA, 599 bp, Homo sapiens
tttagcccctgatctgccacaactctcatctctttctttcttactattagaaagattgccttttctttctaaatgatgcagagaaagagtccagactggtgtcaatctcaccctgttggccttggttacctatccagtatccatgccccctaccaatcgtttctcccattccacggatcctggattgggctcagggacaactgcctcctccggaaggtgaagggcttccccgggatggtcacaacagtcctatgtcccttgctaattgttttagaaagggatctttgaataatcctggcyaatgagatgtgagagaaagtctgctgggaggcatgaataatgatctcacattgatttttaaacattgcaggaagagagctggtctccctcagaatagagtgcagctgatggaggagcagcagagccaaagcagcccaccttgtgccctggatcccgccccacctctgggtttcttgtcctgtgagatgataaagctccttcttgtttgggctggatgacctacagccagaggcatgcgctctgataatcaccagtccatgaggaaaaggccaggggaccctctgggcaccccctcctca

SEQ ID NO: 142 - DNA, 599 bp, Homo sapiens; identical to SEQ ID NO: 93

SEQ ID NO: 143 - DNA, 599 bp, Homo sapiens; identical to SEQ ID NO: 94

SEQ ID NO: 144 - DNA, 599 bp, Homo sapiens; identical to SEQ ID NO: 95

SEQ ID NO: 145 - DNA, 599 bp, Homo sapiens
ctctcacagagctgccctgatcccacagtgagctgcaagccctccccaggtgcttttacccaggaaggcctccctgactcccagcctgccttgatctccatgctctgaccccttcagccctgccttgctgcatctccctgctccctgggccacacccctgtctgtccactctaacaggagcatcctgagaacaaagccctgtttcttcctgaaaataggcacttacattcaagctggaagacctggaggtaaaggacagtggctgaggatgctgctgcaggtggcatttgctggtaaacyataaaggcaagttaggggctcaggaacccagagccaagtgcaggagcagaaattaggaatcctgggatggagactcagccttgactctgtctcactgtgcgaccttaggcaagtgtttttacctcacctgcagagccttactttccttatctataaaataagaatattcattcatacaagttcaaccatggggatgatttttggggtggggccttaaaaatgtaaggaattctgattcagctttcaaattaggaatagacagagggagcgaagcctgcacacctggttggatttcccat

SEQ ID NO: 146 - DNA, 599 bp, Homo sapiens
ttaattttgaaataagtgtagacttcaagaagttgcacaaatagtacaaagagtcctcaagtacccatcaccaatcttcccctagtgacatgtgggaatgtggggatagctgcagcacaatattaaaaccaggagattgactttgcataatatgattaactaatctatagatcttactttgctgttaccagttttttgcaggcactcttttttgtatgtgtatatgtatagttctagaagtttatcacatccatattgccaccactacaatcaaggcatagaatgactgatcaccacatrggagttccctctagagcacatttttttgtttgtttgttttttgaaacagggtcttgctctgctgcccaggctggagtgcagtagcacagtcacaacttgcttcagcctcaaccttctggactcaagcgatcctcccacctcagcctcccaagtagctgggactagagactaccaccatgcccagttaatgtttgtattttttgtagagacagagttttgccatgtttcccaggctggtctcgaactcttgggctcaagcaatccacctgcctcggcctctcaaagtgctggaattacag

SEQ ID NO: 147 - DNA, 599 bp, Homo sapiens
aagggaaagtggcaaagatactcaagagctcacaggtacctagcacacattgcccatgggcacacatatcactctgcaaactatacacgaaacccctgaaattcaggagtacacaagcccccacaaaatgtaggaccacatgcttcaatattcactttccaacatattcagatccacagaaacacaaatagccccgcgtaatgtgcatacaaacacatctatatcatgctgcaacatgcccacacttacacgtttcccccaaaacagcatgaaatcaatctgggcacctcacttctatgsaaaacttgctctctgaagtgtgaacatttgtgcacacacacaaatacacctaccctgtaggtccaaatgcctgggcagcatgccaggaatggcacccctgggccttgccagtcatggcagcaattcacagatgattcccttctccctggagagatcccaagctgggggccaccgagggggctgggcagtgggcaaagggctgaccctggggacttattaggggtcagggttcatgagggcagagcaggagaccccaggggcctggagtgatggaagagacagacgaagctggggctggg

SEQ ID NO: 148 - DNA, 599 bp, Homo sapiens
ccaaccccactgttatatctgttccaggtggggaggaaacagggtccacccgctgctgcacaagaggggtgtgtgcagaccgtcaccttgtgtctgctgtagcaggagacccctggccatgcgggactgaacccatgattgcagctgatcttactctgtctcttctctgtgtgagtaaagtgttgttccatctagtgcctgtgtgagtcgtgtctttcgtggcgacacctataaaccatggagtgggctgacatcctggttctgctgctcctggtggcgggcattgttgtgctgtttgcygtcctctacacagtgcgaatcttctctaccctgcccgagtctcggcatctccacatgtgccatgaggatggtaatgctttacagaactgaaggtaattgaagcatggccctggagcgcaggaggcacccaatgtgtggtagttattattatcttcagcatcgccagtattattactaatgcctaggacttgtccacaaaaatcactatgaaaatttacttctgtgaactagactcacatcaaaatgctgcctgcttccatgaggcttgtagtgacatccatgtacaagttcaaagaccc

SEQ ID NO: 149 - DNA, 599 bp, Homo sapiens
ctgaagcagaagaatcgcttgaacctgggaggcggagattgcagtgagccaagatcatggcactgcactccagcctgggtgacagagcaaggctccttctcaaataaatagataaataaataaatgaaggcaaagcagcattgttaactaaaaataagagcctacaggctccaaattgggataatttctactaactgtatatttgatggaaatggattgagaaacacatttttaataactcatgtatgccctgtcatctgaaccaaaataggacacatgggccaagaagttacaaatctyggtgtgggcctaaccctgggaagcagggtctgccctgatgtgttccccacctgcatagacactgccactcatcagagcaagctttctgcccctctgattgtcatcatgttctcccaacctacactcattcagaaactgcaacactcagcatgcaatgtggcaggaagcagaagggaacaggcaaagctgggctgtgtcccgggatccttaactataaaaggaaatgctttaactttagaaatgcatttccataaggattttagtcaatttgtccatttctataataaaggcagctgtga

SEQ ID NO: 150 - DNA, 599 bp, Homo sapiens
tatagtacatagagaaaatgctagatatacaattctgtattctgcttttttctgttaacatttgtattttttctgattattattcattccttgtaaacaacacttatatgcctcaaaatattccattaagcacataaaccacagtttacttaactacccccttattgcagaacatttagagtgcttgcatttttcatgttggtgagtaatgtgcatgaaaattattgtgcatacaggattttttctatatattttcagtaatgtcctcaagatagatttccgaagtaagtcaggctgtgsgctttgatacatattgcaaagtccagaacatattatttaaaatcacacttaaaagccgaagccttctaacataaggcagaactggttgctgcttttgccaggtgaagagaaaaccacccattgacaagacagaatgggacagcttctttgatgagagtggccacttggccaaatcacgggacttcatttgtgttaacatcctggaaagggtgggtccccaagctacccttggccttgccctggaactgggagcatggatgggcagggggcaggtgtggcgttggatgaagcccttttgg

SEQ ID NO: 151 - DNA, 599 bp, Homo sapiens
aattctgtattctgcttttttctgttaacatttgtattttttctgattattattcattccttgtaaacaacacttatatgcctcaaaatattccattaagcacataaaccacagtttacttaactacccccttattgcagaacatttagagtgcttgcatttttcatgttggtgagtaatgtgcatgaaaattattgtgcatacaggattttttctatatattttcagtaatgtcctcaagatagatttccgaagtaagtcaggctgtgcgctttgatacatattgcaaagtccagaacrtattatttaaaatcacacttaaaagccgaagccttctaacataaggcagaactggttgctgcttttgccaggtgaagagaaaaccacccattgacaagacagaatgggacagcttctttgatgagagtggccacttggccaaatcacgggacttcatttgtgttaacatcctggaaagggtgggtccccaagctacccttggccttgccctggaactgggagcatggatgggcagggggcaggtgtggcgttggatgaagcccttttggtaggcatcgatagagcccatgacctccacc

SEQ ID NO: 152 - DNA, 599 bp, Homo sapiens
acagctggcatgcttggctgtctgtgtgcagtggccagaccccacactcattcgttcacacacacttcaacactccatgtctggctctcccttggcaagtgtgggctccaggccaatagtgcgagccgaatgcctgccaggctgagtgggcaaacaagcccagtaggcccgagcaaaactcaggcaaaggcgtcactgtccacacaggtttcaagctggaaaggaatacctcaagaattccatgacaataacacaataatgtcagtgatgatggcggtgatggtgatggtgatgtcggcracgtcagtgatggtggtagtaatgtcagtgatggtggtgaagctaattatatggcggtgttggtggcagaggtgtgatgtcatttatgatggtggaggtggtagtgatggtgacatcagtggcagaggtgatatagaatctggtggtggtaatgatgtcatcactggtggtggtgatgatagtgctgacttttcattctgccaacaaaaactgactgtctctaatggtactccttaaatagtgccagaggaaaagatgagattcaaggaagggactgaaggggattgcagtggggaggg

SEQ ID NO: 153 - DNA, 599 bp, Homo sapiens
ctgccaggctgagtgggcaaacaagcccagtaggcccgagcaaaactcaggcaaaggcgtcactgtccacacaggtttcaagctggaaaggaatacctcaagaattccatgacaataacacaataatgtcagtgatgatggcggtgatggtgatggtgatgtcggcgacgtcagtgatggtggtagtaatgtcagtgatggtggtgaagctaattatatggcggtgttggtggcagaggtgtgatgtcatttatgatggtggaggtggtagtgatggtgacatcagtggcagaggtgatwtagaatctggtggtggtaatgatgtcatcactggtggtggtgatgatagtgctgacttttcattctgccaacaaaaactgactgtctctaatggtactccttaaatagtgccagaggaaaagatgagattcaaggaagggactgaaggggattgcagtggggagggctctgatattgatgatggtgttggtggtgagggcaatcatggtggagatgaaaacagtgactaattcattctgaaaaaactagtgcccagcacacttccaatgagcaaggcctagacataatatctttaaatg

SEQ ID NO: 154 - DNA, 599 bp, Homo sapiens
aggccagcccaggcaatatggtgggacccagtctctaaataaataaattaaaaaaaaataaaattaaaaaaagtttaaaacactcatctttaaagtaggagtacagagaaaagcaaacacacacccccacacacaaacccacatgcacacacatatacttggaaatacacagagcctttgaaaagactctgcatgaggcaagaagagaacgtggccctaaaagggcagagaaaagtaattcctttgaaaagatttatcacataaacataaataacttgcagaccacatctgttctgtackcaattcatttaattaaagtgcgcactctaagaaatgagagttgaaaaataagatggaaagaatgaaatgagaaggctgctgactgagaggcaggtggaaatcaaaaacaaaccagaggagagaaggaaagactgcaaagaaactaaaaatgcagtagaattttaatttccatcaagatggtaaagaacagaattggaactacagaaaatggaatctgtaaatgtgaaggacagataggagaagttctctgagactgcagaggaaaagcacaacaggataaaaaacagagagacaacatg

SEQ ID NO: 155 - DNA, 599 bp, Homo sapiens
gcagaccacatctgttctgtacgcaattcatttaattaaagtgcgcactctaagaaatgagagttgaaaaataagatggaaagaatgaaatgagaaggctgctgactgagaggcaggtggaaatcaaaaacaaaccagaggagagaaggaaagactgcaaagaaactaaaaatgcagtagaattttaatttccatcaagatggtaaagaacagaattggaactacagaaaatggaatctgtaaatgtgaaggacagataggagaagttctctgagactgcagaggaaaagcacaacaggwtaaaaaacagagagacaacatgataggtatccgggcttacagaagacactaaacaaatgaaataaaagcaagaatcacaggagaaagatgtccctgagctgatgagaagcctgggcttatacatttaaaagattcctccttaagtcaaattaataaaaataaatgaacacttagaaatgccatgtagaattttcaaattgcaagggtaaagaaagaattctatcagcacccatgcagagagaaatatggagaaatatctgcaacattcatttttgttgttgttgttgctgagaccgggt

SEQ ID NO: 156 - DNA, 599 bp, Homo sapiens
ctctgagactgcagaggaaaagcacaacaggataaaaaacagagagacaacatgataggtatccgggcttacagaagacactaaacaaatgaaataaaagcaagaatcacaggagaaagatgtccctgagctgatgagaagcctgggcttatacatttaaaagattcctccttaagtcaaattaataaaaataaatgaacacttagaaatgccatgtagaattttcaaattgcaagggtaaagaaagaattctatcagcacccatgcagagagaaatatggagaaatatctgcaacattyatttttgttgttgttgttgctgagaccgggtctcactttgtggcccaggctggagtgcagtggtgcgatcacagctcactgcagcctcaacctcctgggctcaagtgatcctcctgcctcagcctcccaagtagcttgggactacagccatgtatcactatgcccaggtaatttttttgatttttgtagatatgggggtctcaccatgttgcctaggctggtcttgaaatcctgggctcaggtgatcttcacaccttggtctcccgaagtactggggttacaggtgtgagccacgacac

SEQ ID NO: 157 - DNA, 599 bp, Homo sapiens
gagtgcagtggtgcgatcacagctcactgcagcctcaacctcctgggctcaagtgatcctcctgcctcagcctcccaagtagcttgggactacagccatgtatcactatgcccaggtaatttttttgatttttgtagatatgggggtctcaccatgttgcctaggctggtcttgaaatcctgggctcaggtgatcttcacaccttggtctcccgaagtactggggttacaggtgtgagccacgacacccagcccaacatttcttaaagtgtataattggaagagagagtccatggaattytgttaactatttgtactgaggaaaaaagtttctgtcataaaataagcttgggaaatgctgaattaaacaaaacaagataaaaccctatgctgaggctttctcagggcttttaacgtattcatgcatgttattaaactggcaatattagcaagcccagttttccaaacttatttgactatgaaatcctttgccatagactatgtggtaagactggtgttcaatagaacacactttgggaaccactcttttacagaattttgagagatcagttgtgaccttacattttaacaatttgcctg

SEQ ID NO: 158 - DNA, 599 bp, Homo sapiens
tcctggtgtagatgagaaaacaggcctagggaggtggcacagaagggtacagccgagcttgaattcaaactgaacctgtgctctctgcatcacagtgagatgacccaggaggatatcatgttcagcctgcgggcagggtggggcagggcatgagcatggacccctctgccagctttgcctctgtggtcctcacctcccacatctgcatccagaggaagaattatgcttgcatttgctgagtgcgttatacatcatctcctttaattcccaggtcctagccaggtactgttatttgtcccrtgttacagatgtggtgacgagctcacttgctgacttttagtagtggaaccaggatttgagtctgtgtgactctctatcctgagttcttaacaaccatgcatttaaaaaaaaaatttatatattattgcttatcttgtacacctcatttattcactcattcatttggtcattcattcattcaacagatatttgagtgctagattctgggcactggttgtgacagggtgagatgggacagtgagtgtgaaagactttggaagcagagtctctccccagtgtggggttagggtgtagcctgg

SEQ ID NO: 159 - DNA, 599 bp, Homo sapiens
cagctttgcctctgtggtcctcacctcccacatctgcatccagaggaagaattatgcttgcatttgctgagtgcgttatacatcatctcctttaattcccaggtcctagccaggtactgttatttgtcccgtgttacagatgtggtgacgagctcacttgctgacttttagtagtggaaccaggatttgagtctgtgtgactctctatcctgagttcttaacaaccatgcatttaaaaaaaaaatttatatattattgcttatcttgtacacctcatttattcactcattcatttggtcrttcattcattcaacagatatttgagtgctagattctgggcactggttgtgacagggtgagatgggacagtgagtgtgaaagactttggaagcagagtctctccccagtgtggggttagggtgtagcctggtcatggtcattgcagattccagaacgtttgtgtaacaaacactcatatggtgcatactaagtgccggacactgttcttggagttttgcagttagaatcacttaatccctgtgacaaccctgggattgttatccccattttgcagatgaggaaactgtggccctgagaag

SEQ ID NO: 160 - DNA, 599 bp, Homo sapiens
ctgagtacaggcaaaagataataccactcacccctgccctcatgggctcttgcctggctgcagaaggcagatatcatctcgtgaggaaatagggcagatatcatctcatgaggaaatagtcatttaacaaatatttattgagcgacttactgtatgctgggcaccgttctagagctgggtattcattcgtgaaaagaaagagacaaaattcttgcccttgtggatcttctattttcatgtataaacataataaatatgtaaaaaaaatagaaggtactaagtgctgtggaaaaatagagwtaggtaaggaagatcgggagtgtaggagagggacaggttgcaaaggtggtcaggcctcatgaaggtgacacttgagcaaagacttgaggcatatgagtgagccatgcagctgtctgcaggaagagcattccagataggaagacagccagtgcaaggccctgaggcaggagttccctggggttttcaggtgtgctccagtgtggttagaacagagtagagtggaaatggaggtataacaggggacaggtcagagaggtaatggggtccgggccatggggagcttgtcggccattgtaagg

SEQ ID NO: 161 - DNA, 599 bp, Homo sapiens
tggagtaagcaaaaagacctcatttgactgggtgcggtggttcacgcctgcaatcccaggactttgggaggctgaggcaggaggatcacttgagcccaggagatcaaggctgcagttagccatgttcacgccactgcattcctgcctggacaacagagcaagaccctgtctcaaagaaaaaagaaaaggtggaggtggcgtggggagctggatatgtaccttcaccaggtccaggatcttcctttaaaatgcatactttttgaggtgagggctgatcacacagtctgataataactggayttcacccaacattggccactctgcttggagggcctgttgtaccctacaagccaaaaattgcagaccagcctccttactcctacactgatcgtgtccactagcccccaactcaatcaacccttctttatccccttccaacatcacataatcccccagccccaagccctgcagaggggccaagagcccaagcctgcaggcctggctgctgacgaaatgtcccctgggagagctgcccagagaaggagccattgttccttcctggaaaaacataccagctatgggagggggtctgctattcc

SEQ ID NO: 162 - DNA, 599 bp, Homo sapiens
tctagcccatcccttcctgcccaccctgactcctgccagcctgcatccacctggctctgcccatgaaccttcagggacacactccaggccagaagcccagggctctcttggtggtttacaaagaatgctgactgcagggatgagaggaacggagggtgtctgggggctgctgtccagaggcctggggagtaaggactcgagggagtgcctctgtcaggaaattacatggttcactcattcctgcctccaggtctttgttcatgctgttttccctgccgtggacaccctttcccgctctcygattctctaaatcctgccccatctcccagatcttgttcatgtccaagcttttccaggaagtcttagcagctcccacaccgcagagctcgagatgtaagctcttgctgcctgacttccctgaccccacacctgggcccaggacctaacacccaaggagaatggcaaagggttttggggtatgagaggatgtttggggtgtgaggaggtctctgggcattagcagtggccaacctgatgctctcaatgtcatgctcttcctccctccccccttctctcccactgcccactccaggtctccc

SEQ ID NO: 163 - DNA, 599 bp, Homo sapiens
agcctgcgcggccctcccagctccccgggggcctgtgggcagcagggggagccaaagcccttccttccaagagctctttacataagtgcttggaaaggccgggccttattagagtgtctcagtctctgtgaccctgaccgcaggcaggcagctgggccagcgtgcacaggaagctggacagagcgttcccagaaaggccacgggactgacgcctgtgggtttccatcgtgtttctggaggaaaaaggaggcttaaaaaaaaaaaagtactgagttagaaaaaacaagaaagtatttccaracagtagcagctctgctaatggcctagaaaacaatctgggactagggcatggggaggggaatgtgctgagactgtgccctgggtgggtgggagtccatcaaacccctccttttggagctgggctgagggggtcggaactttctcttgagggaaatgtaggcccatgcctgccctgccacgagggatctgggctgtgggagggacgccctggctgaggaggccacgggggcctactttgcagcccctcattgacccactgtctttccttcctcagatcctgatctccgggagggacagat

SEQ ID NO: 164 - DNA, 599 bp, Homo sapiens
aacaaaaaaaccataggtgccatcttcactcctgacccagcacctccttctgggctgcacagaccaagagggtctctgctggtacactgggcactggtgtgcaggaatccacctgggctgtaagggcctcaagtcacctgcccagcatccctccctgtcctcttcttgctcagccccagggctggggagctcactgcttctcgagacatttgctgttagaaacaccttgaggtgaaacagacccccaccccctgccccgcagccttcccttgggctggtcctcggggtctactaagtaamtgactctggtggatgtgactggacagcaggaaggacagagacagcagaggcgacacctggcagggggcacagctgtggccctgactctgtccttcagtgggcctcagtctccctatctgtcctgagggggctgagtgtcttgctagtccttacctggcagagaagaaggggcaggggaagagaagagagggaggggaggaaatgggaggtagaaggaggctgggtagggagtgataggcaaagtccagcctgggggaaggtgggcaagcagcagggggactgcctcagaaggggagcag

SEQ ID NO: 165 - DNA, 599 bp, Homo sapiens
ctgtcctgagggggctgagtgtcttgctagtccttacctggcagagaagaaggggcaggggaagagaagagagggaggggaggaaatgggaggtagaaggaggctgggtagggagtgataggcaaagtccagcctgggggaaggtgggcaagcagcagggggactgcctcagaaggggagcagggagccagcagaggcactgctagggctgcagcctggtgagggctggggcgttatctggcttggaggatgccacagagagaggcctgaagtgggggtactggacctcaaaccagggcyggcctgaaacccttggaagggcctgctaatagagacagaggtgccctgggcccttggcagggcggggtgggctctgaaagccagcccagacctgacccctctgcggcctaccatcttggccacagccttgaccttctccctccccctgcacctcagatgcccacaccccctgtgcattacactgtgcgctcccacccagggcagcagccccctgcagggagggcccaggctaaaatcccacccattctgtttcctctccttccactagaatcccacctgttttgagaagttggcagggc

SEQ ID NO: 166 - DNA, 599 bp, Homo sapiens
agtccttacctggcagagaagaaggggcaggggaagagaagagagggaggggaggaaatgggaggtagaaggaggctgggtagggagtgataggcaaagtccagcctgggggaaggtgggcaagcagcagggggactgcctcagaaggggagcagggagccagcagaggcactgctagggctgcagcctggtgagggctggggcgttatctggcttggaggatgccacagagagaggcctgaagtgggggtactggacctcaaaccagggctggcctgaaacccttggaagggcctgctratagagacagaggtgccctgggcccttggcagggcggggtgggctctgaaagccagcccagacctgacccctctgcggcctaccatcttggccacagccttgaccttctccctccccctgcacctcagatgcccacaccccctgtgcattacactgtgcgctcccacccagggcagcagccccctgcagggagggcccaggctaaaatcccacccattctgtttcctctccttccactagaatcccacctgttttgagaagttggcagggcctgtgttagggaagcacctgataaccaa

SEQ ID NO: 167 - DNA, 599 bp, Homo sapiens
tctggtttaaattttcatttccctagtgactaacaatgttgagcatgtgttcatgtggttgtttgccaggatatctcttctttgatgaaatttctgttcagatcctttgcccatttttaattggcttgtttgtctcattattgagttggaagaattctttatctattctggatacaagttattcatcagtgatatgttttgcaaatattttttcccaatctgtcgtttgtcttctcattttttactagtaccttctaaagagcggaagtttttaattttgataagagccaatttatcggktttttttccttttatgggctgtgttttttgtcctatctaaatcttaacttaaacaatgtcacaaagattttctgtaagcactgcaataaatggtatcccatgagtttttaggactaagttctaatttttgattttgcccaaatttctatctaagggatctagggagtcatgccctacaaatcctaaattctcatcagatgggttttatttatatattgtgacttacttttcaatctgactctggtataacattacgagacaaggaaaaaatatttaaccccaaaatatatttccctgcc

SEQ ID NO: 168 - DNA, 599 bp, Homo sapiens
gaggtgggatgatcactggagcccccccagaaaagaatgggaatgatgttcccttgtggtttttctatatcctaagaggaaataaaacactcttctctttcacttttaacatatgcaaatgagtgactctaaatgccttctagggcaatgtctcatggcttacagtgcctcgtggtttttctcatttctttcatgattgcagctgtatttgctaccacttgtgcagctgacatttaccaagcccttactgtgttccagcatgctttcatttagaaccatccctgactcctcttttctctyaaacccacgttcaatttgtcagataatcctgttggccccatgttcaaaatataaccagttcctttcaaaaataaaaatggacttatcataaaacccggcaattgcactgtcatgcatttttcccagagaaatgaaaacttattttcatggtggaacttgtacacaaatgttcatagaagctttatttataatagccctaaaccagaaaatactcacaaactatggtatattcataccacaggatactattctgctgtagaaggaatgaactattggtacaagcaacaacttggatgaac

SEQ ID NO: 169 - DNA, 599 bp, Homo sapiens
cagctgctgtcaggctgtttctgtgggtcctgtctctccagtcaggaagccagcagggcagggcaggcctggctctcctcctggaatcacaccgtgcacccagctcagcgctgggcactcagctggggaccgggatggacaaagccttgtccagagaaccactgtaggggagacgtgcatggcagccggactcagcctcctgcagcctccattcatgggctcttggctgggcctgaaatttgttagggcctcaactctctgtggtgcagatgaacacctcctcctccaagaggcccctcrggcttggtgccaggcttgggactgggctctggaaagtcagccaaggctgctacttcaaggccctgaggacagcctccagcttcatggccatagccaggtcgcccttcaccttcagccgtccactcatgtaggcccccaggggccgcagctctctgcatagcagggcccgcaggtctgcctcggccatctccaccaccacatcagggatgccatcaggcaccccgtgtcccactcttcctcgtcctgtggggcaaaagaaaaccgagtgtcagtaggggaatggaggaacgtgaaggaga

SEQ ID NO: 170 - DNA, 599 bp, Homo sapiens
attgagaaatgaaggcccagagaggcaaagaaacctgttcaaggtcacatggcatttgtttctgtggcagttttagaactaggatataaggccatttaaggacccccttgcagaccacaccaaacccagaggttttctagagagggactgttgcctagggctacacaaagggcaaggcctccttgggcttccaccttcccccagctttcacgtgcgatgctgaactatagctccatggttcatccttttcccactgtaccaccccttatggaaaatgaggcctgaattcctattatgcaytaccagctgggctaagttgggtgttttcatttctaatttattctaacccaaagagaagataatttggcaaagacatctgcgtgtaccttaaagccagtgtccagatcccaggacttaggagtttgggacttgcttctaccccctactggctcccagtatgactttagacaagcccaagccttagtttcctcccctgaccaatgtgaatgaatggttcttgcctagcccattacacaaaggggtaataatatttagggaaaggggacagaggataggcaaatttattcatcaatgccaaa

SEQ ID NO: 171 - DNA, 599 bp, Homo sapiens
aagagctatcttttctgtttcaagtgtcccccgtaccccaccacctacaaccctaaatccctggtgaatttccactgcttttgcaggtcccagctgtgggctctcctttccagaatgtctccgtttgcaggcttgctcttccattacatctacggctcttagaaagcaggggctgcgtgtggctcatctttgtacgcccagctcccggcaggacgtggcacgaagcagtgccagtgtgaacggatgaatggatcaaagccgggagcagggctgcccccctgcagctctgccctacctctyccgctttaccgtaagtcagcggtaggtctgcagctctccgcctctacctcctccccgctctgggcgtgtctttaaaacccacagtcggcctctctgccccctagaaccgcccccagcttctgtctcacttcctctccagaggcgggccctgagccggcacctcccctttcggacagctcaagggactcagccaactggctcacgcctccccttcagcttctcttcacgcactccaagatctaaaccgagaatcgaaactaagctggggtccatggagcctgcacccgcccgatctccga

SEQ ID NO: 172 - DNA, 599 bp, Homo sapiens
tcaggtaatccacccacctgggcctcccaaagtgcttggattacaggcagaagccaccatgcccagccccatatcgtttgtttttaatgaaatgcttctattttacatggtagtaactgagtctctgcaatgaaagatcaagatggttggcttccatccttctagttttggttccatgtcaataggactgtaaggttcaccactggtctttttactttaacatcacttttcccaagttttgtattttgattgagctcttaattggatagatattatcaaaaagcaattttttttttccargaaggacttttttgttctgaattccttgagtctcttggtgttgagaatgtctttgggttactctgatacttgaataatatctcggctgggtataatattcttaggtcacaatcactttcacaattttgtagacactgctgcattatcttccagcatcaaatgttcattaggacagctctggggtcaaactgacttgctttttctgtctttatgcctaaagatattttttattcttgaaatttattaatttaaccaagctgtcttggtgtttaacattttgtatcaaattattctggaac

SEQ ID NO: 173 - DNA, 599 bp, Homo sapiens
cctcaggactgctgaccctatatctaaccatacatctgacaaccatagtttctcatggctggaaggcactaatatgccatctaatgctggaattccctctgcagcagccctgatctgtggccgactgtctaccccacatctaacccccactgaacctggctcatggcccatcaagagcaactttggcatttaggagcctagagtcagagccagctagccagccaacccttctttaatgtcttcctttacttcctctcactgaaatttccagtccagccacttgctgtccctcactcagaygctctccctgcacacacagggacagcaggactcacaacctgcctgacctgctgggccccaccaccaccccttcagacctcaaacccatctcaccaggtcaacgtcaatagggtccctgggagtgctggcagcctctgggctggctttcttggatacagctgcatctgtagaacacaaagaaggtcacaggcaggtcaaggacatgctggtaagggacatcctgacctgggacagtgatagagaggaggtccctgttgcttttccaggatacctattttccagggcatcccaaagcactg

SEQ ID NO: 174 - DNA, 599 bp, Homo sapiens
tcaaccatcctgccaatgcccaggaacatcctgcccagctgcaaaggggcatcagcccaccccaccggatacgaggggctgtgcgatcccgcagccgctccctccggggctcctcccatttatcccagtggctcaacaacttttttgccctccccttctcctccatggcttcccagcttgacatgtcttccgtggtgggggcaggggaaagcagagcccagactcttggagcaggtgttccccctggggactctgtcagaggctccatggaggcctctcaagtccaagtgcctctggaascctctccaattacattcccaccaccctgtgccccagaaaggccccccatcagcccagtcccaggcgcccgtcaagcaggcctctgagagtgctacccttctcttgtaaccttgcagccaacacccctgcccggcccctgagctgcctcctccagcccatgctcttacaggccctgcacagagtagcactcattaattcttggttaaggaatgaatcaacgaatgaatggctatgcatggacctctgggcagggagacctgggtcttctctggctgagaggggaaggctaaggcatggct

SEQ ID NO: 175 - DNA, 599 bp, Homo sapiens
gagagccagaggaccgctgcttttgtgtatgtagctttaagacacaggttttaaaaattgttataggagtcccaaactttagaaaatatatattttaaaaccccaacaaattaggtcccccctatgcatttctcacccaacttctgggccctttccagacccctggtgcccccagagtacacccagcaccaccactgcaaattcctactccccagcttggagggggtggggtgatattcccaaggtgatcccggctcccacctaacccatcctccccagctctgcagcttatatccagcyttgcagtgaggaaagttgcagaatactggtctgctacattactactttccaagtgtatcagggcatttaggaacccctcccttgagcttcacctgtgtcctagggattcagtgagagactgtttgtgcaagaaggagcgagaggcagcagggcatagtcatggggtggagcagtgagtcccccaggcctggttgtggagtacctggggtgaggactttgcttaaaaactgccagagtgggtcagcagtgcgcctgccctggggcccctgctttaggatccaaagtggaaaagataggtg

SEQ ID NO: 176 - DNA, 599 bp, Homo sapiens
gcagtgaggaaagttgcagaatactggtctgctacattactactttccaagtgtatcagggcatttaggaacccctcccttgagcttcacctgtgtcctagggattcagtgagagactgtttgtgcaagaaggagcgagaggcagcagggcatagtcatggggtggagcagtgagtcccccaggcctggttgtggagtacctggggtgaggactttgcttaaaaactgccagagtgggtcagcagtgcgcctgccctggggcccctgctttaggatccaaagtggaaaagataggtgttytcacccactcttgtgacagattactggctccggtgccttggtggcagcagcaacaggagggggccaaggtgagggggcagcctgtggcagaagtggcagtggtgacggcggaggaagggaggcctgtggggaatggctgatccaggggaggggccgtggcactggtgagtgtgcatctgagaaaatgaaacggaggtgctggccaagaggctggagctaggcagaagggcagcagaggccacaggctggccgagtttatggtccagttcactcataacatgtgcagtattcctggggtc

SEQ ID NO: 177 - DNA, 599 bp, Homo sapiens
acagaggcactaaggcctagaaaaaggaagcctcataatttggtgacctaggcagatcactctatgtccccccatgcatgtgcatgttgtgtgtgtgtgcgcgcgcacacacacacacacacacatgcacgcacgcagtcccaccagctgtccaaagctccttccataaccctatcttctcaacagcctctctctcccaggacacccacaggcttaaccaagtcccaccaggagtgccccgagtttcctcccagctattccagcaggctgctggggggaatacagtgtcgggaccaagayagactctgcccctcttggtgggatggctacagcccacaaccacagctgaccacagcagcccccgactgcctctctcctctcagagcctgtttccgggttcagaccttcttcctccatctctctttctgggtctttctcccttgcttcctttctctacctccagaaaggaggctgtgattctctgtttttccctctctttcctggtgtctctctcatttctctgtttctctccagttttctgtctctgctgggcttgtccctctcgctccatcttctcatcaggatctttttttttgcct

SEQ ID NO: 178 - DNA, 599 bp, Homo sapiens
tatcttctcaacagcctctctctcccaggacacccacaggcttaaccaagtcccaccaggagtgccccgagtttcctcccagctattccagcaggctgctggggggaatacagtgtcgggaccaagatagactctgcccctcttggtgggatggctacagcccacaaccacagctgaccacagcagcccccgactgcctctctcctctcagagcctgtttccgggttcagaccttcttcctccatctctctttctgggtctttctcccttgcttcctttctctacctccagaaaggaggytgtgattctctgtttttccctctctttcctggtgtctctctcatttctctgtttctctccagttttctgtctctgctgggcttgtccctctcgctccatcttctcatcaggatctttttttttgccttccaggaaaagctgacgtggtgcccctggtcagtgggagtgaggccttctggggtgggggagtcctggttgagcagccacagctggggtgcggctggcgccctggctaggggcagcaggagcaggagcagagcgagaggcagcggaatgggaggagagagggctggacctct

SEQ ID NO: 179 - DNA, 599 bp, Homo sapiens
caggcaccctgtctggggcagggagggcacaggcctgcacatcgaaggtggggtgggaccaggctgcccctcgccccagcatccaagtcctcccttgggcgcccgtggccctgcagactctcagggctaaggtcctctgttgctttttggttccaccttagaagaggctccgcttgactaagagtagcttgaaggtaagccagtggggaggagggctccagggccagcggcgggagcgggaggcctgttggacataggggctggttccctcttggtccatccctgctggtctgaggtgcrtgggacaatccctagcttggagccgtccagggggcatctgcttcttccacaacccacaactgaggccccagaaatcccagctgcgtttgggctgagcctctggcctcacccaagtcagctgagaggtcctggcgggggtttatttaggcagctgcctggctaagtttgaacagaacaggccacgggtgtgattccacagaaaaggcctggtgtctgctgcggtcatggccggaggagcgggagagggcgggtggagtggatgggggtggtgtgcactgcacaaggggcctcgtctgggc

SEQ ID NO: 180 - DNA, 599 bp, Homo sapiens
tacacgctgctgcacaacccaaccctgcaggtcttccgcaagacggccctgttgggtgccaatggtgcccagccctgagggcagggaaggtcaacccacctgcccatctgtgctgaggcatgttcctgcctaccatcctcctccctccccggctctcctcccagcatcacaccagccatgcagccagcaggtcctccggatcactgtggttgggtggaggtctgtctgcactgggagcctcaggagggctctgctccacccacttggctatgggagagccagcaggggttctggagaaaraaactggtgggttagggccttggtccaggagccagttgagccagggcagccacatccaggcgtctccctaccctggctctgccatcagccttgaagggcctcgatgaagccttctctggaaccactccagcccagctccacctcagccttggccttcacgctgtggaagcagccaaggcacttcctcaccccctcagcgccacggacctctctggggagtggccggaaagctcccgggcctctggcctgcagggcagcccaagtcatgactcagaccaggtcccacactgagctgccca

SEQ ID NO: 181 - DNA, 599 bp, Homo sapiens
gttttcccaggggttggggctcaccatggctaagaagtccccttactcccagaaacacacgggccaatgccactgggaccctggcctctcagttctttgccactgcccacccagccaagctcccttcccagccactgaaggacaatagaacccactggccatgaggacaagcttctttctttccctccattcactctggaacctcatcaccaccagagggcaggcgagggaggactgagagcctcattggctgggtgggatatcgaagcggcaagggcatagcccagtgggagaggctgygctcctgagacacagagggcagggttagggaggtgagggcagggtggtctgtgcgatgtggcattgccctgggggtgggagcctggaggaggcaatggcactggtgggtccccttctccacctgtcaccaccccagggtggccccagcttcccaaaccccattttggactctggcatatatctcccttccacctgctccccagcagggcctggcagctgctgtttgtacccctaagctcagctctgttgggggtggggagaccctgcactcagagagttcccggtgggaggcaggcctt

SEQ ID NO: 182 - DNA, 599 bp, Homo sapiens
ccataggcctcctcccctgtgtccacacctggggtttccccctgagttggtatcacaggacgtgatgtggccccggggagagaacattagtgtggctgccctcacatggccaggatgggtgaggatcaagtcataaccacccttccagggagctgctaagacttagggccatggcttggggctcctggtgctcactggccttgtgggggacccactgactttcaagtgtagatgtggatgtgggaccagggttgctatggggcagtgggctcagctctgggctctgggatccacaggagrtgaacaactaccggcgggccatgcagaagatggcagaggacatcctgtctctgcggagacaggccagcatcctggaaggagagaaccgcatactgaggagccgcctggcccagcaggaggaggaagaggggcagggcaaagccagtgaggcccagaacacgggtgaggatgtgcaggccaccaagggcacccgggcttctgcctgggcccaggctccaggttgtgcaatagggtggctgggagcttctcccagatgtgcatgctgctggactctggcaaataggagcccctcgcccaca

SEQ ID NO: 183 - DNA, 599 bp, Homo sapiens
aatatatatgttggggaaataaatgaatcaagatctgaataatgagcaggcattgtatgaccttcattgtttctttcttgaatattaagacactatcacaaaaaggacaactctacaaatatatataaaattcataaaattcctattatatcccagcagctggggcaaagaaaaaaaaagaaaaaaattccagtataatcccaacagttttttttttcccaaccaccccagatacatcagacagatggtatacatctcttcgtgactcaatctgctgaacgatgcaactgtcacagtatycaacaggtttctttttaattggattaaattatctgatgatttatttggaaagaataaagtacttgtaaatagccaataaatgtagaaagaagtacagtaagggtgcacttggcttactagatctcagaagacactataaagataccacaatagcatcaatataatattggcctgggcatagacaaataaaccagtggaatagaaaagagaatccaggaatagatatctgtttttaagaacataaaatgtgaacagtgcaattttaattcagtgggatatggatggattatataataaat

SEQ ID NO: 184 - DNA, 599 bp, Homo sapiens
ttatgaaacttttgggcaagtttccaacctcttaggtctgtttcctttctgtcaccagcgggtccattgaagtgttcttgtgctgggctctccaactctgactttgtgcaactctgtgtggagcctctgtggtgtggatggatgtcagcaaccattgtggaagttcaggtggtagctcaagggttggagtaagccgtagtgctgccaagtcagattctgcagagaaaaggagctaggacttacttacctctgtggctctcggacttggacttggctgggccagttcatgccttggtatcrgggtgtcctcatcaccaacaatgtattttagtttttctgccacctttggtaggaacatctatttctgggaagtcttcttgatcattgcatggagtgattctgtgcttagtggaaaggtgggaccgctgccctctcttcctcccctgctctgcccgctttctacagcttaatcagagaagtgtcctttagctcttttccaagccactgagtttgaattgatctccgctgctagcaagaggtgggaaggctctttctggttcctgctaaagttacctgacagcggaaggtgtgcatctgat

SEQ ID NO: 185 - DNA, 599 bp, Homo sapiens
tcctgtgactggtccacccacctggtgtgatgtgagggccacagagtcatttgccaccaaaacttcccatttcaccctctgcctagaaccccgttagtaccccgtagttaggactctgatgtttcaggcctgctccttccttccctccctccctccttttcccctttcctttctccttcatacccttgcaggcagtctgagcatacaggaaatgttaaaaggtagttaagagagcccctccaacttccccaaaccccttcagcaggagggaagaggcagagatggttgcctgtcccaccmcctttcaccctcaggggctctggttccacaatgcagatttcagggaaaccctgcccaaggagcctgcagtcccctgctcctaccttctacccacccggacagctcaggcccagttgccagcaggaagagggtgccaagagaacaagtgactttcccaaggccagctggtccccaggaaggaactggggagggtgaccagggtttgcagcatggaggatctcctcttccctcaacagccagatggttcctcctcagacaccggaggccccaggcaggcgctaaggacacctgggacatgc

SEQ ID NO: 186 - DNA, 599 bp, Homo sapiens
ttgctcggcactgcgctgtggctcgtgtccccgtccttcactctctcacttgctcggcactgcgctgtggctcgtgtgtctgcctctcatcccttgtgctgccttcacatcccacagatgagggaatctcgctgggaagctgacaccctggacaaagagggactgtcggaatctgttcgtagctgtgagtcctgccctggggtcgatcctccatccaaggaggggtgagggctgggggccctgggttctgcctcccttaccaaagacctgagactgaccctcccccattgtccccctcayccccgtagcttgcacccttcagtgaccctagaagaatgattggacagatgtgagccatctggagcagaggggcactaacccaggctgacgccaagaatgaagtggcccactgcagccctggcgagcaggcttcttggatggacagtgctgagacccccatatcccagagtccccagcctccctcaggttactctgcaccccacagatggtttgatggctgtgctgtatactggaggggagggcaggactctgggagaacagcacttctttcatgagacctttgttactcggtggttact

SEQ ID NO: 187 - DNA, 599 bp, Homo sapiens
tccttcctgcctgggctgggaataagcctctcacaggttctggtggacagatctgttccccaggtcactccagtggtctccaggcttccagagaaggctggttgcctcaagctcttctctgcctcataaacggatccagagaaggctggttgccttaagctcttccctgcctcgtgttcctgagaaacggattaatagccctttatccccctgcaccctcctgcaggggatggcactttgagccctctggagccctccccttgctgagccttactctcttcagactttctgaatgtacartgccgttggttgggatttggggactggaagggaccaaggacactgaccccaagctgtcctgcctagcgtccagcgtcttctaggagggtggggtctgcctgtcctggtgtggttggtttggccctgtttgctgtgactacccccccccctccccgaaccgagggacggctgcctttgtctctgcctcagatgccacctgccccgcccatgctccccatcagcagcatccagactttcaggaagggcagggccagccagtccagaaccgcatccctcagcagggactgataagccatctc

SEQ ID NO: 188 - DNA, 599 bp, Homo sapiens
aatcacttgaacctgggaggcggggagattgcagtgagtcgagatcactccattgcactccagcctgggcaacagagtgagactccgtctcaaaaaaaaaaaaaaatcagccaggtctcgtcgtgggtacctgtggtcccagctacactggaggctgaggcaggagaactgcctgagcccaggaggtcgaggctgcagtgagccatgattgcgctactgcaccctggcctgggtgacagagacagaccccatctccaaaaaaaaaaaaaacagtaagtgagcaaggtacactcaggcctygcttcctgccaccggctgcaatgtgtggaacccttcagaaaagcaagtctgagtcacctggccgggcattcaaggccttccaggacctggcaccaatcctctaaaggaatctaccctcaaaatgggacacttactatctcctcgcacctctgtgtagtctctccatacacaaaccattccatgccaagcactgggctagggaaaccaagaaagaaccttgattcctgcctttgagaagcaaatagtacagagaatcacacagaagccagggcaagaggtgatggcacatgatcagcact

SEQ ID NO: 189 - DNA, 599 bp, Homo sapiens
cccccacctgaagttgggtggtctcttactcggtgtggctgtgtctgcggcttttataggctcagaatggcagagtacatgctgattggtttgtgagtatgcaaaaaaggctaaaacaaaggcatcactcaaaggtgggcacaacagtgtaagaaaccaattagggaagggtaggtatatgtaaaataggtgaggggtggggatcaatcagaggaaagtgcaccaaagtggaagagaggttctcactccagtccgtggatttacccaggacttgtagcttggctttcaggctttaaactstctttggtttgaaggttggttttcaccggggacctgtccctgtctgcctaggatttgtctgcctcctgccactctcataacaataaaagttagctgcagccaggtgcggtggcttacacctgtaatcccagcactttgggaggctgaggccagtggactccttgggctcaggaatttgagaccagctgggctacatggcaaaaccccgtctctataaaaaatacaaaaattagccaagtgtggtggtgtgtgcctgcagtgccagctactgaggaggctaaggtgggaagatcacttga

SEQ ID NO: 190 - DNA, 599 bp, Homo sapiens
gttatttgtctgacaaccatccttacgatggtaatcctgtgagtgcctgatggacatctatctttgtgtttttctcaaaccatggaaggttgagaggaatatacagtatctgggggctgacttactctactagctaggagcatcacagcagagagataggacagagaccagagtcatgccagcttggggtcttgggaagagaggcagatggttactctgttctggaatgggactctgcagctctatgcagtggccaacattctgagcactttgtgcaaatgcttgtagagctcaagaacrtctccatccagaaccccttccccatctgcagagaccatgtccaggcctttcttgtgtctgcagaggtgaagtgcacatgtggaaagggatcaaatgtagcacagtgtaaagaaagacagcaacacaggtagagggacccagtgtgtgctgggaggtggctgtggaaggtaagtatagacctacatctctgttggggaggtggtcagggcagaatggcctgggtgtttagcagcttctaggggtgagggtggcaggaccccaaaaccaaggccctctgcatcgttgttgtaataatcagc

SEQ ID NO: 191 - DNA, 599 bp, Homo sapiens
agcaaaagaaacaggccccagaagttaaagaaccttgtgaaggtgacacatctagaggcagtgcttgcgctgatccatctgacgccaaaccccaccttctttccaacaccaattggtgcctgtgacattggacaggacctgaccctctctaggcctcagtttccacatttaaaaatagagccaatgtcactactcattgtacagtgaggcaaataggcgttgaaaaggagggtgaaagtgcactctgagtctgtgtgtggtaggctatgtgtcagggctcttgacttctctagcccccargggatgtgcttgtgactggctggaaagaaactccagatccttgtagaactcccaaatctcatgaggctgtcccttgactaacttactggcttcaatagggtaggtcaacagtgtgcaacctccgagctcccagggccacgcagaccgttgggtggcactatgccttgggcaagagaggtgaaaaggaagaatttagggctgaagttttcaaaaatcaaaagcacaaaggaatccttaccattttggtagattccccccactccccatgatgttagactgtttttgcctccagaaacagc

SEQ ID NO: 192 - DNA, 599 bp, Homo sapiens
ccaggaggcggaggttgcagggagccgagatcgtgccactgcactccagcctgggcaacagagtgagactccgtctcaaaacaaacaaacaaacaaacaaacaaacaaaaactatctctgctttcttgaagtgtggatttagccaaggagccaaaaaacaaaaaaacatagcagtggaaatttactagttatagggatagtcttagatatatatttatgtgtgaatatatattatctgtatagataactaggtattaatgtcacataagatggtgtgaccacacacacacacaaacacaygtgcagagagagggaattcagccacgtggtgtaggttggttaattacttgagataaatgagaagcaggcaggtctgtgctgagctgtgtcatcagtgaagatcacacttggaggtgacattgaagctgattcctcaaagaaccacgaaggaggcacgtggagacccagacagggaacatcagaggctccagaaagagcaggtcccagaaaagtctcagcctgttcttcagaaaggaatgaccactttgtctgtggggtatagtgggaggcagaggaggagatggagctgagattcaggg

SEQ ID NO: 193 - DNA, 599 bp, Homo sapiens
gaactgtgttaattctctggaaaccatctgacatgaaaatcagcttcattaatatgtttgggtgccctagcttgtaaattagctgaaggctagaagtcatttatatagaattttcttgttgacaccctaggcttaggggactggttgtctgtggggatagatcaaattgtgtacctttccaaaacaggttttaatgatgtttaaaagtggtcactgagatagtcttgaggatgtgctatgcagtagccaagccctcttacggtgtgttcctctcttagtgcccctttcctttcctacctrttcttcccttgcagtggtgtattagccttggacattaaaacatttgctttgtaaatttggaggcattctgattaaacagctgagtaaaacaaatcctgcttgtcttagaagcgccataatactagaaccagctgctggataaagaggttctagatctgggtgacttctagtatagccagtccagactgcagtattagggaaattgaattaatattagcttgaaatttagatgtcttctctcctagagccatgtaagggattgttattaaaatcagttttggtgaagttctaatactagt

SEQ ID NO: 194 - DNA, 599 bp, Homo sapiens
aacttggactttaataaaagggaaatgagtttgaactgaaattaggtatttgtttcatgtgtttgcattaataagcagaaaattaagtgctgtattggtttcatggttttgctgccctcttacatgctgagagaactgtgttaattctctggaaaccatctgacatgaaaatcagcttcattaatatgtttgggtgccctagcttgtaaattagctgaaggctagaagtcatttatatagaattttcttgttgacaccctaggcttaggggactggttgtctgtggggatagatcaaatygtgtacctttccaaaacaggttttaatgatgtttaaaagtggtcactgagatagtcttgaggatgtgctatgcagtagccaagccctcttacggtgtgttcctctcttagtgcccctttcctttcctacctgttcttcccttgcagtggtgtattagccttggacattaaaacatttgctttgtaaatttggaggcattctgattaaacagctgagtaaaacaaatcctgcttgtcttagaagcgccataatactagaaccagctgctggataaagaggttctagatctgggtgacttc

SEQ ID NO: 195 - DNA, 599 bp, Homo sapiens
caaaaaacccaaaccatcacccaaaggggaagggaaccaaatgactgctttgtgtaaattcttttttttttttctgagacagagtctcactctgttgcccaggctggagtgcagtggcgcgatctcggctcactgcaagctctgcctcccgggttcacgccattctcctgcctcagcctcccgagtagctgtgactacaggcgcccgccaccacgtccggctaatttttttgtatttttagtagagacggggtttcaccatgttagccagaatggtcttgatctcctgacctcgtgatcygcccaccctggcctcctaaagtgctgggattacaggcgtgagccaccgcgcccgtgctatgtaaattgtttgaaatgtacaaaactgttaccttttgctcatctagccttattgctatagcagtggttttcaattaagggtgacttcatccctcaggagacactgggtaatgcctagaaacatttttggttgtcatgatacgaatgtgtgcatactggctcctagcaggtagaggccaaaggtgatgctaaacaccctacagtgtaaaggacagtcccctacaacaaagaattatctgg

SEQ ID NO: 196 - DNA, 599 bp, Homo sapiens
ctggatagctataatgtgaaagaaccataacttcaatgtacacaaatagtacttgatatattttgttcaatgcattaaattttgagttgactatatatgttgtggcagcaaaaatattgaacaggtcaaatgaaatacatcgtatacattgatacagaaagatatattttaaattaagcaaataaaatcaagtagaacaaaaagtattacataggcatgtttatacttttctccctaatgtataatactcgcttgaaattattaacagtggttccctctgattttaaattaacagtgatwatcatgggacgtaaaaactactttatatacttccatattgtttgaatttttacacataaccagagtattaattcctaagtaaaaatgtcccaggtgggcatagtggggcacacacctgtagtgccagctactcagaggctgaggcaggaagattacccgagcccaggagttcaaggtcgtagggtgctaacaatatgacagaacctgataagatagccattgcattccagcctgggcaacacagcgacaccccacttctaaaaactaagaataggccaggcgcagtggctcacgcctgt

SEQ ID NO: 197 - DNA, 599 bp, Homo sapiens
ttgaagagatggggtttcacaatgttggccaggctggtctcaaactcctgacctcaggtgatccacctgcctcggccttccaaagtgctgggattgattacaggcatgagccaccatgccaagccagagtttccaaataaggtatcagggattaggacttcaacatgtcttggtgggggggcacgaaattcaattcatagcacctataatattcaggtgtcagaaacaacactggaatcctcaagctgaatatacaaacccaattgccatggtgaccctagcttctccaaaggcaggggyacccaagggctcagatgggatgagaagctgtggaagagataggggcagctttggccattggttggatggttaagaattgtaatgtgacatcagaacagttcagaaacctggctcagaaagtcttttgttggtttgaaagagtggagtgatcatggctcactgcagactggacctcccagactcaagcaatcctcccacctcagtctcctgagtaactgaactgcaggcacacaccaccacatccaactatttaattttttttttttttttttttttttttttttgtagagatggtatct